(19) United States
(12) Patent Application Publication
(10) Pub. No.: US 2008/0194561 A1
(43) Pub. Date: Aug. 14, 2008

(54) Compounds

(76) Inventors: Jerry Leroy Adams, Collegeville, PA (US); David Harold Drewry, Durham, NC (US); James Andrew Linn, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/996,749

(22) PCT Filed: Jul. 13, 2006
(86) PCT No.: PCT/US2006/027138

§ 371 Date: Jan. 25, 2008

Related U.S. Application Data
(60) Provisional application No. 60/702,799, filed on Jul. 26, 2005.

(51) Int. Cl. A61K 31/5377; C07D 409/04; C07D 413/14; A61P 35/00; C07D 409/14; A61K 31/506

(52) U.S. Cl. 514/235.8; 544/331; 544/122; 544/295; 514/252.14; 514/275

(57) ABSTRACT

A compound of formula (I):

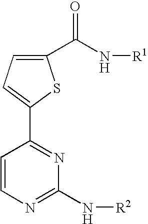

compositions and medicaments containing the same as well as processes for the preparation and use of such compounds, compositions and medicaments, particularly in diseases associated with inappropriate Aurora activity.

### BACKGROUND OF THE INVENTION

The present invention relates to pyrimidyl-thiophene derivatives, compositions and medicaments containing the same, as well as processes for the preparation and use of such compounds, compositions and medicaments. Such pyrimidyl-thiophene derivatives are potentially useful in the treatment of diseases associated with inappropriate Aurora kinase activity.

An important large family of enzymes is the protein kinase enzyme family. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis.

The protein kinase family of enzymes is typically classified into two main subfamilies: Protein Tyrosine Kinases and Protein Serine/Threonine Kinases, based on the amino acid residue they phosphorylate. Aberrant protein serine/threonine kinase activity has been implicated or is suspected in a number of pathologies such as rheumatoid arthritis, psoriasis, septic shock, bone loss, many cancers and other proliferative diseases. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Accordingly, both kinase subfamilies and the signal transduction pathways which they are part of are important targets for drug design.

The three known mammalian family members, Aurora-A (“2”), B (“1”) and C (“3”), are highly homologous proteins responsible for chromosome segregation, mitotic spindle function and cytokinesis. Aurora expression is low or undetectable in resting cells, with expression and activity peaking during the G2 and mitotic phases in cycling cells. In mammalian cells proposed substrates for the Aurora A and B kinases include histone H3, CENP-A, myosin II regulatory light chain, protein phosphatase 1, TPX2, INCENP, p53 and survivin, many of which are required for cell division. Since its discovery in 1997, the mammalian Aurora kinase family has been closely linked to tumorigenesis.

The Aurora kinases have been reported to be over-expressed in a wide range of human tumors. Elevated expression of Aurora-A has been detected in colorectal, ovarian and pancreatic cancers and in invasive duct adenocarcinomas of the breast. High levels of Aurora-A have also been reported in renal, cervical, neuroblastoma, melanoma, lymphoma, pancreatic and prostate tumor cell lines. Amplification/over-expression of Aurora-A is observed in human bladder cancers and amplification of Aurora-A is associated with aneuploidy and aggressive clinical behavior. Moreover, amplification of the Aurora-A locus (20q13) correlates with poor prognosis for patients with node-negative breast cancer. In addition, an allelic variant, isoleucine at amino acid position 31, is reported to be a low-penetrance tumor-susceptibility gene and displays greater transforming potential than the phenylalanine-31 variant and is associated with increased risk for advanced and metastatic disease. Aurora-B is highly expressed in multiple human tumor cell lines, including leukemic cells. Levels of this enzyme increase as a function of Duke's stage in primary colorectal cancers. Aurora-C, which is normally only found in germ cells, is also over-expressed in a high percentage of primary colorectal cancers and in a variety of tumor cell lines including cervical adenocarinoma and breast carcinoma cells.

Based on the known function of the Aurora kinases, inhibition of their activity should disrupt mitosis leading to cell cycle defects and eventual cell death. In vivo, an Aurora inhibitor therefore should slow tumor growth and induce regression. Recent reports in the literature support this hypothesis. For example, Hauf et al. describe an Aurora B inhibitor, Hesperadin, that causes defects in chromosomal segregation and a block in cytokinesis thereby resulting in polyploidy [Hauf, S et al. JCB 161(2), 281-294 (2003)]. Ditchfield et al. have described an equipotent inhibitor of Aurora A and B (ZM447439) that causes defects in chromosome alignment, chromosome segregation and cytokinesis [Ditchfield, C. et al., JCB 161(2), 267-280 (2003)]. Furthermore, they show that proliferating cells, but not cell-cycle arrested cells, are sensitive to the inhibitor. Efficacy of an Aurora A selective inhibitor in mouse and rat xenograft models was recently reported [Harrington, E.A. et al., Nature Medicine 10(3), 262-267, (2004)]. These results demonstrate that inhibition of Aurora kinases can provide a therapeutic window for the treatment of proliferative disorders such as cancer (see Nature, Cancer Reviews, Vol. 4, p 927-936, December 2004, for a review by N. Keen and S Taylor, which outlines the therapeutic potential of Aurora kinase inhibitors for the treatment of cancer).

The present inventors have identified novel pyrimidyl-thiophene compounds, which are inhibitors of kinase activity, in particular Aurora kinase activity. Such pyrimidyl-thiophene derivatives are therefore potentially useful in the treatment of disorders associated with inappropriate kinase, more particularly inappropriate Aurora kinase activity, in particular in the treatment and prevention of various disease states mediated by Aurora kinase mechanisms, such as diseases of cell proliferation including cancer.

### BRIEF SUMMARY OF THE INVENTION

In one aspect of the present invention, there is provided a compound of formula (I):

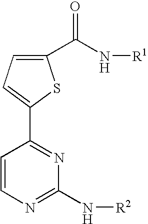

wherein

R¹ represents:

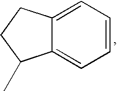

(C₁₋₃ alkylene)ₘ³¹-C₄₋₇cycloalkyl (where m is 0 or 1 and the cycloalkyl group is optionally substituted by C₁₋hydroxyalkyl), a 5 membered heteroaryl group (optionally substituted by one or more C₁₋₃alkyl), —C₁₋₃ alkyleneCN, —C₁₋₃alkylenepyridinyl, —C₁₋₃alkyleneindolyl, —(C₁₋₃alkylene)ₙphenyl (where n is 0 or 1, the phenyl group is optionally fused to a 5 or 6 membered heterocyclic group or is substituted by one or more substituents independently selected from —C₁₋₆hydroxyalkyl, —C₁₋₆alkyl, —C₁₋₆haloalkyl, —C₁₋₆alkoxy, —C₁₋₆haloalkoxy, -halogen, —OH, —COOH, —COOC₁₋₃alkyl, —NHCOC₁₋₃alkyl, —NHSO₂C₁₋₃alkyl, —CONR^{a}R^{b}, —NR^{c}R^{d}, —SO₂NR^{e}R^{f}), —(CH₂)₄OH, —C₁₋₆alkyleneNR⁶R⁷ (wherein the alkylene group is optionally substituted by phenyl);

R^{a}, R^{b}, R^{c}, R^{d}, R^{e} and R^{f} are each independently selected from H or —C₁₋₃alkyl;

R⁶ and R⁷ are independently HC₁₋₃ alkyl or R⁵ and R⁶ together with the nitrogen to which they are joined form a 6 membered heterocyclic ring, (optionally containing a further heteroatom selected from O or N and optionally substituted by C₁₋₃alkyl).

R² is

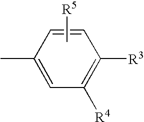

wherein

R³ and R⁴ together form a group selected from:

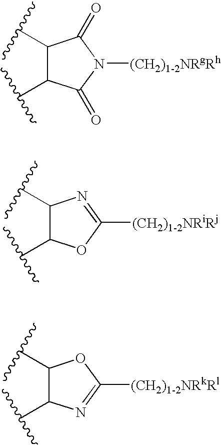

wherein R^{g}R^{h}, Rⁱ, R^{j}, R^{k} and R^{l} are independently H or —C₁₋₃alkyl;

or one of R³ and R⁴ is H, CH₃ or halogen and the other is a substituent selected from —OH, -phenyl (substituted by —C₁₋₃alkyleneNR^{m}Rⁿ), halogen or a group R⁸R⁹;

R^{m}Rⁿ are independently H or —C₁₋₃alkyl;

R⁸ is a bond (i.e. is absent), —O—, —CO—, —COO—, —C₁₋₃alkyleneNHCO—, —NHCO—, —SO₂—, —CONHC₁₋₃ alkylene, —NHCOC₁₋₃ alkylene-, —OC₁₋₃alkylene-, —C₁₋₃ alkylene-;

R⁹ is

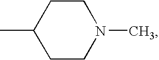

-pyridinyl, —C₁₋₆ alkyl, —C₁₋₆ haloalkyl, —NR¹⁰R¹¹;

R¹⁰ and R¹¹, are independently H, —C₁₋₃ alkyl, —(CH₂)₁₋₃NR^{o}R^{p}, or R¹⁰ and R¹¹, together with the N to which they are joined form a 5 or 6 membered heterocyclic or heteroaryl ring (each of which heterocyclic or heteroaryl ring optionally contains further heteroatoms independently selected from O or N and optionally substituted by C₁₋₃ alkyl, ═O, OH, C₁₋₃ hydroxyalkyl, —SO₂C₁₋₃alkyl);

R^{o}R^{p} are independently H or C₁₋₃ alkyl;

R⁵ is H or methyl;

or a salt or solvate thereof.

In a second aspect of the present invention, there is provided a pharmaceutical composition comprising a compound of formula (I) or a salt, or solvate thereof and one or more of pharmaceutically acceptable carriers, diluents and excipients.

In a third aspect of the present invention, there is provided a compound of formula (I), or a salt, or solvate thereof for use in therapy, in particular in the treatment of a disorder mediated by inappropriate AURORA kinase activity.

In a fourth aspect of the present invention, there is provided a method of treating a disorder in a mammal, said disorder being mediated by inappropriate AURORA kinase activity, comprising administering to said mammal a compound of formula (I) or a salt, or solvate thereof.

In an fifth aspect of the present invention, there is provided the use of a compound of formula (I), or a salt, or solvate thereof in the preparation of a medicament for use in the treatment of a disorder mediated by inappropriate AURORA kinase activity.

In a sixth aspect there is provided a method of treating cancer in a mammal comprising administering to said mammal a compound of formula (I) or a salt, or solvate thereof.

In a seventh aspect there is provided a compound of formula (I) or a salt, or solvate thereof in the manufacture of a medicament for the treatment of cancer.

In an eighth aspect of the invention there is provided a compound of formula (I) or a salt, or solvate thereof for use in the treatment of a disorder mediated by inappropriate AURORA kinase activity such as diseases of cell proliferation including cancer.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example as used herein, the terms “C₁-C₃ alkyl” and “C₁-C₆ alkyl” refer to an alkyl group, as defined above, containing at least 1, and at most 3 or 6 carbon atoms respectively. Examples of “alkyl” as used herein include, but are not limited to, methyl, ethyl, n-propyl, isopropyl, n-butyl, isobutyl, t-butyl, n-pentyl, isopentyl, n-hexyl and the like.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “C₁-C₆ haloalkyl” refers to an alkyl group as defined above containing the specified number of 6 carbon atoms respectively substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “alkylene” refers to a straight or branched chain divalent hydrocarbon radical having the specified number of carbon atoms. Thus for example, the term “C₁₋C₃ alkylene” refers to an alkylene group, as defined above, which contains at least 1, and at most 3, carbon atoms respectively. Examples of “alkylene” as used herein include, but ate not limited to, methylene, ethylene, n-propylene and n-butylene.

As used herein, the term “alkoxy” refers to the group RₐO—, where Rₐ is alkyl as defined above and the terms “C₁₋C₄ alkoxy” and “C₁₋C₆ alkoxy” refer to an alkoxy group as defined herein wherein the alkyl moiety contains at least 1, and at most 4 or 6, carbon atoms. Exemplary “C₁₋C₃ alkoxy” and “C₁₋C₆ alkoxy” groups useful in the present invention include, but are not limited to, methoxy, ethoxy, n-propoxy, isopropoxy, n-butoxy, and t-butoxy.

As used herein, the term “haloalkoxy” refers to the group RₐO—, where Rₐ is haloalkyl as defined above and the term “C₁₋C₆ haloalkoxy” refers to a haloalkoxy group as defined herein wherein the haloalkyl moiety contains at least 1, and at most 6, carbon atoms. Exemplary C₁₋C₆ haloalkoxy groups useful in the present invention include, but is not limited to, trifluoromethoxy.

As used herein, the term “heterocyclic” or the term “heterocyclyl” refers to a non-aromatic ring having the specified number of ring members being saturated or having one or more degrees of unsaturation containing one or more heteroatomis selected from S, SO, SO₂, O, or N. Examples of “heterocyclic” moieties include, but are not limited to, tetrahydrofuran, pyran, 1,4-dioxane, 1,3-dioxane, piperidine, pyrrolidine, morpholine, tetrahydrothiopyran, tetrahydrothiophene, di-oxo tetrahydrothiophene, and the like.

As used herein, the term “heteroaryl” refers to an aromatic ring, having the specified number of ring members. These heteroaryl rings contain one or more hydrogen, sulfur, and/or oxygen heteroatoms. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridyl, pyridazyl, pyrazinyl, pyrimidyl.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing, (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

The term “AURORA inhibitor” is used to mean a compound which inhibits AURORA activity. In one embodiment AURORA is AURORA A. In a further embodiment AURORA is AURORA B.

The term “AURORA mediated disease” or a “disorders or diseases mediated by inappropriate AURORA activity” is used to mean any disease state mediated or modulated by AURORA, kinase mechanisms, in particular those mediated by Aurora A and/or Aurora B including cancer.

As used herein, the term “substituted” refers to substitution with the named substituent or substituents, multiple degrees of substitution being allowed unless otherwise stated.

In one aspect, R¹ may be selected from the R¹ groups in the specific examples below.

In one aspect, R¹ is —(CH₂)₀₋₁ cyclohexyl (wherein the cyclohexyl is substituted by —CH₂OH), —(CH₂)₀₋₃ phenyl (wherein the phenyl group is optionally mono or disubstituted by substituents independently selected from —C₁₋₃alkoxy, —C₁₋₃haloalkoxy, —OH, —F, —Cl, —C₁₋₃ hydroxyalkyl, —N(CH₃)₂, —NHCOCH₃, —NHSO₂CH₃, —COOCH₃, —COOH, —CONH₂, —CONH CH₃), —CH(CH₃)phenyl, —C₁₋₆alkyleneN(CH₃)₂, —CH₂indolyl, —(CH₂)₄OH, —CH₂CN, C₀₋₃alkylenepyridyl,

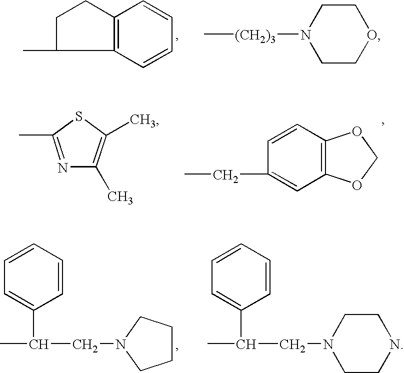

In a further aspect, R¹ is —C₁₋₃alkylene phenyl, wherein the phenyl is optionally substituted by one or more substituents independently selected from —C₁₋₃alkoxy, —C₁₋₃haloalkoxy, —OH, —F, —Cl, —C₁₋₃ hydroxyalkyl, —N(CH₃)₂, —NHCOCH₃, —NHSO₂CH₃, —COOCH₃, —COOH, —CONH₂, —CONH CH₃),

In a further aspect, R¹ is —CH₂phenyl, wherein the phenyl is optionally mono substituted by —OMe.

In one embodiment R² may be selected from the R² groups in the specific examples below.

In one aspect, R² is

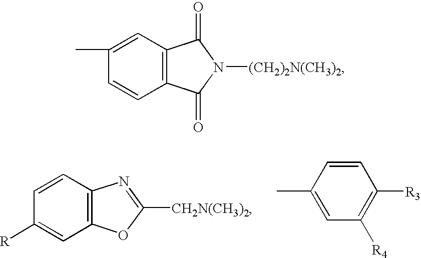

wherein one of R³ and R⁴ is H and the other is selected from —F, —Cl, —OH, -phenylCH₂N(CH₃)₂, —R⁸R⁹, wherein R⁸ and R⁹ are as defined above.

In one aspect R⁸ is a bond (ie is absent), —O—, NHCO(CH₂)₂, —OCH₃—, —CO—, NHCOCH₂—, CH2-, OCH₂CH₂—, —CONHCH₂CH₂—CONHCH₂, —CON(CH₃)—, —SO₂—, —COO—

In one aspect, R⁸ is —O—, —C₁₋₃ alkylene-, —OC₁₋₃ alkylene -,

In one aspect R⁹ is —CH₃, —N(CH₃)₂, Cl, F, OH,

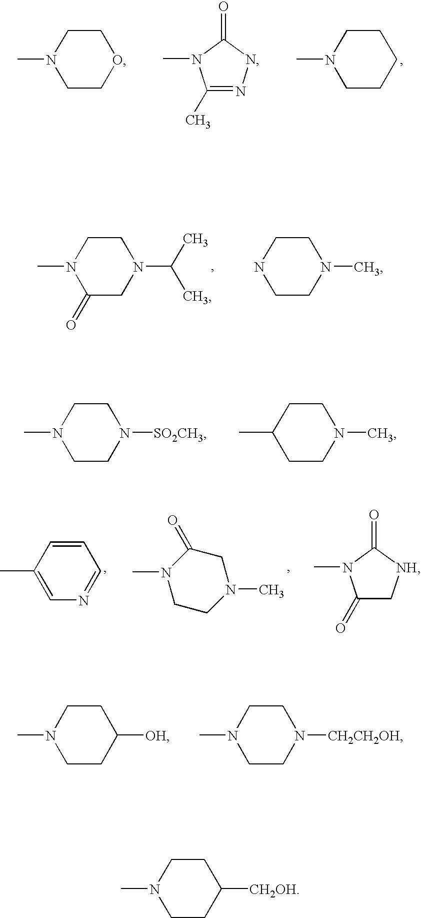

In one aspect R⁹ is

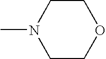

In one aspect, R⁸R⁹ is —OCH₃.

While embodiments for each variable have generally been listed above separately for each variable, preferred compounds of this invention include those in which several or each variable in Formula (1) is selected from all embodiments for each variable. Therefore, this invention is intended to include all combinations of embodiments for each variable.

Specific examples of compounds of the present invention include the compounds described in the Examples Section below.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formula (I) are included within the scope of the compounds of formula (I).

It is to be understood that reference to compounds of formula (I) above, following herein, refers to compounds within the scope of formula (I) as defined above unless specifically limited otherwise.

The present invention also covers salts of the compounds of formula (I). Typically, the salts of the present invention are pharmaceutically acceptable salts. For a review on suitable salts, see Berge et al, J. Pharm. Sci. 1977, 66, 1-19. Salts encompassed within the term “pharmaceutically acceptable salts” refer to non-toxic salts of the compounds of this invention. A pharmaceutically acceptable acid addition salt can be formed by reaction of a compound of formula (I) with a suitable inorganic or organic acid (such as hydrobromic, hydrochloric, sulfuric, nitric, phosphoric, succinic, malaleic, formic, acetic, propionic, furmaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, napthalenesulfonate (e.g. 2-naphthalenesulfonate) or hexanoate salt.

A pharmaceutically acceptable base addition salt can be formed by a reaction of a compound of formula (I) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystrallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I).

Other non-pharmaceutically acceptable salts, e.g. oxalates, may be used, for example in the isolation of compounds of the invention, and are included within the scope of this invention.

The invention includes within its scope all possible stoichiometric and non-stoichiometric forms of the salts of the compounds of formula (I). Typically, a pharmaceutical acceptable salt may be readily prepared by using a desired acid or base as appropriate. The salt may precipitate from solution and be collected by filtration or may be recovered by evaporation of the solvent. Other salts, which are not pharmaceutically acceptable, may be useful in the preparation of compounds of this invention and these form a further aspect of the invention.

Since the compounds of formula (I) are intended for use in pharmaceutical compositions it will be readily understood that they are each preferably provided in substantially pure form, for example, at least 60% pure, more suitably at least 75% pure and preferably at least 85% pure, especially at least 98% pure (% in a weight for weight basis).

While it is possible that, for use in therapy, a compound of formula (I), as well as salts, solvates and physiological functional derivatives thereof, may be administered as the raw chemical, it is possible to present the active ingredient as a pharmaceutical composition. Accordingly, the invention further provide a pharmaceutical composition comprising a compound of the formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the composition and not deleterious to the recipient thereof. In accordance with another aspect of the invention there is also provided a process for the preparation of a pharmaceutical composition including admixing a compound of the formula (I), or salts, solvates and physiological functional derivatives thereof, with one or more pharmaceutically acceptable carriers, diluents or excipients.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle.

Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of composition in question, for example those suitable for oral administration may include flavouring agents.

A therapeutically effective amount of a compound of the present invention will depend upon a number of factors including, for example, the age and weight of the animal, the precise condition requiring treatment and its severity, the nature of the composition, and the route of administration, and will ultimately be at the discretion of the attendant physician or veterinarian, However, an effective amount of a compound of formula (I) for the treatment of diseases associated with inappropriate AURORA activity, will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) per se. It is envisaged that similar dosages would be appropriate for treatment of the other conditions referred to above.

The compounds of formula (I) and salts, solvates and physiological functional derivatives thereof, are believed to have utility in proliferative diseases including cancer as a result of inhibition of the protein kinase AURORA.

The present invention thus also provides compounds of formula (I) and pharmaceutically acceptable salts or solvates thereof, or physiologically functional derivatives thereof, for use in medical therapy, and particularly in the treatment of disorders mediated by AURORA activity.

The inappropriate AURORA activity referred to herein is any AURORA activity that deviates from the normal AURORA activity expected in a particular mammalian subject. Inappropriate AURORA activity may take the form of, for instance, an abnormal increase in activity, or an aberration in the timing and or control of AURORA activity. Such inappropriate activity may result then, for example, from over expression or mutation of the protein kinase leading to inappropriate or uncontrolled activation.

The present invention is directed to methods of regulating, modulating, or inhibiting AURORA for the prevention and/or treatment of disorders related to unregulated AURORA activity. In particular, the compounds of the present invention can also be used in the treatment of various disease states mediated by AURORA kinase mechanisms, including cancer.

A further aspect of the invention provides a method of treatment of a mammal suffering from a disorder mediated by AURORA activity, which includes administering to said subject a compound of formula (I) or a pharmaceutically acceptable salt, solvate, or a physiologically functional derivative thereof. In one embodiment, the disorder is cancer.

A further aspect of the present invention provides the use of a compound of formula (I), or a pharmaceutically acceptable salt or solvate thereof, or a physiologically functional derivative thereof, in the preparation of a medicament for the treatment of a disorder characterized by AURORA activity, in particular, a proliferative disorder including cancer.

The compound of formula (1) for use in the instant invention and their salts, solvates and physiologically functional derivatives thereof may be used in combination with one or more other therapeutic agents. The invention thus provides in a further aspect the use of a combination comprising a compound of formula (1) with a further therapeutic agent or agents in the treatment of diseases associated with inappropriate AURORA activity.

The compounds of the present invention and their salts and solvates, and physiologically functional derivatives thereof, may be employed alone or in combination with other therapeutic agents for the treatment of the above-mentioned conditions. In particular, combination with at least one other anti-cancer therapy is envisaged. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of formula (I) or a pharmaceutically acceptable salt or solvate thereof, or a physiologically functional derivative thereof, and the use of at least one other cancer treatment method. Preferably, combination therapies according to the present invention comprise the administration of at least one compound of formula (I) or a pharmaceutically acceptable salt or solvate thereof, or a physiologically functional derivative thereof, and at least one other pharmaceutically active agent, preferably an anti-neoplastic agent. The compound(s) of formula (I)) and the other pharmaceutically active agent(s) may be administered together or separately and, when administered separately this may occur simultaneously or sequentially in any order and by any convenient route. The amounts of the compound(s) of formula (I) and the other pharmaceutically active agent(s) and the relative timings of administration will be selected in order to achieve the desired combined therapeutic effect.

In one embodiment, an other anti-cancer therapy is at least one additional chemotherapeutic therapy. Such chemotherapeutic therapy may include one or more of the following categories of anti-cancer agents.

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristrine, vinblastine, vindesine and vinorelbine and taxoids like taxol and taxotere); and topoisomerase inhibitors (for example epipodophyllotoxins like etoposide and teniposide, amsacrine, topotecan and camptochecin);

(ii) cytostatic agents such as antioestrogens (for example tamoxifen, toremifine, raloxifine, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate) aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbbl antibody cetuximab [C225], farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl-7-methoxy-6-(3-morpholinoproproxy)quinazolin-4-amine (gefitinib, AZD1839), N-3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinoproproxy)quinazolin-4-amine (Cl-1033)), for example inhibitors of the platelet-derived growth factor family and for example inhibitors of the hepatocyte growth factor family;

(v) antiangiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function and angiostatin);

(vi) vascular damaging agents such as Combretastatin A4;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

When a compound of formula (1) is used in combination with a second therapeutic agent active against the same disease, the dose of each compound may differ from that when the compound is used alone. Appropriate doses will be readily appreciated by those skilled in the art.

The compounds of this invention may be made by a variety of methods, including standard chemistry, Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Compounds of Formula I can be prepared according to the synthetic sequences illustrated in Schemes 1-3 and further detailed in the Examples section following. Compounds of type C (i.e. of formula (I)), can be made, for example, by the routes shown in schemes 1, 2, and 3. The thiophene acid can be converted to the amides A by standard amide bond forming conditions, known to one skilled in the art. For example, the amides could be made from the acid and appropriate amines using coupling reagents such as EDCI, DCC, or HATU in the presence of appropriate additives and in a suitable solvent such as CH₂Cl₂, THF, or DMF. The ketone of compounds A can be converted to intermediates B by reactions well known in the literature, such as reaction with DMF-DMA in the presence or absence of an additional solvent such as toluene at temperatures from 0° C. to reflux. Enaminones B can be condensed with guanidines in an appropriate solvent to give the pyrimidines C. Pyrimidine forming reactions of this sort are well known in the literature and may require the addition of an additional base and elevated temperatures.

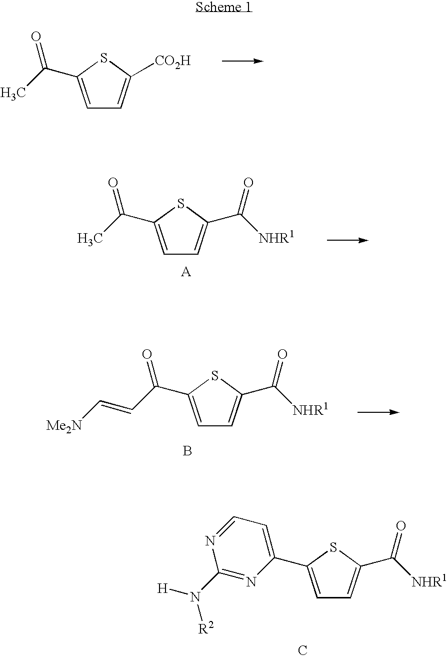

Alternatively, the target compounds C can be made by the route shown in scheme 2. The commercially available thiophene boronic acid can be protected as the pinacol ester under standard conditions to give intermediate D. This acid can be converted to amide intermediates E using standard conditions known to one skilled in the art. For example, compounds E can be synthesized by reaction of D with an appropriate amine, a coupling reagent such as EDCI, DCC, or HATU in the presence of appropriate additives and in a suitable solvent such as CH₂Cl₂, THF, or DMF. Compounds F can be made by reaction of boronate esters E with 2,4-di-chloropyrimidine under Suzuki reaction conditions. The Suzuki reaction is well describe in the synthetic chemistry literature, and is a method for preparing biaryl compounds from aryl halides and either boronate esters or boronic acids. The reaction may be performed in a variety of solvents or mixtures of solvents (including but not limited to DMF, EtOH, DME, toluene, dioxane, THF, water) in the presence of a catalyst (including but not limited to Pd(Ph₃P)₄ and Pd(Ph₃P)₂Cl₂) and a base (including but not limited to Et₃N, K₂CO₃, Na₂CO₃) at temperatures ranging from room temperature to 200° C.

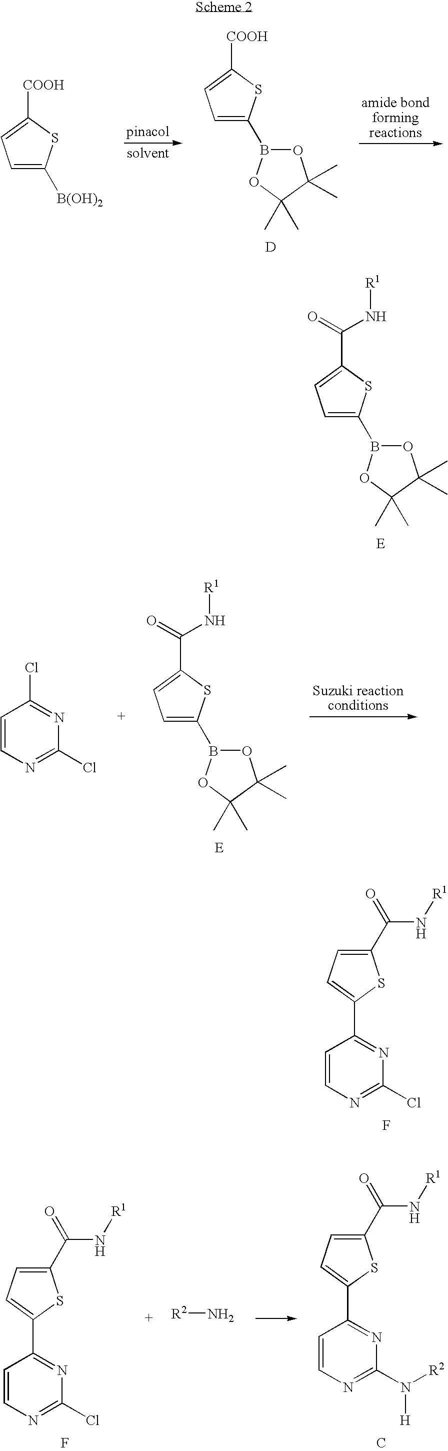

Compounds of type C can also be made by the route illustrated in scheme 3. 2-thiomethyl-uracil (reference) can be treated with amines at elevated temperatures in an appropriate solvent to give compounds G. Compounds G can then be converted into chloropyrimidines H by treatment with an appropriate chlorinating reagent such as POCl3 either neat, or in an appropriate solvent. The reaction may require elevated temperatures. A variety of such chlorination conditions are described in the literature, and are well known to one skilled in the art. Compounds C can then be synthesized by a Suzuki reaction between boronates of type E and chloro-pyrimidines of type H. The Suzuki reaction is well describe in the synthetic chemistry literature, and is a method for preparing bi-aryl compounds from aryl or heteroaryl halides and either boronate esters or boronic acids. The reaction may be performed in a variety of solvents or mixtures of solvents (including but not limited to DMF, EtOH, DME, toluene, dioxane, THF, water) in the presence of a catalyst (including but not limited to Pd(Ph₃P)₄ and Pd(Ph₃P)₂Cl₂) and a base (including but not limited to Et₃N, K₂CO₃, Na₂CO₃) at temperatures ranging from room temperature to 200° C.

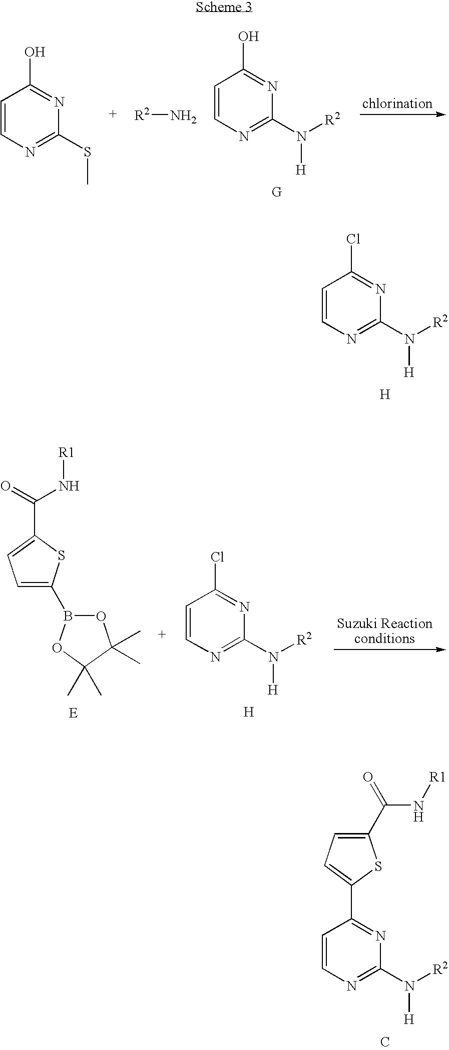

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); rt (room temperature);
min (minutes); h (hours);
mp (melting point); TLC (thin layer chromatography);
Tᵣ (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); AcOEt (ethyl acetate);
DME (1,2-dimethoxyethane); DCM (dichloromethane);
DCE (dichloroethane); DMF (N,N-dimethylformamide);
DMPU (N,N′-dimethylpropyleneurea); CDI (1,1-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid;
EDC (1-[3-dimethylamino)propyl]-3-ethylcarbodiimide hydrochloride);
BOC (teft-butyloxycarbonyl); FMOC (9-fluorenylmethoxycarbonyl);
DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
ATP (adenosine triphosphate); HRP (horseradish peroxidase);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU(O-Benzotriazole-1-yl-N,N,N′,N′-tetramethyluroniumhexafluoro phosphate).
HEPES (4-(2-hydroxyethyl)-1-piperazine ethane sulfonic acid);
DPPA (diphenylphosphoryl azide);
fHNO₃ (fuming HNO₃); and
EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded for example on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

HPLC were recorded for example on a Gilson HPLC or Shimazu HPLC system by the following conditions. Column: 50×4.6 mm (id) stainless steel packed with 5 μm Phenomenex Luna C-18; low rate: 2.0 mL/min; Mobile phase: A phase=50 mM ammonium acetate (pH 7.4), B phase=acetonitrile, 0-0.5 min (A: 100%, B: 0%), 0.5-3.0 min (A:100-0%, B:0-100%), 3.0-3.5 min (A: 0%, B: 100%), 3.5-3.7 min (A: 0-100%, B: 100-0%), 3.7-4.5 min (A: 100%, B: 0%); Detection : UV 254 nm; Injection volume: 3 μL.

Low-resolution mass spectra (MS) were recorded for example on a JOEL JMS-AX505HA, JOEL SX-102, or a SCIEX-APliii spectrometer; LC-MS were recorded for example on a micromass 2MD and Waters 2690; high resolution MS were obtained using a JOEL SX-102A spectrometer. All mass spectra were taken under electrospray ionization (ESI), chemical ionization (Cl), electron impact (El) or by fast atom bombardment (FAB) methods. Infrared (IR) spectra were obtained for example on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed for example on silica gel (230-400 mesh, Merck).

### Example 1

### Method A (see Scheme 1)

### 5-{2-[(4-fluorophenyl)amino]-4-pyrimidinyl}-N-(phenylmethyl)-2-thiophenecarboxamide

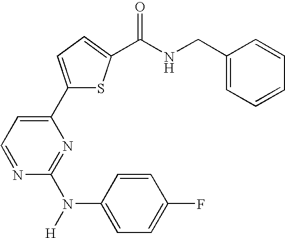

### (a Preparation of 5-acetyl-N-(phenylmethyl)-2-thiophenecarboxamide

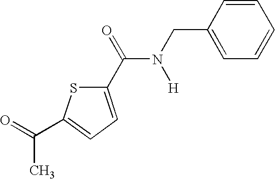

A mixture of 5-acetyl-2-thiophenecarboxylic acid (5.00 g, 29.4 mmol), HOBt (4.77 g, 35.3 mmol), EDC hydrochloride (6.77 g, 35.3 mmol) and DMF (50 mL) was stirred for 15 minutes at r.t. Next, benzylamine (3.53 mL, 32.3 mmol) was added and the reaction was stirred for 18 h at r.t. The DMF was removed by rotary evaporation under reduced pressure and the oil was partitioned between AcOEt:water (100 mL:10 mL). The phases were separated and the aqueous phase was extracted with AcOEt (100 mL). The combined organic layer was washed with 1N aq. sodium hydroxide (3×30 mL), water (20 mL), saturated brine (20 mL), and then dried (MgSO₄) for several hours. The volatiles were removed to give 5-acetyl-N-(phenylmethyl)-2-thiophenecarboxamide (6.63 g) as a light tan solid.

1H NMR (300 MHz, DMSO-d6) δ ppm 2.59 (s, 3 H), 4.50 (d, J=6.0 Hz, 2H), 7.26-7.40 (m, 5H), 7.88 (d, J=4.1 Hz, 1 H), 7.96 (d, J=4.0 Hz, 1 H), 9.33 (t, J=5.9 Hz, 1 H); MS m/z 260 (M+1)⁺.

### (b Preparation of 5-[(2E)-3-(dimethylamino)-2-propenoyl]-N-(phenylmethyl)-2-thiophenecarboxamide

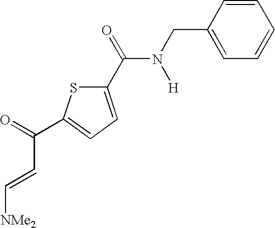

A mixture of 5-acetyl-N-(phenylmethyl)-2-thiophenecarboxamide (2.00 g, 7.71 mmol) and dimethylformamide dimethylacetal (10.2 mL, 77.1 mmol) was heated at reflux for 2 h and then the volatiles were removed by rotary evaporation under reduced pressure. The residual solids were triturated in ether (50 mL), followed by filtration of the solids to give 5-[(2E)-3-(dimethylamino)-2-propenoyl]-N-(phenylmethyl)-2-thiophene-carboxamide (2.33 g) as a rust-colored solid.

1H NMR (400 MHz, DMSO-d6) δ ppm 2.90/3.13 (2×s, 6H), 4.43 (d, J=6.0 Hz, 2H), 5.76 (d, J=12.2 Hz, 1H), 7.23-7.34 (m, 5H), 7.68 (d, J=12.2 Hz, 1H), 7.74 (d, J=4.0 Hz, 1 H), 7.75 (d, J=4.0 Hz, 1 H), 9.11 (t, J=6.0 Hz, 1 H); MS m/z 315 (M+1)⁺.

### (c) Preparation of 5-(2-(4-fluoroanilino)-4-pyrimidinyl)-N-(phenylmethyl)-2-thiophenecarboxamide

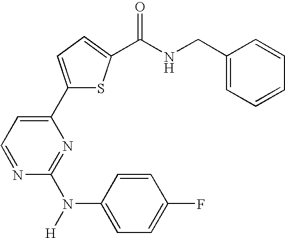

A small sphere of sodium (11 mg, 0.48 mmol) was dissolved in EtOH (0.5 mL). 1-(4-Fluorophenyl)guanidine carbonate (88 mg, 0.24 mmol) was added to the sodium ethoxide/EtOH solution with shaking for 30 minutes. This mixture was transferred to a mixture of 5-[(2E)-3-(dimethylamino)-2-propenoyl]-N-(phenylmethyl)-2-thiophenecarbox-amide (100 mg, 0.32 mmol) in EtOH (2.0 mL) and the reaction was heated at reflux for 48 h. Additional sodium ethoxide solution (5 mg sodium dissolved in 0.5 mL EtOH) was added to the reaction along with 1-(4-fluorophenyl)guanidine carbonate (44 mg, 0.12 mmol) and the reaction was refluxed for another 24 h. The volatiles were removed by rotary evaporation under reduced pressure and the residual solids were treated with water (4 mL). The mixture was adjusted to pH 6 with concentrated HCl, sonicated, and the solids were collected by filtration, then rinsed with a small amount of ethanol, followed by AcOEt. The solids were dried to give 5-(2-(4-fluoroanilino)-4-pyrimidinyl)-N-(phenylmethyl)-2-thiophenecarboxamide (71 mg).

1H NMR (400 MHz, DMSO-d6) δ ppm 4.46 (d, J=5.9 Hz, 2 H), 7.14 (dd, J=8.9 Hz, 2 H), 7.25 (m, 1 H), 7.32-7.36 (m, 5 H), 7.77-7.80 (m, 2 H), 7.86 (d, J=3.9 Hz, 1 H), 7.98 (d, J=4.1 Hz, 1 H), 8.51 (d, J=5.1 Hz, 1 H), 9.20 (t, J=5.8 Hz, 1 H), 9.73 (s, 1 H); MS m/z 405 (M+1)⁺.

### Example 2

### 5-(2-(4-chloroanilino)-4-pyrimidinyl)-N-(phenylmethyl)-2-thiophenecarboxamide

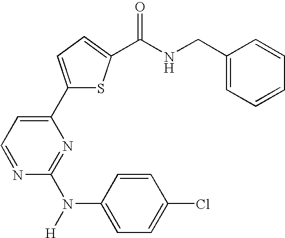

### (a) Preparation of 5-(2-(4-chloroanilino)-4-pyrimidinyl)-N-(phenylmethyl)-2-thiophenecarboxamide

In a similar manner as described for Example 1c, sodium (11 mg, 0.48 mmol) in EtOH (0.5 mL), 1-(4-chlorophenyl)guanidine carbonate (96 mg, 0.24 mmol) and 5-[(2E)-3-(dimethylamino)-2-propenoyl]-N-(phenylmethyl)-2-thiophenecarboxamide (100 mg, 0.32 mmoL) were reacted for 48 h at reflux, followed by additional sodium ethoxide (7 mg Na^{o} in 0.5 mL EtOH) and 1-(4-chlorophenyl)guanidine carbonate (57 mg, 0.14 mmol) and refluxing for 24 h. An aqueous workup/extraction with AcOEt gave a crude solid which was purified by normal phase silica gel chromatography using AcOEt:hexanes as eluant to give 5-(2-(4-chloroanilino)-4-pyrimidinyl)-N-(phenylmethyl)-2-thiophene-carboxamide (41 mg) as a solid.

1H NMR (400 MHz, DMSO-d6) δ ppm 4.48 (d, J=5.9 Hz, 2 H), 7.27 (m, 1 H), 7.33-7.38 (m, 6 H), 7.42 (d, J=5.3 Hz, 1 H), 7.83-7.88 (m, 3 H), 8.01 (d, J=4.0 Hz, 1 H), 8.56 (d, J=5.4 Hz, 1 H), 9.22 (t, J=6.0 Hz, 1 H), 9.87 (s, 1 H); MS mlz 421/423 (M+1)⁺.

### Example 3

### 5-(2-(4-methoxyanilino)-4-pyrimidinyl)-N-(phenylmethyl)-2-thiophenecarboxamide

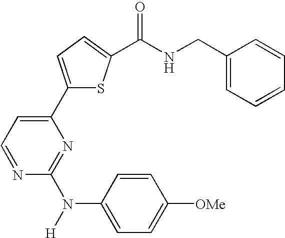

### (a) Preparation of 5-(2-(4-methoxyanilino)-4-pyrimidinyl)-N-(phenylmethyl)-2-thiophenecarboxamide

In a similar manner as described for Example 1c, sodium (11 mg, 0.48 mmol) in EtOH (0.5 mL), 1-(4-methoxyphenyl)guanidine carbonate (94 mg, 0.24 mmol) and 5-[(2E)-3-(dimethylamino)-2-propenoyl]-N-(phenylmethyl)-2-thiophenecarboxamide (100 mg, 0.32 mmoL) were reacted for 48 h at reflux, followed by additional sodium ethoxide (4 mg Na^{o} in 0.5 mL EtOH) and 1-(4-methoxyphenyl)guanidine carbonate (15 mg, 0.038 mmol) and refluxing for 24 h. An aqueous workup/extraction with AcOEt gave a crude solid which was purified by normal phase silica gel chromatography using AcOEt:hexanes as eluant to give 5-(2-(4-methoxyanilino)-4-pyrimidinyl)-N-(phenylmethyl)-2-thiophenecarboxamide (71 mg) as a solid.

1H NMR (400 MHz, DMSO-d6) δ ppm 3.74 (s, 3 H), 4.48 (d, J=5.8 Hz, 2 H), 6.90 (d, J=9.0 Hz, 2 H), 7.26-7.28 (m, 1 H), 7.28-7.35 (m, 5 H), 7.69 (d, J=9.0 Hz, 2 H), 7.87 (d, J=3.9 Hz 1 H), 7.97 (d, J=4.0 Hz, 1 H), 8.48 (d, J=5.1 Hz, 1 H), 9.21 (t, J=6.0 Hz, 1 H), 9.53 (s, 1 H); MS m/z 417 (M+1)⁺.

### Example 4

### Method A

### N-[3-(dimethylamino)-2,2-dimethylpropyl]-5-(2-{[4-(methyloxy)phenyl]amino}-4-pyrimidinyl)-2-thiophenecarboxamide

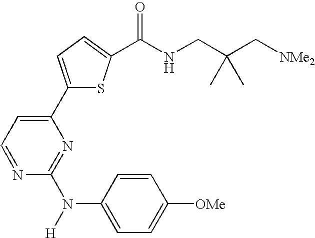

### (a) 5-acetyl-N-[3-(dimethylamino)-2,2-dimethylpropyl]-2-thiophenecarboxamide

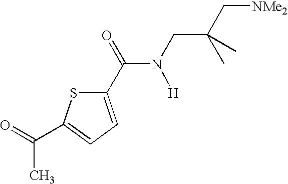

In a similar manner as described for Example 1a, 5-acetyl-2-thiophenecarboxylic acid (5.00 g), HOBt (4.77 g), EDC hydrochloride (6.77 g), DMF (50 mL) and N,N,2,2-tetramethyl-1,3-propanediamine (5.14 mL) gave 5-acetyl-N-[3-(dimethylamino)-2,2-dimethylpropyl]-2-thiophenecarboxamide (2.99 g) as a solid.

1H NMR (300 MHz, DMSO-d6) δ ppm 0.90 (s, 6H), 2.21 (s, 2H), 2.30 (s, 6H), 2.59 (s, 3 H), 3.20 (d, J=6.1 Hz, 2H), 7.83 (d, J=3.9 Hz, 1 H), 7.95 (d, J=4.0 Hz, 1 H), 8.75 (t, J=6.0 Hz, 1 H); MS m/z 283 (M+1)⁺.

### b) N-[3-(dimethylamino)-2,2-dimethylpropyl]-5-[(2E)-3-(dimethylamino)-2-propenoyl]-2-thiophenecarboxamide

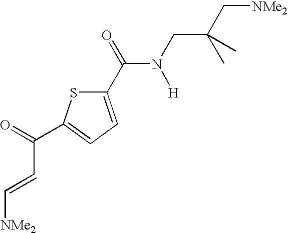

In a similar manner as described for Example 1b, 5-acetyl-N-(2,2-dimethyl-3-dimethylaminopropyl)-2-thiophenecarboxamide (2.99 g) and dimethylformamide dimethylacetal (14.1 mL) gave N-[3-(dimethylamino)-2,2-dimethylpropyl]-5-[(2E)-3-(dimethylamino)-2-propenoyl]-2-thiophenecarboxamide (3.25 g) as a yellow solid.

1H NMR (300 MHz, DMSO-d6) δ ppm 0.90 (s, 6 H), 2.19 (s, 2 H), 2.29 (s, 6 H), 2.96/3.19 (2×s, 6H), 3.19 (d, 2 H), 5.81 (d, J=12.2 Hz, 1H), 7.71-7.78 (m, 3 H), 8.56 (t, 1H).

### (c) preparation of N-[3-(dimethylamino)-2,2-dimethylpropyl]-5-(2-{[4-(methyloxy)phenyl]amino}-4-pyrimidinyl)-2-thiophenecarboxamide

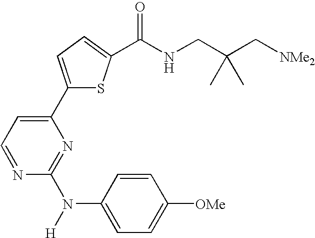

In a similar manner as described for Example 1c, sodium (9.2 mg, 0.4 mmol)) dissolved in EtOH (1.0 mL), 1-(4-methoxyphenyl)guanidine hydrochloride (81 mg, 0.40 mmol)) and N-[3-(dimethylamino)-2,2-dimethylpropyl]-5-[(2E)-3-(dimethylamino)-2-propenoyl]-2-thiophenecarboxamide (135 mg, 0.40 mmol) in EtOH (3.0 mL) were refluxed for 20 h, followed by semi-preparative reversed-phase chromatography to give N-[3-(dimethylamino)-2,2-dimethylpropyl]-5-(2-{[4-(methyloxy)phenyl]amino}-4-pyrimidinyl)-2-thiophenecarboxamide (34 mg) as a solid.

1H NMR (400 MHz, DMSO-d6) δ ppm 0.86 (s, 6 H), 2.16 (s, 2 H), 2.25 (s, 6 H), 3.16 (d, 2 H), 3.72 (s, 3H), 6.88 (d, J=9.1 Hz, 2H), 7.29 (d, J=5.1 Hz, 1 H), 7.67 (d, J=9.2 Hz, 2H), 7.78 (d, J=4.0 Hz, 1 H), 7.94 (d, J=3.9 Hz, 1 H), 8.46 (d, J=5.1 Hz, 1 H), 8.61 (t, J=6.0 Hz, 1 H), 9.50 (s, 1 H); MS m/z 440 (M+1)⁺.

### Example 5

### Method A

### 5-(2-{[4-(methyloxy)phenyl]amino}-4-pyrimidinyl)-N-[3-(4-morpholinyl)propyl]-2-thiophenecarboxamide

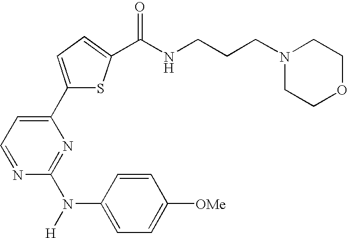

### (a Preparation of 5-acetyl-N-[3-(4-morpholinyl)propyl]-2-thiophenecarboxamide

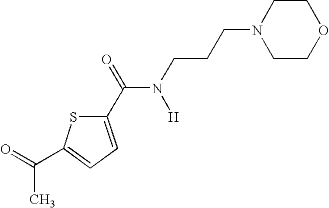

In a similar manner as described for Example 4a, 5-acetyl-2-thiophenecarboxylic acid (5.00 g), HOBt (4.77 g), EDC hydrochloride (6.77 g), DMF (50 mL) and 4-(3-aminopropyl)morpholine (4.72 mL) gave 5-acetyl-N-[3-(4-morpholinyl)propyl]-2-thiophenecarboxamide (2.75 g) as a yellow solid.

1H NMR (300 MHz, DMSO-d6) δ ppm 1.71 (m, 2H), 2.31-2.47 (m, 6H), 2.58 (s, 3 H), 3.32 (m, 2 H), 3.60 (dd, 4 H), 7.81 (d, J=4.0 Hz, 1 H), 7.95 (d, J=4.1 Hz, 1 H), 8.76 (t, J=5.4 Hz, 1 H); MS m/z 297 (M+1)⁺.

### (b) Preparation of 5-[(2E)-3-(dimethylamino)-2-propenoyl]-N-[3-(4-morpholinyl)propyl]-2-thiophenecarboxamide

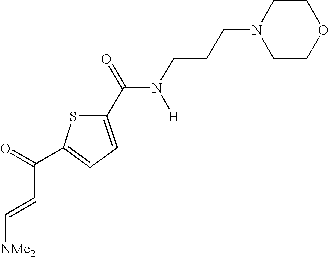

In a similar manner as described for Example 4b, 5-acetyl-N-[3-(4-morpholinyl)propyl]-2-thiophenecarboxamide (2.75 g) and dimethylformamide dimethylacetal (11.1 mL) gave 5-[(2E)-3-(dimethylamino)-2-propenoyl]-N-[3-(4-morpholinyl)propyl]-2-thiophenecarboxamide (2.56 g) as a yellow solid.

1H NMR (300 MHz, DMSO-d6) δ ppm 1.70 (m, 2H), 2.33-2.38 (m, 6H), 2.95/319 (2×s, 6 H), 3.29 (m, 2 H), 3.60 (dd, 4 H), 5.81 (d, J=12.2 Hz, 1 H), 7.71-7.78 (m, 3 H), 8.59 (t, J=5.6 Hz, 1 H).

### (c) Preparation of 5-(2-{[4-(methyloxy)phenyl]amino}-4-pyrimidinyl)-N-[3-(4-morpholinyl)propyl]-2-thiophenecarboxamide

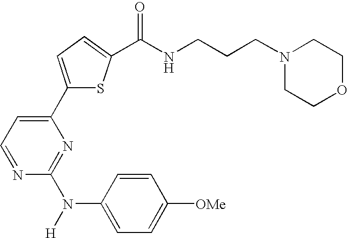

In a similar manner as described for Example 4c, sodium (9.2 mg) dissolved in EtOH (1.0 mL), 1-(4-methoxyphenyl)guanidine hydrochloride (81 mg) and 5-[(2E)-3-(dimethylamino)-2-propenoyl]-N-[3-(4-morpholinyl)propyl]-2-thiophenecarboxamide (141 mg) in EtOH (3.0 mL) gave 5-(2-{[4-(methyloxy)phenyl]amino}-4-pyrimidinyl)-N-[3-(4-morpholinyl)propyl]-2-thiophenecarboxamide (39 mg) as a solid.

1H NMR (400 MHz, DMSO-d6) δ ppm 1.69 (m, 2H), 2.29-2.33 (m, 6H), 3.26 (m, 2 H), 3.55 (dd, 4 H), 3.72 (s, 3 H), 6.89 (d, J=8.9 Hz, 2 H), 7.28 (d, J=5.2 Hz, 1 H), 7.67 (d, J=9.1 Hz, 2 H), 7.76 (d, J=3.8 Hz, 1 H), 7.93 (d, J=4.1 Hz, 1 H), 8.46 (d, J=5.1 Hz, 1 H), 8.63 (t, J=5.6 Hz, 1 H), 9.50 (s, 1 H); MS m/z 454 (M+1)⁺.

### Example 6

### Method A

### N-[2-(dimethylamino)ethyl]-5-(2-{[4-(methyloxy)phenyl]amino}-4-pyrimidinyl)-2-thiophenecarboxamide

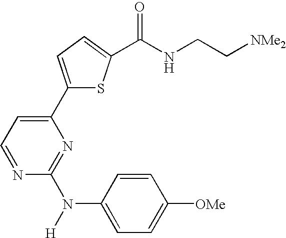

### (a) Preparation of 5-acetyl-N-[2-(dimethylamino)ethyl]-2-thiophenecarboxamide

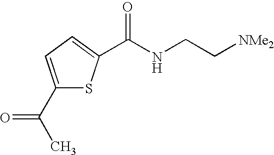

In a similar manner as described for Example 4a, 5-acetyl-2-thiophenecarboxylic acid (5.00 g), HOBt (4.77 g), EDC hydrochloride (6.77 g), DMF (50 mL) and N,N-dimethylethylenediamine (3.55 mL) gave 5-acetyl-N-[2-(dimethylamino)ethyl]-2-thiophenecarboxamide (1.16 g) as an amber oil.

1H NMR (300 MHz, DMSO-d6) δ ppm 2.20 (s, 6H), 2.42 (t, J=6.8 Hz, 2 H), 2.58 (s, 3 H), 3.38 (m, 2 H), 7.82 (d, J=4.1 Hz, 1 H), 7.95 (d, J=4.0 Hz, 1 H), 8.71 (t, J=5.5 Hz, 1 H); MS m/z 241 (M+1)⁺.

### (b) Preparation of N-[2-(dimethylamino)ethyl]-5-[(2E)-3-(dimethylamino)-2-propenoyl]-2-thiophenecarboxamide

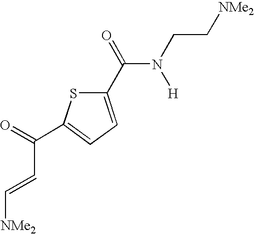

In a similar manner as described for Example 4b, 5-acetyl-N-[2-(dimethylamino)ethyl]-2-thiophenecarboxamide (1.16 g) and dimethylformamide dimethylacetal (6.41 mL) gave N-[2-(dimethylamino)ethyl]-5-[(2E)-3-(dimethylamino)-2-propenoyl]-2-thiophenecarboxamide (1.18 g) as an amber oil.

1H NMR (300 MHz, DMSO-d6) δ ppm 2.21 (s, 6H), 2.43 (t, J=6.8 Hz, 2 H), 2.95/319 (2×s, 6 H), 3.35 (m, 2 H), 5.81 (d, J=12.2 Hz, 1 H), 7.71-7.78 (m, 3 H), 8.52 (t, J=5.6 Hz, 1 H).

### (c) Preparation of N-[2-(dimethylamino)ethyl]-5-(2-{[4-(methyloxy)phenyl]amino}-4-pyrimidinyl)-2-thiophenecarboxamide

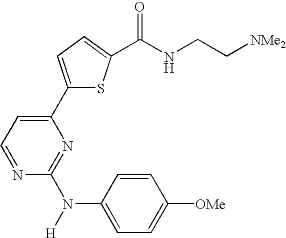

In a similar manner as described for Example 4c, sodium (9.2 mg) dissolved in EtOH (1.0 mL), 1-(4-methoxyphenyl)guanidine hydrochloride (81 mg) and N-[2-(dimethylamino)ethyl]-5-[(2E)-3-(dimethylamino)-2-propenoyl]-2-thiophenecarboxamide (118 mg) in EtOH (3.0 mL) gave N-[2-(dimethylamino)ethyl]-5-(2-{[4-(methyloxy)phenyl]amino}-4-pyrimidinyl)-2-thiophenecarboxamide (56 mg) as a solid.

1H NMR (400 MHz, DMSO-d6) δ ppm 2.30 (s, 6H), 2.57 (t, J=6.6 Hz, 2 H), 3.38 (m, 2 H), 3.72 (s, 3 H), 6.89 (d, J=9.0 Hz, 2 H), 7.29 (d, J=5.1 Hz, 1 H), 7.68 (d, J=9.0 Hz, 2 H), 7.79 (d, J=4.0 Hz, 1 H), 7.94 (d, J=4.0 Hz, 1 H), 8.46 (d, J=5.2 Hz, 1 H), 8.68 (t, J=5.6 Hz, 1 H), 9.51 (s, 1 H); MS m/z 398 (M+1)⁺.

### Example 7

### 5-{2-[(4-hydroxyphenyl)amino]-4-pyrimidinyl}-N-(2-phenylethyl)-2-thiophenecarboxamide

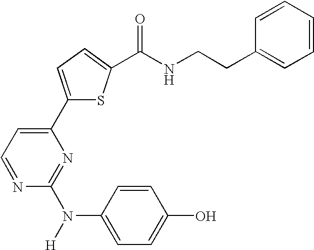

### (a) Preparation of 5-acetyl-N-(2-phenylethyl)-2-thiophenecarboxamide

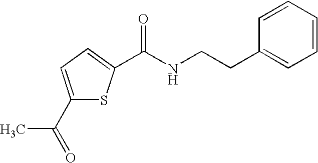

In a similar manner as described for Example 4a, 5-acetyl-2-thiophenecarboxylic acid (2.00 g), HOBt (2.16 g), EDC hydrochloride (2.72 g), DMF (25 mL) and 2-phenethylamine (1.63 mL) gave 5-acetyl-N-(2-phenylethyl)-2-thiophenecarboxamide (3.14 g) as a solid.

1H NMR (400 MHz, DMSO-d6) δ ppm 2.53 (s, 3 H), 2.82 (t, J=7.5 Hz, 2 H), 3.44 (m, 2 H), 7.17-7.30 (m, 5 H), 7.74 (d, J=3.9 Hz, 1 H), 7.89 (d, J=3.8 Hz, 1 H), 8.83 (t, J=5.5 Hz, 1 H); MS m/z 274 (M+1)⁺.

### (b) Preparation of 5-[(2E)-3-(dimethylamino)-2-propenoyl]-N-(2-phenylethyl)-2-thiophenecarboxamide

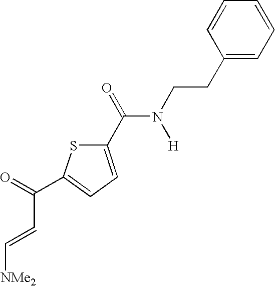

In a similar manner as described for Example 4b, 5-acetyl-N-(2-phenylethyl)-2-thiophenecarboxamide (3.14 g) and dimethylformamide dimethylacetal (25 mL) gave 5-[(2E)-3-(dimethylamino)-2-propenoyl]-N-(2-phenylethyl)-2-thiophenecarboxamide (3.53 g) as a yellow solid.

1H NMR (400 MHz, DMSO-d6) δ ppm 2.81 (t, J=7.4 Hz, 2 H), 2.90/3.13 (2×s, 6 H), 3.43 (m, 2 H), 5.76 (d, J=12.3 Hz, 1 H), 7.19-7.30 (m, 5 H), 7.65-7.72 (m, 3 H), 8.66 (t, J=5.5 Hz, 1 H); MS m/z 329 (M+1)⁺.

### (c) Preparation of 5-(2-{[4-(methyloxy)phenyl]amino}-4-pyrimidinyl)-N-(2-phenylethyl)-2-thiophenecarboxamide

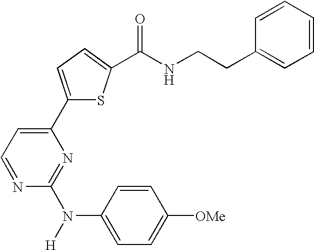

In a similar manner as described for Example 4c, sodium (9.2 mg) dissolved in EtOH (1.0 mL), 1-(4-methoxyphenyl)guanidine hydrochloride (81 mg) and 5-[(2E)-3-(dimethylamino)-2-propenoyl]-N-(2-phenylethyl)-2-thiophenecarboxamide (131 mg) in EtOH (3.0 mL) gave 5-(2-{[4-(methyloxy)phenyl]amino}-4-pyrimidinyl)-N-(2-phenylethyl)-2-thiophenecarboxamide (78 mg) as a solid.

1H NMR (400 MHz, DMSO-d6) δ ppm 2.81 (t, J=7.4 Hz, 2 H), 3.43 (m, 2 H), 3.74 (s, 3 H), 6.90 (d, J=9.0 Hz, 2 H), 7.26-7.28 (m, 1 H), 7.28-7.35 (m, 5 H), 7.69 (d, J=9.0 Hz, 2 H), 7.87 (d, J=3.9 Hz, 1 H), 7.97 (d, J=4.0 Hz, 1 H), 8.48 (d, J=5.1 Hz, 1 H), 9.21 (t, J=6.0 Hz, 1 H), 9.53 (s, 1 H); MS m/z431 (M+1)⁺.

### (d) Preparation of 5-{2-[(4-hydroxyphenyl)amino]-4-pyrimidinyl}-N-(2-phenylethyl)-2-thiophenecarboxamide

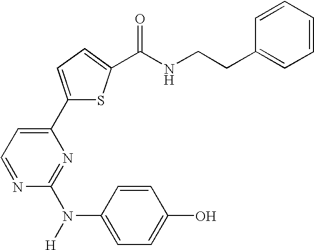

A mixture of 5-(2-{[4-(methyloxy)phenyl]amino}-4-pyrimidinyl)-N-(2-phenylethyl)-2-thiophenecarboxamide (76 mg, 0.18 mmol) in DCM (5.0 mL) was magnetically stirred while cooling to 0° C. with an ice slurry. A 1M solution of boron tribromide in DCM (0.54 mL, 0.54 mmol) was added dropwise via syringe to the stirring mixture. The reaction was warmed to rt and after 48 h the mixture was cooled to 0° C. with an ice slurry and quenched with dropwise addition of MeOH (4 mL). The volatiles were removed by rotary evaporation under reduced pressure, followed by addition of MeOH (2×15 mL) and rotary evaporation under reduced pressure after each addition to remove the volatiles. Purification by reversed-phase silica gel chromatography gave 5-{2-[(4-hydroxyphenyl)amino]-4-pyrimidinyl}-N-(2-phenylethyl)-2-thiophenecarboxamide (21 mg) as a yellow solid.

1H NMR (400 MHz, DMSO-d6) δ ppm 2.83 (t, J=7.2 Hz, 2 H), 3.46 (m, 2 H), 6.70 (d, J=9.0 Hz, 2 H), 7.18-7.31 (m, 6 H), 7.52 (d, J=9.0 Hz, 2 H), 7.75 (d, J=4.0 Hz, 1 H), 7.91 (d, J=4.0 Hz, 1 H), 8.43 (d, J=5.1 Hz, 1 H), 8.73 (t, J=5.7 Hz, 1 H), 9.08 (br s, 1 H), 9.36 (s, 1 H); MS m/z 417 (M+1)⁺.

### Synthesis of Intermediates used for Examples 8-42

### Preparation of 5-(4,4,5,5-tetramethyl-1,3,2-dioxaborolan-2-yl)-2-thiophenecarboxylic acid

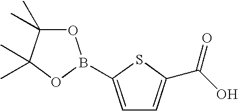

2-Carboxy-5-thiopheneboronic acid (1.04 g, 6.05 mmol) and pinacol (0.715 g, 6.05 mmol) were dissolved in a mixture of THF (15 mL) and toluene (15 mL). The volatiles were removed by rotary evaporation under reduced pressure. The solids were again treated three times with THF:toluene (10 mL:10 mL) followed by evaporation after each time to give 5-(4,4,5,5-tetramethyl-1,3,2-dioxaborolan-2-yl)-2-thiophene-carboxylic acid (1.43 g) as a white solid.

1H NMR (400 MHz, DMSO-d6) δ ppm 1.27 (s, 12H), 7.51 (d, J=3.6 Hz, 1H), 7.71 (d, J=3.7 Hz, 1 H), 13.26 (br s,1 H).

### Preparation of N-(3-methoxybenzyl)-5-(4,4,5,5-tetramethyl-1,3,2-dioxaborolan-2-yl)-2-thiophenecarboxamide

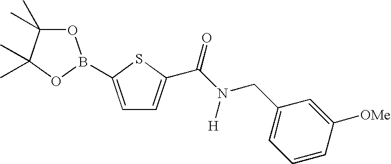

A mixture of 5-(4,4,5,5-tetramethyl-1,3,2-dioxaborolan-2-yl)-2-thiophenecarboxylic acid (10.1 g, 39.7 mmol), HOBt (6.43 g, 47.6 mmol), and EDC (9.13 g, 47.6 mmol), in DMF (100 mL) was treated with 3-methoxybenzylamine (5.6 mL, 43.7 mmol) and stirred at room temperature for 20 hours. The reaction mixture was poured onto ice water (300 mL) and extracted with EtOAc (3×150 mL). The combined organic layers were washed with brine, dried (MgSO₄), filtered and concentrated to afford the product, N-(3-methoxybenzyl)-5-(4,4,5,5-tetramethyl-1,3,2-dioxa-borolan-2-yl)-2-thiophenecarboxamide, (12.9 g) as a yellow solid.

1H NMR (400 MHz, DMSO-d6) δ ppm 1.26 (s, 12H), 3.71 (s, 3 H), 4.39 (d, J=6.0 Hz, 2H), 6.78-6.85 (m, 3H), 7.21 (t, J=8.0 Hz, 1H), 7.51 (d, J=3.7 Hz, 1H), 7.80 (d, J=3.7 Hz, 1 H), 9.09 (t, J=6.0 Hz, 1 H).

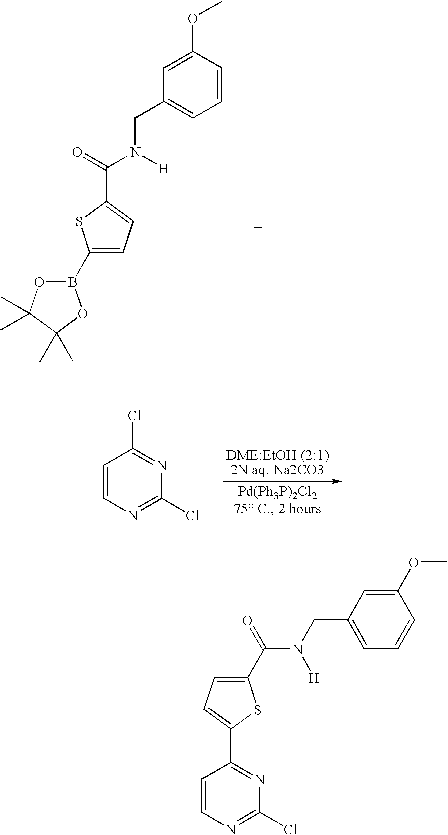

N-(3-methoxybenzyl)-5-(4,4,5,5-tetramethyl-1,3,2-dioxa-borolan-2-yl)-2-thiophenecarboxamide (2 g, 5.4 mmol), 2,4-dichloropyrimidine (2.4 g, 16.2 mmol), and palladium bis-triphenylphosphine dichloride (190 mg, 0.27 mmol) were combined, slurried in DME (20 mL) and EtOH (10 mL), and treated with Na₂CO₃ (4.1 mL of a 2N aqueous solution). The mixture was heated at 75° C. for 2 hours, at which time LC-MS analysis showed a product peak and consumption of starting boronate. The reaction mixture was partitioned between EtOAc (200 mL) and saturated aqueous NaHCO₃ (100 mL). The organic layer was dried (MgSO₄), filtered and concentrated to give dark solids. The solids were suspended in Et₂O (50 mL), stirred vigorously, and then filtered to give 1.6 g of purple solids. This material was dissolved in CH₂Cl₂ and filtered through a plug of silica. The silica plug was washed with EtOAc:Hexanes (50:50) and the combined organics concentrated to yield the product as gray solids (1.2 g).

### General Procedure for Synthesis of Examples 8-42

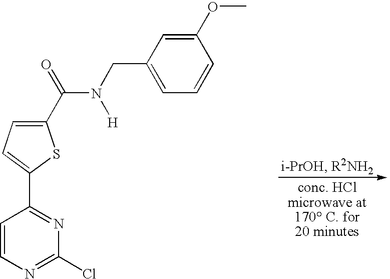

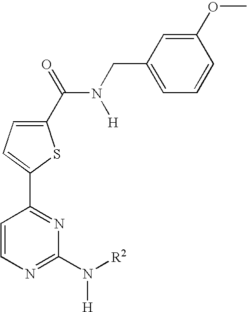

| Example | |
|---|---|
| number | R²NH₂ used in scheme |
| 8 | 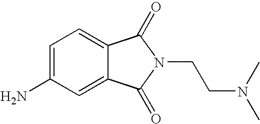 |
| 9 | 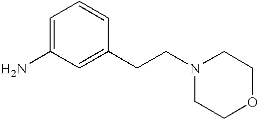 |
| 10 | 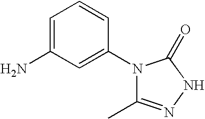 |
| 11 | 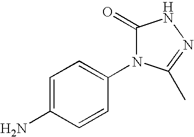 |
| 12 | 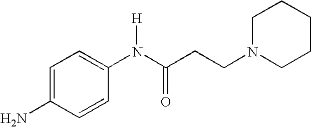 |
| 13 | 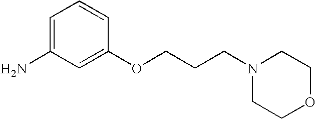 |
| 14 | 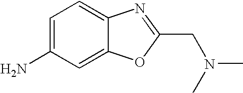 |
| 15 | 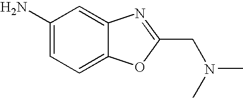 |
| 16 | 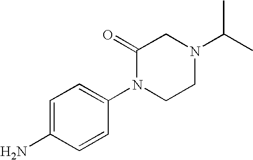 |
| 17 | 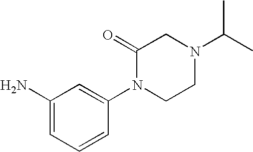 |
| 18 | 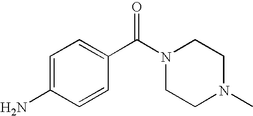 |
| 19 | 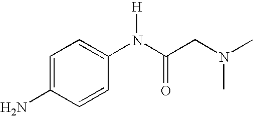 |
| 20 | 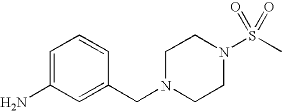 |
| 21 | 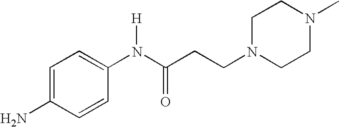 |
| 22 | 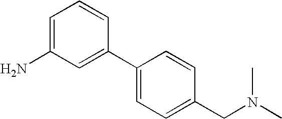 |
| 23 | 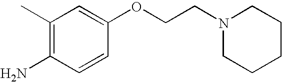 |
| 24 | 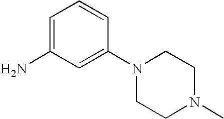 |
| 25 | 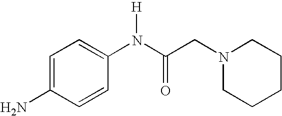 |
| 26 | 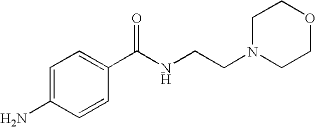 |
| 27 | 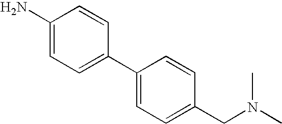 |
| 28 | 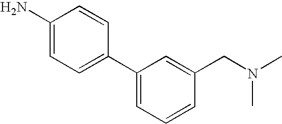 |
| 29 | 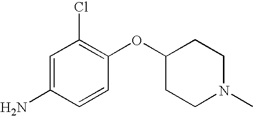 |
| 30 | 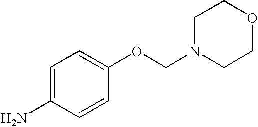 |
| 31 | 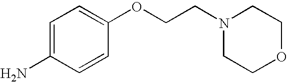 |
| 32 | 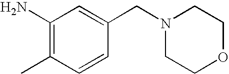 |
| 33 | 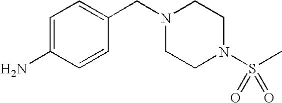 |
| 34 | 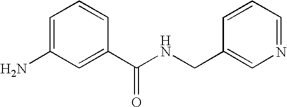 |
| 35 | 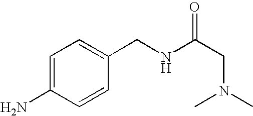 |
| 36 | 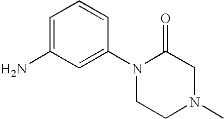 |
| 37 | 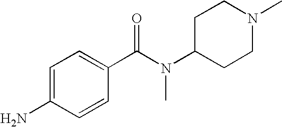 |
| 38 | 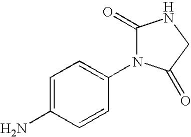 |
| 39 | 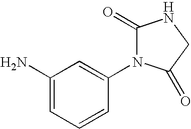 |
| 40 | 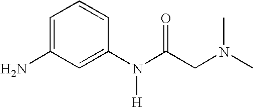 |
| 41 | 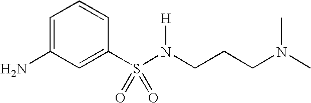 |
| 42 | 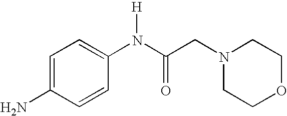 |

The starting pyrimidyl chloride (54 mg, 0.15 mmol) was placed in a 2-5 mL microwave reaction vessel from Personal Chemistry, suspended in iPrOH (2 mL), and treated with concentrated HCl (0.075 mL) and an aniline monomer (0.3 mmol). The vial was sealed and the reaction was heated in the Smith Synthesizer at 170° C. for 20 minutes. The cap was removed and Et₃N (0.5 mL) and CH₂Cl₂ (2 mL) were added. The solvent was evaporated and the residue purified by reverse phase mass directed prep HPLC [conditions: 4×20 mm Phenomenex Luna C18(2) 3 micron column, eluted with 10-100% Methanol (0.075% Formic Acid)/Water (0.1% Formic Acid), 3 minute gradient time, 4 minute run, 2 ml/minute]. The appropriate fractions were combined and concentrated to give the final products. Compounds with greater than 80% purity by peak area were submitted for screening and are shown in the table below.

| | | Retention | |
|---|---|---|---|
| Example # | MW | time | Area % Total |
| 8 | 556.19 | 1.87 | 100 |
| 9 | 529.21 | 1.81 | 100 |
| 10 | 513.16 | 2.41 | 100 |
| 11 | 513.16 | 2.39 | 100 |
| 12 | 570.24 | 1.84 | 92.16 |
| 13 | 559.23 | 1.88 | 100 |
| 14 | 514.18 | 1.79 | 100 |
| 15 | 514.18 | 1.81 | 80.44 |
| 16 | 556.23 | 1.8 | 100 |
| 17 | 556.23 | 1.82 | 80.06 |
| 18 | 542.21 | 1.74 | 100 |
| 19 | 516.19 | 1.76 | 100 |
| 20 | 592.19 | 1.87 | 100 |
| 21 | 585.25 | 1.78 | 80.34 |
| 22 | 549.22 | 2.02 | 100 |
| 23 | 557.25 | 1.83 | 100 |
| 24 | 514.22 | 1.82 | 88.45 |
| 25 | 556.23 | 1.84 | 100 |
| 26 | 572.22 | 1.83 | 100 |
| 27 | 549.22 | 1.99 | 88.16 |
| 28 | 549.22 | 2.04 | 86.33 |
| 29 | 563.18 | 1.96 | 100 |
| 30 | 529.21 | 1.78 | 100 |
| 31 | 545.21 | 1.78 | 100 |
| 32 | 529.21 | 1.82 | 100 |
| 33 | 592.19 | 1.81 | 100 |
| 34 | 550.18 | 2.13 | 100 |
| 35 | 530.21 | 1.83 | 100 |
| 36 | 528.19 | 1.8 | 88.09 |
| 37 | 570.24 | 1.76 | 100 |
| 38 | 514.14 | 2.29 | 100 |
| 39 | 514.14 | 2.29 | 100 |
| 40 | 516.19 | 1.8 | 100 |
| 41 | 580.19 | 1.84 | 100 |
| 42 | 558.21 | 1.89 | 100 |

### Synthesis of R²NH₂ Intermediates used for Examples 8-42

### R²NH₂ for Example 8

### Preparation of 5-amino-2-(2-dimethylamino-ethyl)isoindole-1,3-dione

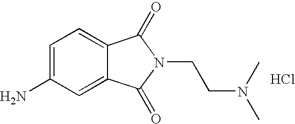

### a. 5-nitro-2-(2-dimethylamino-ethyl)isoindole-1,3-dione monohydrochloride

The reaction was carried out in a 6 L 3-necked RB flask, equipped with a mechanical stirrer, thermometer, condenser and CaCl₂ tube. N,N-dimethylethylenediamine (234.8 g, 2.59 mol) was added to a stirred suspension of 4-nitro-phthalic anhydride (500 g, 2.66 mol) in glacial acetic acid (3,383 ml) over 30 minutes. The temperature rose from 15 to 32° C. during this addition. The resulting solution was heated to reflux (ca. 112° C.) for 48 hours. The reaction mixture was then allowed to cool to room temperature and filtered.

The filtrate was concentrated to ca. 2 L and filtered again. The resulting latter filtrate was concentrated to dryness. The solid residue was taken up in methanol (250 ml) and Et₂O (2,250 ml), and treated with methanolic HCl (from MeOH (500 ml) and AcCl (160 ml)). The precipitated salt was collected by filtration, dried and recrystallised from MeOH (3 I) to give a cream coloured solid (254 g, 33 % theory).

### b. 5-amino-2-(2-dimethylamino-ethyl)isoindole-1,3-dione monohydrochloride

The reduction was carried out in a 2 I 3-necked flask provided with magnetic stirring. To a warm solution (35° C.) of the nitro compound (105 g, 0.35 mol) in methanol (1,575 ml), purged with N₂, was added 5-10% Pd on C (21 g, water wet). The reaction mixture was stirred magnetically and purged with H₂. A modest exotherm maintained the temperature at 33-37° C., and theoretical uptake of hydrogen occurred in 5 hours.

This reaction was repeated for a second batch under identical conditions.

The two reaction mixtures were then combined, heated to ca. 50° C. and filtered hot through Celite. The cake was washed further with MeOH (6 L, and the combined mother and wash liquors concentrated to dryness, giving a bright yellow solid (160 g). The filter cake was again washed with further hot methanol (1.5 L, 50° C.) and the filtrate used to slurry wash the crude product.

Filtration gave the target compound as a bright yellow solid of m.p 248.8-249.9° C. (156 g, 82% theory).

1H NMR (300 MHz, DMSO-d6) δ ppm 10.41 (br s, I H), 7.52 (d, 1H, J=8.2), 6.98 (s, 1H), 6.85 (dd, 1H, J=8.2, 2), 6.6 (br s, 2H), 3.88 (t, 2H, J=6.2), 3.34 (t, 2H, J=5.9), 2.83 (s, 6H)

### R²NH₂for Example 9

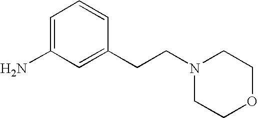

### a. 3-nitro-phenyl acetonitrile

Sodium cyanide (82 g, 1.667 mole) was taken up in water (1 L) and toluene (2 L) and heated at 60° C. A solution of 3-nitro-benzyl bromide (300 g, 1.389 moles) in toluene (1 L) was added, and the mixture heated to reflux overnight. The reaction was diluted with water, and the layers separated. The organic layer was washed with water and brine, dried (Na₂SO₄), filtered and concentrated to give the product as a brown liquid (220 g).

### b. 2-(3-nitrophenyl)ethanol

A solution of 3-nitro-phenyl acetonitrile (220 g, 1.078 moles) in water (440 mL), H₂SO₄ (440 mL), and acetic acid (440 mL) was heated at 110° C. for 4 hours. The reaction mixture was diluted with water, and extracted with EtOAc (3×1 L). The combined organics were washed with water and brine, dried (Na₂SO₄), filtered and concentrated to give the product carboxylic acid (200 g). This crude acid was dissolved in THF (3 L), cooled to 0° C., and treated with borane dimethyl sulfide (150 mL, 1.618 moles), allowed to warm to room temperature, and stirred overnight. The reaction mixture was evaporated to dryness and the resulting syrup was dissolved in EtOAc (3 L), washed with water and brine, dried (Na₂SO₄), filtered and concentrated to a brown liquid (175 g).

### c.

A solution of 2-(3-nitrophenyl)ethanol (175 g, 1.047 moles) in DCM (2 L) was treated with Et3N (220 mL, 1.570 moles), cooled to 0° C., treated with mesyl chloride (99 mL, 1.256 moles), and stirred for 4 hours. The reaction mixture was washed with water (3×1 L), brine (2×1 L), dried (Na₂SO₄), filtered and concentrated to a brown liquid (200 g).

### d.

A solution of the crude mesylate (200 g, 0.8156 moles) in morpholine (400 mL) was heated to 140° C., and kept at that temperature overnight. The reaction mixture was allowed to cool, and then diluted with water, and extracted with EtOAc (3×1 L). The combined organic layers were washed with water and brine, dried (Na₂SO₄), filtered and concentrated to give the crude product. The crude product was purified by chromatography on silica using Hexane: EtOAc as eluent (gradient from 3% to 20% EtOAc) to give the desired material as a yellow liquid (120 g).

### e.

A solution of the nitro compound (70 g, 0.2963 moles) in MeOH (1 L) was treated with charcoal (7 g) and ferric chloride (3.5 g) and heated to reflux. When the reaction was at reflux, hydrazine hydrate (70 mL) was added and the reaction was heated at reflux overnight. The reaction mixture was filtered through celite, and the filtrate was concentrated to a solid. Cold water was added to the solid and the solid was collected by filtration. The solid was washed with cold water, and dried to give the desired compound as an off white solid (55 grams).

1H NMR (300 MHz, DMSO-d6) δ ppm 6.93 (t, 1H, J=7.8), 6.39 (m, 3H), 4.95 (s, 2H), 3.6 (t, 4H, J=4.6), 2.41-2.61 (m, 8H)

### R²NH₂ for Example 10

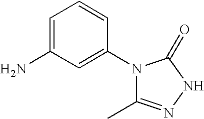

### a.

A stirred solution of m-phenylenediamine (120 g, 1.11 mol) in dioxane (1.2 L) and aqueous NaOH (570 mL of a 1 N solution) was cooled to 0° C. and treated dropwise with a solution of (Boc)2O (252 g, 1.15 mol)in dioxane (600 mL) over 60 minutes. The reaction mixture was stirred overnight, and the inorganics were removed by filtration. The filtrate was concentrated, taken up in DCM, washed with water and brine, dried (MgSO4), filtered and concentrated. The product was recrystallized from toluene and cyclohexane, yielding 150.64 g.

### b.

A solution of phenylchloroformate (18.1 mL, 0.144 mol) in THF (350 mL) was treated dropwise with a solution of the product from part a (30.64 g, 0.144 mol) in THF (60 mL) at a rate so as to keep the temperature below 30° C. Et₃N (20.24 mL) was then added at a rate that allowed the temperature to stay below 30° C. The reaction was stirred at room temperature overnight, then filtered to remove triethylamine hydrochloride. The filtrate was evaporated to give an amber oil. The oil was taken up in cyclohexane (400 mL) and warmed until complete dissolution had taken place. Toluene (50-100 mL) was added and the mixture heated to boiling, then allowed to cool slowly to give white crystals which were collected by filtration and dried to give the product (43.29 g).

### c.

The phenylcarbamate obtained in step b (38 g, 0.127 mol) was dissolved in dioxane (200 mL), treated with hydrazine hydrate (6.8 mL, 0.127 mol) and heated at reflux for 2 hours. The reaction mixture was allowed to cool to room temperature and poured into water (1400 mL). The precipitate was collected and washed with water (about 200-250 mL) and dried to give the desired product (27.38 g).

### d.

To acetamidine hydrochloride (10.79 g, 0.114 mol) in n-butanol (145 mL) is added NaOAc (9.35 g, 0.114 mol) and this is stirred for 30 minutes. The NaCl is filtered off through Celite, and the filtrate is added to a solution of the product from step c (27 g, 0.1037 mol) in DMF (210 mL). The reaction mixture is heated overnight at 130° C., and then the solvent is evaporated. The residue is partitioned between water and EtOAc, and the organic layer is washed again with water, dried (MgSO₄), filtered, and concentrated to a brown oil. The oil was crystallized from hot toluene to give the product (8 g).

### e.

The product from part d (2 g, 0.0069 mol) was treated with HCl in dioxane (10 mL). To aid dissolution, MeOH was added (20 mL). Upon completion of the reaction (TLC, 2% MeOH in EtOAc), the reaction was evaporated to dryness. The residue was taken up in water and the pH made alkaline, and then the reaction mixture was again evaporated to dryness. The residue was treated with ether, and then extracted into isopropanol. The mixture was filtered through Celite and the filtrate concentrated to give the product.

1H NMR (300 MHz, DMSO-d6) δ ppm 7.14 (t, 1H, J=8), 6.62 (dd, 1H, J=8, 2), 6.52 (s,1H), 6.45 (d, 1H, J=7.5), 5.36 (br s, 2H), 2.05 (s, 3H)

### R²NH₂ for Example 11

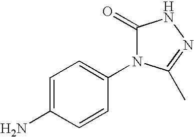

### a.

Aniline (74.5 g, 0.8 mol), methyl carbazate (79.26 g, 0.88 mol), p-toluenesulfonic acid (2 g), and triethyl orthoacetate (161.4 mL, 0.88 mol) were combined in IMS (800 mL) and heated at reflux for 1 day. The solution was cooled to room temperature and NaOCH₃ (43.22 g, 0.8 mol) was added. This was then heated at reflux for 3 days, and then the solvent was removed. Water was added (1.2 L), and the pH was adjusted to 5 with acetic acid. The precipitate was collected, washed with water and dried to afford the target compound (68 g).

### b.

The product from step a (26 g, 0.1484 mol) was added in portions to H₂SO₄ (169 mL) chilled to 2-5° C. After the material had dissolved, nitric acid (165 mL) was added slowly. Upon completion of the addition, the reaction was stirred for 10 minutes in the ice bath, and then 20 minutes at room temperature. The reaction mixture was poured cautiously onto 600 g of ice diluted with some water. The precipitate was filtered and recrystallized from IMS to give the desired material (17.23 g).

### c.

The nitro compound from step b (35 g) was dissolved in IMS and treated with wetted 10% Pd—C. The reaction mixture was placed on a hydrogenator, which was then charged with 40 atmospheres of H2. The vessel was recharged 3 times in half hour intervals. After 3 hours, the reaction mixture was filtered through Celite to remove the catalyst, and the Celite washed with hot IMS. The filtrate was concentrated to dryness and the product obtained by recrystallization from IMS.

1H NMR (300 MHz, DMSO-d6) δ ppm 11.45 (s, 1H), 6.98 (d, 2H, J=8.5), 6.65 (d, 2H, J=8.6), 5.4 (s, 2H), 1.99 (s, 3H)

### R²NH₂for Example 12

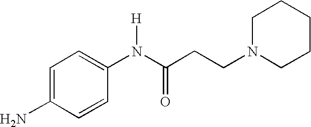

### a.

4-nitro-aniline (150 g) was suspended in toluene (1 L) and stirred. 3-Cl-propionyl chloride (104 mL) dissolved in toluene (500 mL) was added dropwise to the aniline suspension. The reaction mixture was stirred overnight at room temperature and then washed with aqueous Na₂CO₃ (2×) and 1N HCl (2×). The organics were dried (Na₂SO₄), filtered and concentrated to give the amide product (61%).

### b.

The amide (100 g) was suspended in toluene (800 mL). Piperidine (87 mL) was added and the solution heated to 100° C. for 3 hours. The solution was cooled and concentrated. The residue was taken up in DCM (300 mL) and 2N aqueous HCl. The resulting precipitate was removed by filtration and washed with 2N HCl. The precipitate was dissolved in water (3 L) and the solution basified with NaOH pellets to pH 14. The aqueous layer was extracted with DCM to give the product (63%).

### c.

A solution of the product from step b (35 g) in EtOH (1 L) was added to pre-wetted (EtOH) Pd/C catalyst. The solution was stirred under 1 atmosphere of hydrogen until all of the nitro compound had been consumed (tlc). The reaction mixture was filtered to remove the catalyst, and the solvent evaporated to yield the product (97%).

1H NMR (300 MHz, DMSO-d6) δ ppm 9.78 (s, 1H), 7.23 (d, 2H, J=8.7), 6.52 (d, 2H, J=8.7), 2.75 (m, 2H), 2.53 (m, 6H), 1.58 (m, 4H), 1.45 (m, 2H)

### R²NH₂for Example 13

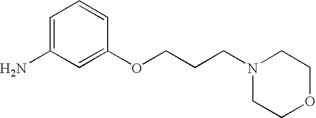

### a. 3-(3-nitrophenoxy)-1-chloropropane

3-nitro-phenol (100 g, 0.719 mole, 1-bromo-3-chloropropane (140 mL, 1.43 mol), and K₂CO₃ (300 g, 2.15 mol) were combined in CH₃CN (1.5 L), stirred with a mechanical stirrer, and heated at reflux overnight. The reaction mixture was diluted with water (3 L) and extracted with EtOAc (3×500 mL). The combined organic layers were washed with water (2×200 mL) and brine, dried (Na₂SO₄), filtered and concentrated to give the crude product (210 g) which was used as is in the next step.

### b.

3-(3-nitrophenoxy)-1-chloropropane (200 g, 0.92 mol), morpholine (162 mL, 1.85 mol), and K₂CO₃ (387 g, 2.78 mol) were combined in DMF (2 L), heated to 75° C., and stirred at this temperature for 36 hours. The reaction mixture was poured into ice water (5 L) and extracted with EtOAc (3×500 mL). The combined EtOAc layers were washed with water and brine, dried (Na₂SO₄), filtered and concentrated to give the crude product (190 g) as a brown liquid which was used as is in the next step.

### c.

The product from step b (150 g, 0.564 mol), FeCl₃ (15 g, 10%), and charcoal (15 g, 10%) were combined in MeOH (1.5 L) and heated to 60° C. Hydrazine hydrate (300 mL) was added to the hot solution over 30 minutes. After the addition was complete, the reaction stirred at room temperature overnight. The reaction mixture was filtered through celite and the celite plug was washed with MeOH. Most of the solvent was then removed by rotary evaporation (about 50 mL were left), and then water (100 mL) was added to the mixture. The solid precipitate was collected on a filter, washed with water, and dried. This solid was taken up in chloroform, dried (Na₂SO₄), filtered and concentrated to give the product as a white solid (110 g).

1H NMR (300 MHz, DMSO-d6) δ ppm 6.9 (t, 1H, J=8.3), 6.14 (m, 2H), 6.05 (m, 1H), 5.04 (s, 2H), 3.91 (t, 2H, J=6.5), 3.6 (t, 4H, J=4.6), 2.39 (m, 6H), 1.85 (m, 2H)

### R²NH₂ for Example 14

### Synthesis of 2-[(dimethylamino)-methyl]-1,3-benzoxazole-6-amine

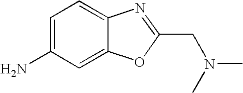

A suspension of methyl chloroacetimidate hydrochloride (170 g, 1.58 mole, prepared by treating a solution of chloroacetonitrile in 1:1 Et₂O:MeOH with HCl gas at 0° C., stirring at this temperature overnight and concentrating the reaction mixture to dryness) in DCM (2 L) was treated with 2-amino-5-nitro phenol (200 g, 1.3 mol). The suspension was stirred at room temperature for 24 hours then refluxed for 48 hours. The reaction mixture was washed with water (2×1 L) and brine (1×1 L), dried (Na₂SO₄), filtered and concentrated to give the crude product. The pure chloromethyl-benzoxazole (90 g, yellow solid) was obtained by chromatography on silica with 0 to 10% MeOH in chloroform as eluent.

### b.

A solution of the chloromethyl benzoxazole (90 g, 0.424 mol, prepared in part a) in MeOH (800 mL) was cooled to 0° C. and treated slowly with dimethyl amine (700 mL of a 40% solution, 0.635 mol). The reaction mixture was stirred for 3 hours, and the solid that formed was collected by filtration, washed with cold MeOH (200 mL), and air dried to give the desired product as a yellow solid (60 g).

### c.

A solution of the product from step b (60 g, 0.2713 mol) in MeOH (600 mL) was treated with iron (76 g, 1.356 mol), and then cooled to 0° C. Methanolic HCl (200 mL) was added and the reaction mixture stirred at room temperature for one day. The excess iron was removed by filtration and the reaction mixture was concentrated. The residue was taken up in water (200 mL) and washed with Et₂O (2×500 mL). The aqueous layer was neutralized with NaOH and extracted with Et₂O (3×500 mL). The combined Et₂O layers were dried (Na₂SO₄), filtered and concentrated to a brownish yellow solid (24 g).

1H NMR (300 MHz, DMSO-d6) δ ppm 6.64 (d, 1H, J=8), 6.1 (m, 2H), 4.74 (s, 2H), 4.63 (s, 2H), 2.94 (s, 6H)

### R²NH₂for Example 15

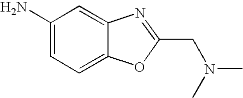

### a.

A suspension of methyl chloroacetimidate hydrochloride (170 g, 1.58 mole, prepared by treating a solution of chloroacetonitrile in 1:1 Et₂O:MeOH with HCl gas at 0° C., stirring at this temperature overnight and concentrating the reaction mixture to dryness) in DCM (100 mL) was cooled to 0° C. and treated with 2-amino-4-nitro phenol (6.4 g, 0.067 mol). The yellow suspension was stirred for 30 minutes at 0° C. and then at room temperature for 2 hours. The reaction was then heated to 45° C. for 16 hours. The reaction mixture was concentrated to dryness, and the product was crystallized from MeOH to yield a pale yellow solid.

### b.

A solution of the chloromethyl compound (60 g, 0.283 mol, prepared as in step a) in MeOH (600 mL) was cooled to 0° C. and treated slowly with dimethyl amine (120 mL of a 40% solution, 0.765 mol) and thenstirred at room temperature for 16 hours. The solid was collected by filtration, washed (cold (MeOH), and dried to five the target compound as a yellow solid (50 g).

### c.

A solution of the nitro derivative from step b (50 g, 0.226 mol)in MeOH (500 mL) was treated with 10% Pd/C (5 g) and stirred under 1 kg pressure of hydrogen for 24 hours. The reaction mixture was filtered and concentrated. The reduced product was purified by chromatography on silica eluting with 5% MeOH in DCM. The product obtained was not pure, so was carried on to the next step as is.

### d.

The product from step c (30 g, 0.157 mol) was dissolved in MeOH (300 mL), treated with Et₃N (23.8 g, 0.235 mol) and cooled to 0° C. Boc anhydride (41 g, 0.1884 mol) was added slowly, and the reaction stirred at room temperature for 3 hours. The reaction mixture was concentrated and taken up in EtOAc (200 mL). This organic solution was washed with water (3×100 mL) and brine (1×150 mL), dried (Na₂SO₄), filtered and concentrated. The product was purified by chromatography on silica using 20% EtOAc:80% hexanes as eluent, to give 20 g white solids.

### e.

The boc protected compound (23 g, 0.0103 mol, prepared as in step d) was dissolved in dry MeOH (40 mL) and treated with HCl in MeOH (60 mL) and stirred overnight at room temperature. The reaction mixture was concentrated to dryness, and the material was slurried in Et₂O (50 mL). The solid was collected by filtration, washed with Et₂O, and dried to give the desired compound (17.3 g).

1H NMR (300 MHz, DMSO-d6) δ ppm 10.8 (br, 2H), 7.8 (s, 1H)m 7.17 (m, 2H), 5.27 (m, 2H), 3.47 (s, 3H), 3.32 (s, 3H),

### R²NH₂ for Example 16

### Preparation of 1-(4-Aminophenyl)-4-isopropylpiperazin-2-one

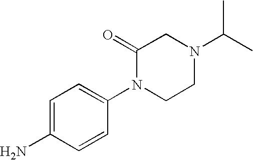

### a. Preparation of 2-Chloro-N-(4-nitrophenyl)acetamide

4-Nitroaniline (250.0 g, 1.8 mol) and triethylamine (276 mL, 1.98 mol) were dissolved in 1,4-dioxane (1.5 L) and cooled to 5° C. This stirred, cooled solution was then added to a solution of chloroacetyl chloride in 1,4-dioxane keeping the temperature <20° C. After stirring overnight, an extra equivalent of triethylamine and chloroacetyl chloride was added and the reaction was stirred once again overnight before being poured into water (5 L). After being stirred for 15 min, the resultant solid was filtered off, washed with water, and dried to give 358.8 g (93%) of 2-chloro-N-(4-nitrophenyl)acetamide: solid; mp 185-187° C.; R_{f} 0.14 (1:2 EtOAc/hexane)

### b. Preparation of N²-(2-Hydroxyethyl)-N-(4-nitrophenyl)glycinamide

2-Chloro-N-(4-nitrophenyl)acetamide (160.9 g, 1 mol) was suspended in methanol (2.25 L) under an atmosphere of argon. The mixture was cooled to 5° C. and ethanolamine (458.1 g, 10 mol) was added. The reaction mixture was stirred overnight and the resultant solid was filtered off, washed in methanol, and dried to give 149.5 g (83%) of N²-(2-hydroxyethyl)-N-(4-nitrophenyl)glycinamide.

### c. Preparation of tert-Butyl 4-(4-Nitrophenyl)-3-oxopiperazine-1-carboxylate

N²-(2-Hydroxyethyl)-N-(4-nitrophenyl)glycinamide (59.8 g, 0.25 mol) was suspended in ethyl acetate (500 mL) and to this was added tri-n-butylphosphine (86.2 mL, 0.32 mol). The reaction mixture was cooled to 5° C. and a solution of diisopropylazodicarboxylate (70.4 g, 0.32 mol) in ethyl acetate was added dropwise, keeping the reaction temperature <5° C. After the addition was complete the reaction was allowed to warm to room temperature and was stirred overnight before being extracted with 1:1 brine/water (3×500 mL). The organic layer was then acidified with 0.1 M hydrochloric acid and the aqueous phase was subsequently separated and neutralized. To each of the two aqueous solutions was then added di-tert-butyl dicarbonate (65.5 g, 0.3 mol) and the reaction mixtures were stirred overnight. The resultant solid was filtered off, dissolved in dichloromethane, dried, and concentrated to a slurry before being filtered off and dried to give 82.7 g (51%) of tert-butyl 4-(4-nitrophenyl)-3-oxopiperazine-1-carboxylate: R_{f} 0.56 (EtOAc)

### d. Preparation of 1-(4-Nitrophenyl)piperazin-2-one

To tert-butyl 4-(4-nitrophenyl)-3-oxopiperazine-1-carboxylate (60.0 g, 0.186 mol) was added 20% trifluoroacetic acid/dichloromethane (600 mL) at room temperature and the reaction mixture was stirred to 2 h. The excess trifluoroacetic acid was then removed under reduced pressure, toluene was added, and the resultant mixture was concentrated to dryness. Dichloromethane was then added and the resultant precipitate was filtered and washed with dichloromethane and then hexane. The solid in dichloromethane was treated with sodium hydroxide solution until the mixture was basic, washed with brine, dried over MgSO₄, and concentrated to give 31 g (76%) of 1-(4-nitrophenyl)piperazin-2-one: solid; mp 150-153° C., R_{f} 0.20 (MeOH).

### e. Preparation of 4-Isopropyl-1-(4-nitrophenyl)piperazin-2-one

1-(4-Nitrophenyl)piperazin-2-one (33 g, 0.149 mol) in methanol (350 mL), dichloromethane (200 mL), acetic acid (9.0 g, 1 eq), and acetone (26.0 g, 3 eq) was stirred at room temperature for 1 h then cooled to 0° C. and sodium triacetoxyborohydride (79.0 g, 2.5 eq) was added portionwise. The resultant mixture was stirred at room temperature overnight and then a solution of sodium bicarbonate was added carefully, followed by a solution of sodium hydroxide to attain a pH of 8-9. The reaction mixture was then extracted with dichloromethane (2×), washed with brine, dried (MgSO₄), and concentrated to give 38.5 g, (98%) of 4-Isopropyl-1-(4-nitrophenyl)piperazin-2-one: solid; mp 87-90° C., R_{f} 0.45 (MeOH).

### f. Preparation of 1-(4-Aminophenyl)-4-isopropylpiperazin-2-one

4-Isopropyl-1-(4-nitrophenyl)piperazin-2-one (20 g, 0.076 mol) was dissolved in MeOH (300 mL) and 5% palladium on carbon (3 g) in toluene (5 mL) was added. The autoclave was sealed and charged with hydrogen to 40 atmospheres and the resultant reaction mixture was stirred at room temperature for 1 h. The reaction mixture was then filtered through a pad of Celite, washed with MeOH (300 mL), and concentrated. The solid obtained was treated with diethyl ether and hexane (3:1) and decanted or filtered. The solid was subsequently vacuum dried to give 17 g (96%) of 1-(4-aminophenyl)-4-isopropylpiperazin-2-one: solid; mp 156-159° C.

1H NMR (300 MHz, DMSO-d6) δ ppm 6.92 (d, 2H, J=8.5), 6.56 (d, 2H, J=8.6), 5.12 (s, 2H), 3.5 (m, 2H), 3.18 (s, 2H), 2.76 (m, 3H), 1.04 (d, 6H, J=6.5)

### R²NH₂ for Example 17

### Preparation of 1-(3-Aminophenyl)-4-isopropylpiperazin-2-one

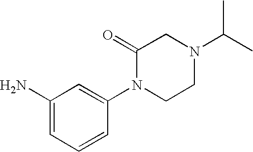

### a. Preparation of 2-Chloro-N-(3-nitrophenyl)acetamide

3-Nitroaniline (13.8 g, 0.1 mol) and triethylamine (11.1 g, 0.11 mol) were dissolved in 1,4-dioxane (50 mL) and cooled to 0° C. A solution of chloroacetyl chloride (11.3 g, 0.1 mol) in 1,4-dioxane (10 mL) was added dropwise over 30 min and the reaction was stirred for 2 h before being poured into water (500 mL) and stirred for a further 30 min. The resultant solid was filtered off, washed with water, and dried ti give 14.05 g (65%) of 2-chloro-N-(3-nitrophenyl)acetamide: solid; mp 110-112° C.; R_{f} 0.61 (EtOAc).

### b. Preparation of N²-(2-Hydroxyethyl)-N-(3-nitrophenyl)glycinamide

2-Chloro-N-(3-nitrophenyl)acetamide (107 g, 0.5 mol) was dissolved in methanol (1.5 L) and cooled to 5° C. To this was added ethanolamine (305 g, 5.0 mol) with stirring over 10 min. The reaction mixture was then stirred at room temperature for 1 h, after which time a solid began to precipitate out. The reaction mixture was stirred overnight and the solid was then filtered off, washed with methanol and hexane (250 mL), and dried under vacuum overnight to give 96.1 g (80%) of N²-(2-hydroxyethyl)-N-(3-nitrophenyl)glycinamide: solid; mp 145-147° C.; R_{f} 0.31 (20% MeOH/DCM).

### c. Preparation of tert-Butyl 4-(3-Nitrophenyl)-3-oxopiperazine-1-carboxylate

N²-(2-Hydroxyethyl)-N-(3-nitrophenyl)glycinamide (59.8 g, 0.25 mol) was suspended in ethyl acetate (500 mL) and cooled to 50° C. To this was added tri-n-butylphosphine (86.2 mL, 0.32 mol) and then a solution of diisopropylazodicarboxylate (70.4 g, 0.32 mol) in EtOAc (160 mL) was added dropwise over 30 min, while the temperature of the reaction mixture was maintained <10° C. The reaction mixture was stirred overnight and then extracted with 1:1 water/brine (3×500 mL). The EtOAc layer was reserved and treated as below(*) To the aqueous solution was added di-tert-butyidicarbonate and the reaction mixture was stirred overnight. The resultant solid was filtered off and taken up into DCM, dried (MgSO₄), concentrated to a slurry, swirled with hexane, filtered and dried. (*) The EtOAc layer was acidified with 0.1 M HCl, and the aqueous layer was removed and neutralized (KOH) and to this was also added di-tert-butyldicarbonate and the above repeated to get a second crop of product. This gave 45 g (56%) of tert-butyl 4-(3-nitrophenyl)-3-oxopiperazine-1-carboxylate: solid; mp 122-123° C.

### d. Preparation of 1-(3-Nitrophenyl)piperazin-2-one

tert-Butyl 4-(3-nitrophenyl)-3-oxopiperazine-1-carboxylate (60 g, 0.19 mol) was added to a solution of trifluoroacetic acid (120 mL, 1.6 mol) in dichloromethane (480 mL) and the resultant reaction mixture was stirred for 2 h. The dichloromethane and trifluoroacetic acid were then removed under reduced pressure and the reaction mixture was triturated with toluene (100 mL). Dichloromethane (750 mL) was added to the residue and the mixture was made basific with sodium hydroxide solution. The organic fraction was separated and concentrated under reduced pressure to a slurry, to which was added hexane. The resultant precipitate was filtered off, washed with hexane, and dried overnight under reduced pressure to gave 36 g (86%) of 1-(3-nitrophenyl)piperazin-2-one: solid; mp 137-139° C.; R_{f} 0.33 (10% MeOH/DCM).

### e. Preparation of 4-Isopropyl-1-(3-nitrophenyl)piperazin-2-one

1-(3-Nitrophenyl)piperazin-2-one (32.0 g, 0.14 mol), acetone (24.4 g, 0.42 mol), acetic acid (8.4 g, 0.14 mol), methanol (350 mL), and dichloromethane (200 mL) were combined and stirred for 30 mins. Sodium triacetoxyborohydride (74.2 g, 0.35 mol) was then added portion wise over 1 h and the resultant reaction mixture was stirred overnight. Dichloromethane (250 mL) was added and the reaction was quenched with saturated sodium bicarbonate solution. The organic layer was separated and concentrated to a slurry and then hexane was added. The resultant precipitate was filtered off and dried to give 34.98 g (95%) of 4-isopropyl-1-(3-nitrophenyl)piperazin-2-one: R_{f} 0.47 (EtOAc).

### f. Preparation of 1-(3-Aminophenyl)-4-isopropylpiperazin-2-one

4-Isopropyl-1-(3-nitrophenyl)piperazin-2-one (26.0 g, 0.11 mol) was dissolved in methanol (500 mL) and to this was added 5% palladium on carbon (3.0 g) as a paste in toluene in a 1 L autoclave. This was charged to 40 atmospheres with hydrogen and stirred for 30 mins by which time the required amount of hydrogen (16 atmospheres) had been consumed. The catalyst was filtered through Celite and the solvent was removed under reduced pressure to leave an oil. This was taken up in dichloromethane, dried (MgSO₄), and concentrated to an oil which was triturated with a small amount of dichloromethane and some hexane to give a white solid. This was filtered off and dried under reduced pressure overnight to give 53.0 g (87%) of 1-(3-aminophenyl)-4-isopropylpiperazin-2-one: white solid; mp 114-115° C.; R_{f} 0.27 (3:1 EtOAc/MeOH).

1H NMR (300 MHz, DMSO-d6) δ ppm 7.03 (t, 1H, J=7.9), 6.46 (m, 3H), 5.17 (s, 2H), 3.54 (m, 2H), 3.2 (s, 2H), 2.78 (m, 3H), 1.05 (d, 6H, J=6.6)

### R²NH₂for Example 18

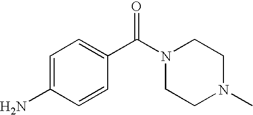

commercial: CAS #55121-99-8 (preparation available in WO2003024967)

### R²NH₂ for Example 19

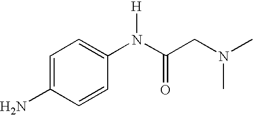

### a.

A solution of 4-nitro-aniline (200 g, 1.4492 mol) in DCM (2 L) was cooled to 0° C., and treated with Et₃N (175 g, 1.739 mol) and then chloroacetyl chloride (180 g, 1.594 mol). After the addition was complete, the temperature was allowed to rise to room temperature, and the reaction mixture was stirred overnight. The reaction mixture was filtered, and the solids washed with water and then dried under vacuum and then by evaporation from toluene to yield the product (190 g).

### b.

A solution of the product from step a (90 g, 0.4196 mol) in DMF (900 mL) was treated with powdered K2CO3 (115.8 g, 0.840 mol), followed by dimethylamine hydrochloride (51.3 g, 0.62937 mol). The reaction mixture was heated to 60° C. and stirred at that temperature for 2 hours. Water was added to the reaction mixture, and the product was extracted with EtOAc (3×350 mL). The organic layers were combined and washed with water (2×250 mL) and brine, dried (Na₂SO₄), filtered and concentrated to a light brown solid (72 g).

### c.

The nitro compound prepared in step a (65 g, 0.27896 mol) was dissolved in MeOH (650 mL) and, under nitrogen, treated with Pd/C (6.5 g). The reaction was stirred under an atmosphere of hydrogen. The next day the reaction was only 60% complete, so the reaction mixture was transferred to a Paar shaker and reacted under 3.0 kg/cm² of hydrogen overnight. The reaction was filtered though celite and evaporated to dryness. The crude product was purified by chromatography on silica using 50% EtOAc in petroleum ether as eluent to provide the desired product as a viscous liquid (50 g).

1H NMR (300 MHz, DMSO-d6) δ ppm 9.28 (s, 1H), 7.27 (d, 2H, J=8.7), 6.52 (d, 2H, J=8.7), 4.91 (br s, 2H), 3.02 (s, 2H), 2.29 (s, 6H)

### R²NH₂ for Example 20

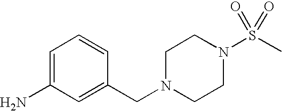

### a.

1-(methylsulfonyl)-piperazine, trifluoroacetate (150 g, 0.539 mole) in CH₃CN (2 L) was treated with K₂CO₃ (164 g, 1.18 mole) and 3-nitro-benzyl bromide (128.2 g, 0.593 mole) and stirred at room temperature overnight. The reaction mixture was poured into ice water (total volume about 10 L), and the precipitate was collected by filtration. The precipitate was then taken up in DCM, dried, filtered and concentrated to a yellow solid (145 g).

### b.

A solution of the nitro compound from step a (140 g, 0.46 mole) in EtOH (500 mL) and THF (500 mL) was treated with Pt/C (146, 10% w/w) and hydrogenated at atmospheric pressure overnight. The reaction mixture was degassed by bubbling nitrogen through the mixture, and then filtered and concentrated to a pale yellow solid (120 g).

1H NMR (300 MHz, DMSO-d6) δ ppm 6.98 (t, 1H, J=7.7), 6.55 (s, 1H), 6.48 (dd, 2H, J=2.7, 8.0), 5.1 (br, 2H), 3.4 (s, 2H), 3.13 (m, 4H), 2.9 (s, 3H), 2.53 (m, 4H)

### R²NH₂ for Example 21

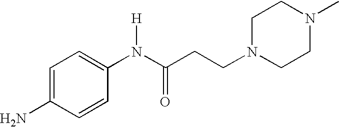

### a. synthesis of N-(4-nitrophenyl)acrylamide

4-nitro-aniline (100 g, 0.7246 mole) was dissolved in MDC (1.5 L), treated with Et₃N (201.5 mL, 1.4492 mole), and cooled to 0° C. Chloropropionyl chloride (83.4 mL, 0.8695 mole) was then added slowly at 0° C. The reaction mixture was then allowed to warm to room temperature and stirred overnight. Water (1 L) was added and the precipitate collected. The solids were washed with water (1.5L) and MDC (1 L), and dried to give the product (120 g).

### b.

The acrylamide (140 g, 0.7253 mole) and N-methyl piperazine (79.9 g, 0.7979 mole) were combined in THF (700 mL) and stirred overnight. The reaction mixture was concentrated and the solid was washed with water and EtOAc (2.5 L), and dried to give the Michael addition product (135 g).

### c.

The piperazine derivative from step b (135 g) was dissolved in MeOH (1 L). To 350 mL of MeOH in a 5 L autoclave nitrogen was passed for 10 minutes. To the methanol, 13.5 g Pd/C was slowly added with constant stirring. The 1 L MeOH solution of the starting material was added to the catalyst slurry and the reaction was stirred at room temperature under 5 kg pressure of hydrogen. The reaction mixture was removed from the autoclave, filtered, and concentrated. The solid was washed with diethyl ether to afford the reduced product (115 g).

1H NNR (300 MHz, DMSO-d6) δ ppm 9.66 (s, 1H), 7.21 (d, 2H, J=8.7), 6.52 (d, 2J, J=8.7), 4.85 (s, 2H), 2.6 (m, 4H), 2.39 (m, 8H), 2.17 (s, 3H)

### R²NH₂ for Example 22

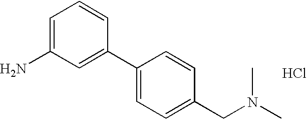

To a 1 L round bottom flask with a stir bar, reflux condenser and gas inlet containing [4-bromophenyl)methyl]dimethyl amine (55 mmol, 11.8 g) in glyme (300 mL) and water (30 mL) was added 3-aminophenyl boronic acid hydrate (110 mmol, 17 g), (Ph₃P)₂PdCl₂ (2.75 mmol, 1.93 g) and sodium carbonate (165 mmol, 17.5 g). The reaction was stirred at reflux under nitrogen until LC-MS analysis indicated consumption of starting material. The reaction mixture was partitioned between diethyl ether and water. The organic layer was washed with brine, dried (Na₂SO₄), filtered, and concentrated to give an oil. The oil was slurried in Et₂O and acidified with 1N HCl in Et₂O to give the HCl salt of the product as a precipitate (12 g, 73%).

1H NMR (300 MHz, DMSO-d6) δ ppm 8.15 (br s, 2H), 7.58 (d, 1H, J=7.3), 7.52 (d, 1H, J=1.8), 7.34 (t, 1H, J=7.7), 7.22 (m, 2H), 7.06 (m, 2H), 6.79 (d, 1H, J=7.8), 4.32 (s, 2H), 2.73 (s, 6H)

### R²NH₂ for Example 23

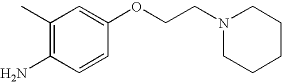

1H NMR (300 MHz, DMSO-d6) δ ppm 6.6 (s, 1H), 6.54 (s, 2H), 4.38 (s, 2H), 3.92 (t, 2H, J=6.0), 2.59 (t, 2H, J=6.0), 2.42 (m, 4H), 2.05 (s, 3H), 1.51 (m, 4H), 1.40 (m, 2H)

### R²NH₂ for Example 24

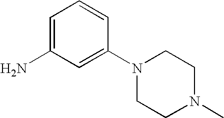

preparation in J Med Chem (2001) 44, 3946-3955.

### R²NH₂ for Example 25

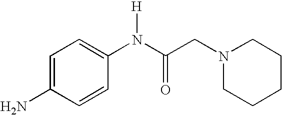

### a.

4-nitro-aniline (150 g) was suspended in toluene (1.5 L) and stirred. Chloroacetyl chloride (87 mL) was dissolved in toluene (100 mL) and added dropwise to the aniline suspension. The reaction mixture was stirred overnight and then washed with aqueous sodium carbonate solution (2×), 1N aqueous HCl (2×), and water. The organics were dried over sodium sulfate and concentrated to give the product (96%).

### b.

The amide from step a (150 g) was suspended in toluene (1.5 L). Piperidine (138 mL) was added slowly and the solution heated to reflux for 1 hour. The solution was cooled and concentrated. DCM (500 mL) was added, and then 2N aqueous HCl, and the resulting precipitate was removed by filtration and washed with a further portion of HCl. The precipitate was dissolved in water (2 L) and basified with NaOH pellets to pH 14. The resulting precipitate was removed by filtration and washed with water to give the product as a white solid. Drying in a vacuum oven gave the product (72%).

### c.

A solution of the nitro material (42 g) was dissolved in EtOH (3 L) and added under nitrogen to a pre-wetted (EtOH) Pd/C catalyst. The solution was stirred under an atmosphere of hydrogen until all of the nitro material had been consumed (tlc). The solution was filtered and the filtrates concentrated to yield the desired product (95%).

1H NMR (300 MHz, DMSO-d6) δ ppm 9.23 (s, 1H), 7.25 (d, 2H, J=8.7), 6.52 (d, J=8.7), 4.89 (s, 2H), 3.0 (s, 2H), 2.46 (m, 4H), 1.58 (m, 4H), 1.42 (m, 2H)

### R²NH₂ for Example 26

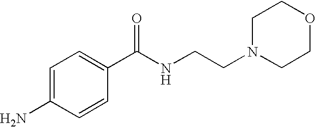

### a.

A solution of 4-nitro benzoyl chloride (20 g, 0.108 mol) in DCM (500 mL) was treated with TEA (16.5 mL) and then 4-(2-aminoethyl)morpholine (30.9 g). The reaction was stirred for 2 days and then concentrated to dryness. The reaction mixture was partitioned between EtOAc and aqueous NaHCO₃. The organic layer was washed with brine, and then concentrated to dryness. This material was slurried in 2N NaOH and filtered to give the product as a yellow solid (23 g).

### b.

The nitro compound from part a (23 g) was hydrogenated at atmospheric pressure in EtOH (400 mL) with 10% Pd/C (2.3 g) as catalyst. The reaction mixture was filtered and concentrated to give the product as a white solid (15.3 g).

1H NMR (300 MHz, DMSO-d6) δ ppm 7.93 (t, 1H, J=5.5), 7.57 (d, 2H, J=8.6), 6.56 (d, 2H, J=8.7), 5.61 (s, 2H), 3.59 (m, 4H), 3.36 (m, 2H), 2.43 (m, 6H)

### R²NH₂ for Example 27

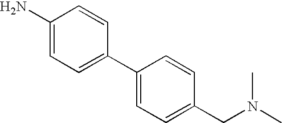

### a.

A solution of (4-bromobenzyl)dimethylamine (120 g, 0.50 mole) in THF (2 L) was cooled to −78° C. and treated dropwise with n-BuLi (47 g, 0.728 mole). The reaction mixture was stirred for 1 hour and then butyl borate (194 g, 0.84 mole) was added dropwise. The reaction mixture was stirred for 1 hour, allowed to warm to 0° C., and then quenched by the addition of water (1.5 L). The reaction mixture was washed with ether (2×1 L), and the aqueous layer taken on to the next step.

### b.

A solution of 4-bromo-nitro-benzene (113 g, 0.5586 mole) in toluene (2 L) was heated under nitrogen to 80-85° C. Pd(Ph₃P)₄ (25.8 g, 0.0223 mole) was added and stirred for 30 minutes, and then the aqueous solution of boronic acid (step a) was added, followed by addition of Na₂CO₃ (118.4 g, 1.1172 mole). The reaction was heated for 24 hours. The reaction mixture was allowed to reach room temperature and then transferred to a separatory funnel. The organic layer was separated and washed with water (1 L). The toluene layer was then treated with 1N HCl (2 L). The aqueous layer was washed with diethyl ether (2×1 L), basified by addition of 50% aqueous NaOH. The precipitated solid was filtered and dried to give the coupled product as a yellow solid (107 g).

### c.

The coupled product from step b (98 g) was dissolved in MeOH (1 L) and treated with FeCl₃ (2 g, and charcoal (10 g). The reaction mixture was stirred with an overhead stirrer at a temperature of 60-65° C. Hydrazine hydrate (200 mL) was added dropwise to the stirring mixture over 30 minutes. The reaction mixture was heated at 60-65° C. for 3 hours. The reaction mixture was allowed to cool to room temperature and filtered through celite, then the celite plug was washed with MeOH. The filtrate was concentrated to give the crude product which was purified by the addition of water followed by filtration of the solids. These solids were air dried to give the desired compound as a whitish yellow solid (78 g).

1H NMR (300 MHz, DMSO-d6) δ ppm 7.50 (d, 2H, J=8.1), 7.37 (d, 2H, J=8.5), 7.29 (d, 2H, J=8.1), 6.65 (d, 2H, J=8.4), 5.22 (s, 2H), 3.39 (s, 2H), 2.17 (s, 6H)

### R²NH₂ for Example 28

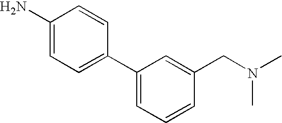

This compound was synthesized in a manner analogous to R²NH₂ for example 27.

1H NMR (300 MHz, DMSO-d6) δ ppm 7.45 (m, 5H), 7.15 (d, 1H, J=7.5), 6.67 (d, 2H, J=8.4), 5.24 (s, 2H), 3.43 (s, 2H), 2.18 (s, 6H)

### R²NH₂ for Example 29

### Preparation of 3-chloro-4-(1-methyl-piperidin-4-yloxy)-phenylamine

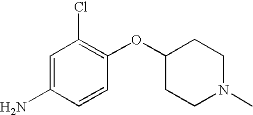

### a. Preparation of 4-(1-butoxycarbonyl-piperidin-4-yloxy)-3-chloro-nitrobenzene

The reaction was carried out in a 5 L 3-necked flask provided with a mechanical stirrer, thermometer, CaCl₂ tube, N₂ atmosphere and chilling facility.

Di-isopropylazodicarboxylate (DIAD, 489.7 g, 2.42 mol) was added over 45 mins to a solution of 2-chloro-4-nitrophenol (300 g, 1.729 mol), N-Boc-4-hydroxypiperidine (382.8 g, 1.902 mol) and triphenylphosphine (Ph₃P, 634.5 g, 2.47 mol) in tetrahydrofuran (THF, 2,160 ml) at 0 to 5° C. After stirring for a further 10 mins at 5° C., the temperature was gradually allowed to rise to 20° C. After 3.75 hours from the end of the addition, the batch was vacuum concentrated to a residual oil (1921 g).

On digestion with 9:1 hexane-ethyl acetate (4.3 l) and stirring, the resulting solid (largely Ph₃PO contaminated with product) was filtered and washed with 9:1 hexane:EtOAc (2 L).

The filtrate was vacuum concentrated to an oil (292 g), dissolved in DCM (500 ml) and washed with 0.08 N NaOH (1.5 l), 0.005 N NaOH (1.0 l) and saturated brine (200 ml). The DCM liquors were then dried with MgSO₄, filtered and vacuum concentrated to an oil (264 g). On dissolution in hexane (400 ml) and standing, a crystalline crop separated and this was filtered, washed with hexane (500 ml) and dried to yield 51.0 g of material, M.Pt. 95-98° C. The hexane mother and wash liquors were combined and charged onto a silica column (1,600 g). Flash chromatography was carried out, eluting with 100% hexane through to 25% EtOAc-hexane to yield 71.8 g of good quality intermediate, M.Pt. 105.0-105.5° C.

The impure “Ph₃PO” crop was further extracted with 50 % EtOAc-hexane (total 1.8 L) until TLC indicated that the residual solid comprised only Ph₃PO. Evaporation of the extract yielded an orange oil (533 g), which was dissolved in DCM (1.5 l) and washed with 0.2 N NaOH (2.6 l), 0.1 N NaOH (1 l). After brine (1×500 ml) washing of the DCM solution, drying, filtration and vacuum evaporation, an orange oil (420 g) was obtained. This oil was flash chromatographed on two columns (total SiO₂ 3,060 g), eluting as previously. Fractions containing product were obtained by vacuum concentration and filtration from a small volume of hexane. The combined column fractions yielded 71.7 g of M.Pt. 106-108.8° C. and 172.9 g of M.Pt. 94-96° C.

Total batch yield: 367.4 g (59.6% theory on 2-Cl-4-NO₂-phenol input)

| | | |
|---|---|---|
| 1) | 143.5 g, M.Pt. | Clean TLC (50% EtOAc: hexane - detection by |
| | 105-108° C. | exposure with TFA and iodoplatinate spray, R_{f} |
| | | 0.69 |
| 2) | 223.9 g, M.Pt. | Similar TLC main product spot, but “ghosting” |
| | 94-96° C. | with |
| | | probably di-isopropyl hydrazodicarboxylate |
| | | deriv. |

### b. Preparation of 3-chloro-4-(piperidin-4-yloxy)nitrobenzene (trifluoroacetate salt)

Batch 1: This reaction was carried out in a 2 L 3-necked flask provided with mechanical stirrer, thermometer, pressure-equalizing funnel, CaCl₂ tube and cooling bath.

Trifluoroacetic acid (TFA, 333.6 g, 2.929 mols) was added over 36 minutes to a solution of 4-(1-butoxycarbonyl-piperidin-4-yloxy)-3-chloro-nitrobenzene (209 g, 0.586 mol) in dry DCM (1,013 ml) at a temperature of 0 to 5° C. The cooling bath was then removed and the batch allowed to warm to room temperature. The reaction appeared to be complete after 2 hours (TLC) from the time of final TFA addition. After a further hour, the batch was evaporated under vacuum to a residual gum (430 g). This residue was dissolved in ethyl acetate (1,350 ml) and treated with K₂CO₃/H₂O (136 g/400 ml) until alkaline (pH 8). The aqueous layer was separated and the organics were washed with brine (1×300 ml). After drying on MgSO₄, the dry extract was concentrated to 300 ml, cooled to ca. 10° C. and a 1^{st} crop filtered, washed with cold EtOAc (50 ml) and dried.

Evaporation of the filtrate yielded an oil residue (65.5 g) which was digested with hot EtOAc (50 ml) and a second crop obtained on cooling.

Yield Crop 1 133.0 g (61.2 % theory), M.Pt. 146.5-148.2° C.
Crop 2 36.0 g (16.6 % theory), M.Pt.134.3-137.0° C.

Total batch yield 169.0 g (77.8% theory)

TLC System: DCM:MeOH:NH₄OH 90:10:1
Product R_{f} 0.11 (uv+iodoplatinate)
Crop 1: One spot
Crop 2: One main spot+minor impurity spots

Batch 2 This reaction was performed in a 1 l flask with similar arrangements to Batch 1.

The reaction conditions were also similar to those used in Batch 1.

4-(4-Butoxycarbonyl-piperidin-4-yloxy)-3-chloro-nitrobenzene (76.45 g, 0.214 mol) was dissolved in dry DCM (370 ml) and trifluoroacetic acid (122 g, 1.068 mol) was added over 24 minutes. After reaction and evaporation, the residual oil (142 g) was dissolved in EtOAc (500 ml) and treated with K₂CO₃/H₂O (79 g/290 ml), bringing the pH to 9 (instead of 8). Above pH 8, product precipitated in the ethyl acetate. The lower aqueous layer was separated and the suspension of product in EtOAc was washed with brine (3×100 ml) to pH 7.5. The suspension was filtered and the cake washed with water (500 ml) and then with EtOAc (400 ml). The solid was dried over P₂O₅ under vacuum at 50° C. Drying of the combined EtOAc liquors, followed by evaporation and then crystallisation from EtOAc-hexane, yielded only 4.6 g (8.4% theory) of impure product of higher M.Pt. than the main product crop.

Yield: 46.8 g (85.1% theory as free base). M.Pt. 105-109° C.

TLC One spot of identical R_{f} to Batch 1 product (same system).

¹H NMR confirmed Batch 2 material to be the free base.

### c. Preparation of 3-chloro-4-(1-methyl-piperidine-4-yloxy)nitrobenzene

Batch 1: This was performed in a 250 ml 3-necked RB flask provided with thermometer and air condenser. A solution of 3-chloro-4-(piperidin-4-yloxy)nitrobenzene trifluoroacetic acid salt (130 g, 0.351 mol), 90% formic acid (129.6 g, 2.53 mol) and 38% formaldehyde (87.9 g, 1.11 mol) was heated on a steam bath (internal temp. 90° C.) for 3.5 hours. Gas evolution was noted to cease after 2 hours. Conc. HCl (46.4 ml) was added and the batch then evaporated to a glassy residue (150 g). This latter material was dissolved in water (200 ml) and basified with 5 N NaOH (30 ml). The solution was heated at 80-90° C. for 15 minutes before cooling and extraction with CHCl₃ (3×600 ml) and back-washing with brine (1×300 ml). Following drying with MgSO₄ and filtration, the solution was evaporated under vacuum to a pale lemon solid residue.

Yield: 91.3 g (96.2% theory) M.Pt. 102.1-102.6° C.

TLC System: DCM:MeOH:NH₄OH 90:10:1 One spot R_{f} 0.24

Batch 2 This reaction was carried out in a 150 ml 3-necked RB flask fitted with a thermometer and air condenser. A solution of 3-chloro-4-(piperidin-4-yloxy)nitrobenzene (46.0 g, 0.179 mol), 90% formic acid (45.8 g, 0.895 mol) and 38% formaldehyde (29.0 g, 0.367 mol) was heated on a steam bath (internal temp. 90° C.) for 4 hours. Gas evolution was noted to cease after 3 hours. Conc. HCl (15.4 ml, 0.179 mol) was added and the batch then evaporated to a residue (63 g). The gum was dissolved in water (100 ml) and then basified with 5 N NaOH (55 ml). Some precipitation was observed. The mixture was heated at 80-90° C. for 20 minutes. Further 5 N NaOH (10 ml) was added, as the pH had wandered to 6. After cooling, the product was extracted with CHCl₃ (3×250 ml) and the organics back-washed with brine (1×100 ml). Following -drying with MgSO₄ and filtration, the solution was evaporated under vacuum to a pale lemon solid residue.

Yield: 41.6 g (85.8% theory). M.Pt. 102.6-103.2° C.

TLC: As per Batch 1

The products from Batches 1 and 2 were combined for subsequent reduction.

### d. Preparation of 3-chloro-4-(1-methyl-piperidin-4-yloxy)phenylamine

This reaction was carried out in a 2 l 3-necked RB flask fitted with a thermometer pocket, thermometer and magnetic follower, the flask being connected to a hydrogen reservoir system.

3-Chloro-(1-methyl-4-piperidin-4-yloxy)nitrobenzene (133.0 g, 0.491 mol) was dissolved in warm ethanol (1,330 ml) and the solution cooled to 20° C. After N₂ purging, 1% Pt/C catalyst (29.3 g wet, 63.66% water, Engelhard Code 43493) was added. The batch was hydrogenated over 6 hours 25 minutes at atmospheric pressure, an exotherm from 20 to 40° C. being noted. (Absorption was essentially complete after 5 hours 45 minutes).

After N₂ purging, the catalyst was removed by filtration, washed well with EtOH (1.0 l) nd the filtrate and washings vacuum concentrated to a pale lemon solid (118.6 g, 100% theory). The solid was digested with diethyl ether (100 ml) and the resulting white product washed with Et₂O (50 ml). The product was vacuum dried at 45° C. Concentration of the mother and wash liquors to 30 ml yielded a second crop of white solid of similar quality.

Total Yield: 105.4 g (89.2% theory) M.Pt. 99.9-100.8° C.

1H NMR (300 MHz, DMSO-d6) δ ppm 6.89 (d, 1H, J=8.7), 6.63 (s, 1H), 6.48 (dd, 1H, J=2.7, 8.7), 4.59 (br s, 2H), 4.06 (m, 1H), 2.63 (m,2H), 2.18 (m, 5H), 1.88 (m, 2H), 1.66 (m, 2H)

### R²NH₂for Example 30

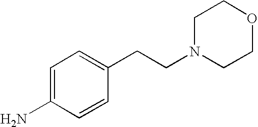

### a. 4-nitro-phenylethanol

A solution of 4-nitrophenyl acetic acid (150 g, 0.829 mole) in THF (1.5 L) was cooled to 0° C. Borane dimethyl sulfide (94.5 g, 118 mL, 1.24 mole) was added slowly over 60 minutes. The reaction was allowed to warm to room temperature and stirred at room temperature for 17 hours. The reaction was quenched by the addition of 1.5 N HCl (350 mL) and concentrated to a viscous liquid. The liquid was dissolved in DCM (1.5 L), washed with water (2×1 L) and brine (1×1 L), dried (Na₂SO₄), filtered and concentrated to provide the desired product as a pale yellow solid (130 g).

### b.

4-nitrophenylethanol (130 g, 0.7665 mole) and TEA (3.1 eq) were dissolved in DCM (2 L) and cooled to 0° C. in an ice bath. Tosyl chloride (175 g, 0.9198 mole) was then added in 5 equal portions over a period of 1 hour. The reaction mixture was allowed to warm to room temperature and stirred at room temperature for 17 hours. Water (1 L) was added and the layers separated. The organic layer was washed with water (3×1 L) and brine (1×1 L), dried (Na₂SO₄), filtered and concentrated to provide the tosylate as an off white solid (230 g).

### c.

The tosylate made in step b (230 g, 0.7165 mole) was dissolved in DMF (1.5 L) and treated with K₂CO₃ (118 g, 0.8598 mole) and then morpholine (126 mL, 1.43 mole). The reaction mixture was heated to 65° C. and stirred at that temperature for 3 hours. The reaction mixture was poured into ice water and extracted with EtOAc (2×1 L). The combined EtOAc layers were washed with water (3×1 L) and brine (1×1 L), dried (Na₂SO₄), filtered and concentrated to give the crude product as an orange liquid (150 g).

### d.

The niro compound made in step c (150 g) was dissolved in MeOH (2 L). FeCl₃ (7.5 g) and activated carbon (15 g) were added and the reaction mixture was heated to reflux. Hydrazine hydrate (300 mL) was added slowly over 1 hour, and then the reaction was refluxed for 4 hours. The reaction mixture was cooled to room temperature and filtered through celite. The filtrate was concentrated to a solid which was taken up in water (1 L) and cooled to 0° C. The precipitated solids were collected by filtration and dried to give the desired product as an off white solid (125 g).

1H NMR (300 MHz, DMSO-d6) δ ppm 6.87 (d, 2H, J=8.3), 6.5 (d, 2H, J=8.3), 4.84 (s, 2H), 3.59 (m, 4H), 2.56 (m, 2H), 2.41 (m, 6H)

### R²NH₂ for Example 31

### Synthesis of 4-(2-morpholin-4-yl-ethoxy)-aniline

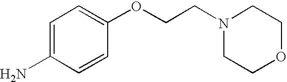

### a. 4-[2-(4-nitro-phenoxy)-ethyl]-morpholine

Sodium hydride (60%, 8.4 g) was suspended in THF (400 mL) and then treated with 4-(2-hydroxyethyl)morpholine (25 g, 0.191 mole) in portions (caution: foaming and gas evolution!). After the addition the mixture was stirred for 1 hour at room temperature, and then cooled to 0° C. 1-fluoro-4-nitro-benzene (26.95 g, 0.191 mole) was dissolved in THF (50 mL) and added dropwise to the stirring alkoxide. The reaction mixture was allowed to warm to room temperature and stirred overnight. The solvent volume was reduced by two thirds and diluted with water (1.5 L). This solution was extracted with DCM, dried (Na₂SO₄), filtered and concentrated to an oil. The oil was triturated with hexanes to give an orange-yellow solid which was filtered and washed with more hexanes (29 g).

### b.

The nitro compound (prepared as in step a, 80 g, 0.32 mole) was dissolved in MeOH (1.5 L) and added to a 4 L autoclave. To this was added 10% Pd/C (5 g) and the autoclave charged with hydrogen (30 atmospheres). The reaction was stirred overnight, then filtered to remove the catalyst. The filtrate was concentrated and distilled under vacuum to give an oil. The oil was taken up in hot cylohexane. The excess solvent was decanted off, and the solution cooled, and filtered. The solids were washed with hexanes to give the product (23.7 g)

1H NMR (300 MHz, DMSO-d6) δ ppm 6.67 (d, 2H, J=8.7), 6.53 (d, 2H, J=8.7), 4.62 (s, 2H), 3.95 (t, 2H, J=5.9), 3.6 (m, 4H), 2.64 (t, 2H, J=5.7), 2.47 (m, 4H)

### R²NH₂ for Example 32

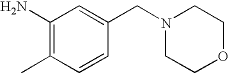

### a.

4-Methyl-3-nitrobenzyl chloride (13.03 g) in DCM (199 mL) was treated with TEA (10.45 mL) and morpholine (6.54 mL) and stirred at room temperature overnight. The organic layer was washed with water and brine and filtered through a hydrophobic frit. The solvent was removed to give the product as a yellow orange oil (16.04 g).

### b.

The product made in part a (16 g) was hydrogenated under standard conditions in EtOH (400 mL) with 10% Pd/C as catalyst (800 mg). Hydrogen uptake was fast, and the reaction was complete within 2 hours. The reaction was filtered and the solvent removed in vacuo to give the desired reduced material as a white solid (12.72 g).

1H NMR (300 MHz, DMSO-d6) δ ppm 6.86 (d, 1H, J=7.4), 6.6 (s, 1H), 6.41 (dd, 1H, J=1.3, 7.5), 4.78 (s, 2H), 3.58 (m, 4H), 3.29 (s, 2H), 2.33 (m, 4H), 2.04 (s, 3H)

### R²NH₂ for Example 33

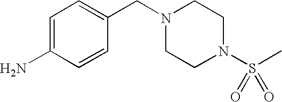

prepared by a method analogous to that found in:

Kuethe, Jeffrey T., et al, Journal of Organic Chemistry (2005), 70(7), 2555-2567

1H NMR (300 MHz, DMSO-d6) δ ppm 6.95 (d, 2H, J=8.3), 6.53 (d, 2H, J=8.3), 5.03 (brs, 2H), 3.35 (s, 2H), 3.11 (m, 4H), 2.89 (s, 3H), 2.53 (m, 4H)

### R²NH₂ for Example 34

### Synthesis of 3-amino-N-(3-pyridinylmethyl)benzamide

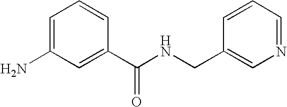

prepared in a manner similar to that found in B. R. Baker et al, J Med Chem (1970) volume 13, p. 280 and EP0596406

1H NMR (300 MHz, DMSO-d6) δ ppm 8.8 (t, 1H, J=6.1), 8.56 (s, 1H), 8.48 (dd, 1H, J=4.6, 1.1), 7.72 (d, 1H, J=7.9), 7.39 (m, 1H), 7.10 (m, 3H), 6.72 (dd, 1H, J=1.4, 7.4), 5.3 (br s, 2H), 4.47 (d, 2H, J=6.0)

### R²NH₂ for Example 35

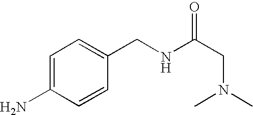

### a.

4-(Aminomethyl)-1-N-Boc-aniline (85 g, 0.3829 mole) was taken up in DCM (900 mL) and cooled to 0° C. TEA (46.4 mL, 0.45945 mole) was added, followed by chloroacetyl chloride (36.9 mL, 0.42117 mole). The temperature was allowed to warm to room temperature and the reaction mixture was stirred overnight. The reaction mixture was partitioned between water and DCM. The aqueous layer was extracted with DCM (2×450 mL). The combined organic layers were washed with water and brine, dried (Na₂SO₄), filtered and concentrated. The residue was purified by chromatography on silica using 10% EtOAc in petroleum ether to give the product (98 g).

### b.

The chloroacetamide intermediate made is step a was taken up in DMF (1 L) and treated with N,N-dimethylamine hydrochloride (39.7 g, 0.3249 mole) and K₂CO₃ (89.6 g, 0.6498 mole). The reaction mixture was heated at 60° C. overnight. The reaction mixture was quenched by the addition of water (200 mL) and then extracted with DCM (3×450 mL). The combined organic layers were washed with water (2×200 mL) and brine (2×200 mL), dried (Na₂SO₄), filtered and concentrated. The residue was purified by chromatography on silica eluting with 50% EtOAc in petroleum ether to afford the dimethyl amino product (65.5 g).

### c.

The intermediate prepared instep b (65 g) was dissolved in a dioxane/HCl solution (450 mL) and stirred at room temperature for 2 hours. The reaction mixture was concentrated and the residue free-based with a concentrated aqueous NaHCO₃ solution. This material was purified by chromatography on silica eluting with 10% MeOH in CHCl₃ to give the product as a light brown semi-solid (26.5 g).

1H NMR (300 MHz, DMSO-d6) δ ppm 7.98 (t, 1H, J=5.5), 6.95 (d, 2H, J=8.4), 6.52 (d, 2H, J=8.5), 4.89 (br s, 2H), 4.12 (d, 2H, J=6.1), 2.90 (s, 2H), 2.21 (s, 6H)

### R²NH₂ for Example 36

### Preparation of 1-(3-Aminophenyl)-4-methylpiperazin-2-one

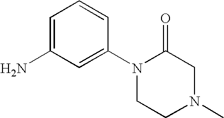

### a. Preparation of 2-Chloro-N-(3-nitrophenyl)acetamide

3-Nitroaniline (13.8 g, 0.1 mol) and triethylamine (11.1 g, 0.11 mol) were dissolved in 1,4-dioxane (50 mL) and cooled to 0° C. A solution of chloroacetyl chloride (11.3 g, 0.1 mol) in 1,4-dioxane (10 mL) was added dropwise over 30 min and the reaction mixture was stirred for 2 h before being poured into water (500 mL) and stirred for a further 30 mins. The resultant solid was filtered off, washed with water, and dried to give 14.05 g (65%) of 2-chloro-N-(3-nitrophenyl)acetamide: solid; mp 110-112° C.; R_{f} 0.61 (EtOAc).

### b. Preparation of N²-(2-Hydroxyethyl)-N-(3-nitrophenyl)glycinamide

2-Chloro-N-(3-nitrophenyl)acetamide (107 g, 0.5 mol) was dissolved in methanol (1.5 L) and cooled to 5° C. To this was added ethanolamine (305 g, 5.0 mol) with stirring over 10 min. The reaction mixture was then stirred at room temperature for 1 h, after which time a solid began to precipitate out. The reaction mixture was stirred overnight and then the solid was filtered off, washed with methanol and then hexane (250 mL), and dried under vacuum overnight to give 96.1 g (80%) of N²-(2-hydroxyethyl)-N-(3-nitrophenyl)glycinamide: mp 145-147° C.; R_{f} 0.31 (20% MeOH/DCM).

### c. Preparation of tert-Butyl 4-(3-Nitrophenyl)-3-oxopiperazine-1-carboxylate

N²-(2-Hydroxyethyl)-N-(3-nitrophenyl)glycinamide (59.8 g, 0.25 mol) was suspended in ethyl acetate (500 mL) and cooled to 5° C. To this was added tri-n-butylphosphine (86.2 mL, 0.32 mol) and then a solution of diisopropylazodicarboxylate (70.4 g, 0.32 mol) in EtOAc (160 mL) was added dropwise over 30 min, while the temperature of the reaction mixture was maintained <10° C. The reaction mixture was stirred overnight and then extracted with 1:1 water/brine (3×500 mL). The EtOAc layer was reserved and treated as below(*) To the aqueous solution was added di-tert-butyldicarbonate and the reaction mixture was stirred overnight. The resultant solid was filtered off and taken up into DCM, dried (MgSO₄), concentrated to a slurry, swirled with hexane, filtered and dried. (*) The EtOAc layer was acidified with 0.1 M HCl, and the aqueous layer was removed and neutralized (KOH) and to this was also added di-tert-butyldicarbonate and the above repeated to get a second crop of product. This gave 45 g (56%) of tert-butyl 4-(3-nitrophenyl)-3-oxopiperazine-1-carboxylate: solid; mp 122-123° C.

### d. Preparation of 1-(3-Nitrophenyl)piperazin-2-one

tert-Butyl 4-(3-nitrophenyl)-3-oxopiperazine-1-carboxylate (60 g, 0.19 mol) was added to a solution of trifluoroacetic acid (120 mL, 1.6 mol) in dichloromethane (480 mL) and the resultant reaction mixture was stirred for 2 h. The dichloromethane and trifluoroacetic acid were then removed under reduced pressure and the reaction mixture was triturated with toluene (100 mL). Dichloromethane (750 mL) was added to the residue and the mixture was made basific with sodium hydroxide solution. The organic fraction was separated and concentrated under reduced pressure to a slurry, to which was added hexane. The resultant precipitate was filtered off, washed with hexane, and dried overnight under reduced pressure to gave 36 g (86%) of 1-(3-nitrophenyl)piperazin-2-one: solid; mp 137-139° C.; R_{f} 0.33 (10% MeOH/DCM).

### e. Preparation of 4-Methyl-1-(3-nitrophenyl)piperazin-2-one

1-(3-Nitrophenyl)piperazin-2-one (2.5 g, 11.3 mmol) and 37% aqueous formaldehyde (0.92 g) were added to dichloromethane (35 mL) and to this was added, portionwise, sodium triacetoxyborohydride (9.58 g, 45.2 mmol). The reaction mixture was stirred for 2 h and was then quenched with saturated sodium bicarbonate solution and extracted with ethyl acetate. The organic layer was washed with saturated sodium bicarbonate and then it was dried and concentrated to an oil. To this was added a small amount of EtOAc and some hexane. The resultant solid was filtered off and dried to give 1.8 g (68%) of 4-methyl-1-(3-nitrophenyl)piperazin-2-one: R_{f} 0.40 (MeOH).

### f. Preparation of 1-(3-Aminophenyl)-4-methylpiperazin-2-one

4-Methyl-1-(3-nitrophenyl)piperazin-2-one (20.0 g. 0.085 mol) was dissolved in warm methanol (500 mL) and to this was added a paste of 5% palladium on carbon (3.0 g) in toluene in a 1 L autoclave. This was charged to 40 atmospheres with H₂ and stirred for 30 min. The catalyst was filtered off and the filtrate concentrated to a solid. The resultant residue was dissolved in dichloromethane, dried (MgSO₄), and concentrated until a solid began to precipitate. Hexane was then added with swirling and the resultant solid was filtered off, washed with hexane, and dried to give 16.2 g (93%) of 1-(3-aminophenyl)-4-methylpiperazin-2-one: solid; mp 139-140° C.; R_{f} 0.18 (3:1 EtOAc/MeOH).

1H NMR (300 MHz, DMSO-d6) δ ppm 7.04 (t, 1H, J=8.0), 6.48 (m, 2H), 6.42 (d, 1H, J=7.4), 5.19 (br s, 2H), 3.57 (t, 2H, J=5.2), 3.09 (s, 2H), 2.71 (t, 2H, J=5.6), 2.3 (s, 3H)

### R²NH₂ for Example 37

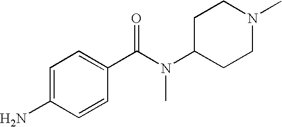

### a.

A solution of 1-BOC-4-methylaminopiperidine (120 g, 0.56 mole) in DCM (1.2 L) was treated with TEA. A solution of 4-nitrobenzoyl chloride (127.9 g, 0.68 mole) in DCM (250 mL) was then added and the reaction mixture was stirred overnight. The reaction mixture was washed with water (3×300 mL) and brine and concentrated to yellow solids (150 g).

### b.

The Boc-protected compound prepared in part a (150 g, 0.41 mole) was added to a solution of HCl in dioxane (750 mL) and the mixture was stirred at room temperature for 30 minutes, and then the solvent was evaporated. The crude product was taken up in dry acetonitrile (1 L) and treated with K₂CO₃ and the reaction mixture cooled to 0° C. Methyl iodide (24.9 mL, 0.493 mole) was added slowly over 30 minutes and the reaction stirred at room temperature overnight. The reaction mixture was filtered and concentrated. The concentrated filtrate was purified by chromatography on silica eluting with MeOH in CHCl₃ to give the product (55 g).

### c.

The material synthesized in part b (55 g, 0.198 mole) was dissolved in MeOH in a Paar shaker bottle and purged with nitrogen for 10 minutes. Pd/C was added and the reaction mixture was shaken under hydrogen (3 kg pressure) overnight. The reaction mixture was filtered through celite and the celite pad washed with MeOH. The solvent was evaporated to give the desired material (45 g).

1H NIR (300 MHz, DMSO-d6) δ ppm 7.11 (d, 2H, J=8.4), 6.56 (d, 2H, J=8.4), 5.47 (s, 2H), 3.87 (br s, 1H), 2.81 (m, 5H), 2.16 (s, 3H), 1.82 (m, 4H), 1.58 (m, 2H)

### R²NH₂ for Example 38

### Preparation of 3-(4-Aminophenyl)-imidazolidine-2,4-dione

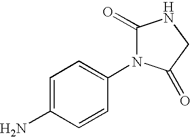

### a. Preparation of N-{[(4-Nitrophenyl)amino]carbonyl}glycine

Potassium hydroxide (6.8 g, 0.103 mol) was dissolved in water (80 mL) and cooled to 10° C. To this was added glycine (7.5 g, 0.1 mol) and then 1,4-dioxane (40 mL). 4-Nitrophenyl isocyanate (18.0 g, 0.11 mol) was added over 30 min and the resultant reaction mixture was stirred overnight. Water (700 mL) was added and the mixture was filtered through Celite. The filtrate was acidified with HCl (aq) and the resultant precipitate was filtered off, washed with water, and dried under reduced pressure over potassium hydroxide to give 21.0 g (88%) of N-{[(4-Nitrophenyl)amino]carbonyl}glycine: solid; mp 213-214° C.; R_{f} 0.55 (MeOH).

### b. Preparation of 3-(4-Nitrophenyl)imidazolidine-2,4-dione

N-{[(4-Nitrophenyl)amino]carbonyl}glycine (105.0 g, 0.44 mol) was suspended in a mixture of concentrated hydrochloric acid (400 mL) and water (500 mL) and refluxed in a 4 L flat bottomed flask for 4 h. The reaction mixture was then cooled and filtered and the solid was washed with water and dried to give 85.0 g (87%) of 3-(4-nitrophenyl)imidazolidine-2,4-dione: solid; mp 250-251° C.; R_{f} 0.43 (4:1 EtOAc/MeOH).

### c. Preparation of 3-(4-Aminophenyl)imidazolidine-2,4-dione

3-(4-Nitrophenyl)imidazolidine-2,4-dione (80.0 g, 0.36 mol) was suspended in methanol (4 L) and placed in a 7.5 L autoclave. To this was added a paste of 5% palladium on carbon (5.0 g) in toluene and the mixture was heated to 50° C. The vessel was then charged to 40 atmospheres with H₂ and stirred for 1 h at 60° C. After the required amount of hydrogen (7.4 atm) had been absorbed, the catalyst was filtered off (Celite) and washed through with lots of warm methanol. The methanol was removed under reduced pressure to a slurry and diethyl ether was added to the residue. The resultant solid was filtered off and dried to give 54.9 g (79%) of 3-(4-Aminophenyl)imidazolidine-2,4-dione: solid; mp 230-231° C.; R_{f} 0.58 (MeOH).

1H NMR (300 MHz, DMSO-d6) δ ppm 8.15 (s, 1H), 6.93 (d, 2H, J=8.6), 6.62 (d, 2H, J=8.7), 5.29 (br s, 2H), 4.03 (s, 2H)

### R²NH₂ for Example 39

### Preparation of 3-(3-Aminophenyl)imidazolidine-2,4-dione

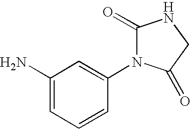

### a. Preparation of N-{[(3-Nitrophenyl)amino]carbonyl}glycine

Potassium hydroxide (10.2 g, 0.154 mol) and glycine (11.25 g, 0.15 mol) were dissolved in water and cooled to 10° C. To this was added 3-nitrophenyl isocyanate (25.0 g, 0.15 mol) over 30 mins. The reaction mixture was stirred overnight. Water (80 mL) was added and the reaction mixture was filtered through Celite and the filtrate acidified with 2M HCl. The product was filtered off and washed with water, then dried to give 31.5 g (88%) of N-{[(3-nitrophenyl)amino]carbonyl}glycine: solid; mp 217-218° C., R_{f} 0.40 (EtOAc/MeOH 3:1).

### b. Preparation of 3-(3-Nitrophenyl)imidazolidine-2,4-dione

N-{[(3-nitrophenyl)amino]carbonyl}glycine (126.0 g, 0.53 mol) was added to a mixture of concentrated HCl (400 mL) and water (400 mL) and refluxed for 4 h. The reaction mixture was cooled to room temperature and the solid was filtered off, washed, and dried to give 100.0 g (85%) of 3-(3-nitrophenyl)imidazolidine-2,4-dione: solid; mp 161-162° C., R_{f} 0.38 (EtOAc/MeOH 3:1).

### c. Preparation of 3-(3-Aminophenyl)imidazolidine-2,4-dione

3-(3-Nitrophenyl)imidazolidine-2,4-dione (95.0 g, 0.43 mol) was suspended in methanol (4 L) and placed in a 7.5 L autoclave. To this was added a paste of 5% palladium on carbon (5.0 g) in toluene and the reaction mixture was heated to 50° C. The vessel was then charged to 40 atmospheres with H₂ and stirred for 1 h @ 60-70° C. after which time the required amount of hydrogen (9 atm) had been absorbed. The catalyst was filtered off and washed through with MeOH. The filtrate was concentrated to a thick slurry, diethyl ether was added, and the solid filtered off, washed with diethyl ether, and dried to give 68.77 g (84%) of 3-(3-aminophenyl)imidazolidine-2,4-dione: solid; mp 155-156° C., R_{f} 0.58 (MeOH).

1H NMR (300 MHz, DMSO-d6) δ ppm 8.22 (s, 1H), 7.09 (t, 1H, J=8.1), 6.52 (m, 2H), 6.45 (d, 1H, J=7.7), 5.28 (s, 2H), 4.06 (s, 2H)

### R²NH₂ for Example 40

### Preparation of 3′-Amino-2-(dimethylamino)acetanilide

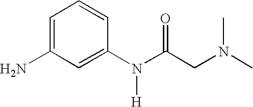

### a.

A solution of 3-nitro-aniline (100 g, 0.724 mole) in DCM (1 L) was treated with TEA (120 mL, 0.8688 mole) and cooled to 0° C. Chloroacetyl chloride (89.9 g, 0.7964 mole) was added dropwise. After the addition was complete, the reaction mixture was allowed to warm to room temperature and stirred overnight. The reaction mixture was diluted with water (1 L). The organic layer was separated and washed with brine (1×500 mL), dried (Na₂SO₄), filtered and concentrated. The crude product was purified by chromatography on silica eluting with 15% EtOAc:petroleum ether to give the desired amide (110 g).

### b.

A solution of the chloroacetamide prepared in step a (135 g, 0.6290 mole) in DMF (600 mL) was treated with K₂CO₃ (217 g, 1.5725 mole) and then dimethylamine hydrochloride (76.8 g, 0.9425 mole). The reaction mixture was heated to 60° C. and stirred for 3 hours. The reactionmixture was cooled to room temperature, diluted with water (2 L), and extracted with EtOAc (2 L). The organic layer was washed with water (1×2 L) brine (1×1 L), dried (Na₂SO₄), filtered and concentrated to a thick brown oil (125 g) which was used in the next step.

### c.

The nitro compound product from step b (120 g, 0.5375 mole) was dissolved in MeOH (700 mL), and treated with Pd/C while under nitrogen. The mixture was stirred in a Parr shaker bottle under 2 kg of hydrogen pressure overnight. The reaction mixture was filtered and concentrated. The thick brown oil obtained was purified by chromatography on silica using chloroform, EtOAc and MeOH to give the desired product (70 g).

1H NMR (300 MHz, DMSO-d6) δ ppm 9.35 (s, 1H), 7.0 (t, 1H, J=2), 6.93 (t, 1H, J=8), 6.7 (d, 1H, J=8.8), 6.28 (dd, 1H, J=1.6, 7.9) 5.07 (s, 2H), 3.03 (s, 2H), 2.29 (s, 6H)

### R²NH₂ for Example 41

### Preparation of 3-Amino-N-(3-dimethylaminopropyl)benzenesulfonamide

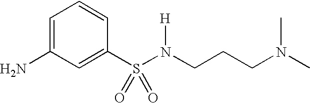

### a. Preparation of N-(3-dimethylaminopropyl)-3-nitrobenzenesulfonamide

A solution of 3-nitrobenzenesulfonyl chloride (110.0 g, 0.50 mol) in tetrahydrofuran (200 mL) was added dropwise to a solution of 3-dimethylaminopropylamine (53.2 g, 0.52 mol) and triethylamine (90 mL) in tetrahydrofuran (600 mL) at 5° C., while the temperature of the reaction mixture was maintained at 5° C. The reaction mixture was then allowed to warm to room temperature and was stirred for 1 h. Water (500 mL) was added, most of the tetrahydrofuran was removed under reduced pressure, and the mixture was extracted with EtOAc (2×1000 mL). The combined organic fractions were washed with brine, dried (MgSO₄), filtered, and concentrated to give an oil Addition of hexane gave a solid, which was filtered off to give 130.0 g (91%) of N-(3-dimethylaminopropyl)-3-nitrobenzenesulfonamide: pale yellow solid; mp 84-87° C., R_{f} 0.20 (MeOH).

### b. Preparation of 3-Amino-N-(3-dimethylaminopropyl)benzenesulfonamide

N-(3-Dimethylaminopropyl)-3-nitrobenzenesulfonamide (65.0 g, 0.23 mol) was dissolved in methanol (750 mL) and the resultant solution was place in a 1 L stainless steel autoclave. A paste of palladium on carbon (1.5 g) in toluene was then added and the autoclave was charged with 70 atmospheres of H₂ gas. After 2 h, the resultant mixture was filtered through celite and the filtrate was evaporated under reduced pressure. The crude off white solid was triturated in toluene and dried for 2 days in a vacuum oven (30° C.) to give 56.8 g (98%) of 3-amino-N-(3-dimethylaminopropyl)benzenesulfonamide: pale yellow solid; mp 89-93° C.; R_{f} 0.10 (MeOH).

1H NMR (300 MHz, DMSO-d6) δ ppm 7.39 (t, 1H, J=5.7), 7.21 (t, 1H, J=7.9), 7.0 (s, 1H), 6.89 (d, 1H, J=7.7), 6.77 (d, 1H, J=1.8, 7.8), 5.59 (s, 2H), 2.76 (m, 2H), 2.18 (m, 2H), 2.08 (s, 6H), 1.5 (m, 2H)

### R²NH₂ for Example 42

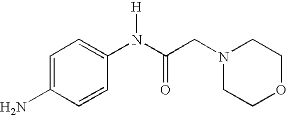

This material was prepared using a method similar to that for the synthesis of the R²NH₂ for example 40, but starting with 4-nitro-aniline instead of 3-nitro-aniline, and using morpholine instead of dimethylamine.

1H NMR (300 MHz, DMSO-d6) δ ppm 9.31 (s, 1H), 7.26 (d, 2H, J=8.7), 6.52 (d, 2H, J=8.8), 4.9 (s, 2H), 3.66 (m, 4H), 3.06 (s, 2H), 2.51 (m, 4H)

### Examples 43-55

### Method C (Scheme 3)

### Preparation for Compounds 43-55

### Step 1: Synthesis of 2-(4-methoxy-aniline)-4-chloro-pyrimidine

### Step 1, Part A

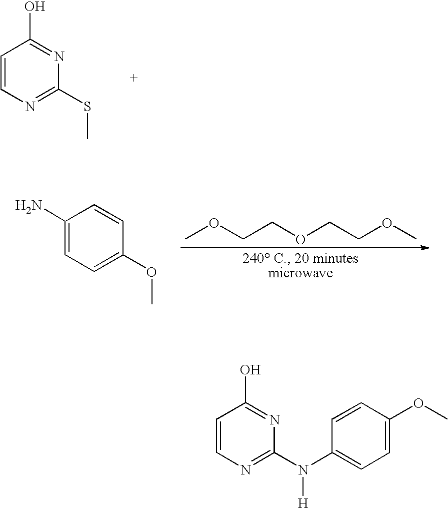

2-(methylthio)pyrimidin-4(3H)-one (800 mg, 5.63 mmol) (preparation in J. Spychala, Synthetic Communications, 1997, 27 (11), 1943) and para-anisidine (768 mg, 6.23 mmol) were combined in bis-(2-methoxyethyl) ether and heated at 240° C. for 20 minutes in a Smith Synthesizer microwave apparatus. The reaction mixture was diluted with 1:1 Et2O:hexanes and the precipitated solids collected by filtration, then washed with hexanes to give the product as white solids (747 mg, 61%).

1H NMR (300 MHz, DMSO-d6) δ ppm 10.8 (br s, 1H), 8.69 (br s, 1H), 7.71 (d, 1H, J=5.7), 7.49 (d, 2H, J=8.9), 6.92 (d, 2H, J=8.8), 5.76 (d, 1H, J=6.4), 3.76 (s, 3H)

The reaction was then repeated 4 more times at the same scale in the Smith Synthesizer microwave apparatus. The reactions were combined and worked up as above to give the product as white solids (3.04 g, 62%).

### Step 1, Part B

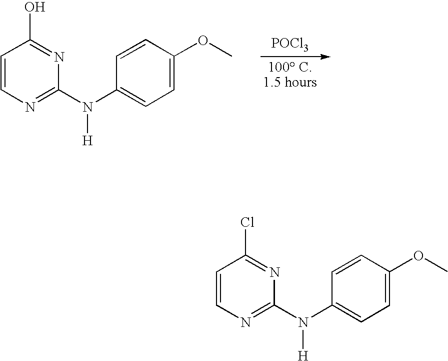

2-(4-methoxy-aniline)-4-hydroxy-pyrimidine (3.8 g, 17.5 mmol) was treated with POCl₃ (25 mL) and heated at 100° C. for 90 minutes. The reaction mixture was allowed to cool and then poured slowly and cautiously into stirring ice water. This mixture was stirred for 1 hour, and then extracted with EtOAc (2×200 mL). The combined organic layers were washed with half saturated aqueous NaHCO₃, dried (MgSO₄), filtered, and concentrated to brown solids. The solids were stirred in Et₂O, and filtered to give tan solids. The Et₂O filtrate was evaporated, and the resulting solids were re-suspended in Et₂O and filtered to give another crop of product. This procedure was repeated to give a third crop. The three crops were combined to give the desired 2-(4-methoxy-aniline)-4-chloro-pyrimidine as tan solids (2.84 g, 69%).

1H NMR (300 MHz, DMSO-d6) δ ppm 9.86 (s, 1H), 8.41 (d, 1H, J5.2), 7.60 (d, 2H), J=8.9), 6.91 (m, 3H), 3.76 (s, 3H)

### Step 2: Synthesis of Boronate Amide Intermediate for Example 43

### Preparation of N-(3-methoxybenzyl)-5-(4,4,5,5-tetramethyl-1,3,2-dioxaborolan-2-yl)-2-thiophenecarboxamide

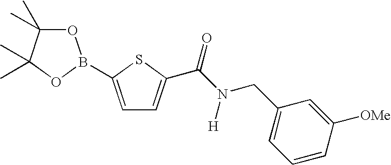

A mixture of 5-(4,4,5,5-tetramethyl-1,3,2-dioxaborolan-2-yl)-2-thiophenecarboxylic acid (10.1 g, 39.7 mmol), HOBt (6.43 g, 47.6 mmol), and EDC (9.13 g, 47.6 mmol), in DMF (100 mL) was treated with 3-methoxybenzylamine (5.6 mL, 43.7 mmol) and stirred at room temperature for 20 hours. The reaction mixture was poured onto ice water (300 mL) and extracted with EtOAc (3×150 mL). The combined organic layers were washed with brine, dried (MgSO₄), filtered and concentrated to afford the product, N-(3-methoxybenzyl)-5-(4,4,5,5-tetramethyl-1,3,2-dioxa-borolan-2-yl)-2-thiophenecarboxamide, (12.9 g) as a yellow solid.

1H NMR (400 MHz, DMSO-d6) δ ppm 1.26 (s, 12H), 3.71 (s, 3 H), 4.39 (d, J=6.0 Hz, 2H), 6.78-6.85 (m, 3H), 7.21 (t, J=8.0 Hz, 1H), 7.51 (d, J=3.7 Hz, 1H), 7.80 (d, J=3.7 Hz, 1 H), 9.09 (t, J=6.0 Hz, 1 H).

### Step 2: Synthesis of Boronate Amide Intermediates for Examples 44-55

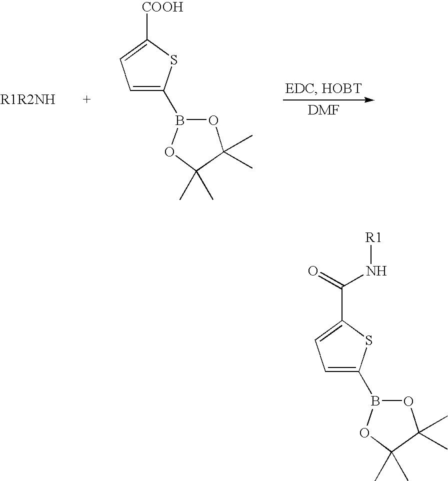

5-(4,4,5,5-tetramethyl-1,3,2-dioxaborolan-2-yl)-2-thiophene-carboxylic acid (4.2 g, 16.8 mmol), EDC (2.73 g, 20.16 mmol), and HOBT (3.86 g, 20.16 mmol) were combined in DMF (70 mL). Aliquots of this solution (5 mL) were put into test tubes equipped with stir bars. The appropriate amine (1.32 mmol) was added, and the reactions were stirred for 20 hours at room temperature. Each reaction was diluted with EtOAc (30 mL) and H₂O (30 mL). The aqueous organic mixture was stirred vigorously and the layers were allowed to separate. The aqueous layer was removed with a pipette. Another aliquot of H₂O (20 mL) was added, and the mixture stirred vigorously again. The aqueous layer was once again removed with a pipette. The organic layer was dried (MgSO₄), filtered, and concentrated to give the crude products as oils, and were used directly in the next step.

### Amines Used and Crude Yields Obtained:

| Amine name | Amount used | Crude yield of amides |
|---|---|---|
| 2-F-benzyl-amine | 1.32 mmol, 165 mg | 381 mg, 88% |
| 3-F-benzyl-amine | 1.32 mmol, 165 mg | 362 mg, 84% |
| 3-F-benzyl-amine | 1.32 mmol, 165 mg | 388 mg, 90% |
| 2-Cl-benzyl-amine | 1.32 mmol, 187 mg | 327 mg, 72% |
| 3-Cl-benzyl-amine | 1.32 mmol, 187 mg | 428 mg, 94% |
| 4-Cl-benzyl-amine | 1.32 mmol, 187 mg | 512 mg, 113% |
| 2-MeO-benzyl-amine | 1.32 mmol, 172 uL | 375 mg, 84% |
| 4-MeO-benzyl-amine | 1.32 mmol, 172 uL | 309 mg, 69% |
| 1-aminoindan | 1.32 mmol, 175 uL | 400 mg, 90% |
| Phenethyl amine | 1.32 mmol, 165 uL | 426 mg, 99% |
| (R) alpha-methyl benzyl | 1.32 mmol, 168 uL | 419 mg, 98% |
| amine | | |
| (S) alpha-methyl benzyl | 1.32 mmol,, 168 uL | 536 mg, 125% |
| amine | | |

### Step 3: Synthesis of Example 43

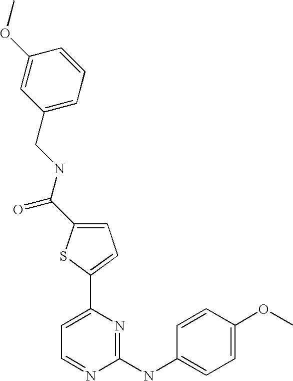

The amide boronate (172 mg, 0.46 mmol) and 2-(4-methoxy-aniline)-4-chloro-pyrimidine (100 mg, 0.42 mmol) in DME (2 mL) and EtOH (1 mL) were treated with 2N aqueous Na₂CO₃ (0.25 mL, 0.5 mmol) and Pd(Ph₃P)₂Cl₂ (14 mg, 0.21 mmol) and heated at 170° C. for 900 seconds in a Smith Synthesizer microwave. Silica gel was added to the reaction vessel and the solvent evaporated, followed by purification by silica gel chromatography eluting with a hexane:EtOAc gradient. The appropriate fractions were combined to give the product as yellow solids (134 mg).

1H NMR (300 MHZ, DMSO-d6) δ ppm 9.55 (s, 1H), 9.21 (m, 1H), 8.51 (d, 1H, J=5.1), 8.01 (d, 1H, J=4), 7.89 (d, 1H, J=3.9), 7.73 (d, 2H, J=9), 7.32 (m, 2H), 6.92 (m, 5H), 4.48 (d, 2H, J=5.9), 3.77 (s, 6H)

### Step 3: Synthesis of Examples 44-55

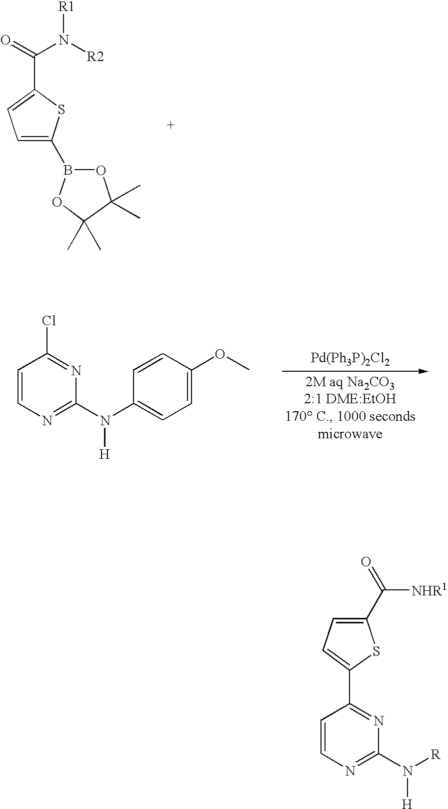

The crude amide boronates prepared in step 2 (<1.32 mmol) in DME (2 mL) in microwave reaction vessels were treated with 2-(4-methoxy-aniline)-4-chloro-pyrimidine (70 mg, 0.3 mmol) and Pd(Ph₃P)₂Cl₂ (10.5 mg, 0.015 mmol). EtOH (1 mL) and 2N aqueous Na₂CO₃ (180 uL, 0.36 mmol) were added to each reaction vessel, and caps were crimped on to the vessels. The reactions were then heated in the microwave (Smith Synthesizer) at 170° C. for 1000 seconds. The crude reaction mixtures were then concentrated in the presence of silica gel, and purified by regular phase chromatography on silica using hexane:EtOAc gradient elution.

### Example 44

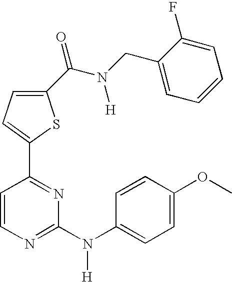

1H NMR (300 MHZ, DMSO-d6) δ ppm 9.56 (s, 1H), 9.22 (t, 1H, J=5.7), 8.52 (d, 1H, J=5), 8.01 (d, 1H, J=4.1), 7.91 (d, 1H, J=4.1), 7.73 (d, 2H, J=9), 7.33-7.46 (m, 3H), 7.23 (m, 2H), 6.93 (d, 2H, J=9.2), 4.55 (d, 2H, J=5.6), 3.77 (s, 3H)

### Example 45

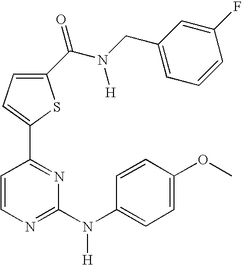

1H NMR (300 MHZ, DMSO-d6) δ ppm 9.56 (s, 1H), 9.28 (t, 1H, J=5.7), 8.52 (d, 1H, J=5.2), 8.01 (d, 1H, J=5), 7.90 (d, 1H, J=3.9), 7.73 (d, 2H, J=8.9), 7.34-7.5 (m, 2H), 7.16 (m, 3H), 6.93 (d, 2H, J=9.1), 4.52 (d, 2H, J=5.9), 3.77 (s, 3H)

### Example 46

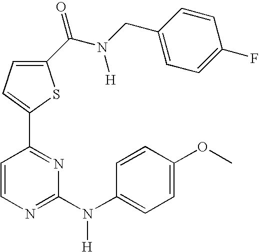

1H NMR (300 MHZ, DMSO-d6) δ ppm 9.55 (s, 1H), 9.24 (t, 1H, J=5.9), 8.52 (d, 1H, J=5.2), 8.0 (d, 1H, J=4), 7.88 (d, 1H, J=4), 7.73 (d, 2H, J=9), 7.33-7.48 (m, 3H), 7.2 (m, 2H), 6.93 (d, 2H, J=9.1), 4.49 (d, 2H, J=5.9), 3.77 (s, 3H)

### Example 47

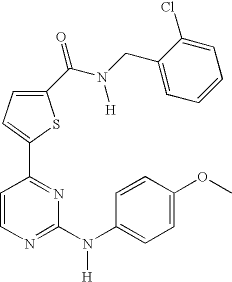

1H NMR (300 MHZ, DMSO-d6) δ ppm 9.56 (s, 1H), 9.25 (d, 1H, J=5.8), 8.53 (d, 1H, J=5.1), 8.01 (d, 1H, J=3.9), 7.94 (d, 1H, J=4.1), 7.72 (d, 2H, J=9), 7.33 7.52 (m, 5H), 6.93 (d, 2H, J=9.2), 4.58 (d, 2H, J=5.8), 3.77 (s, 3H)

### Example 48

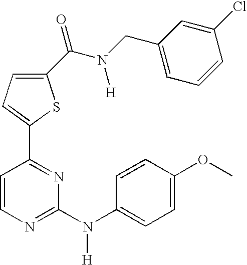

1H NMR (300 MHZ, DMSO-d6) δ ppm 9.56 (s, 1H, 9.28 (t, 1H, J=6.1), 8.52 (D, 1h, j=5), 8.01 (D, 1h, j=3.9), 7.89 (d, 1H, J=3.9), 7.72 (d, 2H, J=9), 7.38 (m, 5H), 6.93 (d,2H, J=9), 4.51 (d, 2H, J=5.8), 3.77 (s, 3H)

### Example 49

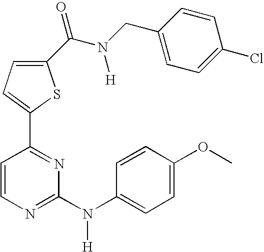

1H NMR (300 MHZ, DMSO-d6) δ ppm 9.56 (s, 1H), 9.26 (t, 1H, J=5.9), 8.52 (d, 1H, J=5), 8.01 (d, 1H, J=3.9), 7.88 (d, 1H, J=4), 7.72 (d, 2H, J=9), 7.33-7.49 (m, 5H), 6.93 (d, 2H, J=9.2), 4.49 (d, 2H, J=5.9), 3.77 (s, 3H)

### Example 50

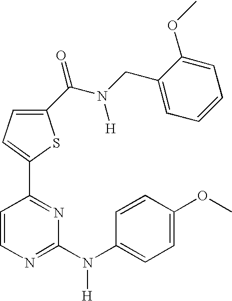

1H NMR (300 MHZ, DMSO-d6) δ ppm 9.55 (s, 1H), 9.07 (t, 1H, J=5.8), 8.52 (d, 1H, J=5.2), 8.01 (d, 1H, J=4.1), 7.93 (d, 1H, J=4.1), 7.73 (d, 2H, J=9.1), 7.24-7.35 (m, 3H), 6.92-7.05 (m, 4H), 4.48 (d, 2H, J=5.8), 3.87 (s, 3H), 3.77 (s, 3H)

### Example 51

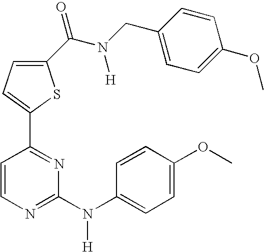

1H NMR (300 MHZ, DMSO-d6) δ ppm 9.55 (s, 1H), 9.17 (t, 1H, J=6), 8.51 (d, 1H, J=5.2), 8.00 (d, 1H, J=3.9), 7.87 (d, 1H, J=4), 7.72 (d, 2H, J=8.9), 7.29 (m, 2H), 6.93 (d, 4H, J=8.8), 4.43 (d, 2H, J=5.8), 3.77 (s, 3H), 3.76 (s, 3H)

### Example 52

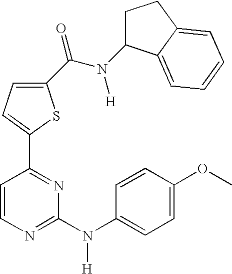

1H NMR (300 MHZ, DMSO-d6) δ ppm 9.56 (s, 1H), 9.0 (d,1H, J=8.4), 8.52 (d, 1H, J=5.2), 8.00 (d, 1H, J=4.1), 7.92 (d, 1H, J=3.9), 7.73 (d, 2H, J=8.9), 7.23-7.33 (m, 5H), 6.95 (d, 2H, J=9), 5.57 (m, 1H), 3.78 (s, 3H), 3.03 (m, 1H), 2.93 (m, 1H), 2.50 (m, 1H), 2.02 (m, 1H)

### Example 53

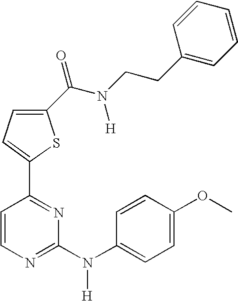

1H NMR (300 MHZ, DMSO-d6) δ ppm 9.55 (s, 1H), 8.79 (t, 1H, J=5.6), 8.52 (d, 1H, J=5.1), 7.99 (d, 1H, J=3.9), 7.80 (d, 1H, J=3.9), 7.73 (d, 2H, J=9), 7.22-7.37 (m, 6H), 6.95 (d, 2H, J=9), 3.78 (s, 3H), 3.51 (m, 2H), 2.89 (t, 2H, J=7.2)

### Example 54

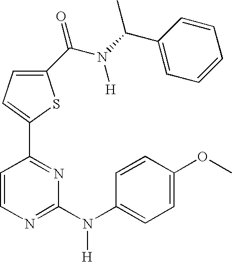

1H NMR (300 MHZ, DMSO-d6) δ ppm 9.54 (s, 1H), 9.0 (d, 1H, J=8), 8.51 (d, 1H, J=5.2), 8.0 (m, 2H), 7.72 (d, 2H, J=8.9), 7.25-7.44 (m, 6H), 6.93 (d, 2H, J=9), 5.17 (m, 1H), 3.76 (s, 3H), 1.53 (d, 3H, J=7.1)

### Example 55

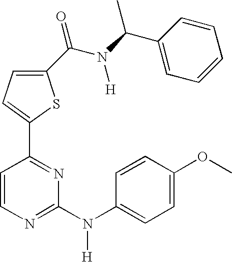

1H NMR (300 MHZ, DMSO-d6) δ ppm 9.54 (s, 1H), 9.0 (d, 1H, J=8), 8.51 (d, 1H, J=5.2), 8.0 (m, 2H), 7.72 (d, 2H, J=9), 7.27-7.44 (m, 6H), 6.92 (d, 2H, J=8.9), 5.17 (m, 1H), 3.76 (s, 3H), 1.53 (d, 3H, J=7.2)

### Example 56

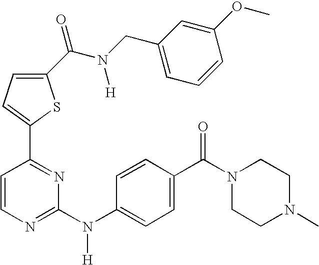

Prepared in a Manner Similar to that Used for Examples 8-42

1H NMR (300 MHZ, DMSO-d6) δ ppm 9.94 (s, 1H), 9.25 (t, 1H, J=5.8), 8.54 (d, 1H, J=5.3), 7.99 (d, 1H, J=4), 7.88 (m, 3H), 7.41 (d, 1H, J=5.3), 7.35 (d, 2H, J=8.4), 7.23 (t, 1H, J=8), 6.87 (m, 2H), 6.8 (m, 1H), 4.41 (d, 2H, J=5.0), 3.71 (s, 3H), 3.3 (br, 4H), 2.5 (br, 4H), 2.3 (br, 3H)

### Example 57

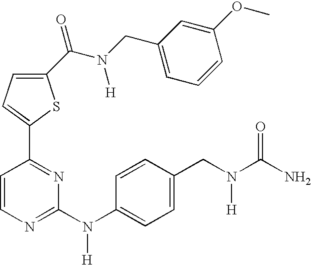

Prepared in a Manner Similar to that Used for Examples 8-42

1H NMR (300 MHZ, DMSO-d6) δ ppm 9.67 (s, 1H), 9.19 (t, 1H, J=6), 8.51 (d, 1H, J=5.1), 7.97 (d, 1H, J=4), 7.86 (d, 1H, J=4.1), 7.73 (d, 2H, J=8.6), 7.35 (d, 1H, J=5.1), 7.25 (t, 1H, J=6.2), 7.18 (d, 2H, J=8.6), 6.83 (m, 3H), 6.33 (t, 1H, J=6.1), 5.48 (s, 2H), 4.44 (d, 2H, J=5.8), 4.11 (d, 2H, J=5.9), 3.72 (s, 3H)

### Example 58

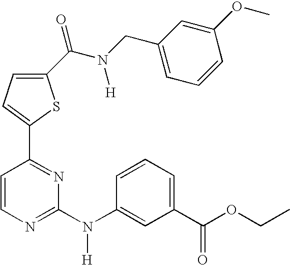

Prepared in a Manner Similar to that Used for Examples 8-42

1H NMR (300 MHZ, DMSO-d6) δ ppm 9.96 (s, 1H), 9.18 (t, 1H, J=6), 8.62 (s, 1H), 8.56 (d, 1H, J=5.1), 8.01 (d, 1H, J=4), 7.95 (d, 1H, J=8.3), 7.86 (d, 1H, J=4), 7.56 (d, 1H, J=7.7), 7.42 (m, 2H), 7.24 (t, 1H, J=8), 6.88 (m, 2H), 6.80 (m, 1H), 4.44 (d, 2H, J=6), 4.35 (q, 2H, J=7.1), 3.72 (s, 3H), 1.29 (t, 3H, J=7.1)

The following Experimental Section also describes compounds of the present invention. This section is divided into the following subsections.

### Subsection 1: Preparation of Intermediates (a)

These amine intermediates relate to the preparation of compounds providing the —NHR² portion of the compound of formula (I). These intermediates may be used in the “Preparation of Examples” subsections below. Other amine intermediates are commercially available.

### Subsection 2: Preparation of Intermediates (b)

These intermediates relate to the preparation of compounds providing the —NHR¹ portion of the compound of formula (I). The intermediates may be used in the “Preparation of Examples” subsections below. Other intermediates are commercially available.

### Subsection 3: Examples Prepared by Guanidine Route

### Subsection 4: Examples Prepared by the Maine Route

### Subsection 5: Examples Prepared by Alternative Route

### Subsection 1: Preparation of Intermediates (a) (Amines)

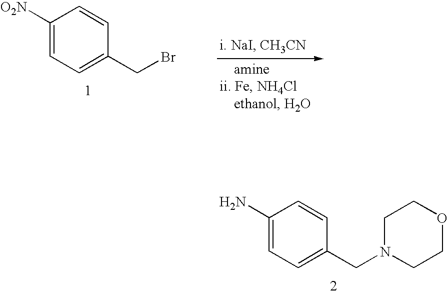

### Synthesis of 2

To a solution of 1 (5.0 g, 23 mmol) in acetonitrile (100 mL) was added amine (4.0 g, 46 mmol) and cat. Nal. The resulting mixture was stirred for 14 h, the solids were filtered and the filtrate was concentrated under reduced pressure. The crude reaction was dissolved in ethanol (100 mL) and H₂O (50 mL) followed by the addition of iron powder (6.4 g, 110 mmol) and ammonium chloride (1.5 g, 28 mmol). The reaction mixture was heated to reflux for 2 h, cooled and filtered to remove the solids. The filtrate was concentrated under reduced pressure dissolved in 6 N HCl (20 mL) and made basic by the addition of 6 N NaOH. The basic aqueous layer was extracted with ethyl acetate (100 mL), dried over Na₂SO₄ and concentrated to obtain 2 (1.9 g, 43%) as a white solid: ¹H NMR (500 MHz, CDCl₃) δ 7.09 (d, J=8.5 Hz, 2H), 6.63 (d, J=8.5 Hz, 2H), 3.72-3.68 (m, 4H), 3.62 (bs, 2H), 3.38 (s, 2H), 2.42-2.40 (m, 4H).

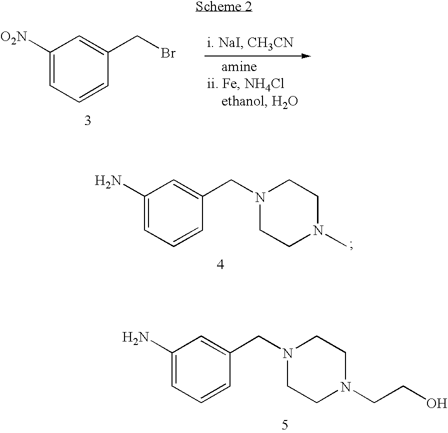

### Synthesis of 4

To a solution of 3 (5.0 g, 23 mmol) in acetonitrile (100 mL) was added amine (4.0 g, 46 mmol) and cat. Nal. The resulting mixture was stirred for 14 h, the solids were filtered and the filtrate was concentrated under reduced pressure. The crude reaction was dissolved in ethanol (100 mL) and H₂O (50 mL) followed by the addition of iron powder (6.4 g, 110 mmol) and ammonium chloride (1.5 g, 28 mmol). The reaction mixture was heated to reflux for 2 h, cooled and filtered to remove the solids. The filtrate was concentrated under reduced pressure dissolved in 6 N HCl (20 mL) and made basic by the addition of 6 N NaOH. The basic aqueous layer was extracted with ethyl acetate (100 mL), dried over Na₂SO₄ and concentrated to obtain 4 (1.7 g, 36%) as a light brown solid: ¹H NMR (500 MHz, CDCl₃) δ 7.08 (t, J=7.5 Hz, 1H), 6.70-6.67 (m, 2H), 6.58-6.55 (m, 1H), 3.62 (bs, 2H), 3.41 (s, 2H), 2.62-2.27 (m, 11H).

### Synthesis of 5

To a solution of 3 (5.0 g, 23 mmol) in acetonitrile (100 mL) was added amine (4.0 g, 46 mmol) and cat. Nal. The resulting mixture was stirred for 14 h, the solids were filtered and the filtrate was concentrated under reduced pressure. The crude reaction was dissolved in ethanol (100 mL) and H₂O (50 mL) followed by the addition of iron powder (6.4 g, 110 mmol) and ammonium chloride (1.5 g, 28 mmol). The reaction mixture was heated to reflux for 2 h, cooled and filtered to remove the solids. The filtrate was concentrated under reduced pressure dissolved in 6 N HCl (20 mL) and made basic by the addition of 6 N NaOH. The basic aqueous layer was extracted with ethyl acetate (100 mL), dried over Na₂SO₄, concentrated and purified by chromatography 9silica gel, 0-20% methanol/methylene chloride) to obtain 5 (1.6 g, 31%) as an off-white solid: ¹H NMR (500 MHz, CDCl₃) δ 7.08 (t, J=7.5 Hz, 1H), 6.70-6.67 (m, 2H), 6.59-6.56 (m, 1H), 3.68-3.58 (m, 4H), 3.42 (s, 2H), 2.88-2.37 (m, 10H).

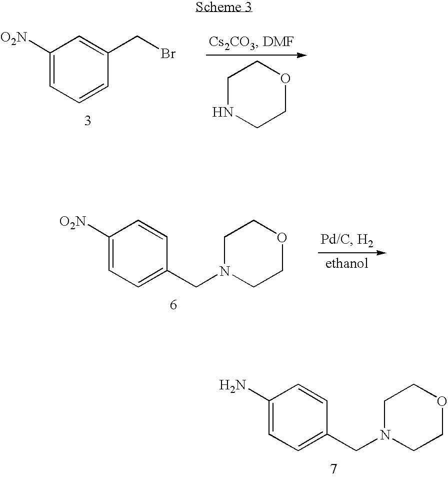

### Synthesis of 6

To a solution of 3 (7.0 g, 32 mmol) in DMF (80 mL) was added cesium carbonate (13 g, 39 mmol) and amine (3.3 g, 39 mmol). The resulting mixture was stirred for 4 d, quenched by the addition of H₂O (150 mL) and extracted with ethyl acetate (2×50 mL). The organics were dried over Na₂SO₄, concentrated and purified by chromatography (silica gel, 0-5% methanol/methylene chloride) to obtain 6 (3.8 g, 53%) as a yellow solid: ¹H NMR (500 MHz, CDCl₃) δ 8.21 (s, 1H), 8.13-8.10 (m, 1H), 7.69-7.67 (m, 1H), 7.51-7.48 (m, 1H), 3.75-3.69 (m, 4H), 3.57 (s, 2H), 2.49-2.43 (m, 4H).

### Synthesis of 7

To a solution of 3 (3.8 g, 17 mmol) in ethanol (100 mL) was added cat. 10 wt % Pd/C and the reaction mixture was hydrogenated (30 psi) for 45 min. The catalyst was filtered through diatomaceous earth and the filtrate was concentrated and purified by chromatography (silica gel, 0-5% methanol/methylene chloride) to obtain 7 (1.4 g, 42%) as an off-white solid: ESI MS m/z 193 [C₁₁H₁₆N₂O+H]⁺;

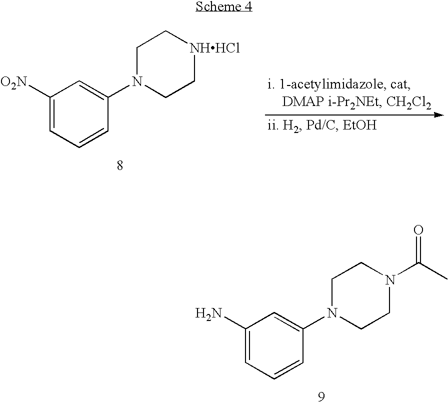

### Synthesis of 9

Step 1. 1-Acetylimidazole (252 mg, 2.60 mmol) was added portion wise at room temperature to a stirred solution of 8 (500 mg, 2.37 mmol) in methylene chloride (17 mL) containing N,N-diisopropylethylamine (1.4 mL, 7.8 mmol) and DMAP (55 mg, 0.26 mmol). The reaction was stirred for 18 h, diluted with methylene chloride and washed with satd. aq. NaHCO₃, water and, brine, dried over sodium sulfate and concentrated under reduced pressure. The residue was purified by chromatography over (silica gel, 0-10% methanol/methylene chloride with 1% ammonium hydroxide) to afford the N-acetyl intermediate (378 mg, 64%) as a yellow oil: ¹H NMR (500 MHz, DMSO-d₆) δ 7.69 (s, 1H), 7.64-7.62 (m, 1H), 7.54-7.50 (m, 1H), 7.45-7.44 (m, 1H), 3.62 (m, 4H), 3.36-3.34 (m, 2H), 3.29-3.27 (m, 2H), 2.08 (s, 3H); ESI MS m/z 250 [C₁₂H₁₅N₃O₃+H]⁺.

Step 2. The intermediate prepared in step 1 (378 mg, 1.51 mmol) was added to a solution of anhydrous ethanol (20 mL) containing 5 wt % Pd/C (400 mg) and the reaction mixture was hydrogenated (50 psi) for 2 hours. The catalyst was removed by vacuum filtration through diatomaceous earth and the filter cake was rinsed with additional ethanol. The filtrate was concentrated under reduced pressure to afford 9 (311 mg, 94%) as a colorless syrup: ¹H NMR (500 MHz, MeOH-d₄) δ 6.97-6.96 (m, 1H), 6.36-6.34 (m, 2H), 6.28-6.27 (m, 1H), 3.68-3.66 (m, 2H), 3.63-3.61 (m, 2H), 3.12-3.10 (m, 2H), 3.06-3.04 (m, 2H), 2.11 (s, 3H); ESI MS m/z 220 [C₁₂H₁₇N₃O+H]⁺.

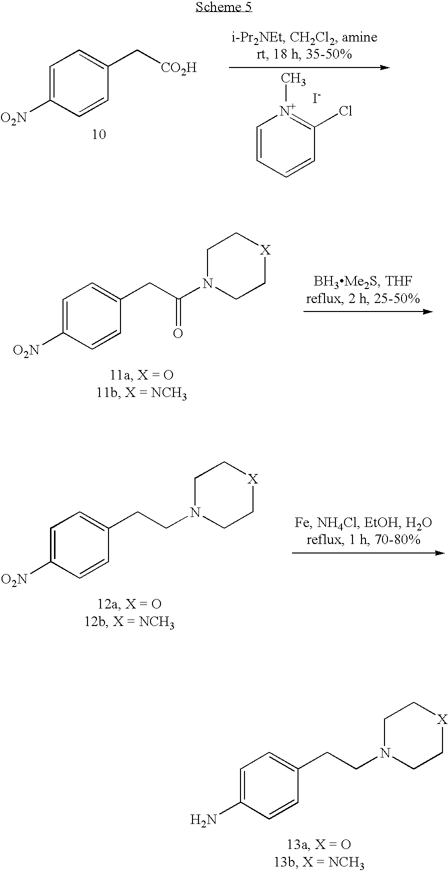

### Synthesis of 13a

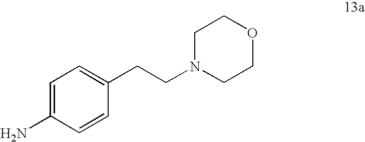

Step 1. Morpholine (3.60 mL, 41.4 mmol) was added dropwise at room temperature to a solution of 10 (5.0 g, 26.62 mmoL) in methylene chloride (185 mL) containing methyl 2-chloropyridinium iodide (8.80 g, 34.5 mmol) and N,N-diisopropylethylamine (48 mL, 276 mmol). The reaction was stirred at room temperature for 18 h, diluted with methylene chloride and the layers were separated. The organic phase was washed with satd. aq. NaHCO₃, water and brine, dried over sodium sulfate and concentrated under reduced pressure. The residue was purified by chromatography (silica gel, 0-10% methanol/methylene chloride with 1% ammonium hydroxide) to afford 11a (2.95 g, 42%) as a yellow oil: ESI MS m/z 251 [C₁₂H₁₄N₂O₄+H]⁺.

Step 2. BH₃.DMS complex (2.8 mL, 29.5 mmol) was added dropwise at room temperature to a well stirred solution of 11a (2.94 g, 11.7 mmol) in tetrahydrofuran (50 mL). The reaction mixture was heated at reflux for 2 h and then cooled to room temperature. Excess BH₃.DMS was quenched by careful addition of methanol. The solvents were removed under reduced pressure and the residue was dissolved in methanol (25 mL) and 2 N HCl (25 mL) and heated at reflux for 2 h. The reaction was cooled to room temperature and poured carefully into dilute NaOH to make it basic. The reaction mixture was extracted with methylene chloride. The organic layer was washed with water and brine, dried over sodium sulfated and concentrated under reduced pressure. The residue was purified by chromatography (silica gel, 0-10% methanol/methylene chloride with 1% ammonium hydroxide) to afford 12a (1.37 g, 49%) as a brown syrup: ¹H NMR (300 MHz, CDCl₃) δ 8.14 (d, J=8.6 Hz, 2H), 7.38 (d, J=8.6 Hz, 2H), 3.74-3.71 (m, 4H), 2.93-2.88 (m, 2H), 2.65-2.60 (m, 2H), 2.53-2.50 (m, 4H); ESI MS m/z 237 [C₁₂H₁₆N₂O₃+H]⁺.

Step 3. To a solution of 12a (1.37 g, 5.80 mmol) in ethanol (40 mL) and water (25 mL) was added iron powder (1.61 g, 29 mmol) and ammonium chloride (341 mg, 6.4 mmol) and the reaction mixture was heated at 60° C. for 1.5 h. The reaction mixture was vacuum filtered through diatomaceous earth and the filtrate was concentrated. The residue was purified by chromatography (silica gel, 0-10% methanol/methylene chloride with 1% ammonium hydroxide) to afford 13a (820 mg, 74%) as a bright yellow solid: ¹H NMR (500 MHz, CDCl₃) δ 6.98 (d, J=8.3 Hz, 2H), 6.61 (d, J=8.3 Hz, 2H), 3.74-3.72 (m, 4H), 3.56 (br s, 2H), 2.70-2.67 (m, 2H), 2.55-2.50 (m, 6H); ESI MS m/z 207 [C₁₂H₁₈N₂O+H]⁺.

### Synthesis of 13b

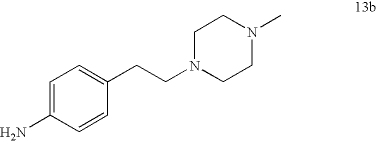

This compound was prepared by the same procedure described for 13a to afford 13b (550 mg, 77%) as a yellow solid: ¹H NMR (500 MHz, CDCl₃) δ 6.99 (d, J=8.3 Hz, 2H), 6.62 (d, J=8.3 Hz, 2H), 3.56 (br s, 2H), 2.72-2.68 (m, 2H), 2.56-2.50 (m, 10H), 2.30 (s, 3H); ESI MS m/z 220 [C₁₃H₂₁N₃+H]⁺.

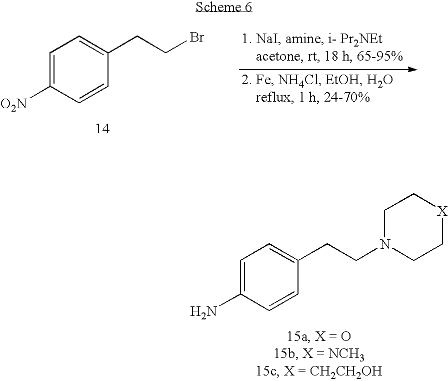

### Synthesis of 15a

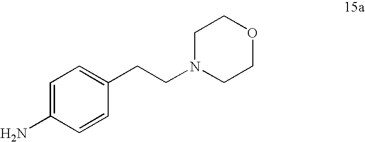

Step 1. To a solution of 14 (3.0 g, 13.0 mmol) in acetone (86 mL) was added morpholine (1.25 mL, 14.3 mmol), sodium iodide (195 mg, 1.3 mmol) and DIPEA (4.5 mL, 26 mmol) and the reaction mixture was stirred at room temperature for 48 h. The reaction mixture was concentrated under reduced pressure and the residue partitioned between ethyl acetate and satd. aq. NaHCO₃. The layers were separated and the organic layer was washed with water and brine, dried over sodium sulfate and concentrated under reduced pressure to afford the intermediate amine (4.11 g, 99%) as a red oil: ESI MS m/z 237 [C₁₂H₁₈N₂O₃+H]⁺.

Step 2. To a solution of the intermediate prepared in step 1 (3.0 g, 12.7 mmol) in ethanol (80 mL) and water (40 mL) was added iron powder (3.54 g, 63.5 mmol) and ammonium chloride (750 mg, 14 mmol) and the reaction mixture was heated at 60° C. for 1 h. The reaction mixture was vacuum filtered through diatomaceous earth and the filtrate was concentrated under reduced pressure and purified by chromatography (silica gel, 0-10% methanol/methylene chloride with 1% ammonium hydroxide) to afford 15a (1.82 g, 69%) as a bright yellow solid: ¹H NMR (500 MHz, CDCl₃) δ 6.98 (d, J=8.3 Hz, 2H), 6.61 (d, J=8.3 Hz, 2H), 3.74-3.72 (m, 4H), 3.56 (br s, 2H), 2.70-2.67 (m, 2H), 2.55-2.50 (m, 6H); ESI MS m/z 207 [C₁₂H₁₈N₂O+H]⁺.

### Synthesis of 15b

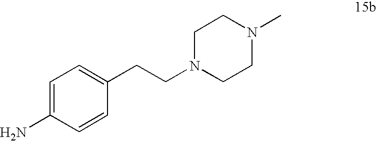

This compound was prepared by the same procedure described for 15a to afford 15b (1.34 g, 62%) as a yellow solid: ESI MS m/z 220 [C₁₃H₂₁N₃+H]⁺.

### Synthesis of 15c

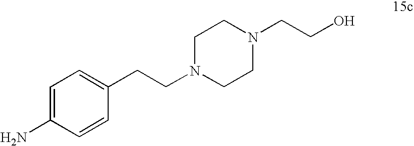

This compound was prepared by the same procedure described for 15a to afford 15c (820 mg, 24%) as a yellow solid: ESI MS m/z 250 [C₁₄H₂₃N₃O+H]⁺.

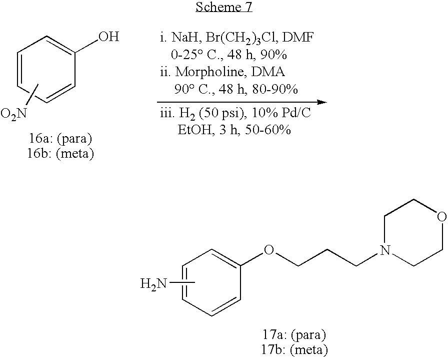

### Synthesis of 17a

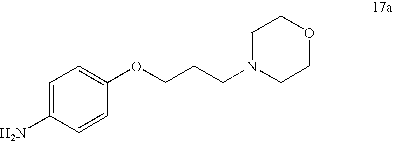

Step 1. To a solution of 16a (5 g, 35.9 mmol) in DMF (200 mL) was added 60 wt % sodium hydride (2.15 g, 53.9 mmol) portion wise and the reaction mixture was stirred at room temperature for 1 hour. The reaction mixture was cooled to 0° C. followed by dropwise addition of a solution of bromo-3-chloropropane (4.62 mL, 46.7 mmol) in N,N-dimethylformamide (40 mL). The cooling bath was removed and the reaction was allowed to stir at room temperature 48 h. The reaction mixture was poured into 3 L of water and extracted several times with ethyl acetate. The combined organic layers were washed with water and brine, dried over sodium sulfate and concentrated under reduced pressure to afford the intermediate alkyl chloride (8.4 g) as a yellow foam. This was carried crude to the next step.

Step 2. To a solution of the intermediate prepared in step 1 (8.1 g) in N,N-dimethylacetamide (180 mL) was added morpholine (9.50 mL, 108 mmol) and the reaction mixture was heated at 90° C. for 48 h. The reaction mixture was poured into 2 L of water and extracted several times with ethyl acetate. The combined organic layers were washed with water and brine, dried over sodium sulfate and concentrated under reduced pressure to afford the intermediate amine (8.1 g, 84%) as a brown solid: ¹H NMR (500 MHz, DMSO-d₆) δ 8.21-8.18 (m, 2H), 6.98-6.94 (m, 2H), 4.14-4.11 (m, 2H), 3.72 (3.70 (m, 4H), 2.53-2.46 (m, 4H), 2.03-1.99 (m, 2H), 1.26-1.24 (m, 2H); ESI MS m/z 267 [C₁₃H₁₈N₂O₄+H]⁺.

Step 3. The a solution of the intermediate prepared in step 2 (8.1 g, 30.4 mmol) in ethanol (50 mL) was added 10 wt % Palladium on carbon (800 mg) and the reaction mixture was hydrogenated (50 psi) for 3 hours. The catalyst was removed by vacuum filtration through diatomaceous earth and the filtrate was concentrated under reduced pressure and purified by chromatography (silica gel, 0-10% methanol/methylene chloride with 1% ammonium hydroxide) to afford 17a (4.4 g, 61%) as a brown syrup: ¹H NMR (500 MHz, CDCl₃) δ 6.73 (d, J=8.8 Hz, 2H), 6.62 (d, J=8.8 Hz, 2H), 3.93 (t, J=6.3 Hz, 2H), 3.72-3.70 (m, 4H), 3.41 (br s, 2H), 2.50 (m, 2H), 2.45 (m, 4H), 1.93-1.90 (m, 2H); ESI MS m/z 237 [C₁₃H₂₀N₂O₂+H]⁺.

### Synthesis of 17b

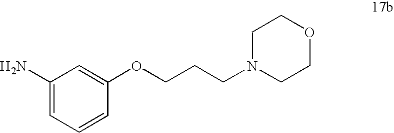

This compound was prepared by the same procedure described for 17a to afford 17b (4.2 g, 56%) as a yellow solid: ESI MS m/z 237 [C₁₃H₂₀N₂O₂+H]⁺.

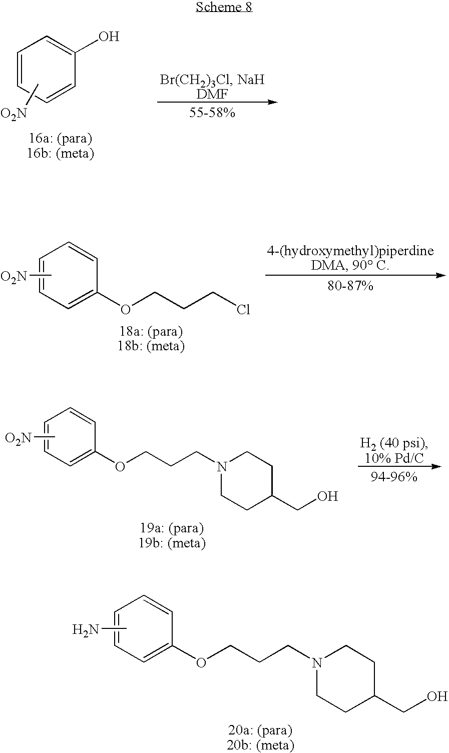

### Synthesis of 18a

To a solution of 16a (4.0 g, 29 mmol) in DMF (85 mL) was added 60 wt % NaH (0.9 g, 37 mmol) in three portions at room temperature and the resulting mixture was stirred for 30 min, cooled in an ice bath, and a solution of 1-bromo-3-chloro propane (5.9 g, 37 mmol) in DMF (15 mL) was added dropwise via an addition funnel. The resulting mixture was stirred, under nitrogen, with gradual warming to room temperature for 24 h. The reaction mixture was diluted in ethyl acetate (500 mL) and washed with a satd. aq. NH₄Cl (250 mL), water (3×200 mL), and 5 wt % LiCl (200 mL). The organic layer was dried over sodium sulfate, filtered, concentrated and the residue was purified by chromatography (silica gel, 4:1 hexanes/ethyl acetate) to afford 18a (3.4 g, 55%) as a yellow oil: ¹H NMR (500 MHz, DMSO-d₆) δ 8.23-8.19 (m, 2H), 7.19-7.16 (m, 2H), 4.25 (t, J=6.0 Hz, 2H), 3.80 (t, J=6.5 Hz, 2H), 2.24-2.19 (m, 2H).

### Synthesis of 19a

To a solution of 18a (3.4 g, 16 mmol) in DMF (80 mL) was added 4-(hydroxymethyl)piperidine (7.3 g, 63 mmol) and the resulting mixture was stirred at 90° C. for 8 h and at room temperature for 8 days. The reaction mixture was diluted in ethyl acetate (500 mL) and washed with a satd. aq. NH₄Cl (250 mL), and water (5×200 mL). The organic layer was dried over sodium sulfate, filtered and concentrated to afford 19a (3.8 g, 80%) as an orange-brown solid: ¹H NMR (500 MHz, DMSO-d₆) δ 8.20-8.18 (m, 2H), 7.14-7.12 (m, 2H), 4.37 (t, J=5.3 Hz, 1H), 4.14 (t, J=6.4 Hz, 2H), 3.24-3.21 (m, 2H), 2.86-2.83 (m, 2H), 2.40 (t, J=7.0 Hz, 2H), 1.90-1.84 (m, 4H), 1.63-1.61 (m, 2H), 1.26-1.24 (m, 1H), 1.12-1.09 (m, 2H).

### Synthesis of 20a

To a solution of 19a (3.8 g, 13 mmol,) in ethanol (150 mL) was added cat. 10 wt % Pd/C (0.80 g) and the reaction mixture was hydrogenated (40 psi) for 40 min. The reaction mixture was filtered through diatomaceous earth and the filter cake was washed with ethanol (100 mL) and ethyl acetate (300 mL). The filtrate was concentrated under reduced pressure to provide 20a (3.2 g, 94%) as a brown solid: ESI MS m/z 265 [C₁₅H₂₄N₂O₂+H]⁺.

### Synthesis of 18b

To a room temperature solution of 16b (4.0 g, 29 mmol) in DMF (85 mL) was added NaH (1.7 g, 43 mmol) in three portions at room temperature. The resulting mixture was stirred for 30 min, cooled in an ice bath, and a solution of 1-bromo-3-chloro propane (5.9 g, 37 mmol) in DMF (15 mL) was added dropwise via an addition funnel. The resulting mixture was stirred, under nitrogen, with gradual warming to room temperature for 24 h. The reaction mixture was diluted in ethyl acetate (500 mL) and washed with satd. aq. NH₄Cl (250 mL), water (3×200 mL), and 5 wt % LiCl (200 mL). The organic layer was dried over sodium sulfate, filtered and concentrated to a crude oil which was purified by flash chromatography (silica gel, 8:1 heptanes/ethyl acetate) to afford 18b (5.3 g, 85%) as a yellow oil: ¹H NMR (500 MHz, DMSO-d₆) δ 7.82-7.81 (m, 1H), 7.73-7.72 (m, 1H), 7.59-7.57 (m, 1H), 7.46-7.45 (m, 1H), 4.22 (t, J=6.0 Hz, 2H), 3.81 (t, J=6.5 Hz, 2H), 2.22-2.19 (m, 2H).

### Synthesis of 19b

To a solution of 18b (5.3 g, 24 mmol) in DMF (120 mL) was added 4-(hydroxymethyl)piperidine (11.2 g, 97 mmol) and the resulting mixture was stirred at 90° C., under nitrogen, for 8 h and then at room temperature for 2 days. The reaction mixture was diluted in ethyl acetate (500 mL) and washed with satd. aq. NH₄Cl (250 mL), and water (5×200 mL). The organic layer was dried over sodium sulfate, filtered and concentrated to afford 19b (6.2 g, 87%) as a orange-brown solid: ¹H NMR (500 MHz, DMSO-d₆) δ 7.80-7.78 (m, 1H), 7.69-7.68 (m, 1H), 7.58-7.55 (m, 1H), 7.41-7.39 (m, 1H), 4.37 (t, J=5.3 Hz, 1H), 4.12 (t, J=6.4 Hz, 2H), 3.24-3.21 (m, 2H), 2.86-2.84 (m, 2H), 2.40 (t, J=7.0 Hz, 2H), 1.90-1.81 (m, 4H), 1.63-1.61 (m, 2H), 1.26-1.24 (m, 1H), 1.12-1.09 (m, 2H).

### Synthesis of 20b

A mixture of 19b (6.2 g, 21 mmol) and 10 wt % Pd/C (1.24 g) in ethanol (150 mL) was hydrogenated (˜40 psi) for 40 minutes. The reaction mixture was then filtered through diatomaceous earth, washing with ethanol (100 mL), and then ethyl acetate (300 mL). The filtrate was concentrated under reduced pressure to provide 20b (5.4 g, 96%) as a light brown solid: ESI MS m/z 265 [C₁₅H₂₄N₂O₂+H]⁺.

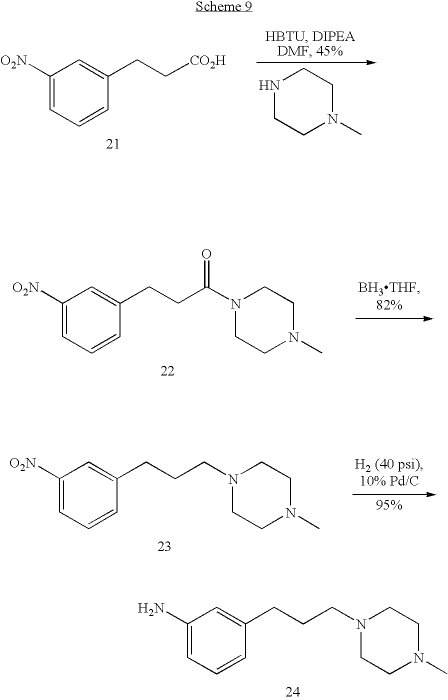

### Synthesis of 22

To a solution of 21 (3.0 g, 15 mmol) and DIPEA (7.9 g, 62 mmol) in DMF (50 mL) was added HBTU (7.0 g, 18 mmol). The reaction mixture was stirred for 30 min and a solution of amine (2.7 g, 15 mmol) in DMF (10 mL) was added. After stirring at room temperature for 7 days, the reaction mixture was diluted in ethyl acetate (500 mL) and washed with satd. aq. NH₄Cl (250 mL), water (3×200 mL), and 5 wt % LiCl (200 mL). The organic layer was dried over sodium sulfate, filtered and concentrated to obtain 22 (1.9 g, 45%) as a brown oil: ¹H NMR (500 MHz, DMSO-d₆) δ 8.13-8.12 (m, 1H), 8.06-8.04 (m, 1H), 7.73-7.72 (m, 1H), 7.59-7.55 (m, 1H), 3.44-3.43 (m, 4H), 2.97-2.94 (m, 2H), 2.73-2.69 (m, 2H), 2.30-2.28 (m, 4H), 2.21 (s, 3H).

### Synthesis of 23

To a room temperature solution of 22 (1.9 g, 6.9 mmol) in tetrahydrofuran (15 mL) was added 1 M BH₃.THF (21 mL) via addition funnel. Once the addition was complete, the reaction mixture was stirred at reflux for 2 h and then at room temperature overnight. The reaction mixture was carefully quenched with methanol (40 mL) and then concentrated to dryness. The crude solid was refluxed for 1 h in a mixture of methanol (40 mL) and 2 N HCl (40 mL), cooled to room temperature and made basic by the careful addition of 6 N NaOH. The aqueous layer was extracted with methylene chloride (2×150 mL) and the combined organic layers washed with saturated sodium chloride solution (50 mL). The organic layer was dried over sodium sulfate and concentrated to afford 23 (1.5 g, 82%) as a yellow oil: ¹H NMR (500 MHz, DMSO-d₆) δ 8.08-8.07 (m, 1H), 8.05-8.03 (m, 1H), 7.70-7.68 (m, 1H), 7.58-7.55 (m, 1H), 2.74-2.71 (m, 2H), 2.30-2.23 (m, 10H), 2.13 (s, 3H), 1.78-1.72 (m, 2H).

### Synthesis of 24

A mixture of 23 (1.5 g, 5.6 mmol) and 10 wt % Pd/C (0.30 g) in ethanol (75 mL) was hydrogenated (˜40 psi) for 20 minutes. The reaction mixture was then filtered through diatomaceous earth, the solid s washed with ethyl acetate (250 mL), and the filtrated concentrated to provide 24 (1.2 g, 95%) as a brown-yellow oil: ESI MS m/z 234 [C₁₄H₂₃N₃O+H]⁺

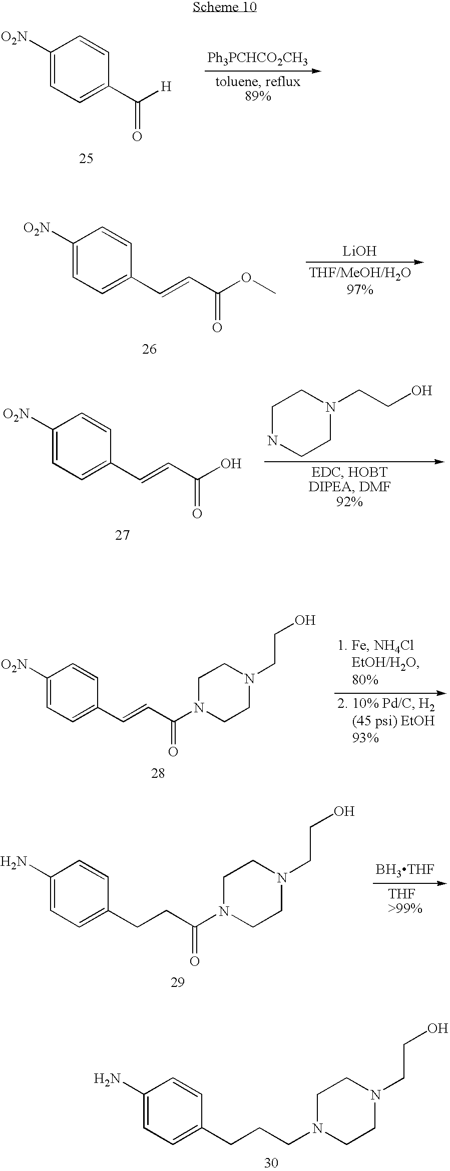

### Synthesis of 26

To a room temperature solution of 25 (10 g, 66 mmol) in toluene (330 mL) was added methyl(triphenylphosphoranylidene)acetate (24 g, 73 mmol). The resulting mixture was stirred at reflux for 14 hours. The reaction was cooled to room temperature, and the precipitate which formed was collected by vacuum filtration and washed with cold methanol (˜50 mL) to afford 26 (12 g, 89%) as a white solid: ¹H NMR (500 MHz, DMSO-d₆) δ 8.25-8.23 (m, 2H), 8.02-8.00 (m, 2H), 7.76 (d, J=16.1 Hz, 1H), 6.86 (d, J=16.1 Hz, 1H), 3.76 (s, 3H).

### Synthesis of 27

A mixture of 26 (13 g, 65 mmol) and lithium hydroxide (4.7 g, 194 mmol) in a mixture of 1,4-dioxane (162 mL), methanol (81 mL) and water (81 mL) was stirred at room temperature for 18 hours. The reaction mixture was concentrated under reduced pressure and the crude solid was dissolved in water (250 mL), cooled in an ice bath, and carefully acidified with a 6 N HCl. The precipitate which formed was collected by vacuum filtration, washed with water (250 mL) and diethyl ether (300 mL), and collected to afford 27 (12 g, 97%) as a light yellow solid: ¹H NMR (500 MHz, DMSO-d₆) δ 12.4 (bs, 1H), 8.24-8.23 (m, 2H), 7.99-7.97 (m, 2H), 7.70 (d, J=16.0 Hz, 1H), 6.75 (d, J=16.0 Hz, 1H).

### Synthesis of 28

To a stirred solution of 27 (4.0 g, 21 mmol) and N,N-diisopropylethylamine (11 g, 83 mmol) in N,N-dimethylformamide (65 mL) was added EDCl.HCl (4.8 g, 25 mmol) followed by 1-hydroxybenzotriazole (3.4 g, 25 mmol). After the resulting mixture was stirred for 20 min, a solution of N-(4-methyl)piperazine (3.6 g, 21 mmol) in N,N-dimethylformamide (15 mL) was added via syringe. The reaction mixture was stirred at room temperature for 2 days, and then diluted with ethyl acetate (500 mL), and washed with water (3×500 mL) and a 5 wt % LiCl (200 mL). The organic layer was dried over sodium sulfate, filtered and concentrated to obtain 28 (5.0 g, 92%) as a yellow solid: ESI MS m/z 276 [C₁₄H₁₇N₃O₃+H]⁺.

### Synthesis of 29

To a solution of 28 (3.6 g, 13 mmol) in a mixture of ethanol (86 mL) and water (43 mL) was added iron (3.7 g, 65 mmol) and ammonium chloride (6.8 g, 14 mmol). The reaction mixture was stirred at 85° C. for 1.5 h, cooled to room temperature, and then filtered through diatomaceous earth washing with ethanol (200 mL). The filtrate was concentrated under reduced pressure, and the crude solid was dissolved in ethanol (150 mL) and 10 wt % Pd/C (0.64 g) was added. The reaction mixture was hydrogenated (˜40 psi) at room temperature for 1.5 h. The reaction mixture was filtered through diatomaceous earth, the solids washed with ethanol (200 mL), and the filtrated concentrated to provide 29 (3.0 g, 93%) as a yellow-orange foam: ESI MS m/z 248 [C₁₄H₂₁N₃O+H]⁺.

### Synthesis of 30

To a room temperature solution of 29(1.5 g, 6 mmol) in 1,4-dioxane (15 mL) was added 1 M BH₃.THF (18 mL) via addition funnel. Once the addition was complete, the reaction mixture was stirred at reflux for 2 h and then at room temperature overnight. The reaction mixture was carefully quenched with methanol (30 mL) and then concentrated to dryness. The crude solid was refluxed for 1 h in a mixture of methanol (30 mL) and 2 N HCl (30 mL), cooled to room temperature, and made basic by the careful addition of 6 N sodium hydroxide. The aqueous layer was extracted with methylene chloride (2×100 mL) and the combined organic layers washed with satd. aq. NH₄Cl (50 mL). The organic layer was dried over sodium sulfate and concentrated to afford 30 (1.8 g, >99%) as an off-white foam: ESI MS m/z 234 [C₁₄H₂₃N₃+H]⁺.

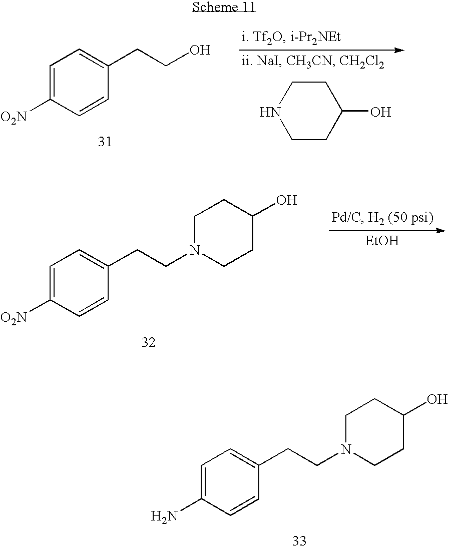

### Synthesis of 32

To a solution of 31 (20.0 g, 120 mmol) and N,N-diisopropylethylamine (31 mL, 180 mmol) in methylene chloride (300 mL) at 0° C. was added trifluoromethanesulfonic anhydride (30 mL, 51 mmol) dropwise via addition funnel and the reaction warmed to ambient temperature over 16 h. One quarter of the reaction mixture was separated, diluted with acetonitrile (100 mL) and cooled to 0° C. Sodium iodide (4.5 g, 30 mmol) was added and the mixture was stirred for 15 minutes. 4-hydroxypiperidine (15.2 g, 150 mmol) was added and the reaction was stirred at rt for 16 h. The reaction was diluted with water (600 mL) and neutralized with 2 N NaOH. The solution was extracted with a solution of 3:1 chloroform/isopropanol (3×1 L) and the combined organics dried over sodium sulfate, filtered through diatomaceous earth, and concentrated under reduced pressure to afford 32 (7.5 g, crude) as a brown solid: ESI MS m/z251 [C₁₃H₁₈N₂O₃+H]⁺.

### Synthesis of 33

A mixture of 32 (7.5 g, 30 mmol) and 10 wt % Pd/C (3.8 g, 1.8 mmol) in ethanol (75 mL) was hydrogenated (50 psi) for 22 h. The reaction mixture was filtered through diatomaceous earth. The filtrate was concentrated under reduced pressure to provide 8 (3.7 g, 56%): ESI MS m/z 221 [C₁₃H₂₀N₂O+H]⁺.

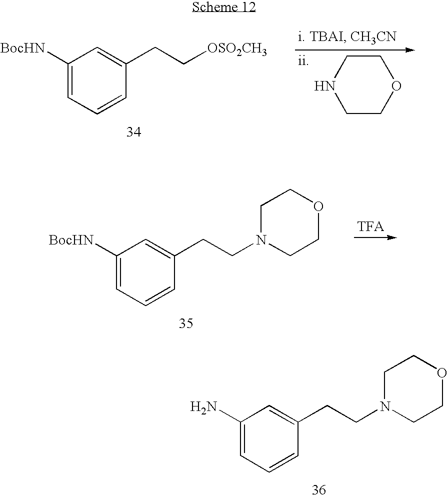

### Synthesis 35

A mixture of 34 (6.0 g, 19 mmol), TBAI (7.0 g, 19 mmol), and morpholine (6.6 g, 76 mmol) were stirred under nitrogen at ambient temperature overnight. The reaction mixture was concentrated under reduced pressure and the residue purified by chromatography (silica gel, 80:18:2 methanol/chloroform/conc. NH₄OH) to afford 35 (3.4 g, 58%) as an orange oil: ESI MS m/z 307 [C₁₇H₂₆N₂O₃+H]⁺.

### Synthesis of 35

A solution of 35 (3.4 g, 11 mmol) in trifluoroacetic acid (5 mL) was stirred under nitrogen overnight. The reaction mixture was concentrated under reduced pressure and purified by chromatography (silica gel, 80:18:2 methanol/chloroform/conc. NH₄OH) to afford 36 (1.43 g, 43%): ESI MS m/z 207 [C₁₂H₁₈N₂O+H]⁺.

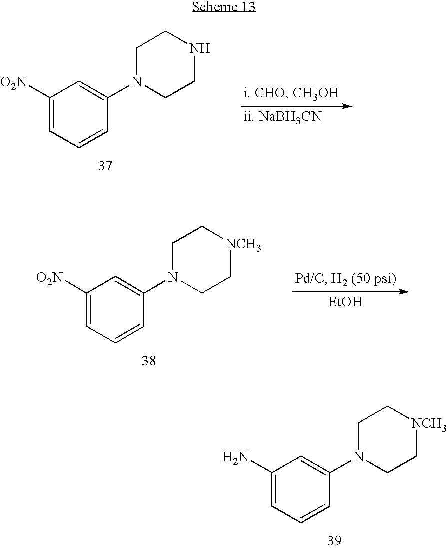

### Synthesis of 38

To a solution of 37 (10.0 g, 48 mmol) in methanol (150 mL) and N,N-dimethylformamide (5 mL) was added dropwise 37 wt % formaldehyde in water (36 mL, 0.48 mol). The mixture was stirred for 5 h and sodium cyanoborohydride (4.5 g, 72 mmol) was added portion wise. The solution was stirred at rt under nitrogen for 16 h. The solution was concentrated under reduced pressure and dissolved in ethyl acetate (1 L), washed with satd. aq. NAHCO₃ (3×600 mL), the organic layer was dried over sodium sulfate, filtered, and concentrated under reduced pressure to give 38(8.6 g, 81%) as an orange oil: ESI MS m/z222 [C₁₁H₁₅N₃O₂+H]⁺.

### Synthesis of 39

To a solution of 38 (8.6 g, 39 mmol) in ethanol (97 mL) was added 10 wt % Pd/C (5.5 g, 1.6 mmol) and the reaction mixture was hydrogenated (50 psi) for 2 h. The reaction mixture was filtered through diatomaceous earth, and the filter cake washed with ethanol (300 mL). The filtrate was concentrated under reduced pressure to provide 39 (6.9 g, 93%): ESI MS m/z 192 [C₁₁H₁₇N₃+H]⁺.

### Subsection 2: Preparation of Intermediates (b)

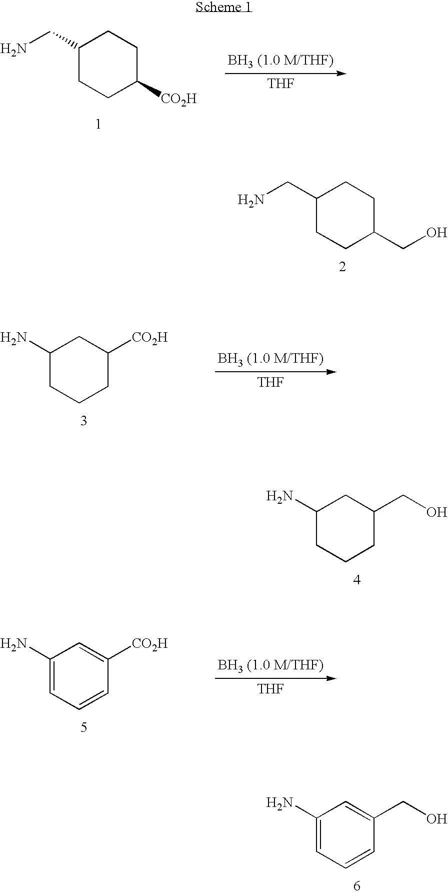

### Synthesis of 2

To a solution of 1 (2.0 g, 13 mmol) in THF (28 mL) was added dropwise via addition funnel 1.0 M BH₃.THF (38 mL, 38 mmol). The solution was stirred at room temperature for 72 h and refluxed for 2 h. The solution was cooled, diluted with methanol (70 mL) and concentrated under reduced pressure. The residue was dissolved in methanol (70 mL) and 2 N HCl (70 mL) and refluxed for 1 h. The solution was cooled and the pH was made basic with 6 N NaOH. The mixture was extracted with methylene chloride (3×500 mL) and the combined organic layers were dried over sodium sulfate, filtered through diatomaceous earth, and concentrated under reduced pressure to afford 2 (3.6 g) which was used without further purification or characterization.

### Synthesis of 4

To a solution of 3 (2.0 g, 14 mmol) in THF (31 mL) was added dropwise via addition funnel 1.0 M BH₃.THF (42 mL, 42 mmol). The solution was stirred at room temperature for 72 h and refluxed for 2 h. The solution was cooled, diluted with methanol (70 mL) and concentrated under reduced pressure. The residue was dissolved in methanol (70 mL) and 2 N HCl (70 mL) and refluxed for 1 h. The solution was cooled and the pH adjusted to 14 with a 6 N aqueous sodium hydroxide solution. The solution was cooled and the pH was made basic with 6 N NaOH. The mixture was extracted with methylene chloride (3×500 mL) and the combined organic layers were dried over sodium sulfate, filtered through diatomaceous earth, and concentrated under reduced pressure to afford 4 (1.7 g) which was used without further purification or characterization.

### Synthesis of 6

To a solution of 5 (2.0 g, 14 mmol) in THF (32 mL) was added dropwise via addition funnel 1.0 M BH₃.THF (44 mL, 44 mmol). The solution was stirred at room temperature for 72 h and refluxed for 2 h. The solution was cooled, diluted with methanol (70 mL) and concentrated under reduced pressure. The residue was dissolved in methanol (70 mL) and 2 N HCl (70 mL) and refluxed for 1 h. The reaction mixture was cooled and the pH was made basic with a 6 N NaOH. The mixture was extracted with methylene chloride (3×500 mL) and the combined organic layers dried over sodium sulfate, filtered through diatomaceous earth, and concentrated under reduced pressure to afford 6 (1.7 g) which was used crude without further purification or characterization.

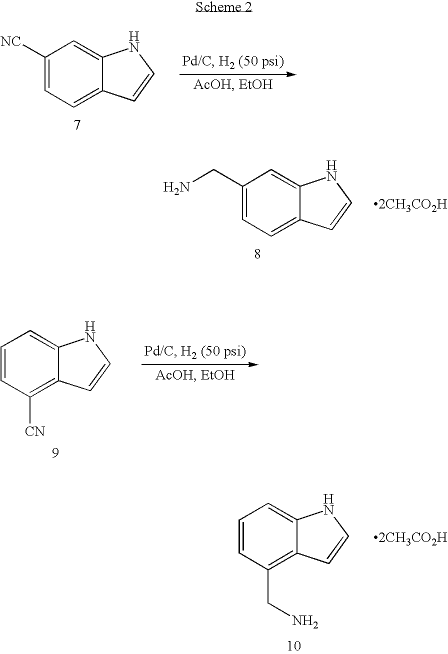

### Synthesis of 8

A mixture of 7 (1.5 g, 11 mmol), glacial acetic acid (1.8 mL, 32 mmol) and 5 wt % Pd/C (14 g, 0.32 mmol) in ethanol (42 mL) was hydrogenated (50 psi) for 3 h. The reaction mixture was filtered through diatomaceous earth. The filtrate was concentrated under reduced pressure to provide 8 (3.0 g, crude) as a brown solid which was used without further purification or characterization.

### Synthesis of 10

A mixture of 9 (1.5 g, 11 mmol), glacial acetic acid (1.8 mL, 32 mmol) and 5 wt % Pd/C (14 g, 0.32 mmol) in ethanol (42 mL) was hydrogenated (50 psi) for 3 h. The reaction mixture was filtered through diatomaceous earth. The filtrate was concentrated under reduced pressure to provide 10 (2.7 g, 95%) as a brown solid: ESI MS m/z 147 [C₁₃H₂₀N₂O+H]⁺.

### Subsection 3: Examples Prepared by Guinidine Route

### Synthesis of the Core:

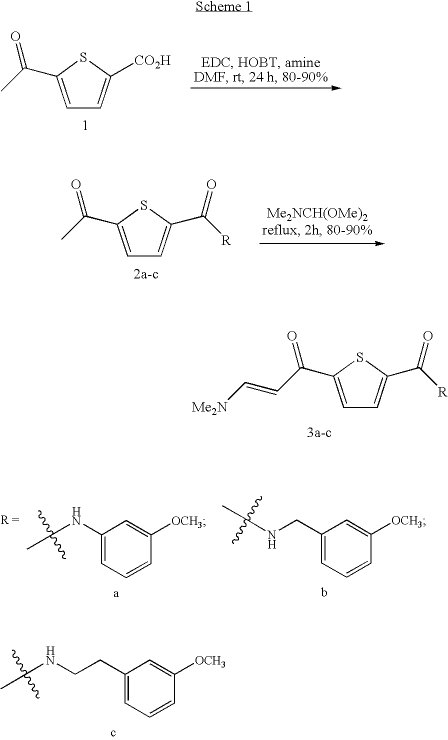

### Synthesis of 2a

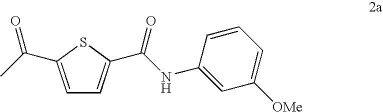

To a solution of 1 in N,N-dimethylformamide (80 mL) was added HOBt (7.62 g, 56.4 mmol) and EDC (10.8 g, 56.4 mmol) and the reaction mixture was stirred for 20 min at room temperature. The amine (5.8 mL, 70.5 mmol) was added and the reaction mixture stirred at room temperature for 18 h. The reaction mixture was concentrated under reduce pressure and the residue was partitioned between ethyl acetate and water. The phases were separated and the organic phase washed with 1 N NaOH, water and brine, dried over sodium sulfate and concentrated to afford 2a (17 g crude) as a brown solid: ¹H NMR (300 MHz, DMSO-d₆) δ 10.39 (br s, 1H), 8.05 (d, J=4 Hz, 1H), 7.99 (d, J=4 Hz, 1H), 7.42-7.40 (m, 1H), 7.32-7.24 (m, 2H), 6.74-6.70 (m, 1H), 3.76 (s, 3H), 2.58 (s, 3H).

### Synthesis of 2b

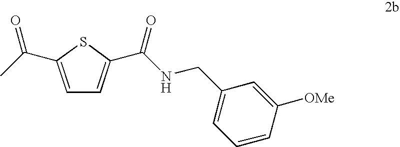

This compound was prepared by the same procedure described for 2a to afford 2b (12.6 g, 93%) as a tan solid: ¹H NMR (500 MHz, DMSO-d₆) δ 9.27-9.24 (m, 1H), 7.93-7.92 (m, 1H), 7.85-7.84 (m, 1H), 7.26-7.23 (m, 1H), 6.89-6.87 (m, 2H), 6.84-6.81 (m, 1H), 4.43 (d, J=6.0 Hz, 2H), 3.73 (s, 3H), 2.55 (s, 3H).

### Synthesis of 2c

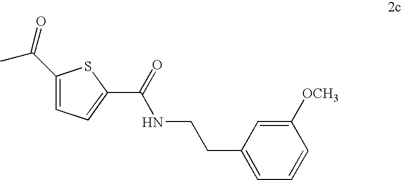

This compound was prepared by the same procedure described for 2a to afford 2c (14.6 g crude) as a brown solid: ESI MS m/z 304 [C₆H₆O₃S+H]⁺

### Synthesis of 3a

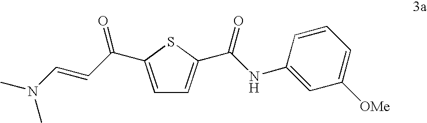

A solution 2a (8.0 g, 29 mmol) in N,N-dimethylformamide dimethylacetal (40 mL) was heated at reflux for 2 h. The reaction mixture was cooled to room temperature and concentrated under reduce pressure. The residue was suspended in ether and vacuum filtered to afford 3a (8.53 g, 89%) as a brown solid: ¹H NMR (300 MHz, DMSO-d₆) δ 10.26 (br s, 1H), 7.79 (d, J=4 Hz, 1H), 7.82 (d, J=4 Hz, 1H), 7.73 (J=12 Hz, 1H), 7.41 (m, 1H), 7.32-7.24 (m, 2H), 6.71-6.68 (m, 1H), 5.82 (d, J=12 Hz, 1H), 3.75 (s, 3H), 3.16 (s, 3H), 2.94 (s, 3H); ESI MS m/z 331 [C₁₇H₁₈N₂O₃S+H]⁺.

### Synthesis of 3b

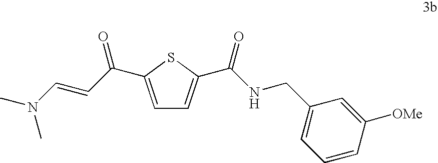

This compound was prepared by the same procedure described for 3a to afford 3b (9.0 g, 95%) as a red-brown solid: ¹H NMR (500 MHz, DMSO-d₆) δ 9.09-9.07 (m, 1H), 7.77-7.74 (m, 2H), 7.70 (d, J12.2 Hz, 1H), 7.26-7.23 (m, 1H), 6.89-6.87 (m, 2H), 6.83-6.81 (m, 1H), 5.77 (d, J12.2 Hz, 1H), 4.41 (d, J=5.9 Hz, 2H), 3.73 (s, 3H), 3.15 (s, 3H), 2.92 (s, 3H).

### Synthesis of 3c

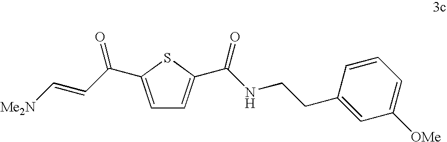

This compound was prepared by the same procedure described for 3a to afford 3c (9.6 g, 93%) as a brown solid: ESI MS m/z 359 [C₁₉H₂₂N₂O₃S+H]⁺

### Synthesis of Guanidine Intermediates

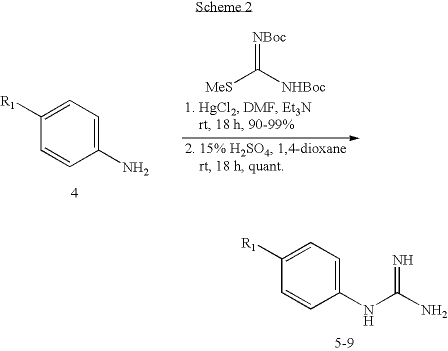

### Synthesis of 5

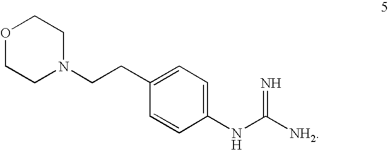

To a solution of aniline (1.0 g, 2.42 mmol) in N,N-dimethylformamide (20 mL) was added triethylamine (2.4 mL, 17 mmol), 1,3-bis(tert-butoxycarbonyl)-2-methyl-2-thiopseudourea (1.54 g, 5.32 mmol) and mercury (II) chloride (1.45 g), 5.32 mmol) and the reaction mixture was stirred at room temperature 18 h. the reaction mixture was poured into ethyl acetate (200 mL) and washed with water (2×100 mL) and brine, dried over Na₂SO₄ and concentrated under reduced pressure to afford the protected guanidine intermediate (2.32 g crude) as a yellow-orange gum. The intermediate (2.32 g) was dissolved in 1,4-dioxane (20 mL) followed by addition of a 15 wt % H₂SO₄ (20 mL) and the reaction mixture was stirred at room temperature for 48 h. The reaction mixture was carefully added to excess satd. aq. NaHCO₃ and extracted with chloroform/2-propanol (3:1). The organic layer was washed with water and brine, dried over Na₂SO₄ and concentrated under reduced pressure to afford 5 (400 mg, 31%) as a yellow solid: ESI MS m/z 249 [C₁₃H₂₀N₄O+H]⁺.

### Synthesis of 6

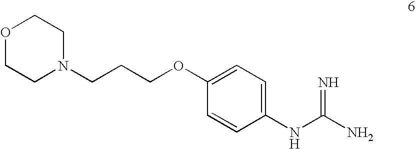

This compound was prepared by the same procedure described for guanidine 5 to afford 6 (684 mg, 62%) as a yellow solid: ESI MS m/z 279 [C₁₄H₂₂N₄O₂+H]⁺.

### Synthesis of 7

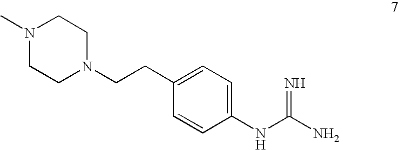

This compound was prepared by the same procedure described for guanidine 5 to afford 7 (1.2 g, 99%) as a yellow solid: ESI MS m/z 262 [C₁₄H₂₃N₅+H]⁺.

### Synthesis of 8

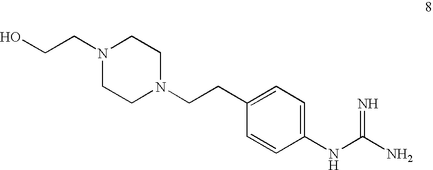

This compound was prepared by the same procedure described for guanidine 5 to afford 8 (300 mg, 27%) as a yellow solid: ESI MS m/z 291 [C₁₅H₂₅N₂O+H]⁺.

### Synthesis of 9

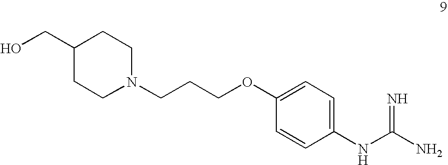

This compound was prepared by the same procedure described for guanidine 5 to afford 9 (1.2 g, quant.) as a light brown syrup: ESI MS m/z 307 [C₁₆H₂₆N₄O₂+H]⁺.

### Synthesis of 10

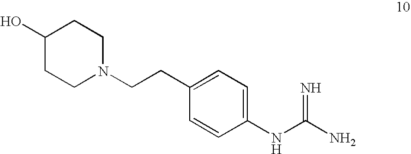

This compound was prepared by the same procedure described for guanidine 5 to afford 10 (1.6 g crude) as a yellow solid: ESI MS m/z 263 [C₁₄H₂₂N₄O+H]⁺

### Synthesis of 10B

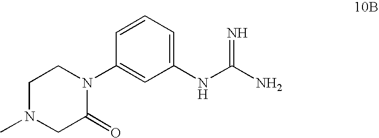

This compound was prepared by the same procedure described for guanidine 5 to afford 10B (663 mg) as a yellow solid: ¹H NMR (300 MHz, DMSO-d₆) δ 7.22-7.15 (m, 1H), 6.82-6.69 (m, 3H), 3.61 (t, J=5.5 Hz, 2H), 3.07 (s, 2H), 2.69 (t, J=5.5 Hz, 2H), 2.26 (s, 3H).

### Syntheses of Examples

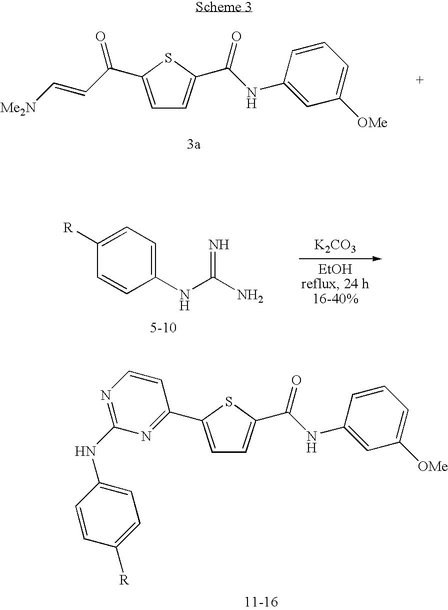

### Synthesis of Example 11

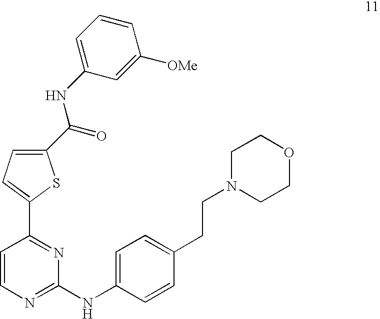

A solution of 5 (75 mg, 0.30 mmol), 3a (150 mg, 0.45 mmol) and potassium carbonate (45 mg, 0.30 mmol) in absolute ethanol (2 mL) was heated at reflux for 18 h. The reaction mixture was cooled to room temperature and concentrated under reduced pressure. The residue was purified by chromatography (silica gel, 0-10% methanol/methylene chloride w/1% conc. ammonium hydroxide) to afford 11 (25 mg 16%) bright yellow solid; mp 178-180° C.; ¹H NMR (500 MHz, DMSO-d₆) δ 10.32 (m, 1H), 9.65 (m, 1H), 8.54-8.53 (m, 1H), 8.08-9.05 (m, 2H), 7.72-7.71 (m, 2H), 7.45-7.44 (m, 1H), 7.39-7.38(m, 1H), 7.33-7.27 (m, 1H), 7.19-7.17 (m, 2H), 6.72-6.70 (m, 1H), 3.77 (s, 3H), 3.58-3.57 (m, 4H), 2.71-2.68 (m, 2H), 2.48-42 (m, 4H), ESI MS m/z 516 [C₂₈H₂₉N₅O₃S+H]⁺; HPLC: >99% (AUC) (Method A), t_{R}=11.85 min

### Synthesis of Example 12

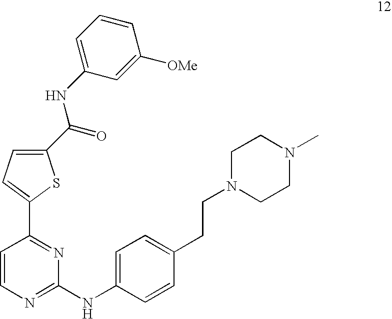

This compound was prepared by the same procedure described for 11 to afford 12 (28 mg 18%) bright yellow solid; mp 170-172° C.; ¹H NMR (500 MHz, DMSO-d₆) δ 10.32 (m, 1H), 9.65 (m, 1H), 8.54-8.53 (m, 1H), 8.08-8.05 (m, 2H), 7.72-7.71 (m, 2H), 7.45-7.44 (m, 1H), 7.39-7.38 (m, 1H), 7.36-7.26 (m, 2H), 7.18-7.16 (m, 2H), 6.72 (m, 1H), 3.77 (s, 3H), 2.69-2.66 (m, 2H), 2.49-31 (m, 8H), 2.14 (s, 3H); ESI MS m/z 529 [C₂₉H₃₂N₆O₂S+H]⁺; HPLC: 98.4% (AUC) (Method A), t_{R}=10.58 min

### Synthesis of Example 13

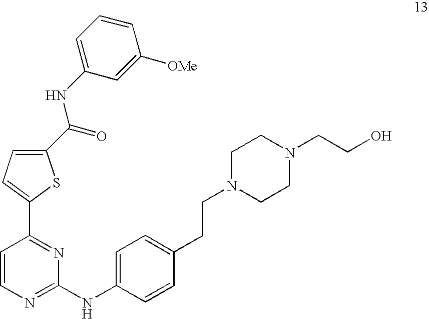

This compound was prepared by the same procedure described for 11 to afford 13 (60 mg 36%) bright yellow solid; mp 198-200° C.; ¹H NMR (500 MHz, DMSO-d₆) δ 10.32 (m, 1H), 9.65 (m, 1H), 8.54-8.53 (m, 1H), 8.08-8.05 (m, 2H), 7.72-7.71 (m, 2H), 7.44 (m, 1H), 7.39-7.36 (m, 2H), 7.33-7.27 (m, 2H), 7.18-7.16 (m, 2H), 6.72 (m, 1H), 3.77 (s, 3H), 3.48-3.46 (m, 2H), 2.69-2.66 (m, 2H), 2.49-31 (m, 10H); ESI MS m/z 559 [C₃₀H₃₄N₆O₃S+H]⁺; HPLC: >99% (AUC) (Method A), t_{R}=10.48 min

### Synthesis of Example 14

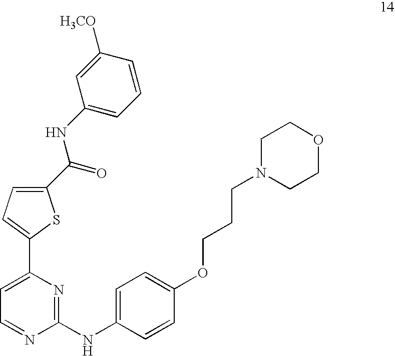

This compound was prepared by the same procedure described for 11 to afford 14 (70 mg, 37%) as a bright yellow solid: mp 128-130° C.; ¹H NMR (500 MHz, DMSO-d₆) δ 10.31 (m, 1H), 9.54 (s, 1H), 8.51-8.50 (m, 1H), 8.07-8.04 (m, 2H), 7.69-7.67 (m, 2H), 7.44-7.43 (m, 1H), 7.35-7.34 (m, 2H), 7.32-7.27 (m, 1H), 6.91-6.89 (m, 2H), 6.60-6.59 (m, 1H), 4.00-3.98 (m, 2H), 3.76 (s, 3H), 3.58-3.56 (m, 4H), 2.49-2.43(m, 2H), 2.41-2.36 (m, 4H), 1.88-1.85 (m, 2H); ESI MS m/z 574 [C₂₉H₃₁N₅O₄S+H]⁺; HPLC (Method A)>99% (AUC), t_{R}=11.69 min.

### Synthesis of Example 15

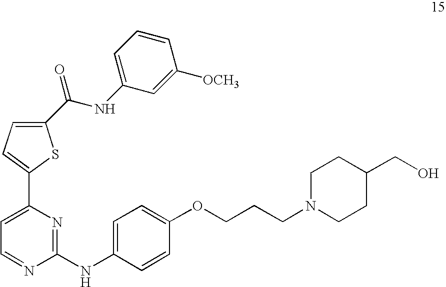

This compound was prepared by the same procedure described for 11 to afford 15 (85 mg, 23%) as a yellow solid: mp 90-94° C.; ¹H NMR (500 MHz, DMSO-d₆) δ 10.31 (s, 1H), 9.54 (s, 1H), 8.51-8.50 (m, 1H), 8.07-8.04 (m, 2H), 7.69-7.67 (m, 2H), 7.44 (s, 1H), 7.35-7.33 (m, 2H), 7.29-7.25 (m, 1H), 6.91-6.89 (m, 2H), 6.72-6.70 (m, 1H), 4.39-4.37 (m, 1H), 3.98-3.96 (m, 2H), 3.76 (s, 3H), 3.24-3.21 (m, 2H), 2.87-2.85 (m, 2H), 2.40-2.38 (m, 2H), 1.86-1.82 (m, 4H), 1.63-1.61 (m, 2H), 1.33-1.29 (m, 1H), 1.14-1.09 (m, 2H); ESI MS m/z 574 [C₃₁H₃₅N₅O₄S+H]⁺; HPLC (Method A) 96.2% (AUC), t_{R}=11.47 min.

### Synthesis of Example 16

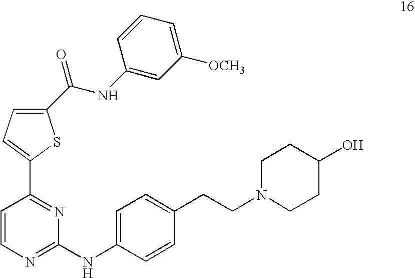

This compound was prepared by the same procedure described for 11 to afford 16 (90 mg, 40%) as an orange solid: mp 109-112° C.; ¹H NMR (500 MHz, DMSO-d₆) δ 10.33 (s, 1H), 9.65 (s, 1H), 8.54 (d, J=5.1 Hz, 1H), 8.09-8.06 (m, 2H), 7.71 (d, J=8.5 Hz, 2H), 7.45 (t, J=2.2 Hz, 1H), 7.38 (d, J=5.1 Hz, 1H), 7.33 (t, J=0.8 Hz, 1H), 7.28 (d, J=8.1 Hz, 1H), 7.16 (d, J=8.5 Hz, 2H), 4.53 (d, J=4.2 Hz, 1H), 3.77 (s, 3H) 6.18 (t, J=2.3 Hz, 1H), 4.53 (d, J=4.3 Hz, 1H), 3.77 (s, 3H), 3.49-3.39 (m, 1H), 2.82-2.72 (m, 2H), 2.70-2.62 (m, 2H), 2.49-2.46 (m, 2H), 2.11-1.99 (m, 2H), 1.74-1.65 (m, 2H), 1.42-1.33 (m, 2H); ESI MS m/z 530 [C₂₉H₃₁N₅O₃S+H]⁺; HPLC >99%, t_{R}=4.0 min.

### Synthesis of Example 16B

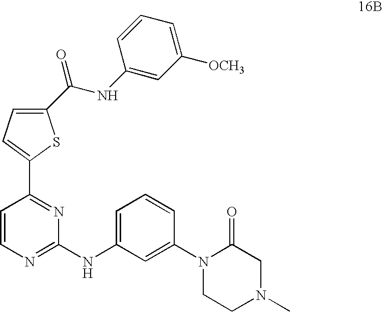

This compound was prepared by the same procedure described for 11 to afford 16B (74 mg, 36%) as a yellow solid: ¹H NMR (300 MHz, DMSO-d₆) □ 10.33 (s, 1H), 9.88 (s, 1H), 8.58 (d, J=5.1 Hz, 1H), 8.11-8.05 (m, 3H), 7.59-7.23 (m, 6H), 6.95-6.90 (m, 1H), 6.73-6.68 (m, 1H), 3.76 (s, 3H), 3.74-3.67 (m, 2H), 3.16 (bs, 2H), 2.83-2.77 (m, 2H), 2.29 (s, 3H); ESI MS m/z 515 [C₂₇H₂₆N₆O₃S+H]⁺; HPLC 94.3%, t_{R}=11.33 min.

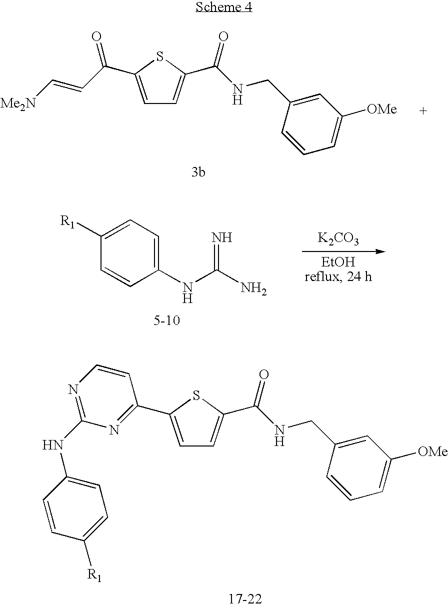

### Synthesis of Example 17

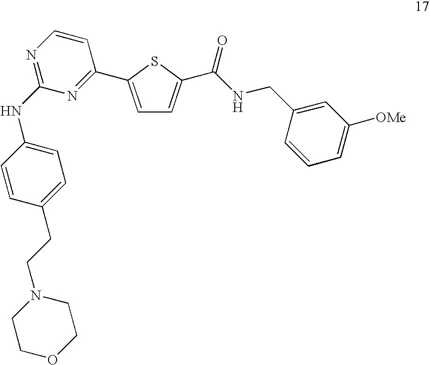

This compound was prepared using 3b by the same procedure described for 11 to afford 17 (40 mg 22%) bright yellow solid; mp 169-171° C.; ¹H NMR (500 MHz, DMSO-d₆) δ 9.63 (m, 1H), 9.19 (m,1H), 8.52-8.51 (m, 1H), 7.99-7.98 (m, 1H), 7.87-7.86 (m, 1H), 7.71-7.70 (m, 2H), 7.35-7.34 (m, 1H), 7.26-7.24 (m, 1H), 7.17-7.16 (m, 2H), 6.91 (m, 2H), 6.89-6.84 (m, 1H), 4.46-4.44 (m, 2H), 3.74 (s, 3H), 3.58-3.56 (m, 4H), 2.69-2.66 (m, 2H), 2.42 (m, 4H); ESI MS m/z 530 [C₂₉H₃₁N₅O₃S+H]⁺; HPLC: >99% (AUC) (Method A), t_{R}=11.49 min

### Synthesis of Example 18

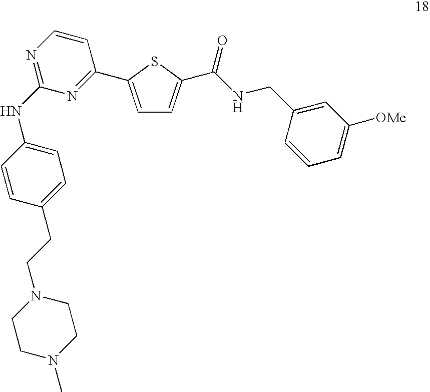

This compound was prepared using 3b by the same procedure described for 11 to afford 18 (65 mg 40%) bright yellow solid; mp 164-166° C.; ¹H NMR (500 MHz, DMSO-d₆) δ 9.62 (m, 1H), 9.20-9.18 (m,1H), 8.52-8.51 (m, 1H), 7.99-7.98 (m, 1H), 7.87-7.86 (m, 1H), 7.71-7.69 (m, 2H), 7.35-7.34 (m, 1H), 7.28-7.24 (m, 1H), 7.16-7.15 (m, 2H), 6.91-6.89 (m, 2H), 6.84-6.82 (m, 1H), 4.46-4.44 (m, 2H), 3.74 (s, 3H), 3.69-3.65 (m, 2H), 2.49-2.36 (m, 6H), 2.14 (m, 3H); ESI MS m/z 543 [C₃₀H₃₄N₆O₂S+H]⁺; HPLC: >99% (AUC) (Method A), t_{R}=10.47 min

### Synthesis of Example 19

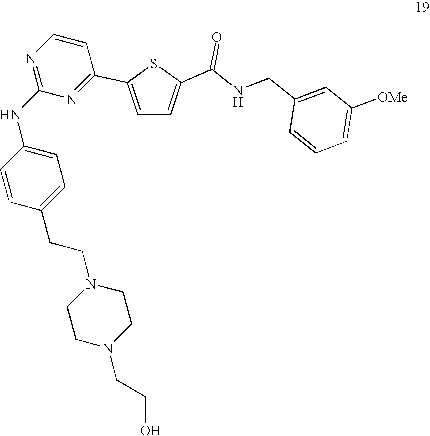

This compound was prepared using 3b by the same procedure described for 11 to afford 19 (41 mg 24%) a bright yellow solid: mp 157-159° C.; ¹H NMR (500 MHz, DMSO-d₆) δ 9.62 (s, 1H), 9.19 (m, 1H), 8.52-8.51 (m, 1H), 7.99-7.98 (m, 1H), 7.87-7.86 (m, 1H), 7.71-7.69 (m, 2H), 7.35-7.34 (m, 1H), 7.26-7.24 (m, 1H), 7.16-7.15 (m, 2H), 6.91-6.84 (m, 3H), 4.46-4.44 (m, 2H), 4.35 (m, 1H), 3.74 (s, 3H), 3.49-3.45 (m, 2H), 2.67-2.65 (m, 2H), 2.49-2.34 (m, 10H); ESI MS m/z 573 [C₃₁H₃₆N₆O₃S+H]⁺; HPLC (Method A) 99% (AUC), t_{R}=10.39 min.

### Synthesis of Example 20

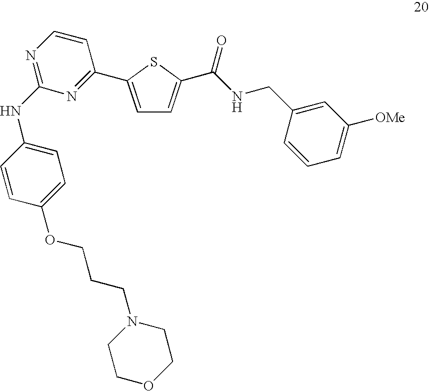

This compound was prepared using 3b by the same procedure described for 11 to afford 20 (34 mg 17%) as a bright yellow powder: mp 150-152° C.; ¹H NMR (500 MHz, DMSO-d₆) δ 9.51 (s, 1H), 9.18 (m, 1H), 8.49-8.48 (m, 1H), 7.98-7.97 (m, 1H), 7.86-7.85 (m, 1H), 7.68-7.66 (m, 2H), 7.31-7.30 (m, 1H), 7.26-7.24 (m, 1H), 6.91-6.89 (m, 4H), 6.88-6.84 (m, 1H), 4.45-4.44 (m, 2H), 3.99-3.97 (m, 2H), 3.74 (s, 3H), 3.58-3.56 (m, 4H), 2.49 (m, 2H), 2.43-2.36 (m, 4H), 1.87-1.84 (m, 2H); ESI MS m/z 560 [C₃₀H₃₃N₅O₄S+H]⁺; HPLC (Method A) 98.7% (AUC), t_{R}=11.26 min.

### Synthesis of Example 21

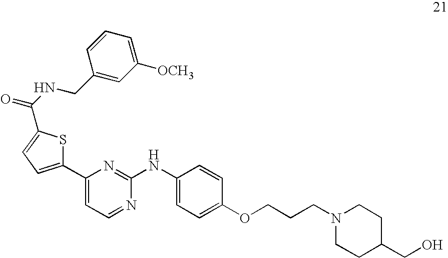

This compound was prepared using 3b by the same procedure described for 11 to afford 21 (95 mg, 25%) as a yellow solid: mp 147-149° C.; ¹H NMR (500 MHz, DMSO-d₆) δ 9.51 (s, 1H), 9.19-9.17 (m, 1H), 8.49-8.47 (m, 1H), 7.98-7.97 (m, 1H), 7.86-7.85 (m, 1H), 7.68-7.66 (m, 2H), 7.31-7.30 (m, 1H), 7.27-7.24 (m, 1H), 6.91-6.88 (m, 4H), 6.84-6.82 (m, 1H), 4.45-4.44 (m, 2H), 4.39-4.37 (m, 1H), 3.97-3.95 (m, 2H), 3.74 (s, 3H), 3.24-3.21 (m, 2H), 2.87-2.85 (m, 2H), 2.40-2.38 (m, 2H), 1.87-1.81 (m, 4H), 1.63-1.61 (m, 2H), 1.33-1.29 (m, 1H), 1.14-1.06 (m, 2H); ESI MS m/z 588 [C₃₂H₃₇N₅O₄S+H]⁺; HPLC (Method A) 96.7% (AUC), t_{R}=11.07 min.

### Synthesis of Example 22

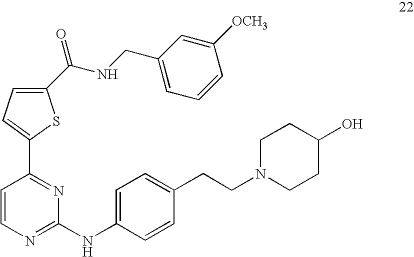

This compound was prepared using 3b by the same procedure described for 11 to afford 22 (106 mg, 51%) as an orange solid: mp 102-105° C.; ¹H NMR (500 MHz, DMSO-d₆) δ 9.62 (s, 1H), 9.19 (t, J=6.2 Hz, 1H), 8.54 (d, J=5.1 Hz, 1H), 7.99 (d, J=4.0 Hz, 1H), 7.87 (d, J=4.0 Hz, 1H), 7.70 (d, J=8.5 Hz, 2H), 7.35 (d, J=5.1 Hz, 1H), 7.26 (t, J=8.2 Hz, 1H), 7.15 (d, J=7.7 Hz, 2H), 6.90 (t, J =7.0 Hz, 2H), 6.85-6.82 (m, 1H), 4.52 (d, J4.2 Hz, 1H), 4.45 (d, J=5.9 Hz, 2H), 3.74 (s, 1H), 3.48-3.38 (m, 1H), 2.77 (d, J=11.4 Hz, 2H), 2.68-2.65 (t, J=7.2 Hz, 2H), 2.49-2.44 (m, 2H), 2.11-1.99 (m, 2H), 1.72-1.69 (m, 2H), 1.43-1.32 (m, 2H); ESI MS m/z 530 [C₂₉H₃₁N₅O₃S+H]⁺; HPLC>99%, t_{R}=4.0 min.

### Synthesis of Example 22B

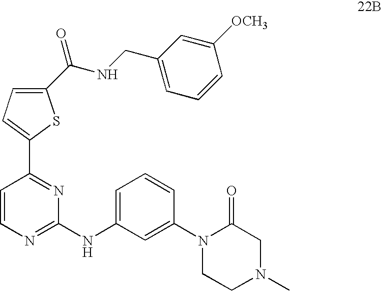

This compound was prepared using 3b by the same procedure described for 11 to afford 22B (14 mg, 7%) as a yellow solid: ¹H NMR (300 MHz, DMSO-d₆) □ 9.84 (s, 1H), 9.17 (t, J=5.8 Hz, 1H), 8.56 (d, J=5.1 Hz, 1H), 8.05-7.99 (m, 2H), 7.86 (d, J=4.0 Hz,1H), 7.59-7.55 (m, 1H), 7.42-7.23 (m, 3H), 6.94-6.82 (m, 4H), 4.45 (d, J=5.8 Hz, 2H), 3.75-3.67 (m, 5H), 3.16 (bs, 2H), 2.78 (bs, 2H), 2.28 (s, 3H); ESI MS m/z 529 [C₂₈H₂₈N₆O₃S+H]⁺; HPLC 95.0%, t_{R}=10.87 min.

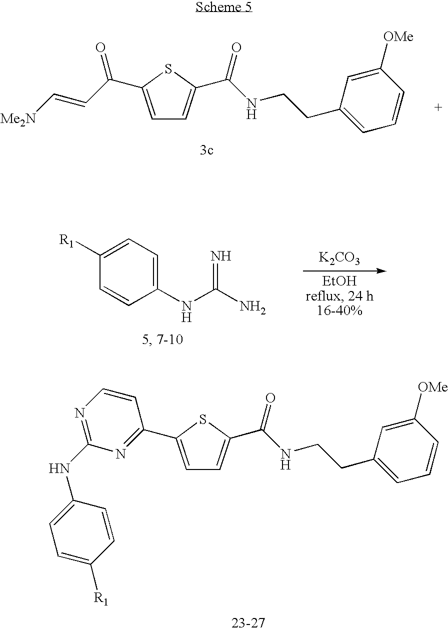

### Synthesis of Example 23

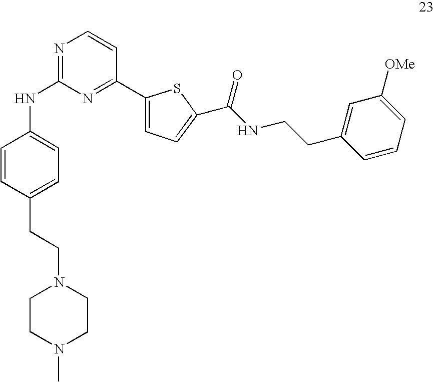

This compound was prepared using 3c by the same procedure described for 11 to afford 23 (40 mg 24%) bright yellow solid; mp 126-128° C.; ¹H NMR (500 MHz, DMSO-d₆) δ 9.61 (m, 1H), 8.74 (m, 1H), 8.51-8.50 (m, 1H), 7.97-7.96 (m, 1H), 7.878-7.77 (m, 1H), 7.71-7.69 (m, 2H), 7.34-7.33 (m, 1H), 7.23-7.20 (m, 1H), 7.16-7.15 (m, 2H), 6.83-6.77 (m, 3H), 3.72 (s, 3H), 3.48-3.47 (m, 2H), 2.84-2.81 (m, 2H), 2.69-2.66 (m, 2H), 2.49-2.31 (m, 8H), 2.14 (m, 3H); ESI MS m/z 543 [C₃₁H₃₆N₆O₂S+H]⁺; HPLC: >99% (AUC) (Method A), t_{R}=10.52 min

### Synthesis of Example 24

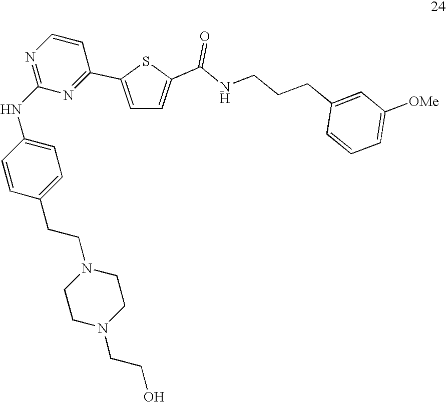

This compound was prepared using 3c by the same procedure described for 11 to afford 24 (30 mg, 17%) as a bright yellow solid: mp 88-90° C.; ¹H NMR (500 MHz, DMSO-d₆) δ 9.61 (s, 1H), 8.74 (m, 1H), 8.51-8.50 (m, 1H), 7.99-7.96 (m, 1H), 7.78-7.77 (m, 1H), 7.71-7.69 (m, 2H), 7.34-7.33 (m, 1H), 7.23-7.21 (m, 1H), 7.16-7.15 (m, 2H), 6.83-6.79 (m, 3H), 4.35 (m, 1H), 3.72 (s, 3H), 3.49-3.32 (m, 4H), 2.84-2.83 (m, 2H), 2.68-2.66 (m, 2H), 2.49-2.34 (m, 10H); ESI MS m/z 587 [C₃₂H₃₈N₆O₃S+H]⁺; HPLC (Method A) 99% (AUC), t_{R}=10.40 min.

### Synthesis of Example 25

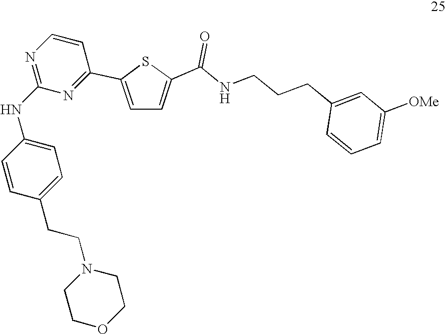

This compound was prepared using 3c by the same procedure described for 11 to afford 25 (43 mg 23%) as a bright yellow solid: mp 130-132° C.; ¹H NMR (500 MHz, DMSO-d₆) δ 9.51 (s, 1H), 8.73(m, 1H), 8.48-8.47 (m, 1H), 7.95 (m, 1H), 7.77-7.76 (m, 1H), 7.68-7.66 (m, 1H), 7.30-7.29 (m, 1H), 7.21 (m, 1H), 6.90-6.88 (m, 2H), 6.82-6.81 (m, 2H), 6.79-6.76 (m, 1 H), 4.00-3.99 (m, 2H), 3.72 (s, 3H), 3.58-3.56 (m, 4H), 3.48-3.47 (m, 2H), 2.84-2.82 (m, 2H), 2.49-2.40 (m, 2H), 2.36 (m, 4H), 1.87-1.86 (m, 2H); ESI MS m/z 574 [C₃₁H₃₅N₅O₄S+H]⁺; HPLC (Method A) 99.6% (AUC), t_{R}=11.49 min.

### Synthesis of Example 26

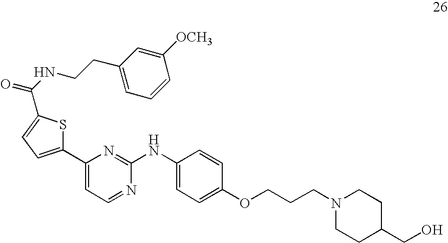

This compound was prepared using 3c by the same procedure described for 11 to afford 26 (105 mg, 27%) as a yellow solid: mp 77-79° C.; ¹H NMR (500 MHz, DMSO-d₆) δ 9.51 (s, 1H), 8.74-8.72 (m, 1H), 8.48-8.47 (m, 1H), 7.96-7.95 (m, 1H), 7.77-7.76 (m, 1H), 7.68-7.66 (m, 2H), 7.30-7.29 (m, 1H), 7.23-7.20 (m, 1H), 6.90-6.88 (m, 2H), 6.82-6.81 (m, 2H), 6.79-6.76 (m, 1H), 4.39-4.37 (m, 1H), 3.98-3.95 (m, 2H), 3.72 (s, 3H), 3.49-3.45 (m, 2H), 3.24-3.21 (m, 2H), 2.87-2.81 (m, 4H), 2.41-2.38 (m, 2H), 1.87-1.82 (m, 4H), 1.63-1.61 9m, 2H), 1.32-1.29 (m, 1H), 1.15-1.07 (m, 2H); ESI MS m/z 602 [C₃₃H₃₉N₅O₄S+H]⁺; HPLC (Method A) 98.1% (AUC), t_{R}=11.30 min.

### Synthesis of Example 27

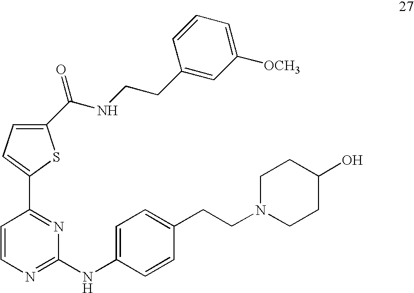

This compound was prepared using 3c by the same procedure described for 11 to afford 27 (89 mg, 42%) as an orange solid: mp 90-92° C.; ¹H NMR (500 MHz, DMSO-d₆) δ 9.62 (s, 1H), 8.75 (t, J=6.1 Hz, 1H), 8.51 (d, J=5.1 Hz, 1H), 7.97 (d, J=4.0 Hz, 1H), 7.78 (d, J=4.0 Hz, 1H), 7.70 (d, J=8.5 Hz, 2H), 7.34 (d, J=5.1 Hz, 1H), 7.21 (t, J=8.0 Hz, 1H), 7.21 (d, J=8.0 Hz, 1H), 7.15 (d, J=8.5 Hz, 2H), 6.83-6.81 (m, 2H) 6.18 (t, J=2.3 Hz, 1H), 4.53 (d, J=4.3 Hz, 1 H), 3.73 (s, 3H, 3.48-3.47 (m, 3H), 2.83 (t, J=7.5 Hz, 2H), 2.81-2.73 (m, 2H), 2.67 (t, J=7.3 Hz, 2H), 2.49-2.43 (m, 2H), 2.05-2.03 (m, 2H), 1.78-1.69 (m, 2H), 1.42-1.31 (m, 2H); ESI MS m/z 558 [C₃₁H₃₅N₅O₃S+H]⁺; HPLC 96.2%, t_{R}=11.4 min.

### Synthesis of Example 27B

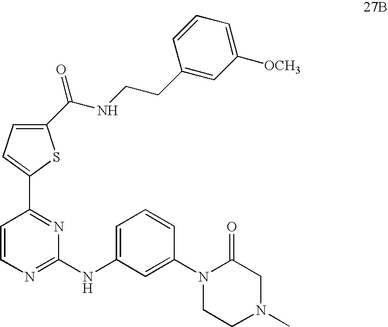

This compound was prepared using 3c by the same procedure described for 11 to afford 27B (29 mg, 13%) as a yellow solid: ¹H NMR (500 MHz, DMSO-d₆) □ 9.82 (s, 1H), 8.73 (t, J=5.5 Hz, 1H), 8.55 (d, J=5.1 Hz, 1H), 8.03-7.98 (m, 2H), 7.77 (d, J=4.0 Hz, 1H), 7.59-7.57 (m, 1H), 7.38 (d, J=5.1 Hz, 1H), 7.33 (d, J=8.1 Hz, 1H), 7.21 (d, J=8.1 Hz, 1H), 6.94-6.91 (m, 1H), 6.84-6.77 (m, 3H), 3.76 (s, 3H), 3.71-3.67 (m, 2H), 3.51-3.46 (m, 2H), 3.15 (bs, 2H), 2.86-2.78 (m, 4H), 2.30 (s, 3H); ESI MS m/z 543 [C₂₉H₃₀N₆O₃S+H]⁺; HPLC >99%, t_{R}=11.14 min.

### Subsection 4—Examples Synthesised by Main Route

### Synthesis of Core:

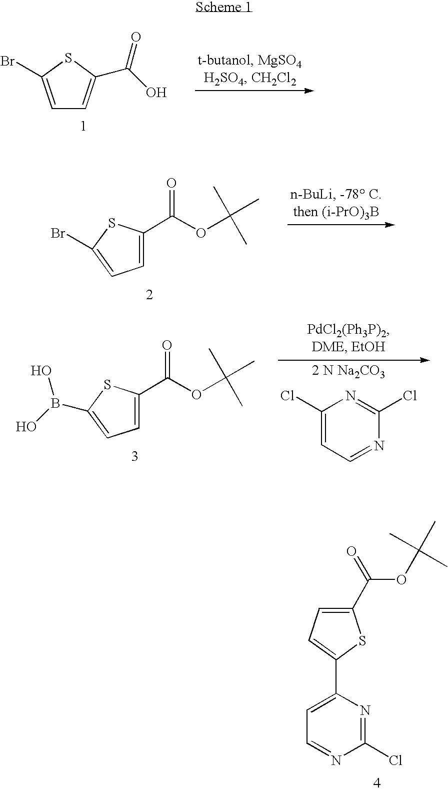

### Synthesis of 2

To a suspension of MgSO₄ (58 g, 484 mmol) in CH₂Cl₂ (300 mL) was added conc. H₂SO₄ (6.6 mL, 121 mmol) dropwise and the resulting slurry was stirred vigorously for 15 min. A slurry of 1 (25 g, 121 mmol) in CH₂Cl₂ and t-butanol (58 mL, 605 mmol) were added and the resulting mixture was capped tightly and allowed to stir vigorously for 3 d. The solids were filtered and the filtrate was washed with 2 N NaOH (100 mL), satd. aq. NaHCO₃ and brine (100 mL). The organics were concentrated and purified by chromatography (silica gel, 100% CH₂Cl₂) to obtain 2 (25 g, 78%) as a yellow oil: ¹H NMR (500 MHz, DMSO-d₆) δ 7.54 (d, J=4.0 Hz, 1H), 7.33 (d, J=4.0 Hz, 1H), 1.51 (s, 9H).

### Synthesis of 3

To 2 (19 g, 72 mmol) in THF (400 mL) at −78° C. was added n-butyllithium (29 mL, 72 mmol) dropwise. Upon complete addition the reaction was stirred for 30 min and triisopropyl borate (15 g, 79 mmol) was added dropwise. After 2 h was quenched by the addition of satd. aq. NH₄Cl (100 mL) and warmed to rt. The layers were separated and the organics were dried over Na₂SO₄, concentrated and purified by chromatography (silica gel, 0-75% ethyl acetate/hexanes) to obtain 3 (8.9 g, 56%) as a brown solid: ¹H NMR (500 MHz, DMSO-d₆) δ 7.71 (d, J=3.5 Hz, 1H), 7.49 (d, J=3.5 Hz, 1H), 1.49 (s, 9H).

### Synthesis of 4

To a solution of 3 (8.9 g, 39 mmol) in DME (100 mL) and ethanol (50 mL) was added 2,4-dichloropyrimidine (8.8 g, 59 mmol), Pd(Ph₃P)₂Cl₂ (1.4 g, 1.9 mmol) and 2 N Na₂CO₃ (30 mL). The resulting mixture was heated at 80° C. for 2 h. The reaction was cooled to rt and partitioned between H₂O (300 mL) and ethyl acetate (100 mL). The layers were separated and the aqueous layer was extracted with ethyl acetate (100 mL). The combined organic layers were dried over Na₂SO₄, filtered and concentrated. The residue was purified by column chromatography (0-50% ethyl acetate/heptane) to obtain crude material (9 g) as a tan solid. The crude solid was suspended in acetonitrile (50 mL) and filtered to obtain 4 (5.5 g, 50%) as a white solid: ¹H NMR (500 MHz, DMSO-d₆) δ 8.84 (d, J=9.0 Hz, 1H), 8.17-8.15 (m, 2H), 7.80 (d, J=7.0 Hz, 1H), 1.55 (s, 9H

### Synthesis of Acids:

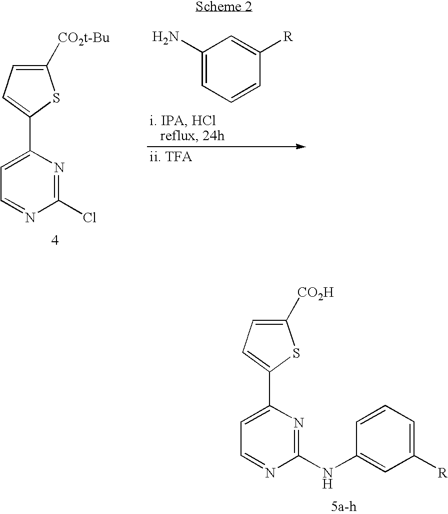

### Synthesis of 5a

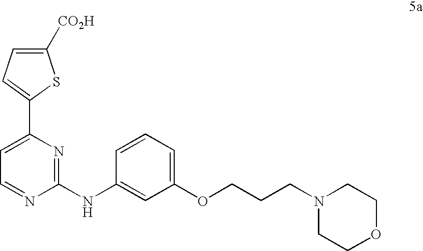

A solution of 4 N HCl in 1,4 dioxane (800 μL, 3.20 mmol) was added dropwise to a solution of aniline (756 mg, 3.20 mmol) and 4 (1.0 g, 3.37 mmol) in 2-propanol (10 mL) at reflux. The reaction mixture was heated at reflux for 18 h. The reaction was cooled to room temperature and concentrated under reduced pressure to afford the crude 2-anilino-pyrimidine (1.6 g) as a yellow solid: ESI MS m/z 497 [C₂₆H₃₂N₄O₄S+H]⁺. The solid (800 mg, 1.60 mmol) was dissolved in trifluoroacetic acid (50 mL) and stirred at room temperature for 3 h. The reaction was concentrated under reduced pressure and residual trifluoroacetic acid was removed by repeated co-distillation with methylene chloride or toluene to afford 5a (200 mg) as a bright yellow solid: ESI MS m/z 441 [C₂₂H₂₄N₄O₄S+H]⁺.

### Synthesis of 5b

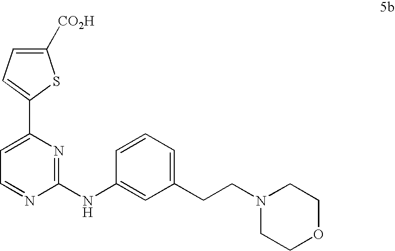

This compound was prepared by Method A described for acid 5a substituting 5 N HCl in 2-propanol as the acid source, to afford 5b (1.2 g, quantitative) as a yellow solid: ESI MS m/z 411 [C₂₁H₂₂N₄O₃S+H]⁺.

### Synthesis of 5c

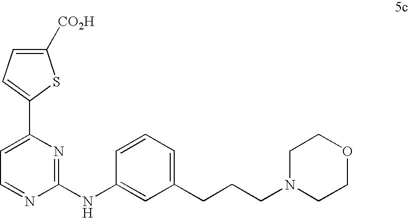

This compound was prepared by the same procedure described for 5a substituting 1 N HCl in diethyl ether as the acid source, to afford 5c (1.5 g, quant.) as a yellow-brown solid: ESI MS m/z 425 [C₂₂H₂₄N₄O₃S+H]⁺.

### Synthesis of 5d

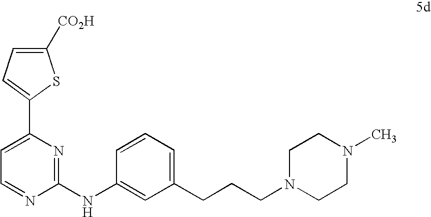

This compound was prepared by the same procedure described for 5a substituting 1 N HCl in diethyl ether as the acid source, to afford 5d (690 mg, 72%) as a yellow solid: ESI MS m/z 438 [C₂₃H₂₇N₅O₂S+H]⁺.

### Synthesis of 5e

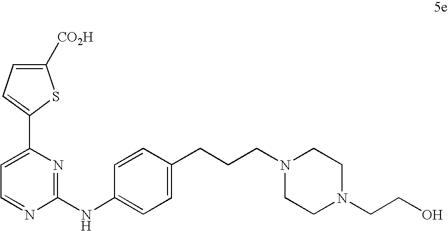

This compound was prepared by the same procedure described for 5a substituting 1 N HCl in diethyl ether as the acid source, to afford 5e (860 mg, 86%) as a brown solid: ESI MS m/z 468 [C₂₄H₂₉N₅O₃S+H]⁺.

### Synthesis of 5f

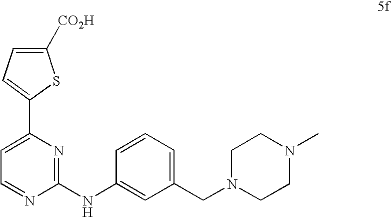

This compound was prepared by the same procedure described for 5a substituting 1 N HCl in diethyl ether as the acid source. This material was purified by chromatography (silica gel, 0-20% methanol/methylene chloride) to afford 5f (0.67 g, 45%) as a yellow solid. This material was used in subsequent reactions without further purification.

### Synthesis of 5g

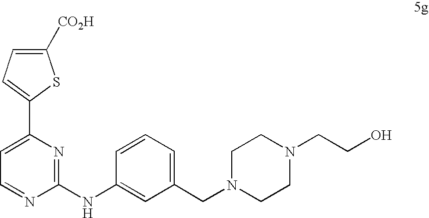

This compound was prepared by the same procedure described for 5a substituting 1 N HCl in diethyl ether as the acid source, to afford 5g (1.0 g crude) as a brown foam. This material was used in subsequent reactions without further characterization or purification.

### Synthesis of 5h

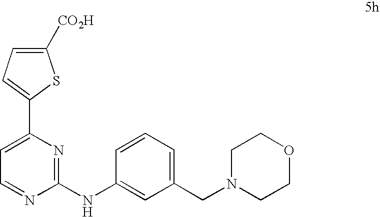

This compound was prepared by the same procedure described for 5a substituting 1 N HCl in diethyl ether as the acid source, to afford 5h (0.9 g crude) as a brown foam. This material was used in subsequent reactions without further characterization or purification.

### Synthesis of 5i

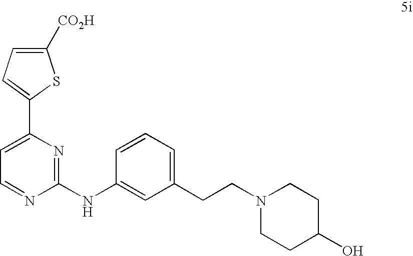

This compound was prepared by the same procedure described for 5a substituting 1 N HCl in diethyl ether as the acid source, to afford 5h (0.9 g crude) as a brown foam. This material was used in subsequent reactions without further characterization or purification.

### Synthesis of 5j

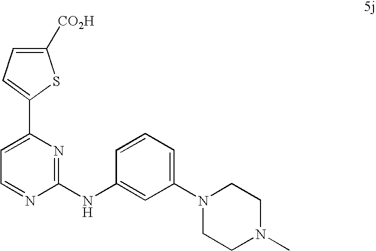

This compound was prepared by the same procedure described for 5a substituting 1 N HCl in diethyl ether as the acid source, to afford 5h (0.9 g crude) as a brown foam. This material was used in subsequent reactions without further characterization or purification.

### Synthesis of Examples:

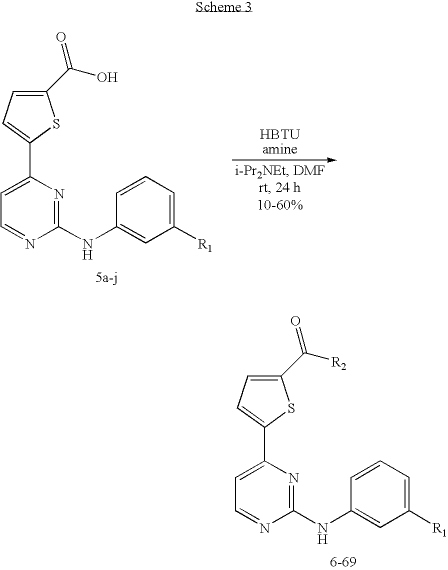

### Synthesis of Example 6

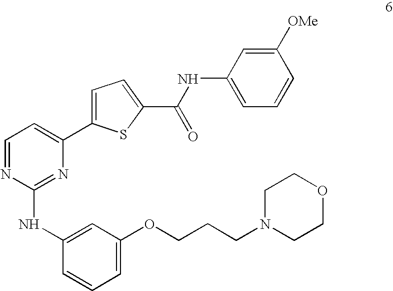

A solution of 5a (50 mg, 0.11 mmol) in N,N-dimethylformamide (1 mL) containing HBTU (47 mg, 0.12 mmol) and N,N-diisopropylethylamine (100 □L, 0.5 mmol) was stirred for 20 min at room temperature. The amine (300 □L, 0.15 mmol) was added and the reaction mixture was stirred at room temperature for 18 h. The reaction mixture was concentrated under reduce pressure and the residue was partitioned between ethyl acetate and dilute aq. NH₄Cl. The layers were separated and the organics were washed with satd. aq. NaHCO₃, H₂O, satd. aq. NaCl, and dried over sodium sulfate. The organics were concentrated and purified by chromatography 9silica gel, 0-10% methanol/methylene chloride with 1% NH₄OH) to afford 6 (36 mg 59%) as a bright yellow solid; mp 173-175° C.; ¹H NMR (500 MHz, DMSO-d₆) δ 10.32 (m, 1H), 9.74 (m, 1H), 8.58-8.57 (m, 1H), 8.07 (m, 2H), 7.73 (m, 1H), 7.44-7.42 (m, 2H), 7.33 (m, 1H), 7.29-7.23 (m, 2H), 7.20-7.18 (m, 1H), 6.72 (m, 1H), 6.55-6.54 (m, 1H), 4.10-4.08 (m, 2H), 3.76 (s, 3H), 3.52 (m, 4H), 2.49-2.44 (m, 2H), 2.35 (m, 4H), 1.94-1.93 (2H); ESI MS m/z 546 [C₂₉H₃₁N₅O₄S+H]⁺; HPLC: 97.7% (AUC) (Method A), t_{R}=12.47 min

### Synthesis of Example 7

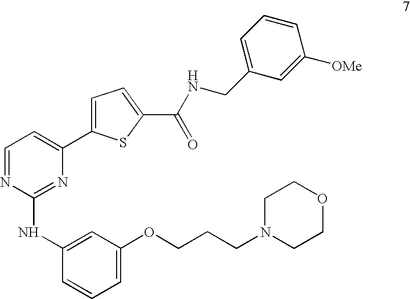

This compound was prepared by the same procedure using described for 6 to afford 7 (36 mg 57%) bright yellow solid; mp 143-144° C.; ¹H NMR (500 MHz, DMSO-d₆) δ 9.71 (m, 1H), 9.18 (m,1 H), 8.55-8.54 (m, 1H), 8.01-8.00 (m, 1H), 7.88-7.87 (m, 1H), 7.70-7.60 (m, 2H), 7.39-7.38 (m, 1H), 7.27-7.24 (m, 2H), 7.19-7.16 (m, 1H), 6.91-6.84 (m, 3H), 6.54 (m, 1H), 4.45-4.44 (m, 2H), 4.08-4.05, (m, 2H), 3.74 (s, 3H), 3.52 (m, 4H), 2.49-2.42 (m, 2H), 2.34 (m, 4H), 1.92-1.89 (m, 2H); ESI MS m/z 530 [C₃₀H₃₃N₅O₄S+H]⁺; HPLC: 96.8% (AUC) (Method A), t_{R}12.09 min

### Synthesis of Example 8

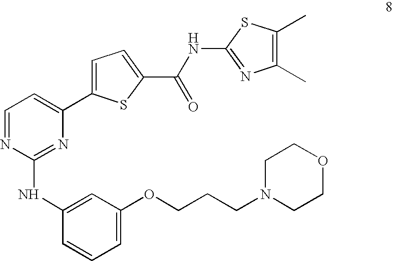

This compound was prepared by the same procedure described for 6 to afford 8 (33 mg, 57%) as a bright yellow powder: mp 181-183° C.; ¹H NMR (500 MHz, CDCl₃) δ 12.68 (m, 1H), 9.75 (m, 1H), 8.58-8.57 (m, 1H), 8.16-8.15 (m, 1H), 8.05 (m, 1H), 7.72 (m, 1H), 7.43-7.42 (m, 1H), 7.24-7.20 (m, 1H), 7.19-7.17 (m, 1H), 6.56-6.544.10-4.08 (m, 1H), 3.54-3.52 (m, 4H), 2.49-2.46 (m, 2H), 2.37 (m, 4H), 2.26 (s, 3H), 2.20 (s, 3H), 1.95-1.94; ESI MS m/z 551 [C₂₇H₃₀N₅₆O₃S+H]⁺; HPLC: 99.0% (AUC) (Method A), tR=12.08 min

### Synthesis of Example 9

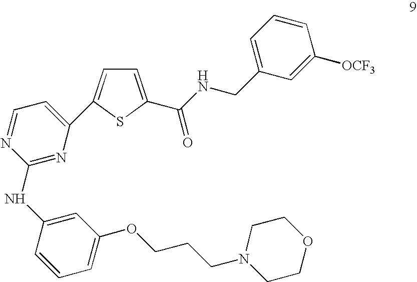

This compound was prepared by the same procedure described for 6 to afford 9 (30 mg, 23%) as a bright yellow powder; mp 138-140° C.; ¹H NMR (500 MHz, DMSO-d₆) δ 9.75 (m, 1H), 9.31 (m, 1H), 8.56-8.55 (m, 1H), 8.03-8.02 (m, 1H), 7.89 (m, 2H), 7.51-7.48 (m, 1H), 7.41-7.36 (m, 1H), 7.28-7.17 (m, 5H), 6.56-6.54 (m, 1H), 4.54-4.53 (m, 2H), 4.11 (m, 2H), 3.90-3.80 (m, 1H), 3.58 (m, 4H), 3.20-3.10 (m, 1H), 2.40-1.80 (m, 5H); ESI MS m/z 544 [C₂₉H₂₉N₅O₄S+H]⁺; HPLC: >99% (AUC) (Method A), tR=13.62 min

### Synthesis of Example Target 10

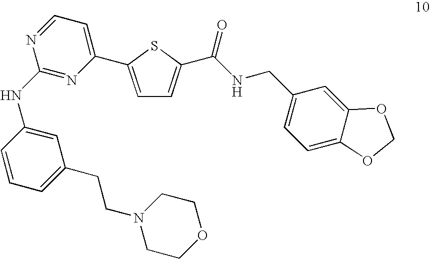

This compound was prepared by the same procedure described for 6 to afford 10 (20 mg, 12%) as a bright yellow powder; mp 144-146° C.; ¹H NMR (500 MHz, DMSO-d₆) δ 9.66 (m, 1H), 9.13 (m, 1H), 8.54-8.53 (m, 1H), 7.99-7.98 (m, 1H), 7.85-7.84 (m, 2H), 7.52 (m, 1H), 7.37-7.36 (m, 1H), 7.21 (m, 1H), 6.90-6.80 (m, 4H), 5.98 (m, 2H), 4.38-4.36 (m, 2H), 3.58 (m, 4H), 2.88 (m,1 H), 2.76 (m, 2H), 2.60 (m, 2H), 2.49-2.46 (m, 4H); ESI MS m/z 544 [C₂₉H₂₉N₅O₄S+H]⁺; HPLC: 98.9% (AUC) (Method A), tR=11.71 min

### Synthesis of Example 11

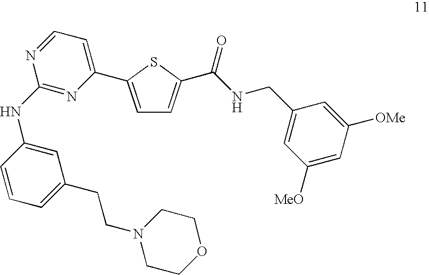

This compound was prepared by the same procedure described for 6 to afford 11 (60 mg, 34%) as a bright yellow powder; mp 176-178° C.; ¹H NMR (500 MHz, DMSO-d₆) δ 9.66 (m, 1H), 9.15 (m, 1H), 8.54-8.53 (m, 1H), 8.0-7.99 (m, 1H), 7.87-7.86 (m, 1H), 7.83 (m, 1H), 7.53-7.47 (m, 1H), 7.38-7.36 (m, 1H), 7.22-7.19 (m, 1H), 6.85-6.84 (m, 1H), 6.49 (m, 2H), 6.40-6.39 (m, 1H), 4.41-4.40 (m, 2H), 3.72 (m, 6H), 3.56 (m, 4H), 2.74 (m, 2H), 2.54 (m, 2H), 2.49-2.44 (m, 4H); ESI MS m/z 560 [C₃₀H₃₃N₅O₄S+H]⁺; HPLC: >99% (AUC) (Method A), tR=11.79 min

### Synthesis of Example 12

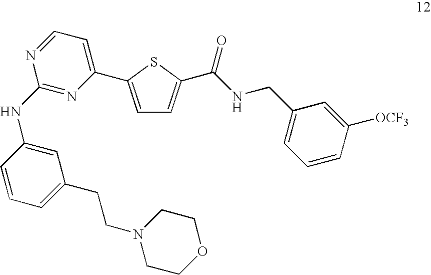

This compound was prepared by the same procedure described for 6 to afford 12 (50 mg, 27%) as a bright yellow powder; mp 156-158° C.; ¹H NMR (500 MHz, DMSO-d₆) δ 9.66 (m, 1H), 9.27 (m, 1H), 8.54-8.53 (m, 1H), 8.01-8.00 (m, 1H), 7.87-7.86 (m, 1H), 7.83 (m, 1H), 7.53-7.47 (m, 1H), 7.38-7.37 (m, 1H), 7.31-7.25 (m, 4H), 7.22-7.19 (m, 1H), 6.85-6.84 (m, 1H), 4.53-4.52 (m, 2H), 3.56-3.54 (m, 4H), 2.74-2.72 (m, 2H), 2.56-2.54 (m, 2H), 2.49-2.44 (m, 4H); ESI MS m/z 584 [C₂₉H₂₈F₃N₅O₃S+H]⁺; HPLC: >99% (AUC) (Method A), tR=12.89 min

### Synthesis of Example 13

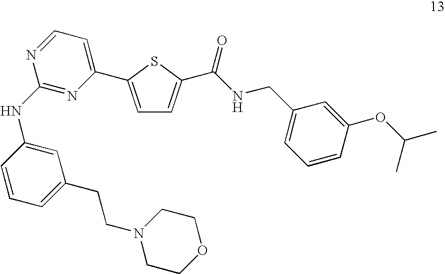

This compound was prepared by the same procedure described for 6 to afford 13 (38 mg, 21%) as a bright yellow powder; mp 129-131° C.; ¹H NMR (500 MHz, DMSO-d₆) δ 9.65 (m, 1H), 9.17 (m, 1H), 8.54-8.53 (m, 1H), 8.00 (m, 1H), 7.87 (m, 2H), 7.54-7.52 (m, 1H), 7.38-7.37 (m, 1H), 7.24-7.21 (m, 2H), 6.87-6.85 (m, 2H), 6.81-6.80 (m, 2H), 4.60-4.55 (m, 1 H), 4.44-4.42 (m, 2H), 3.56 (m, 4H), 2.74 (m, 2H), 2.55-2.53 (m, 2H), 2.49-2.45 (m, 4H), 1.26-1.22 (m, 6H); ESI MS m/z 558 [C₃₁H₃₅N₅O₃S+H]⁺; HPLC: 98.6% (AUC) (Method A), tR=12.61 min

### Synthesis of Example 14

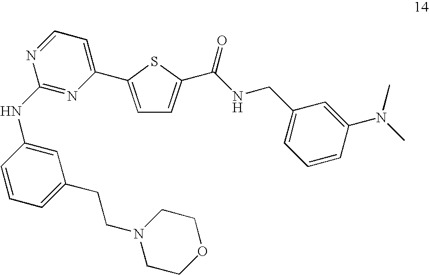

This compound was prepared by the same procedure described for 6 to afford 14 (18 mg, 10%) as a light orange powder; mp 100-102° C.; ¹H NMR (500 MHz, DMSO-d₆) δ 9.66 (m, 1H), 9.12 (m, 1H), 8.54-8.53 (m, 1H), 7.99 (m, 1H), 7.87-7.86 (m, 1H),7.83 (m, 1H), 7.54-7.52 (m, 1H), 7.37-7.36 (m, 1H), 7.21 (m, 1H), 7.15-7.12 (m, 1H), 6.86-6.84 (m, 1H), 6.70 (m, 1H), 6.63-6.61 (m, 2H), 4.42-4.40 (m, 2H), 3.60-3.50 (m, 4H), 2.90 (m, 3H), 2.88 (m, 3H), 2.74 (m, 2H), 2.55 (m, 2H), 2.45 (m, 2H), 1.27-1.22 (m, 2H); ESI MS m/z 543 [C₃₀H₃₄N₆O₂S+H]⁺; HPLC: 98.3% (AU C) (Method A), tR=9.33 min

### Synthesis of Example 15

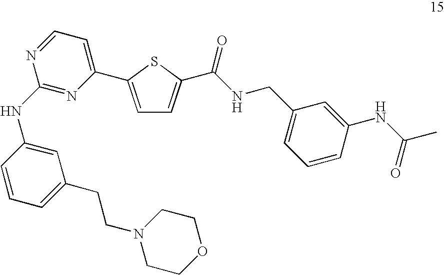

This compound was prepared by the same procedure described for 6 to afford 15 (58 mg, 33%) as a light orange solid; mp 234-236° C.; ¹H NMR (500 MHz, DMSO-d₆) δ 9.92 (m, 1H), 9.65 (m, 1H), 9.20 (m, 1H), 8.54-8.53 (m, 1H), 8.00-7.99 (m, 1H), 7.87-7.86 (m, 1H), 7.81 (m, 1H), 7.55-7.50 (m, 3H), 7.37-7.36 (m, 1H), 7.25-7.19 (m, 2H), 7.00-6.99 (m, 1H), 6.85-6.84 (m, 1H), 4.44-4.43 (m, 2H), 3.56-3.55 (m, 4H), 2.75-2.72 (m, 2H), 2.56-2.53 (m, 2H), 2.49-2.44 (m, 4H), 2.01(s, 3H), ESI MS m/z 557 [C₃₀H₃₂N₆O₃S+H]⁺; HPLC (Method A)>99% (AUC), t_{R}=10.62 min

### Synthesis of Example 16

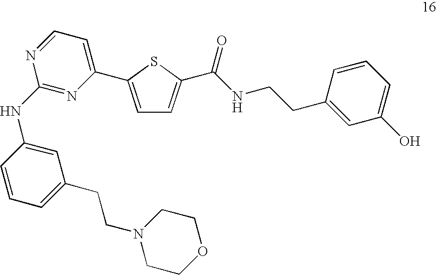

This compound was prepared by the same procedure described for 6 to afford 16 (55 mg, 47%) as a bright yellow powder; mp 164-166° C.; ¹H NMR (500 MHz, DMSO-d₆) δ 10.72 (m, 1H), 9.79 (m, 1H), 9.31 (m, 1H),8.88-8.87 (m, 1H), 8.56-8.55 (m, 1H), 8.02 (m, 2H), 7.83-7.82 (m, 1H), 7.40-7.39 (m, 1H), 7.29 (m, 1H), 7.08-7.07 (m, 1H), 6.92-6.90 (m, 1H), 6.66-6.65 (m, 2H), 6.62-6.60 (m, 1H), 4.02-4.00 (m, 2H), 3.80 (m, 2H), 3.58-3.56 (m, 2H), 3.44 (m, 4H), 3.34 (m, 2H), 3.10-3.08 (m, 2H), 2.77-2.74 (m, 2H);ESI MS m/z 530 [C₂₉H₃₁N₅O₃S+H]⁺; HPLC (Method A)>99% (AUC), t_{R}=11.23 min

### Synthesis Example 17

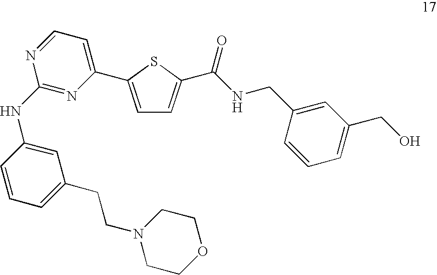

This compound was prepared by the same procedure described for 6 to afford 17 (45 mg, 39%) as a bright yellow powder; mp 110-112° C.; ¹H NMR (500 MHz, DMSO-d₆) δ 9.73 (m, 1H), 9.21-9.18 (m, 1H), 8.54-8.53 (m, 1H), 8.00-7.99 (m, 1H), 7.87-7.86 (m, 1H), 7.82 (m, 1H), 7.55-7.54 (m, 1H), 7.37-7.36 (m, 1H), 7.30-7.27 (m, 2H), 7.22-7.19 (m, 3H), 6.85-6.84 (m, 1H), 5.19-5.17 (m, 1H), 4.49-4.46 (m, 4H), 3.56 (m, 4H), 2.74-2.73 (m, 2H), 2.56-2.54 (m, 2H), 2.49-2.44 (m, 4H); ESI MS m/z 530 [C₂₉H₃₁N₅O₃S+H]⁺; HPLC (Method A) 98.1% (AUC), t_{R}=10.63 min

### Synthesis of Example 18

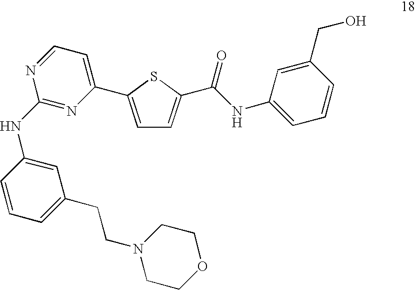

This compound was prepared by the same procedure described for 6 to afford 18 (12 mg, 6%) as a yellow powder; mp 237-239° C.; ¹H NMR (500 MHz, DMSO-d₆) δ 10.34 (m, 1H), 9.69 (m, 1H), 8.56-8.55 (m, 1H), 8.09-8.06 (m, 2H), 7.90 (m, 1H), 7.74 (m, 1H), 7.66-7.64 (m, 1H), 7.52-7.50 (m, 1H), 7.41-7.40 (m, 1H), 7.31 (m, 1H), 7.22 (m, 1H), 7.07-7.06 (m, 1H),6.86-6.85 (m, 1H), 5.25-5.23 (m, 1H), 4.52-4.51 (m, 2H), 3.51 (m, 4H), 2.76 (m, 2H), 2.57 (m, 2H), 2.47 (m, 4H); ESI MS m/z 516 [C₂₈H₂₉N₅O₃S+H]⁺; HPLC (Method A) 92.9% (AUC), t_{R}=10.67 min

### Synthesis of Example 19

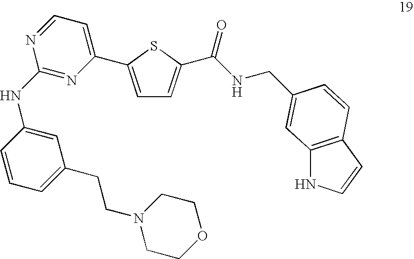

This compound was prepared by the same procedure described for 6 to afford 19 (12 mg, 6%) as a yellow powder; mp 199-200° C.; ¹H NMR (500 MHz, DMSO-d₆) δ 11.02 (m, 1H), 9.65 (m, 1H), 9.17 (m, 1H), 8.53-8.52 (m, 1H), 7.99-7.98 (m, 1H), 7.88-7.87 (m, 1H), 7.82 (m, 1H), 7.56-7.55 (m, 1H), 7.49-7.48 (m, 1H), 7.37-7.35 (m, 2H), 7.30 (m, 1H), 7.21 (m, 1H), 7.00-6.98 (m, 1H), 6.85-6.84 (m, 1H), 6.38 (m, 1H), 4.56-4.55 (m, 2H), 3.57 (m, 4H), 2.76-2.73 (m, 2H), 2.55 (m, 2H), 2.45 (m, 4H); ESI MS m/z 539 [C₃₀H₃₀N₆O₂S+H]⁺; HPLC (Method A) 98.2% (AUC), t_{R}=11.64 min

### Synthesis of Example 20

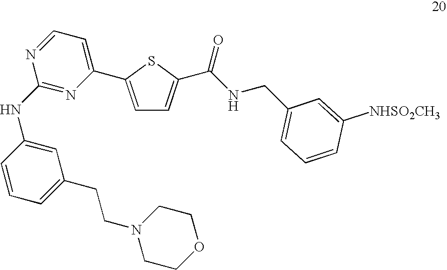

This compound was prepared by the same procedure described for 6 to afford 20 (17 mg, 7%) as a yellow powder; mp 237-239° C.; ¹H NMR (500 MHz, DMSO-d₆) δ 9.75 (m, 1H), 9.66 (m, 1H), 9.21 (m, 1H),8.54-8.53 (m, 1H), 8.00 (m, 1H), 7.87-7.86 (m, 1H), 7.83-7.82 (m, 1H), 7.54 (m, 1H), 7.38-7.37 (m, 1H), 7.30 (m, 1H), 7.20-7.18 (m, 2H), 7.12-7.10 (m, 1H), 7.08-7.06 (m, 1H), 6.85-6.84 (m, 1H), 4.45-4.44 (m, 2H), 3.55 (m, 4H), 2.98 (s, 3H), 2.75-2.72 (m, 2H), 2.56-2.50 (m, 2H), 2.44 (m, 4H); ESI MS m/z 593 [C₂₉H₃₂N₆O₄S+H]⁺; HPLC (Method A) 97.5% (AUC), t_{R}=10.97 min

### Synthesis of Example 21

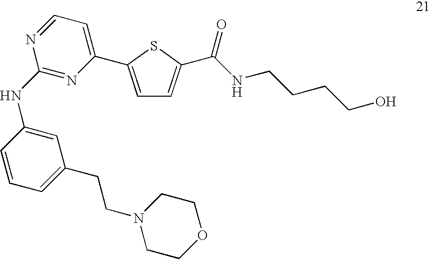

This compound was prepared by the same procedure described for 6 to afford 21 (22 mg, 11%) as a yellow powder; mp 189-191° C.; ¹H NMR (500 MHz, DMSO-d₆) δ 9.65 (m, 1H), 8.62 (m, 1H), 8.53-8.52 (m, 1H), 7.98-7.97 (m, 1H), 7.83 (m, 1H), 7.79-7.78 (m, 1H), 7.54-7.53 (m, 1H), 7.36-7.35 (m, 1H), 7.22-7.19 (m, 1H), 6.85-6.84 (m, 1H), 4.43-4.41 (m, 1H), 3.58-3.56 (m, 4H), 3.42-3.40 (m, 2H), 3.26-3.24 (m, 2H), 2.74-2.72 (m, 2H), 2.56-2.53 (m, 2H), 2.49-2.45 (m, 4H), 1.55-1.54 (m, 2H), 1.48-1.46 (m, 2H); ESI MS m/z 482 [C₂₅H₃₁N₅O₃S+H]⁺; HPLC (Method A) 98.8% (AUC), t_{R}=9.62 min

### Synthesis of Example 22

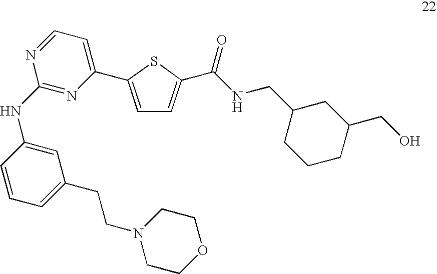

This compound was prepared by the same procedure described for 6 to afford 22 (16 mg, 8%) as a yellow powder; mp 120-122° C.; ¹H NMR (500 MHz, DMSO-d₆) δ 9.65 (m, 1H), 8.53 (m, 1H), 8.52-8.38 (m, 1H), 7.97-7.96 (m, 1H), 7.84-7.83 (m, 2H), 7.54-7.53 (m, 1H), 7.36-7.35 (m, 1H), 7.22-7.19 (m, 1H), 6.85-6.84 (m, 1H),4.45-4.43 (m, 1H),3.80-3.70 (m, 1H), 3.58-3.56 (m, 4H), 3.25-3.20 (m, 2H), 2.76-2.73 (m, 2H), 2.56-2.50 (m, 2H), 2.46-2.44 (m, 4H), 1.90-1.50 (m, 4H), 1.45-1.40 (m, 1H), 1.30-0.90 (m, 4H); ESI MS m/z 522 [C₂₈H₃₅N₅O₃S+H]⁺; HPLC (Method A) 95.9% (AUC), t_{R}=10.50 min

### Synthesis of Example 23

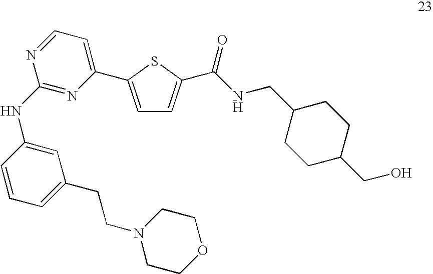

This compound was prepared by the same procedure described for 6 to afford 23 (17 mg, 8%) as a yellow powder; mp 200-202° C.; ¹H NMR (500 MHz, DMSO-d₆) δ 9.65 (s, 1H), 8.60 (m, 1H), 8.53 (m, 1H), 7.98-7.97 (m, 1H), 7.84-7.81 (m, 2H), 7.54-7.52 (m, 1H), 7.36-7.35 (m, 1H), 7.22-7.19 (m, 1H), 6.85-6.84 (m, 1H), 4.36-4.34 (m, 1H), 3.58-3.56 (m, 4H), 3.32 (s, 2H), 3.21-3.19 (m, 2H), 3.11-3.08 (m, 2H), 2.74-2.73 (m, 2H), 2.56-2.54 (m, 2H), 2.49-2.44 (m, 4H), 1.76-1.75 (m, 4H), 1.50-1.40 (m, 1H), 1.40-1.20 (m, 1 H), 0.90-0.70 (m, 4H); ESI MS m/z 536 [C₂₉H₃₇N₅O₃S+H]⁺; HPLC (Method A) 96.4% (AUC), t_{R}=10.65 min

### Synthesis of Example 24

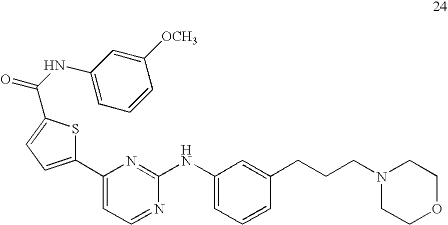

This compound was prepared by the same procedure described for 6 above. The crude product was purified by chromatography (silica gel, 94.5:4.5:0.5 methylene chloride/methanol/concentrated ammonium hydroxide) to afford 24 (84 mg, 42%) as a brown solid: mp 80-84° C.; ¹H NMR (500 MHz, DMSO-d₆) δ 10.31 (s, 1H), 9.67 (s, 1H), 8.56-8.55 (m, 1H), 8.07 (s, 2H), 7.81 (s, 1H), 7.56-7.54 (m, 1H), 7.44 (s, 1H), 7.41-7.40 (m, 1H), 7.34-7.33 (m, 1H), 7.29-7.25 (m, 1H), 7.23-7.20 (m, 1H), 6.84-6.83 (m, 1H), 6.72-6.70 (m, 1H), 3.76 (s, 3H), 3.54-3.52 (m, 4H), 2.63-2.60 (m, 2H), 2.33-2.31 (m, 6H), 1.79-1.76 (m, 2H); ESI MS m/z 530 [C₂₉H₃₁N₅O₃S+H]⁺; HPLC (Method A) 97.3% (AUC), t_{R}=12.48 min.

### Synthesis of Example 25

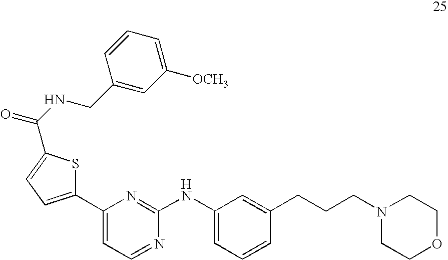

This compound was prepared by the same procedure described for 6 above. The crude product was purified by chromatography (silica gel, 94.5:4.5:0.5 methylene chloridelmethanol/concentrated ammonium hydroxide) to afford 25 (114 mg, 59%) as a brown solid: mp 65-70° C.; ¹H NMR (500 MHz, DMSO-d₆) δ 9.64 (s, 1H), 9.19-9.17 (m, 1H), 8.53-8.52 (m, 1H), 8.00-7.99 (m, 1H), 7.87-7.86 (m, 1H), 7.77 (s, 1H), 7.52-7.50 (m, 1H), 7.37-7.36 (m, 1H), 7.27-7.25 (m, 1H), 7.22-7.20 (m, 1H), 6.91-6.90 (m, 2H), 6.85-6.83 (m, 2H), 4.46-4.44 (m, 2H), 3.74 (s, 3H), 3.54-3.52 (m, 4H), 2.60-2.58 (m, 2H), 2.31-2.28 (m, 6H), 1.79-1.76 (m, 2H); ESI MS m/z 544 [C₃₀H₃₃N₅O₃S+H]⁺; HPLC (Method A) 98.8% (AUC), t_{R}=12.09 min.

### Synthesis of Example 26

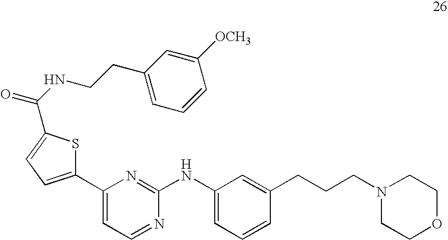

This compound was prepared by the same procedure described for 6 above. The crude product was purified by chromatography (silica gel, 94.5:4.5:0.5 methylene chloride/methanol/concentrated ammonium hydroxide) to afford 26 (104 mg, 49%) as a brown solid: mp 55-58° C.; ¹H NMR (500 MHz, DMSO-d₆) δ 9.64 (s, 1H), 8.74-8.72 (m, 1H), 8.53-8.52 (m, 1H), 7.98-7.97 (m, 1H), 7.78-7.77 (m, 2H), 7.58-7.56 (m, 1H), 7.36-7.35 (m, 1H), 7.22-7.19 (m, 2H), 6.83-6.77 (m, 4H), 3.72 (s, 3H), 3.55-3.54 (m, 4H), 3.48-3.47 (m, 2H), 2.84-2.81 (m, 2H), 2.62-2.59 (m, 2H), 2.33-2.30 (m, 6H), 1.78-1.75 (m, 2H); ESI MS m/z 558 [C₃₁H₃₅N₅O₃S+H]⁺; HPLC (Method A) 95.5% (AUC), t_{R}=12.46 min.

### Synthesis of Example 27

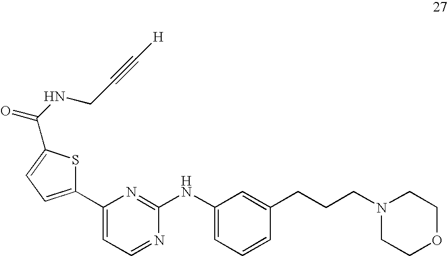

This compound was prepared by the same procedure described for 6 above. The crude product was purified by chromatography (silica gel, 94.5:4.5:0.5 methylene chloride/methanol/concentrated ammonium hydroxide) to afford 27 (89 mg, 50%) as a yellow solid: mp 68-72° C.; ¹H NMR (500 MHz, DMSO-d₆) δ 9.65 (s, 1H), 9.12-9.11 (m, 1H), 8.54-8.53 (m, 1H), 7.99-7.98 (m, 1H), 7.84-7.83 (m, 1H), 7.76 (s, 1H), 7.58-7.56 (m, 1H), 7.37-7.36 (m, 1H), 7.22-7.19 (m, 1H), 6.83-6.82 (m, 1H), 4.07-4.06 (m, 2H), 3.55-3.54 (m, 4H), 3.19-3.18 (m, 1H), 2.62-2.59 (m, 2H), 2.32-2.29 (m, 6H), 1.78-1.75 (m, 2H); ESI MS m/z 462 [C₂₅H₂₇N₅O₂S+H]⁺; HPLC (Method A) >99% (AUC), t_{R}=10.65 min.

### Synthesis of Example 28

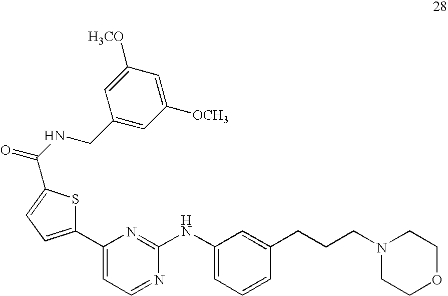

This compound was prepared by the same procedure described for 6 above. The crude product was purified by chromatography (silica gel, 94.5:4.5:0.5 methylene chloride/methanol/concentrated ammonium hydroxide) to afford 28 (90 mg, 45%) as a yellow solid: mp 65-70° C.; ¹H NMR (500 MHz, DMSO-d₆) δ 9.62 (s, 1H), 9.14-9.12 (m, 1H), 8.53-8.52 (m, 1H), 7.99-7.98 (m, 1H), 7.87-7.86 (m, 1H), 7.76 (s, 1H), 7.58-7.57 (m, 1H), 7.36-7.35 (m, 1H), 7.22-7.19 (m, 1H), 6.83-6.81 (m, 1H), 6.50-6.49 (m, 2H), 6.40-6.39 (m, 1H), 4.41-4.40 (m, 2H), 3.72 (s, 6H), 3.54-3.52 (m, 4H), 2.61-2.58 (m, 2H), 2.31-2.28 (m, 6H), 1.79-1.73 (m, 2H); ESI MS m/z 574 [C₃₁H₃₅N₅O₄S+H]⁺; HPLC (Nethod A) >99% (AUC), t_{R}=12.33 min.

### Synthesis of Example 29

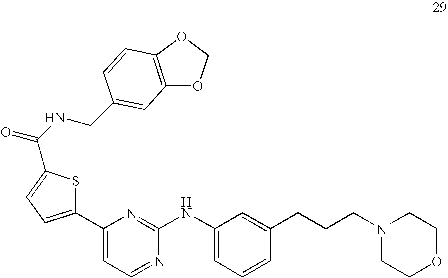

This compound was prepared by the same procedure described for 6 above. The crude product was purified by chromatography (silica gel, 94.5:4.5:0.5 methylene chloride/methanol/concentrated ammonium hydroxide) to afford 29 (127 mg, 65%) as a yellow solid: mp 74-80° C.; ¹H NMR (500 MHz, DMSO-d₆) δ9.64 (s, 1H), 9.13-9.11 (m, 1H), 8.53-8.52 (m, 1H), 7.99-7.98 (m, 1H), 7.85-7.84 (m, 1H), 7.77 (s, 1H), 7.58-7.56 (m, 1H), 7.36-7.35 (m, 1H), 7.22-7.19 (m, 1H), 6.90-6.86 (m, 2H), 6.83-6.80 (m, 2H), 5.98 (s, 2H), 4.38-4.37 (m, 2H), 3.54-3.53 (m, 4H), 2.61-2.58 (m, 2H), 2.32-2.29 (m, 6H), 1.77-1.74 (m, 2H); ESI MS m/z 558 [C₃₀H₃₁N₆O₄S+H]⁺; HPLC (Method A) 98.4% (AUC), t_{R}=12.10 min.

### Synthesis of Example 30

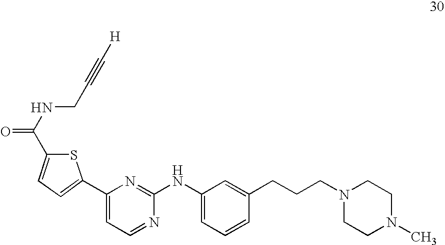

This compound was prepared by the same procedure described for 6 above. The crude product was purified by chromatography (silica gel, 94.5:4.5:0.5 methylene chloride/methanol/concentrated ammonium hydroxide) to afford 30 (81 mg, 53%) as a yellow solid: mp 72-76° C.; ¹H NMR (500 MHz, DMSO-d₆) δ 9.64 (s, 1H), 9.12-9.10 (m, 1H), 8.54-8.53 (m, 1H), 7.99-7.98 (m, 1H), 7.84-7.83 (m, 1H), 7.74 (s, 1H), 7.58-7.56 (m, 1H), 7.37-7.36 (m, 1H), 7.22-7.19 (m, 1H), 6.82-6.81 (m, 1H), 4.08-4.06 (m, 2H), 3.19-3.18 (m, 1H), 2.60-2.57 (m, 2H), 2.30-2.27 (m, 10H), 2.21 (s, 3H), 1.78-1.75 (m, 2H); ESI MS m/z 475 [C₂₆H₃₀N₆OS+H]⁺; HPLC (Method A) 98.6% (AUC), t_{R}=9.74 min.

### Synthesis of Example 31

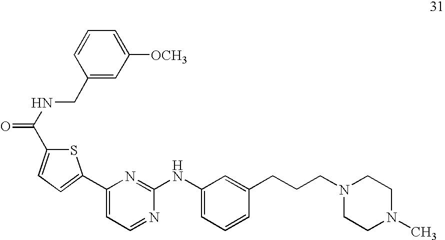

This compound was prepared by the same procedure described for 6 above. The crude product was purified by chromatography (silica gel, 94.5:4.5:0.5 methylene chloride/methanol/concentrated ammonium hydroxide) to afford 31 (80 mg, 45%) as a yellow solid: mp 62-68° C.; ¹H NMR (500 MHz, DMSO-d₆) δ 9.63 (s, 1H), 9.19-9.17 (m, 1H), 8.53-8.52 (m, 1H), 8.00-7.99 (m, 1H), 7.87-7.86 (m, 1H), 7.75 (s, 1H), 7.59-7.57 (m, 1H), 7.37-7.36 (m, 1H), 7.27-7.24 (m, 1H), 7.21-7.20 (m, 1H), 6.91-6.90 (m, 2H), 6.84-6.80 (m, 2H), 4.46-4.44 (m, 2H), 3.74 (s, 3H), 2.60-2.57 (m, 2H), 2.29-2.27 (m, 10H), 2.11 (s, 3H), 1.75-1.72 (m, 2H); ESI MS m/z 557 [C₃₁H₃₆N₆O₂S+H]⁺; HPLC (Method A) 96.9% (AUC), t_{R}=10.86 min.

### Synthesis of Example 32

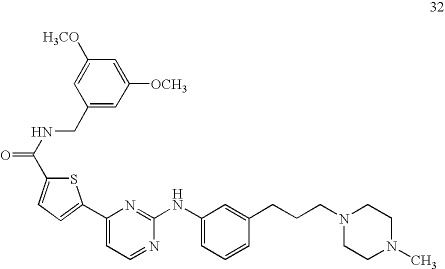

This compound was prepared by the same procedure described for 6 above. The crude product was purified by chromatography (silica gel, 94.5:4.5:0.5 methylene chloride/methanol/concentrated ammonium hydroxide) to afford 32 (65 mg, 51%) as a yellow solid: mp 69-80° C.; ¹H NMR (500 MHz, DMSO-d₆) δ 9.63 (s, 1H), 9.16-9.13 (m, 1H), 8.53-8.52 (m, 1H), 8.00-7.99 (m, 1H), 7.87-7.86 (m, 1H), 7.75 (s, 1H), 7.59-7.57 (m, 1H), 7.37-7.36 (m, 1H), 7.21-7.18 (m, 1H), 6.82-6.80 (m, 1H), 6.50-6.49 (m, 2H), 6.40-6.39 (m, 1H), 4.41-4.40 (m, 2H), 3.72 (s, 6H), 2.60-2.57 (m, 2H), 2.30-2.27 (m, 10H), 2.11 (s, 3H), 1.77-1.71 (m, 2H); ESI MS m/z 587 [C₃₂H₃₃N₆O₃S+H]⁺; HPLC (Method A) 98.2% (AUC), t_{R}=10.86 min.

### Synthesis of Example 33

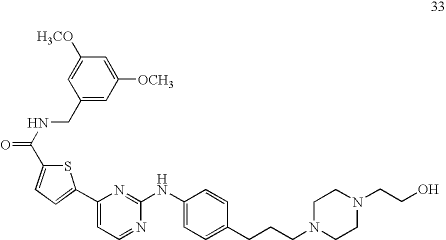

This compound was prepared by the same procedure described for 6 above. The crude product was purified by chromatography (silica gel, 94.5:4.5:0.5 methylene chloride/methanol/concentrated ammonium hydroxide) to afford 33 (12 mg, 6%) as a yellow solid: ¹H NMR (500 MHz, DMSO-d₆) δ 9.60 (s, 1H), 9.15-9.13 (m, 1H), 8.51-8.50 (m, 1H), 7.99-7.98 (m, 1H), 7.86 (s, 1H), 7.70-7.69 (m, 2H), 7.34-7.33 (m, 1H), 7.14-7.12 (m, 2H), 6.49 (s, 2H), 6.40 (s, 1H), 4.42-4.40 (m, 2H), 4.33-4.30 (m, 1H), 3.72 (s, 6H), 3.47-3.45 (m, 2H), 2.36-2.24 (m, 14H), 1.71-1.69 (m, 2H); ESI MS m/z 617 [C₃₃H₄₀N₆O₄S+H]⁺; HPLC (Method A) 95.6% (AUC), t_{R}=10.72 min.

### Synthesis of Example 34

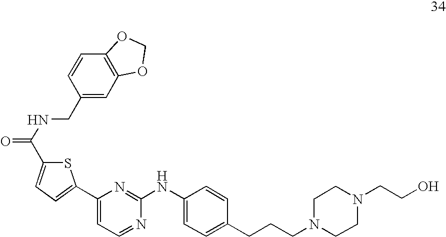

This compound was prepared by the same procedure described for 6 above. The crude product was purified by chromatography (silica gel, 94.5:4.5:0.5 methylene chloride/methanol/concentrated ammonium hydroxide) to afford 34 (20 mg, 11%) as a yellow solid: ¹H NMR (500 MHz, DMSO-d₆) δ 9.60 (s, 1H), 9.15-9.13 (m, 1H), 8.51-8.50 (m, 1H), 7.98-7.97 (m, 1H), 7.85-7.86 (m, 1H), 7.71-7.69 (m, 2H), 7.34-7.33 (m, 1H), 7.14-7.13 (m, 2H), 6.90-6.86 (m, 2H), 6.82-6.80 (m, 1H), 4.38-4.37 (m, 3H), 3.47-3.45 (m, 2H), 2.55-2.52 (m, 3H), 2.41-2.25 (m, 13H), 1.71-1.69 (m, 2H); ESI MS m/z 601 [C₃₂H₃₆N₆O₄S+H]⁺; HPLC (Method A) 92.4% (AUC), t_{R}=10.56 min.

### Synthesis of Example 35

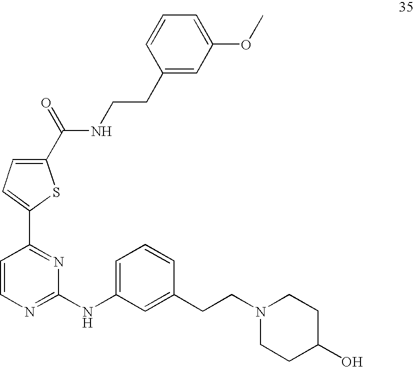

This compound was prepared by the same procedure described for 6 to afford 35 (52 mg, 40%) as a yellow solid: mp 97-99° C.; ¹H NMR (500 MHz, DMSO-d₆) δ 9.64 (s, 1H), 8.73 (t, J=5.6 Hz, 1H), 8.53 (d, J=5.1 Hz, 1H), 7.97 (d, J=4.0 Hz, 1H), 7.77 (d, J=3.9 Hz, 2H), 7.56 (d, J=8.1 Hz, 1H), 7.36 (d, J=5.2 Hz, 1H), 7.23-7.19 (m, 2H), 6.84-6.77 (m, 4H), 4.51 (d, J=4.3 Hz, 1H), 3.73 (s, 3H), 3.49-3.41 (m, 3H), 2.85-2.79 (m, 4H), 2.73-2.70 (m, 2H), 2.10-2.06 (m, 2H), 1.72-1.70 (m, 2H), 1.41-1.34 (m, 2H); ESI MS m/z 558 [C₃₁H₃₅N₅O₃S+H]⁺; HPLC (Inertsil ODS2 C18 Column) >99%, t_{R}=2.0 min.

### Synthesis of Example 36

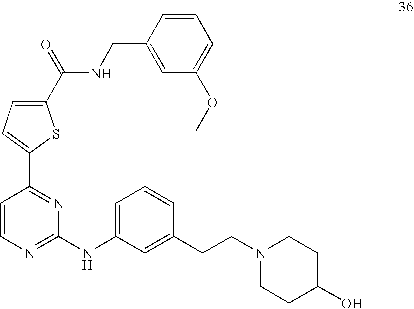

This compound was prepared by the same procedure described for 6 to afford 36 (62 mg, 49%) as a yellow solid: mp 98-100° C.; ¹H NMR (500 MHz, DMSO-d₆) δ 9.64 (s, 1H), 9.17 (t, J=5.9 Hz, 1H), 8.53 (d, J=5.1 Hz, 1H), 7.97 (d, J=4.0 Hz, 1H), 7.86 (d, J=4.1 Hz, 1H), 7.76 (s, 1H), 7.57 (d, J=8.1 Hz, 1H), 7.37 (d, J=5.1 Hz, 1H), 7.26 (t, J=8.1 Hz, 1H), 7.20 (t, J=7.8Hz, 1H), 6.91 (d, J=7.5Hz, 2H), 6.83 (d, J=7.6 Hz, 2H), 4.51 (d, J=4.3 Hz, 1H), 4.45 (d, J=6.0 Hz, 2H), 3.74 (s, 3H), 3.45-3.40 (m, 1H), 2.81-2.77 (m, 2H), 2.73-2.70 (m, 2H), 2.53-2.50 (m, 2H), 2.07(t, J=10.7 Hz, 2H), 1.72-1.69 (m, 2H), 1.38-1.35 (q, J=10.0 Hz, 2H); ESI MS m/z 544 [C₃₀H₃₃N₅O₃S+H]⁺; HPLC>99%, t_{R}=2.0 min.

### Synthesis of Example 37

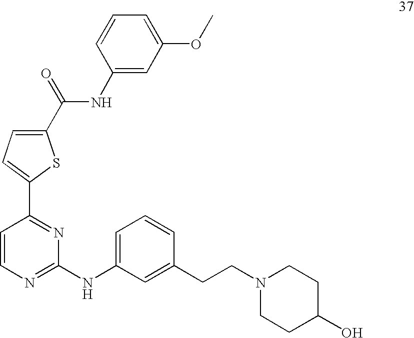

This compound was prepared by the same procedure described for 6 to afford 37 (65 mg, 53%) as a yellow solid: mp 208-210° C.; ¹H NMR (500 MHz, DMSO-d₆) δ 10.31 (s, 1H), 9.68 (s, 1H), 8.56 (t, J=5.1 Hz, 1H), 8.08-8.06 (dd, J=5.4, 4.2 Hz, 2H), 7.82 (s, 1H), 7.54 (d, J=8.1 Hz, 1H), 7.45 (d, J=2.1 Hz, 1H), 7.43 (d, J=15.1 Hz, 1H), 7.35 (d, J=8.2Hz, 1H), 7.27 (t, J=7.1 Hz, 1H), 7.22 (t, J=7.8 Hz, 1H), 6.84 (d, J=7.5 Hz, 1H), 6.72-6.70 (dd, J=8.1, 2.1 Hz, 1H), 4.50 (d, J=3.2 Hz, 1H), 3.77 (s, 1H), 3.43-3.41 (m, 1H), 2.89-2.81 (m, 2H), 2.79-2.71 (m, 2H), 2.57-2.51 (m, 2H), 2.13-2.03 (m, 2H), 1.77-1.65 (m, 2H), 1.43-1.31 (m, 2H); ESI MS m/z 530 [C₂₉H₃₁N₅O₃S+H]⁺; HPLC (Inertsil ODS2 C18 Column) 97.2%, t_{R}=2.0 min.

### Synthesis of Example 38

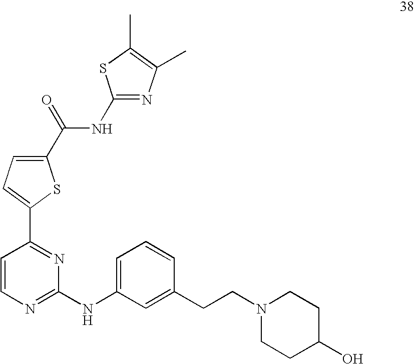

This compound was prepared by the same procedure described for 6 to afford 38 (56 mg, 52%) as an orange solid: mp 158-160° C.; ¹H NMR (500 MHz, DMSO-d₆) δ 9.67 (s, 1H), 8.56 (d, J=5.1 Hz, 1H), 8.14 (d, J=3.8 Hz, 1H), 8.04 (d, J=4.0 Hz, 1H), 7.81 (s, 1H), 7.54 (d, J=8.0 Hz, 1H), 8.08-8.06 (dd, J=5.4, 4.2 Hz, 2H), 7.82 (s, 1H), 7.54 (d, J=8.1 Hz, 1H), 7.40 (d, J=5.1 Hz, 1H), 7.22 (t, J=7.7 Hz, 1H), 6.85 (d, J=7.6 Hz, 1H), 4.57-4.46 (m, 1H), 2.87-2.79 (m, 2H), 2.77-2.73 (m, 2H), 2.60-2.53 (m, 2H), 2.26 (s, 3H), 2.20 (s, 3H), 2.13-2.08 (m, 3H), 1.73-1.70 (m, 2H), 1.41-1.35 (m, 2H); ESI MS m/z 535 [C₂₇H₃₀N₆O₂S₂+H]⁺; HPLC 93.9%, t_{R}=11.5 min.

### Synthesis of Example 39

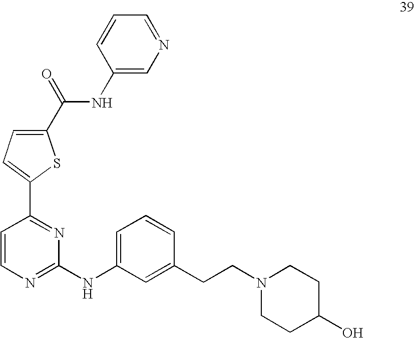

This compound was prepared by the same procedure described for 6 to afford 39 (18 mg, 18%) as a yellow solid: mp 156-158° C.; ¹H NMR (500 MHz, DMSO-d₆) δ 10.56 (s, 1H), 9.69 (s, 1H), 8.92 (d, J=2.5 Hz, 1H), 8.57 (d, J=3.8 Hz, 1H), 8.35-8.33 (dd, J=4.7, 1.4 Hz, 1H), 8.19-8.16 (m, 1H), 8.09 (s, 2H), 7.82 (s, 1H), 7.53 (d, J=8.0 Hz, 1H), 7.43-7.41 (m, 2H), 7.21 (t, J=7.7 Hz, 1H), 6.84 (d, J=7.5 Hz, 1H), 4.55 (s, 1H), 4.50 (d, J=4.3 Hz, 1H), 3.40 (t, J=3.6 Hz, 1H), 2.80 (d, J=11.0 Hz, 2H), 2.74-2.71 (m, 2H), 2.60-2.52 (m, 2H), 2.08 (t, J=9.4 Hz, 2H), 1.70 (t, J=2.8 Hz, 2H), 1.40-1.35 (q, J=3.6 Hz, 2H); ESI MS m/z 501 [C₂₇H₂₈N₆O₂S+H]⁺; HPLC>99%, t_{R}=9.1 min.

### Synthesis of Example 40

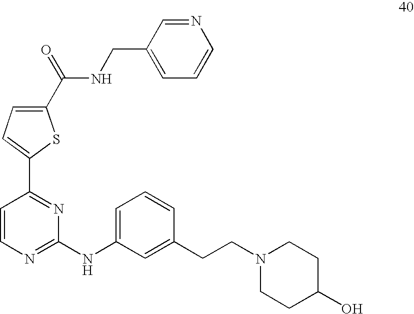

This compound was prepared by the same procedure described for 6 to afford 40 (54 mg, 52%) as a yellow solid: mp 148-151° C.; ¹H NMR (500 MHz, DMSO-d₆) δ 9.65 (s, 1H), 9.24 (t, J=5.8 Hz, 1H), 8.57 (d, J=1.9 Hz, 1H), 8.54 (d, J=5.2 Hz, 1H), 8.48-8.47 (dd, J=4.7, 1.5 Hz, 1H), 8.00 (d, J=4.8 Hz, 1H), 7.85 (d, J=4.0 Hz, 1H), 7.76-7.73 (m, 2H), 7.56 (t, J=8.1 Hz, 1H), 7.40-7.36 (m, 2H), 7.20 (t, J=7.8 Hz, 1H), 6.83 (d, J=7.6 Hz, 1H), 4.52-4.50 (dd, J=6.1, 4.5 Hz, 1H), 3.49-3.38 (m, 1H), 2.79 (d, J=11.2 Hz, 2H), 2.51-2.49 (m, 2H), 2.15-2.02 (m, 2H), 1.71-1.68 (m, 2H), 1.42-1.32 (m, 2H); ESI MS m/z 515 [C₂₈H₃₀N₆O₂S+H]⁺; HPLC>99%, t_{R}=8.6 min.

### Synthesis of Example 41

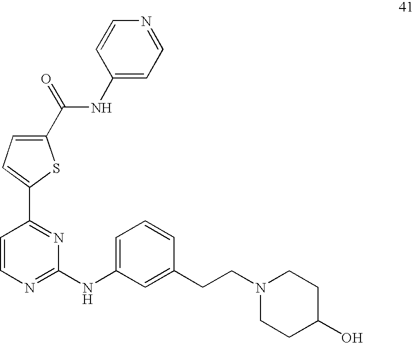

This compound was prepared by the same procedure described for 6 to afford 41 (27 mg, 27%) as a yellow solid: mp 165-170° C.; ¹H NMR (500 MHz, DMSO-d₆) δ 10.67 (s, 1H), 9.70 (s, 1H), 8.58 (d, J=5.1 Hz, 1H), 8.50 (d, J=6.3 Hz, 2H), 8.13-8.09 (dd, J=10.5, 4.1 Hz, 2H), 7.84 (s, 1H), 7.77 (d, J=6.3 Hz, 2H), 7.52 (d, J=8.1 Hz, 1H), 7.42 (d, J=5.2 Hz, 1H), 7.21 (d, J=7.7 Hz, 1H), 6.85 (d, J=7.5 Hz, 1H), 4.51 (d, J=3.8 Hz, 1H), 3.41-3.37 (m, 1H), 2.82-2.80 (m, 2H), 2.74 (t, J=7.4 Hz, 2H), 2.12-2.03 (m, 2H), 1.77-1.65 (m, 2H), 1.41-1.30 (m, 2H); ESI MS m/z 501 [C₂₇H₂₈N₆O₂S+H]⁺; HPLC>99%, t_{R}=8.6 min.

### Synthesis of Example 42

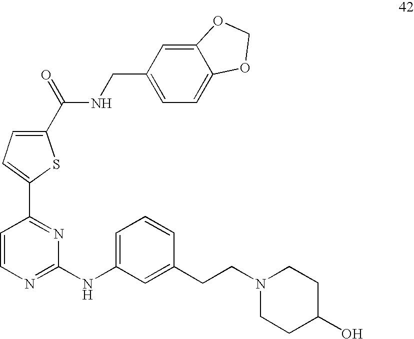

This compound was prepared by the same procedure described for 6 to afford 42 (13 mg, 11%) as a yellow solid: mp 193-196° C.; ¹H NMR (500 MHz, DMSO-d₆) δ 9.63 (s, 1H), 9.11 (t, J=5.9 Hz, 1H), 8.53 (d, J=5.1 Hz, 1H), 7.98 (d, J=4.0 Hz, 1H), 7.84 (d, J=4.0 Hz, 1H), 7.76 (s, 1H), 7.56 (d, J=8.2 Hz, 1H), 7.36 (d, J=5.2 Hz, 1H), 7.20 (t, J=7.8 Hz, 1H), 6.90-6.87 (dd, J=9.7, 1.3 Hz, 2H), 6.84-6.80 (dd, J=17.2, 8.9 Hz, 2H), 5.99 (s, 2H), 4.50 (d, J=4.2 Hz, 1H), 4.38 (d, J=5.9 Hz, 2H), 3.44-3.37 (m, 1H), 2.80-2.78 (m, 2H), 2.73-2.70 (m, 2H), 2.53-2.51 (m, 2H), 2.07 (t, J=9.8 Hz, 2H), 1.72-1.69 (m, 2H), 1.40-1.33 (m, 2H); ESI MS m/z 558 [C₃₀H₃₁N₅O₄S+H]⁺; HPLC>99%, t_{R}=11.6 min.

### Synthesis of Example 43

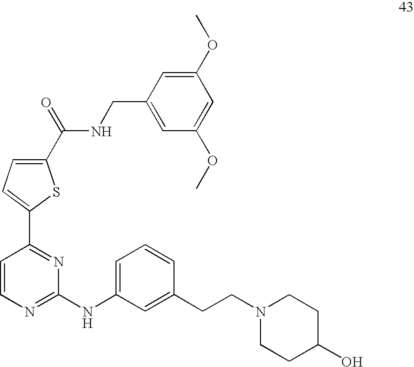

This compound was prepared by the same procedure described for 6 to afford 43 (79 mg, 69%) as a yellow solid: mp 90-95° C.; ¹H NMR (500 MHz, DMSO-d₆) δ 9.36 (s, 1H), 9.14 (t, J=6.3 Hz, 1H), 8.53 (d, J=5.2 Hz, 1H), 8.00 (d, J=4.0 Hz, 1H), 7.87 (d, J=4.1 Hz, 1H), 7.75 (s, 1H), 7.58 (d, J=8.2 Hz, 1H), 7.37 (d, J=5.1 Hz, 1H), 7.20 (t, J=7.8 Hz, 1H), 6.83 (d, J=6.4 Hz, 1H), 6.52-6.49 (m, 3H), 6.40 (t, J=2.2 Hz, 1H), 4.49 (d, J=4.2 Hz, 1H), 4.41 (d, J=5.9 Hz, 1H), 3.73 (s, 9H), 3.48-3.38 (m, 1H), 2.80-2.78 (m, 2H), 2.73-2.70 (m, 2H), 2.06 (t, J=6.9 Hz, 2H), 1.71 (d, J=3.4 Hz, 2H), 1.38-1.35 (m, 2H); ESI MS m/z 574 [C₃₁H₃₅N₅O₄S+H]⁺; HPLC>99%, t_{R}=11.6 min.

### Synthesis of Example 44

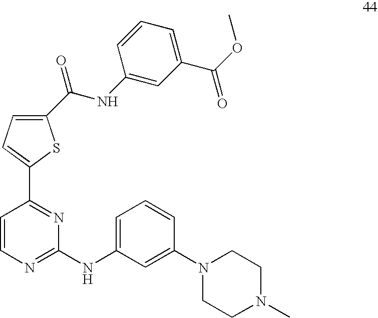

This compound was prepared by the same procedure described for 6 to afford 44 (31 mg, 24%) as a yellow solid: mp 235-238° C.; ¹H NMR (500 MHz, DMSO-d₆) δ 10.56 (s, 1H), 9.58 (s, 1H), 8.56 (d, J=5.1 Hz, 1H), 8.46 (t, J=1.8 Hz, 1H), 8.11 (d, J=4.1 Hz, 1H), 8.08-8.05 (m, 2H), 7.73-7.69 (m, 2H), 7.54 (t, J=8.0 Hz, 1H), 7.41 (d, J=5.1 Hz, 1H), 7.14-7.12 (m, 2H), 6.59-6.57 (m, 1H), 3.88 (s, 3H), 3.20 (t, J=4.7 Hz, 3H), 2.51-2.49 (m, 3H), 2.22 (s, 3H); ESI MS m/z 529 [C₂₈H₂₈N₆O₃S+H]⁺; HPLC 96.9%, t_{R}=12.0 min.

### Synthesis of Example 45

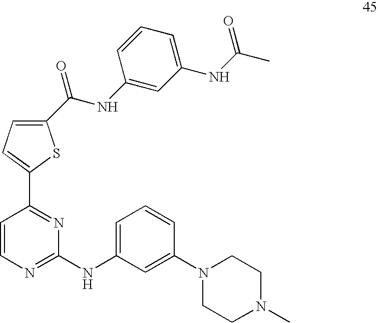

This compound was prepared by the same procedure described for 6 to afford 45 (74 mg, 28%) as a yellow solid: mp 249-252° C.; ¹H NMR (500 MHz, DMSO-d₆) δ 9.92 (s, 1H), 9.53 (s, 1H), 9.19 (t, J=6.1 Hz, 1H), 8.53 (t, J=5.3 Hz, 1H), 7.98 (d, J=4.0 Hz, 1H), 7.87 (d, J=4.0 Hz, 1H), 7.61 (s, 1H), 7.53-7.50 (m, 2H), 7.36 (d, J=5.4 Hz, 1H), 7.25 (t, J=7.8 Hz, 1H), 7.18 (d, J=8.3 Hz, 1H), 7.12 (t, J=8.0 Hz, 1H), 7.00 (d, J=7.6 Hz, 1H), 6.58-6.55 (dd, J=8.1, 1.5 Hz, 1H), 4.45 (d, J=6.0 Hz, 1H), 3.19-3.18 (m, 4H), 2.50-2.48 (m, 5H), 2.21 (s, 3H), 2.02 (s, 3H); ESI MS m/z 542 [C₂₉H₃₁N₇O₃S+H]⁺; HPLC 98.9%, t_{R}=10.6 min.

### Synthesis of Example 46

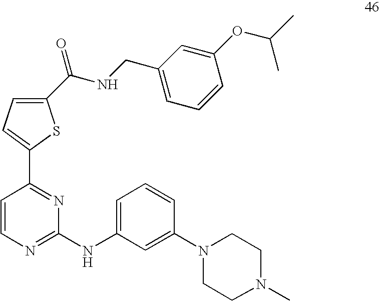

This compound was prepared by the same procedure described for 6 to afford 46 (55 mg, 42%) as a yellow solid: mp 179-178° C.; ¹H NMR (500 MHz, DMSO-d₆) δ 9.54 (s, 1H), 9.15 (t, J=6.0 Hz, 1H), 8.53 (d, J=5.1 Hz, 1H), 7.65 (s, 1H), 7.36 (d, J=5.1 Hz, 1H), 7.23 (t, J=8.2 Hz, 1H), 7.17-7.11 (m, 2H), 6.87 (d, J=7.1 Hz, 2H), 6.82-6.80 (dd, J=7.7, 2.2 Hz, 1H), 6.57 (d, J=7.9 Hz, 1H), 4.61-4.56 (m, 1H), 4.45-4.30 (q, J=5.9 Hz, 2H), 3.22-3.20 (m, 4H), 2.60-2.56 (m, 3H), 2.28 (s, 3H), 1.25 (d, J=6.0 Hz, 6H); ESI MS m/z 543 [C₃₀H₃₄N₆O₂S+H]⁺; HPLC 97.8%, t_{R}=12.6 min.

### Synthesis of Example 47

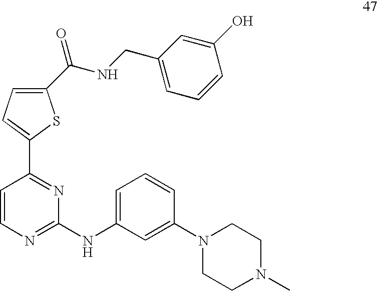

This compound was prepared by the same procedure described for 6 to afford 47 (12 mg, 10%) as a yellow solid: mp 146-149° C.; ¹H NMR (500 MHz, DMSO-d₆) δ 9.54 (s, 1H), 9.36 (s, 1H), 9.14 (t, J=6.0 Hz, 1H), 8.53 (d, J=5.1 Hz, 1H), 7.99 (d, J=4.0 Hz, 1H), 7.87 (t, J=4.1 Hz, 1H), 7.36 (d, J=5.2 Hz, 1H), 7.18-7.11 (m, 3H), 7.75 (s, 1H), 7.74 (s, 1H), 6.65-6.63 (dd, J=7.5, 1.9 Hz, 1H), 6.58-6.56 (dd, J=8.0, 1.4 Hz, 1H), 3.19 (s, 1H), 2.50-2.48 (m, 3H), 2.23 (s, 3H); ESI MS m/z 501 [C₂₇H₂₈N₆O₂S+H]⁺; HPLC 96.1%, t_{R}=10.7 min.

### Synthesis of Example 48

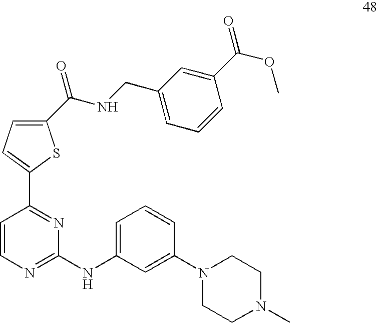

This compound was prepared by the same procedure described for 6 to afford 48 (414 mg, 54%) as a yellow solid: mp 135-140° C.; ¹H NMR (500 MHz, DMSO-d₆) δ 9.63 (s, 1H), 9.37 (t, J=6.0 Hz, 1H), 8.65 (dd, J=14.4, 5.1 Hz, 1H), 8.11 (t, J=4.0 Hz, 1H), 8.03 (d, J=6.0 Hz, 1H), 7.95 (d, J=4.2 Hz, 2H), 7.72 (m, 2H), 7.59 (t, J=7.7 Hz, 1H), 7.45 (d, J=5.2 Hz, 1H), 7.26-7.20 (m, 2H), 6.65 (d, J=7.2 Hz, 1H), 4.63 (d, J=5.9 Hz, 2H), 3.93 (s, 3H), 3.39 (s, 4H), 2.69 (s, 4H), 2.37 (s, 3H); ESI MS m/z 543 [C₂₉H₃₀N₆O₃S+H]⁺; HPLC 96.7%, t_{R}=11.5 min.

### Synthesis of Example 49

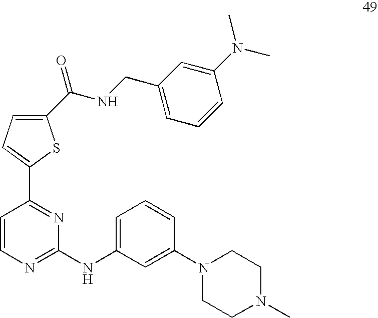

This compound was prepared by the same procedure described for 6 to afford 49 (47 mg, 37%) as a yellow solid: mp 138-140° C.; ¹H NMR (500 MHz, DMSO-d₆) δ 9.56 (s, 1H), 9.13 (t, J=5.9 Hz, 1H), 8.53 (d, J=5.1 Hz, 1H), 7.99 (d, J=4.0 Hz, 1H), 7.87 (d, J=4.1 Hz, 1H), 7.67 (s, 1H) 7.36 (d, J=5.1 Hz, 1H), 7.19-7.12 (m, 3H), 6.71 (s, 1H), 6.64-6.57 (m, 3H), 4.43-4.26 (m, 2H), 3.24 (s, 4H), 2.90-2.86 (m, 6H), 2.66 (s, 4H), 2.33 (s, 3H); ESI MS m/z 528 [C₂₉H₃₃N₇OS+H]⁺; HPLC 95.0%, t_{R}=9.4 min.

### Synthesis of Example 50

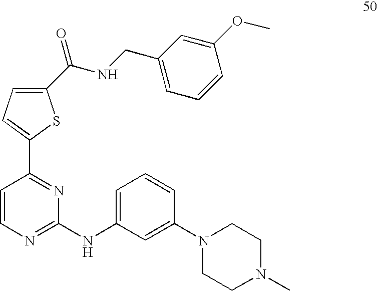

This compound was prepared by the same procedure described for 6 to afford 50 (12 mg, 11%) as a yellow solid: mp 163-165° C.; ¹H NMR (500 MHz, DMSO-d₆) δ 9.54 (s, 1H), 9.17 (d, J=5.5 Hz, 1H), 8.53 (d, J=4.9 Hz, 1H), 7.99 (d, J=3.6 Hz, 1H), 7.87 (d, J=3.7 Hz, 1H), 7.63 (s, 1H) 7.36 (d, J=5.0 Hz, 1H), 7.20 (t, J=8.0 Hz, 1H), 7.17-7.12 (m, 2H), 6.92-6.83 (m, 1H), 6.56 (d, J=7.6 Hz, 1H), 4.46 (d, J=5.5 Hz, 2H), 3.74 (s, 3H), 3.18 (s, 4H), 2.52-2.49 (m, 4H), 2.21 (s, 3H); ESI MS m/z 515 [C₂₈H₃₀N₆O₂S+H]⁺; HPLC 96.8%, t_{R}=11.7 min.

### Synthesis of Example 51

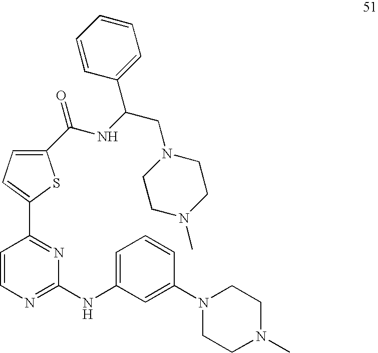

This compound was prepared by the same procedure described for 6 to afford 51 (20 mg, 14%) as a yellow solid: ¹H NMR (500 MHz, DMSO-d₆) δ 9.52 (s, 1H), 8.85 (d, J=8.3 Hz, 1H), 8.52 (d, J=5.1 Hz, 1H), 8.00 (d, J=4.0 Hz, 1H), 7.94 (d, J=4.0 Hz, 1H), 7.60 (s, 1H) 7.42-7.40 (m, 2H), 7.38-7.33 (m, 3H), 7.26-7.23 (m, 1H), 7.16 (d, J=8.4 Hz, 1H), 7.11 (t, J=8.0 Hz, 1H), 6.57-6.55 (q, J=8.1 Hz, 1H), 5.23-5.19 (m, 1H), 3.17 (t, J=4.8 Hz, 4H), 2.86-2.81 (q, J=12.7 Hz, 1H), 2.64-2.45 (m, 7H), 2.31-2.21 (m, 3H), 2.20 (s, 4H), 2.12 (s, 4H); ESI MS m/z 597 [C₃₃H₄₀N₈OS+H]⁺; HPLC 95.8%, t_{R}=9.7 min.

### Synthesis of Example 52

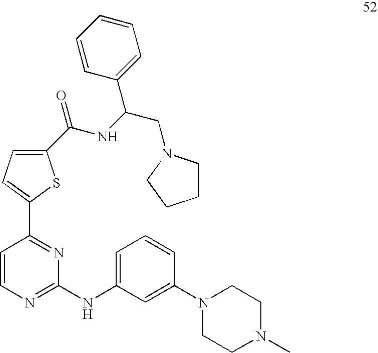

This compound was prepared by the same procedure described for 6 to afford 52 (17 mg, 11%) as a yellow solid: mp 255-260° C.; ¹H NMR (500 MHz, DMSO-d₆) δ 9.63 (s, 1H), 9.54 (s, 1H), 8.55 (d, J=5.1 Hz, 1H), 8.18 (s, 1H), 8.05 (d, J=3.8 Hz, 1H), 7.71 (s, 1H), 7.52 (d, J=7.4 Hz, 2H), 7.45-7.39 (m, 3H), 7.33 (t, J=6.3 Hz, 1H), 7.22-7.15 (m, 2H), 6.62 (d, J=7.2 Hz, 1H), 5.47 (s, 1H), 4.10-2.92 (m, 12H), 2.51-2.49 (m, 4H), 2.05-1.83 (m, 5H); ESI MS m/z 568 [C₃₂H₃₇N₇OS+H]⁺; HPLC>99%, t_{R}=9.7 min.

### Synthesis of Example 53

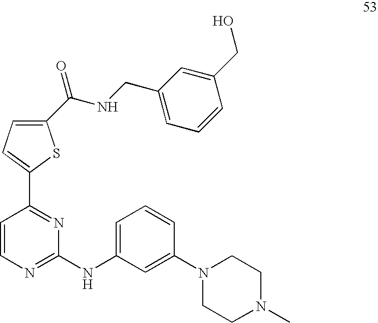

This compound was prepared by the same procedure described for 6 to afford 53 (64 mg, 62%) as a yellow solid: mp 165-170° C.; ¹H NMR (500 MHz, DMSO-d₆) δ 9.62 (s, 1H), 9.24 (t, J=5.9 Hz, 1H), 8.54 (d, J=5.2 Hz, 1H), 8.00 (d, J=4.0 Hz, 1H), 7.89 (d, J=4.1 Hz, 1H), 7.75 (s, 1H), 7.38 (d, J=5.1 Hz, 1H), 7.31-7.28 (m, 2H), 7.20 (d, J=7.8 Hz, 1H), 7.16 (d, J=5.2 Hz, 2H), 6.61 (t, J=3.7 Hz, 1H), 4.61 (s, 1H), 4.49 (d, J=4.7 Hz, 4H), 3.32-3.30 (m, 3H), 3.17-2.95 (m, 4H), 2.56-2.52 (m, 3H); ESI MS m/z 515 [C₂₈H₃₀N₆O₂S+H]⁺; HPLC 98.9%, t_{R}=10.5 min.

### Synthesis of Example 54

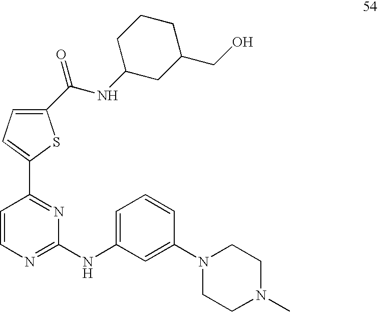

This compound was prepared by the same procedure described for 6 to afford 54 (22 mg, 21%) as a yellow solid: mp 216-220° C.; ¹H NMR (500 MHz, DMSO-d₆) δ 9.52 (s, 1H), 8.51 (d, J=5.1 Hz, 1H), 8.36 (d, J=8.0 Hz, 1H), 7.96 (d, J=4.0 Hz, 1H), 7.82 (d, J=4.0 Hz, 1H), 7.61 (s, 1H), 7.34 (d, J=5.1 Hz, 1H), 7.17-7.10 (m, 2H), 6.57 (d, J=6.5 Hz, 1H), 4.42 (t, J=5.4 Hz, 1H), 3.75 (t, J=3.9 Hz, 1H), 3.27-3.24 (q, J=9.0 Hz, 2H), 3.19 (s, 4H), 2.51-2.50 (m, 2H), 2.24 (s, 3H), 1.92 (d, J=2.8 Hz, 1H), 1.86 (d, J=11.6 Hz, 1H), 1.79-1.76 (q, J=9.7 Hz, 1H), 1.68 (d, J=11.0 Hz, 1H), 1.49-1.48 (m, 1H), 1.33-1.23 (m, 2H), 1.01-0.95 (m, 1H), 0.87-0.78 (m, 1H); ESI MS m/z 507 [C₂₇H₃₄N₆O₂S+H]⁺; HPLC>99%, t_{R}=10.4 min.

### Synthesis of Example 55

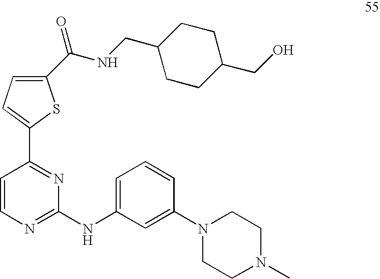

This compound was prepared by the same procedure described for 6 to afford 55 (30 mg, 44%) as a yellow solid: mp 229-231° C.; ¹H NMR (500 MHz, DMSO-d₆) δ 9.53 (s, 1H), 8.58 (t, J=5.8 Hz, 1H), 8.52 (d, J=5.1 Hz, 1H), 7.97 (d, J=4.0 Hz, 1H), 7.81 (d, J=4.0 Hz, 1H), 7.63 (s, 1H), 7.35 (d, J=5.1 Hz, 1H), 7.17-7.10 (m, 2H), 6.56 (d, J=7.8 Hz, 1H), 4.34 (t, J=5.3 Hz, 1H), 3.21-3.17 (m, 6H), 3.11 (t, J=6.4 Hz, 2H), 2.22 (s, 3H), 1.78-1.76 (m, 5H), 1.54-1.49 (m, 1H), 1.44-1.27 (m, 1H), 0.88-0.83 (m, 5H); ESI MS m/z 521 [C₂₈H₃₆N₆O₂S+H]⁺; HPLC 98.8%, t_{R}=10.6 min.

### Synthesis of Example 56

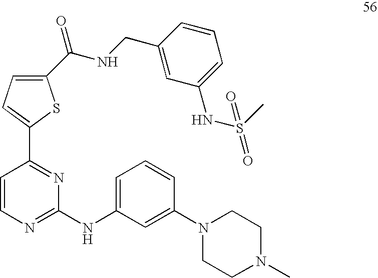

This compound was prepared by the same procedure described for 6 to afford 56 (34 mg, 30%) as a yellow solid: mp 133-137° C.; ¹H NMR (500 MHz, DMSO-d₆) δ 9.75 (s, 1H), 9.54 (s, 1H), 9.21 (t, J=5.9 Hz, 1H), 8.53 (d, J=5.1 Hz, 1H), 8.00 (d, J=4.0 Hz, 1H), 7.86 (d, J=4.0 Hz, 1H), 7.61 (s, 1H), 7.36 (d, J=5.1 Hz, 1H), 7.31 (d, J=6.9 Hz, 1H), 7.29-7.07 (m, 5H), 6.57 (d, J=1.4 Hz, 1H), 4.46 (d, J=5.8 Hz, 1H), 3.18 (t, J=4.6 Hz, 4H), 2.98 (s, 3H), 2.52-2.50 (m, 4H), 2.21 (s, 3H); ESI MS m/z 578 [C₂₈H₃₁N₇O₃S₂+H]⁺; HPLC 99.0%, t_{R}=10.8 min.

### Synthesis of Example 57

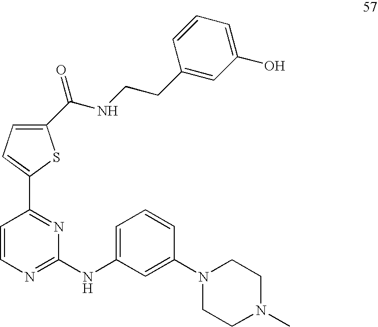

This compound was prepared by the same procedure described for 6 to afford 57 (42 mg, 36%) as a yellow solid: mp 122-124° C.; ¹H NMR (500 MHz, DMSO-d₆) δ 9.53 (s, 1H), 9.28 (s, 1H), 8.73 (t, J=5.6 Hz, 1H), 8.52 (d, J=4.2 Hz, 1H), 7.97 (d, J=4.0 Hz, 1H), 7.77 (d, J=4.0 Hz, 1H), 7.63 (s, 1H), 7.35 (d, J=5.1 Hz, 1H), 7.18-7.07 (m, 3H), 6.67-6.56 (m, 4H), 3.46-3.42 (m, 2H), 3.17 (t, J=10.9 Hz, 5H), 2.76 (t, J=7.8 Hz, 2H), 2.54-2.52 (m, 3H), 2.25 (s, 3H); ESI MS m/z 515 [C₂₈H₃₀N₆O₂S+H]⁺; HPLC 97.7%, t_{R}=10.9 min.

### Synthesis of Example 58

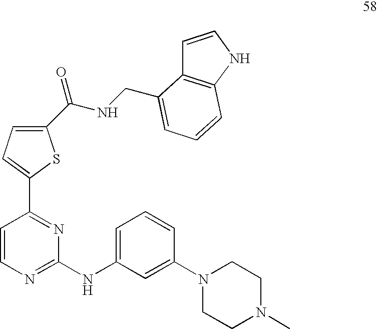

This compound was prepared by the same procedure described for 6 to afford 58 (42 mg, 36%) as a yellow solid: mp 145-147° C.; ¹H NMR (500 MHz, DMSO-d₆) δ 11.10 (s, 1H), 9.55 (s, 1H), 9.13 (t, J=6.2 Hz, 1H), 8.52 (d, J=5.1 Hz, 1H), 7.97 (d, J=4.1 Hz, 1H), 7.87 (d, J=4.1 Hz, 1H), 7.65 (s, 1H), 7.36-7.31 (m, 2H), 7.15-7.11 (q, J=6.7 Hz, 2H), 7.05-7.04 (m, 1H), 6.96 (t, J=7.2 Hz, 1H), 6.59-6.58 (m, 2H), 4.74 (d, J=6.2 Hz, 2H), 2.19 (t, J=4.8 Hz, 4H), 2.52-2.50 (m, 4H), 2.22 (s, 3H); ESI MS m/z 524 [C₂₉H₂₉N₇OS+H]⁺; HPLC 96.5%, t_{R}=10.9 min.

### Synthesis of Example 59

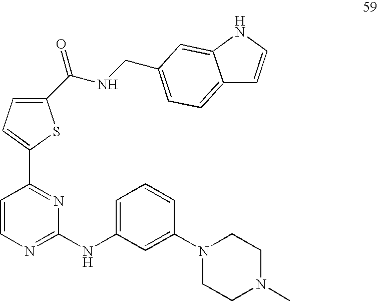

This compound was prepared by the same procedure described for 6 to afford 59 (22 mg, 20%) as a yellow solid: mp 136-140° C.; ¹H NMR (500 MHz, DMSO-d₆) δ 11.02 (s, 1H), 9.53 (s, 1H), 9.16(t, J=5.9 Hz, 1H), 8.52 (d, J=5.1 Hz, 1H), 7.98 (d, J=4.0 Hz, 1H), 7.87 (d, J=4.0 Hz, 1H), 7.62 (s, 1H), 7.49 (d, J=8.1 Hz, 1H), 7.35 (d, J=5.0 Hz, 2H), 7.30 (t, J=2.7 Hz, 1H), 7.17 (d, J=8.3 Hz, 1H), 7.13(t, J=8.0 Hz, 2H), 7.00 (dd, J=8.2, 1.4 Hz, 1H), 6.57 (dd, J=5.1, 1.7 Hz, 1H), 6.39-6.38 (m, 1H), 4.57 (d, J=5.9 Hz, 1H), 3.20-3.16 (m, 4H), 2.51-2.50 (m, 4H), 2.21 (s, 3H); ESI MS m/z 524 [C₂₉H₂₉N₇OS+H]⁺; HPLC 98.1%, t_{R}=11.6 min.

### Synthesis of Example 60

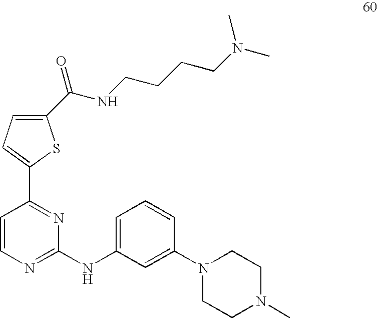

This compound was prepared by the same procedure described for 6 to afford 60 (30 mg, 30%) as a yellow solid: mp 177-180° C.; ¹H NMR (500 MHz, DMSO-d₆) δ 9.52 (s, 1H), 8.61 (t, J=5.6 Hz, 1H), 8.52 (d, J=6.1 Hz, 1H), 7.96 (d, J=4.0 Hz, 1H), 7.78 (d, J=4.1 Hz, 1H), 7.63 (s, 1H), 7.34 (d, J=5.2 Hz, 1H), 7.17-7.11 (m, 2H), 6.58-6.55 (m, 1H), 3.26 (q, J=6.8 Hz, 2H), 3.18 (t, J=4.7 Hz, 4H), 2.50-2.48 (m, 4H), 2.23-2.18 (m, 4H), 2.11 (s, 6H), 1.57-1.51 (m, 2H), 1.47-1.41 (m, 2H); ESI MS m/z 494 [C₂₆H₃₅N₇OS+H]⁺; HPLC>99%, t_{R}=8.5 min.

### Synthesis of Example 61

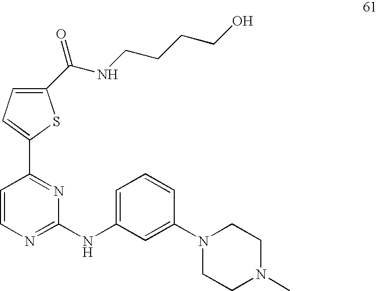

This compound was prepared by the same procedure described for 6 to afford 61 (17 mg, 12%) as a yellow solid: mp 110-115° C.; ¹H NMR (500 MHz, DMSO-d₆) δ 9.53 (s, 1H), 8.61 (t, J=5.5 Hz, 1H), 8.52 (d, J=5.0 Hz, 1H), 7.97 (d, J=3.9 Hz, 1H), 7.79 (d, J=4.0 Hz, 1H), 7.62 (s, 1H), 7.35 (d, J=5.1 Hz, 1H), 7.17-7.11 (m, 2H), 6.57 (d, J=7.7 Hz, 1H), 4.42 (t, J=4.9 Hz, 1H), 3.43 (q, J=6.0 Hz, 2H), 3.29-3.24 (m, 2H), 3.18 (m, 4H), 2.51-2.49 (m, 4H), 2.23 (s, 3H), 1.58-1.55 (m, 2H), 1.48-1.45 (m, 2H); ESI MS m/z 467 [C₂₄H₃₀N₆O₂S+H]⁺; HPLC>99%, t_{R}=9.7 min.

### Synthesis of Example 62

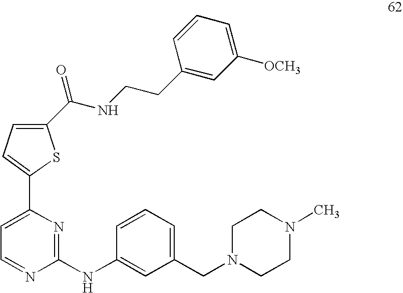

This compound was prepared by the same procedure described for 6 above. The crude product was purified by chromatography (silica gel, 0-20% methanol/methylene chloride w 1% conc. ammonium hydroxide) to afford 62 (45 mg, 21%) as a yellow solid: ¹H NMR (500 MHz, DMSO-d₆) δ 9.69 (s, 1H), 8.74 (t, J=3.3 Hz, 1H), 8.53 (d, J=5.0 Hz, 1H), 7.99 (d, J=4.0 Hz, 1H), 7.85 (s, 1H), 7.79 (d, J=4.0 Hz, 1H), 7.68-7.66 (m, 1H), 7.35 (d, J=5.0 Hz, 1H), 7.27-7.08 (m, 2H), 6.93-6.75 (m, 4H), 3.72 (s, 3H), 3.52-3.46 (m, 4H), 2.83 (t, J=7.5 Hz, 2H), 2.54-2.34 (m, 8H), 2.23 (bs, 3H); ESI MS m/z 543 [C₃₀H₃₄N₆O₂S+H]⁺; HPLC (Method A) 98.4% (AUC), t_{R}=10.71 min.

### Synthesis of Example 63

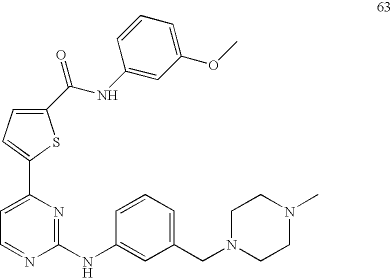

This compound was prepared by the same procedure described for 6 above. The crude product was purified by chromatography (silica gel, 0-20% methanol/methylene chloride w 1% conc. ammonium hydroxide) to afford 63 (14 mg, 8%) as a yellow solid: ¹H NMR (500 MHz, CDCl₃) δ 8.46 (d, J=5.0 Hz, 1H), 7.80 (bs, 1H), 7.75-7.62 (m, 4H), 7.43 (t, J=2.5 Hz, 1H), 7.37-7.18 (m, 3H), 7.12-7.02 (m, 3H), 6.75-6.71 (m, 1H), 3.84 (s, 3H), 3.52 (bs, 2H), 2.82-2.45 (m, 8H), 2.34 (bs, 3H); ESI MS m/z 515 [C₂₈H₃₀N₆O₂S+H]⁺; HPLC (Method A) 94.3% (AUC), t_{R}=10.59 min.

### Synthesis of Example 64

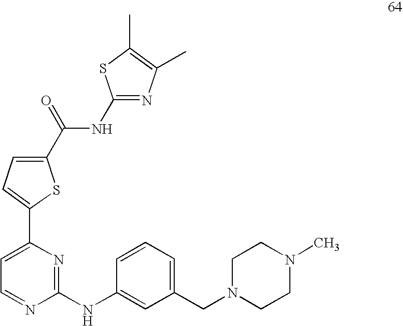

This compound was prepared by the same procedure described for 6 above. The crude product was purified by chromatography (silica gel, 0-20% methanol/methylene chloride w 1% conc. ammonium hydroxide) to afford 64 (45 mg, 32%) as a yellow solid: ¹H NMR (500 MHz, CDCl₃) δ 8.40 (d, J=5.5 Hz, 1H), 8.21 (bs, 1H), 7.79 (d, J=4.0 Hz, 1H), 7.66 (d, J=4.0 Hz, 1H), 7.34-7.29 (m, 3H), 7.13-7.05 (m, 3H), 3.58 (bs, 2H), 3.05-2.45 (m, 8H), 2.33 (bs, 3H), 2.25 (s, 3H), 2.20 (s, 3H); ESI MS m/z 520 [C₂₆H₂₉N₇OS₂+H]⁺; HPLC (Method A) 94.6% (AUC), t_{R}=10.26 min.

### Synthesis of Example 65

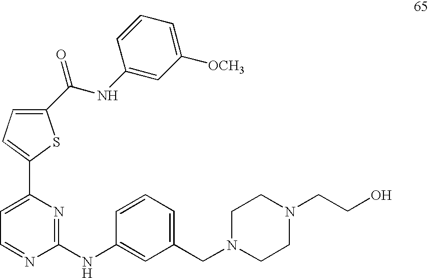

This compound was prepared by the same procedure described for 6 above. The crude product was purified by chromatography (silica gel, 0-20% methanol/methylene chloride w 1% conc. ammonium hydroxide) to afford 65 (35 mg, 19%) as a yellow solid: ¹H NMR (500 MHz, DMSO-d₆) δ 10.32 (s, 1H), 9.72 (s, 1H), 8.56 (d, J=5.5 Hz, 1H), 8.08-8.06 (m, 2H), 7.85 (bs, 1H), 7.69-7.67 (m, 1H), 7.43-7.23 (m, 5H), 6.92-6.89 (m, 1H), 6.72-6.70 (m, 1H), 4.35 (bs, 1H), 3.77 (s, 3H), 3.46 (bs, 4H), 2.53-2.32 (m, 10H); ESI MS m/z 545 [C₂₉H₃₂N₆O₃S+H]⁺; HPLC (Method A) 94.3% (AUC), t_{R}=10.50 min.

### Synthesis of Example 66

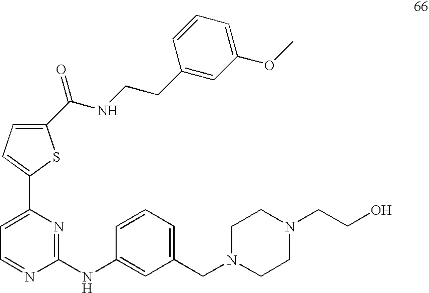

This compound was prepared by the same procedure described for 6 above. The crude product was purified by chromatography (silica gel, 0-20% methanol/methylene chloride w 1% conc. ammonium hydroxide) to afford 66 (19 mg, 10%) as a yellow solid: ¹H NMR (500 MHz, DMSO-d₆) δ 9.70 (s, 1H), 8.74 (t, J=5.5 Hz, 1H), 8.53 (d, J=5.0 Hz, 1H), 7.99 (d, J=4.0 Hz, 1H), 7.88 (bs, 1H), 7.79 (d, J=4.0 Hz, 1H), 7.66-7.64 (m, 1H), 7.37 (d, J=5.0 Hz, 1H), 7.30-7.20 (m, 2H), 6.97-6.91 (m, 1H), 6.83-6.77 (m, 3H), 4.45 (d, J=6.0 Hz, 2H), 4.33 (bs, 1H), 3.72 (s, 3H), 3.59-3.44 (m, 4H), 2.83 (t, J=7.5 Hz, 2H), 2.52-2.35 (m, 10H); ESI MS m/z 573 [C₃₁H₃₆N₆O₃S+H]⁺; HPLC (Method A) 98.3% (AUC), t_{R}=10.60 min.

### Synthesis of Example 67

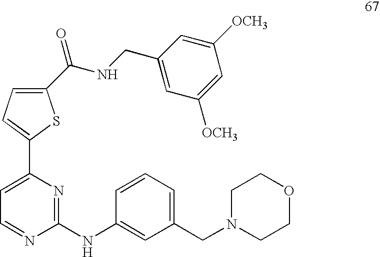

This compound was prepared by the same procedure described for 6 above. The crude product was purified by chromatography (silica gel, 0-20% methanol/methylene chloride w 1% conc. ammonium hydroxide) to afford 67 (29 mg, 13%) as a yellow solid: ¹H NMR (500 MHz, DMSO-d₆) δ 9.70 (s, 1H), 9.26 (t, J=6.0 Hz, 1H), 8.54 (d, J=5.0 Hz, 1H), 8.01 (d, J=6.5 Hz, 1H), 7.90-7.84 (m, 2H), 7.66-7.48 (m, 1H), 7.37 (d, J=5.5 Hz, 1H) 7.25 (t, J=7.5 Hz, 1H), 6.92-6.91 (m, 1H), 6.53-6.45 (m, 2H), 6.40-6.39 (m, 1H), 4.42 (d, J=6.0 Hz, 2H), 3.72 (s, 6H), 3.57-3.55 (m, 4H), 3.46 (bs, 2H), 2.40-2.35 (m, 4H); ESI MS m/z 546 [C₂₉H₃₁N₅O₄S+H]⁺; HPLC (Method A) 98.4% (AUC), t_{R}=11.90 min.

### Synthesis of Example 68

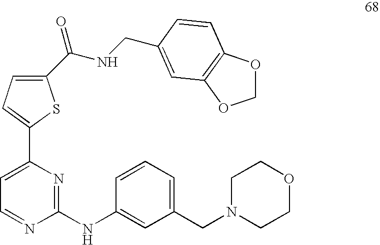

This compound was prepared by the same procedure described for 6 above. The crude product was purified by chromatography (silica gel, 0-20% methanol/methylene chloride w 1% conc. ammonium hydroxide) to afford 68 (50 mg, 23%) as a yellow solid: ¹H NMR (500 MHz, DMSO-d₆) δ 9.70 (s, 1H), 9.12 (t, J=6.0 Hz, 1H), 8.54 (d, J=5.0 Hz, 1H), 8.00 (d, J=4.0 Hz, 1H), 7.90 (bs, 1H), 7.85 (d, J=4.0 Hz, 1H), 7.66-7.64 (m, 1H), 7.37 (d, J=5.5 Hz, 1H), 7.25 (t, J=7.5 Hz, 1H), 6.93-6.81 (m, 4H), 5.98 (s, 2H), 4.38 (d, J=6.0 Hz, 2H), 3.57-3.55 (m, 4H), 3.50 (bs, 2H), 2.40-2.35 (m, 4H); ESI MS m/z 530 [C₂₈H₂₇N₅O₄S+H]⁺; HPLC (Method A) 98.8% (AUC), t_{R}=11.70 min.

### Synthesis of Example 69

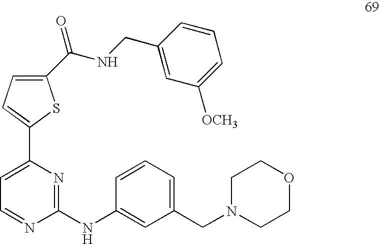

This compound was prepared by the same procedure described for 6 above. The crude product was purified by chromatography (silica gel, 0-20% methanol/methylene chloride w 1% conc. ammonium hydroxide) to afford 69 (20 mg, 9%) as a yellow solid: ¹H NMR (500 MHz, DMSO-d₆) δ 9.70 (s, 1H), 9.27 (t, J=6.0 Hz, 1H), 8.55(d, J=5.0 Hz, 1H), 8.00 (d, J=4.0 Hz, 1H), 7.90 (bs, 1H), 7.85 (d, J=4.0 Hz, 1H), 7.65-7.63 (m, 1H), 7.53-7.47 (m, 1H), 7.38-6.36 (m, 2H), 7.32 (bs, 1H), 7.27-7.21 (m, 2H), 6.92-6.91 (m, 1H), 4.53 (d, J=6.0 Hz, 2H), 3.56-3.55 (m, 4H), 3.42 (bs, 2H), 2.42-2.36 (m, 4H); ESI MS m/z 570 [C₂₈H₂₆F₃N₅O₃S+H]⁺; HPLC (Method A) 97.9% (AUC), t_{R}=13.15 min.

### Synthesis of Example 70

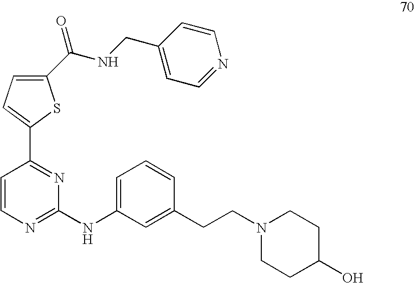

This compound was prepared by the same procedure described for 6 to afford 70 (37 mg, 36%) as a yellow solid: mp 133-136° C.; ¹H NMR (500 MHz, DMSO-d₆) δ 9.65 (s, 1H), 9.29 (t, J=5.9 Hz, 1H), 8.55-8.52 (m, 3H), 8.02 (d, J=4.0 Hz, 1H), 7.89 (d, J=4.1 Hz, 1H), 7.76 (s, 1H), 7.58-7.56 (m, 1H), 7.38 (d, J=5.1 Hz, 1H), 7.33 (d, J=6.1 Hz, 2H), 7.20 (t, J=7.7 Hz, 1H), 6.83 (d, J=7.6 Hz, 1H), 4.51 (d, J=5.2 Hz, 3H), 3.48-3.38 (m, 1H), 2.79 (d, J=11.4 Hz, 2H), 2.71 (t, J=7.4 Hz, 2H), 2.06 (t, J=9.9 Hz, 2H), 1.71-1.68 (m, 2H), 1.40-1.35 (q, J=12.9, 3.6 Hz, 2H); ESI MS m/z 515 [C₂₈H₃₀N₆O₂S+H]⁺; HPLC>99%, t_{R}=14.0 min.

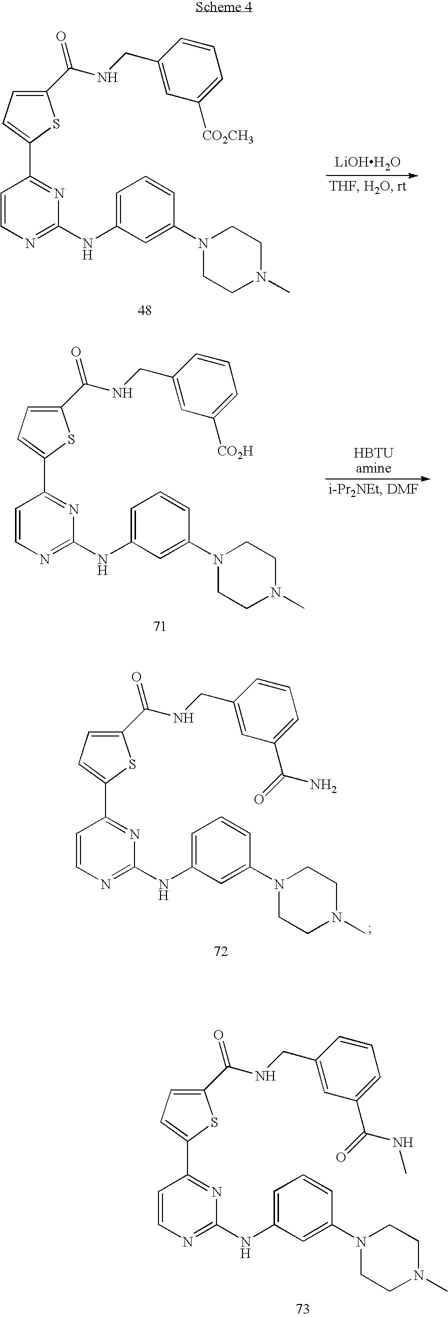

Synthesis of Example 71 To solution of 48 (400 mg, 0.73 mmol) in tetrahydrofuran (3.5 mL) and water (3.5 mL) was added lithium hydroxide monohydrate (92 mg, 2.2 mmol). The solution was stirred at ambient temperature for 6 h. The reaction was diluted with water (20 mL) and treated with 6 N HCl (20 mL) and a red solid precipitated. The water was decanted, the solid dissolved in methanol (40 mL), and the solution concentrated under reduced pressure to give 71 (390 mg, >99%) as a red solid: mp 180-185° C.; ¹H NMR (500 MHz, DMSO-d₆) δ 11.07 (s, 1H), 9.80 (s, 1H), 9.50 (t, J=5.9 Hz, 1H), 8.56 (t, J=5.2 Hz, 1H), 8.05 (d, J=4.0 Hz, 1H), 7.97 (d, J=4.0 Hz, 1H), 7.94 (s, 1H), 7.85 (d, J=8.0 Hz, 1H), 7.78 (s, 1H), 7.61 (d, J=6.7 Hz, 1H), 7.49 (d, J=7.6 Hz, 1H), 7.43 (d, J=5.2 Hz, 1H), 7.21 (s, 2H), 6.67-6.65 (m, 1H), 4.55 (d, J=4.9 Hz, 1H), 3.83 (d, J=10.0 Hz, 2H), 3.53 (d, J=9.0 Hz, 2H), 3.26-3.17 (m, 4H), 2.79 (d, J=4.6 Hz, 3H); ESI MS m/z 529 [C₂₈H₂₈N₆O₃S+H]⁺; HPLC 95.5%, t_{R}=10.6 min.

### Synthesis of Example 72

This compound was prepared by the same procedure described for 6 to afford 72 (47 mg, 53%) as a yellow solid: mp 228-232° C.; ¹H NMR (500 MHz, DMSO-d₆) □ 9.54 (s, 1H), 9.22 (t, J=5.9 Hz, 1H), 8.53 (d, J=5.1 Hz, 1H), 8.00-7.96 (m, 2H), 7.87 (t, J=4.1 Hz, 2H), 7.77 (d, J=7.7 Hz, 1H), 7.62 (s, 1H), 7.49 (d, J=7.7 Hz, 1H), 7.42 (t, J=7.6 Hz, 1H), 7.36 (d, J=5.2 Hz, 2H), 7.17 (d, J=8.2 Hz, 1H), 7.12 (t, J=8.0 Hz, 1H), 6.57-6.55 (m,1H), 4.53 (d, J=5.9 Hz, 2H), 3.18 (t, J=4.6 Hz, 4H), 2.52-2.49 (m, 4H), 2.20 (s, 3H); ESI MS m/z 528 [C₂₈H₂₉N₇O₂S+H]⁺; HPLC 97.2%, t_{R}=10.0 min.

Synthesis of Example 73 This compound was prepared by the same procedure described for 6 to afford 73 (55 mg, 61 %) as a yellow solid: mp 263-267° C.; ¹H NMR (500 MHz, DMSO-d6) δ 9.54 (s, 1H), 9.23 (t, J=5.9 Hz, 1H), 8.52 (d, J=5.1 Hz, 1H), 8.44 (d, J=4.3 Hz, 1H), 8.00 (d, J=4.0 Hz, 1H), 7.87 (d, J=4.1 Hz, 1H), 7.82 (s, 1H), 7.71 (d, J=7.6 Hz, 1H), 7.62 (s, 1H), 7.48 (d, J=7.7 Hz, 1H), 7.43 (t, J=7.6 Hz, 1H), 7.36 (d, J=5.1 Hz, 1H), 7.18-7.12 (m, 2H), 6.57-6.55 (m, 1H), 4.53-4.33 (m, 2H), 3.18 (t, J=4.7 Hz, 4H), 2.77 (d, J=4.6 Hz, 3H), 2.52-2.49 (m, 4H), 2.20 (s, 3H); ESI MS m/z 542 [C₂₉H₃₁N₇O₂S+H]⁺; HPLC 95.9%, t_{R}=10.1 min.

### Subsection 5—Examples Via Alternative Route

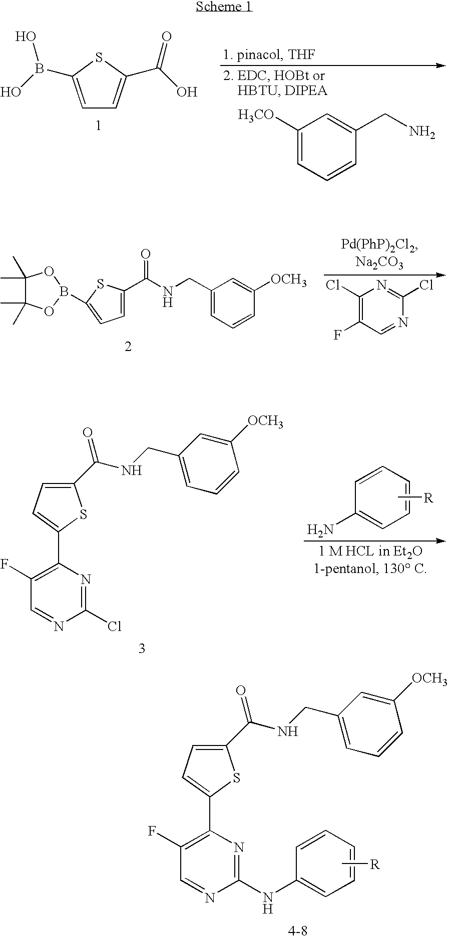

### Synthesis of 2

To a solution of 1 (2.0 g, 12 mmol) in THF (20 mL) and toluene (20 mL) was added pinacol (1.4 g, 12 mmol) and the resulting mixture was concentrated under reduced pressure to dryness. The solids obtained was dissolved in THF (20 mL) and toluene (20 mL) and concentrated under reduced pressure two more times. The intermediate solid was dissolved in DMF (40 mL) followed by the addition of EDC (2.3 g, 12 mmol), HOBt (1.6 g, 12 mmol), DIPEA (4.2 mL, 24 mmol) and amine (1.6 g, 12 mmol). The resulting reaction mixture was stirred for 14 h. The reaction mixture was diluted with H₂O (50 mL) and ethyl acetate (100 mL). The layers were separated and the organic layer was dried over Na₂SO₄, filtered and concentrated. The residue was purified by column chromatography (silica gel, 0-50% ethyl acetate/heptane) to obtain 2 as a pale yellow solid (0.37 g, 8.4% for two steps): ¹H NMR (500 MHz, DMSO-d₆) δ 9.10 (t, J=6.0 Hz, 1H), 7.84 (d, J=4.0 Hz, 1H), 7.54 (d, J=4.0 Hz, 1H), 7.24 (t, J=8.0 Hz, 1H), 6.88-6.80 (m, 3H), 4.42 (d, J=6.0 Hz, 2H), 3.73 (s, 3H), 1.29 (s, 12H).

### Synthesis of 3

To a solution of 2 (1.0 g, 2.3 mmol) in DME (10 mL) and ethanol (25 mL) was added 5-fluoro-2,4-dichloropyrimidine (1.0 g, 6 mmol), Pd(Ph₃P)₂Cl₂ (0.10 g, 0.15 mmol) and 2 N Na₂CO₃ (2 mL). The resulting mixture was heated at 80° C. for 4 h. The reaction was cooled to rt and partitioned between H₂O (50 mL) and ethyl acetate (100 mL). The layers were separated and the organic layer was dried over Na₂SO₄, filtered and concentrated. The residue was purified by column chromatography (silica gel, 0-70% ethyl acetate/heptane) to obtain 3 (0.55 g, 63%) as a yellow solid: ESI MS m/z 378 [C₁₇H₁₃ClFN₃O₂S+H]⁺.

### Synthesis of Example 4

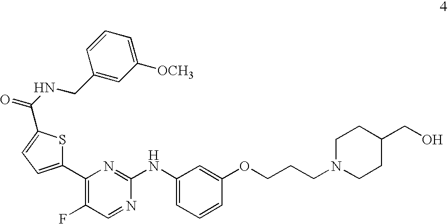

A solution of 1 N HCl in diethyl ether (0.5 mL, 0.49 mmol) was added dropwise to a solution of aniline (120 mg, 0.44 mmol) and 3 (200 mg, 0.53 mmol) in 1-pentanol (5 mL). The reaction mixture was heated to reflux and stirred for 18 h. The reaction was cooled to room temperature, concentrated under reduced pressure, and the crude solid was dissolved in a mixture of methylene chloride (6 mL) and trifluoroacetic acid (1 mL). The solution was stirred for 15 min and neutralized carefully with satd. aq. NaHCO₃. The organic layer was separated, dried over Na₂SO₄ and concentrated under reduced pressure. The crude product was purified by flash chromatography (silica gel, 94.5:4.5:0.5 methylene chloride/methanol/concentrated ammonium hydroxide) to afford 4 (67 mg, 25%) as a bright yellow solid: mp 77-80° C.; ¹H NMR (500 MHz, DMSO-d₆) δ 9.79 (s, 1H), 9.28-9.25 (m, 1H), 8.68-8.67 (m, 1H), 7.93-7.88 (m, 2H), 7.56 (s, 1H), 7.27-7.24 (m, 2H), 7.17 (t, J=8.1 Hz, 1H), 6.91-6.90 (m, 2H), 6.84-6.82 (m, 1H), 6.54-6.53 (m, 1H), 4.46-4.45 (m, 2H), 4.37-4.35 (m, 1H), 4.05-4.03 (m, 2H), 3.74 (s, 3H), 3.21-3.19 (m, 2H), 2.85-2.83 (m, 2H), 2.41-2.38 (m, 2H), 1.91-1.78 (m, 4H), 1.59-1.57 (m, 2H), 1.29-1.28 (m, 1H), 1.10-1.04 (m, 2H); ESI MS m/z 606 [C₃₂H₃₆FN₅O₄S+H]⁺; HPLC (Method A) 91.8% (AUC), t_{R}=13.26 min.

### Synthesis of Example 5

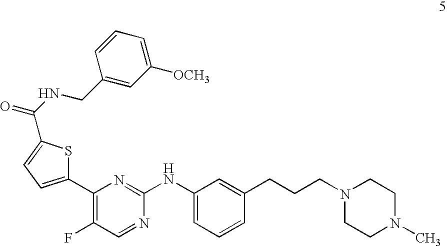

This compound was prepared by the same procedure described for 4 to afford 5 (62 mg, 24%) as a yellow solid: mp 62-64° C.; ¹H NMR (500 MHz, DMSO-d₆) δ 9.73 (s, 1H), 9.27-9.25 (m, 1H), 8.67-8.66 (m, 1H), 7.93-7.88 (m, 2H), 7.72 (s, 1H), 7.53-7.52 (m, 1H), 7.27-7.19 (m, 2H), 6.91-6.90 (m, 2H), 6.84-6.81 (m, 2H), 4.46-4.45 (m, 2H), 3.74 (s, 3H), 2.60-2.57 (m, 2H), 2.39-2.11 (m, 13H), 1.75-1.72 (m, 2H); ESI MS m/z 575 [C₃₁H₃₅FN₆O₂S+H]⁺; HPLC (Method A) 96.2% (AUC), t_{R}=11.26 min.

### Synthesis of Example 6

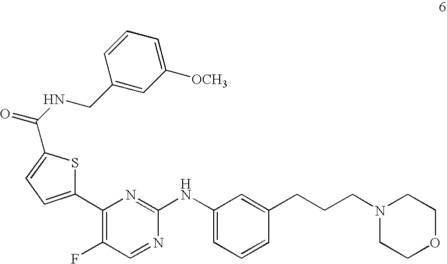

This compound was prepared by the same procedure described for 4 to afford 6 (64 mg, 26%) as a yellow solid: mp 62-64° C.; ¹H NMR (500 MHz, DMSO-d₆) δ 9.73 (s, 1H), 9.27-9.24 (m, 1H), 8.67-8.66 (m, 1H), 7.92-7.88 (m, 2H), 7.74 (s, 1H), 7.53-7.51 (m, 1H), 7.27-7.19 (m, 2H), 6.91-6.90 (m, 2H), 6.84-6.82 (m, 2H), 4.46-4.45 (m, 2H), 3.74 (s, 3H), 3.53 (s, 4H), 2.62-2.59 (m, 2H), 2.31-2.30 (m, 6H), 1.77-1.74 (m, 2H); ESI MS m/z 562 [C₃₀H₃₂FN₅O₃S+H]⁺; HPLC (Method A) 97.0% (AUC), t_{R}=13.70 min.

### Synthesis of Example 7

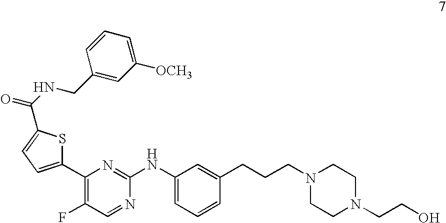

This compound was prepared by the same procedure described for 4 to afford 7 (38 mg, 12%) as a yellow solid: mp 60-70° C.; ¹H NMR (500 MHz, DMSO-d₆) δ 9.72 (s, 1H), 9.11-9.10 (m, 1H), 8.67-8.66 (m, 1H), 7.93-7.88 (m, 2H), 7.71 (s, 1H), 7.48-7.46 (m, 1H), 7.15-7.09 (m, 2H), 6.91-6.90 (m, 2H), 6.84-6.82 (m, 2H), 4.46-4.45 (m, 2H), 4.23-4.22 (m, 1H), 3.74 (s, 3H), 3.46-3.45 (m, 2H), 2.60-2.58 (m, 2H), 2.34-2.28 (m, 12H), 1.74-1.72 (m, 2H); ESI MS m/z 605 [C₃₂H₃₇FN₆O₃S+H]⁺; HPLC (Method A) 96.6% (AUC), t_{R}=10.99 min.

### Synthesis of Example 8

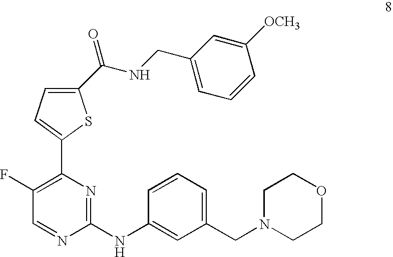

This compound was prepared by the same procedure described for 4 to afford 8 (50 mg, 17%) as a yellow solid: ¹H NMR (500 MHz, DMSO-d₆) δ 9.79 (s, 1H), 9.26 (t, J=6.0 Hz, 1H), 8.67 (d, J=3.5 Hz, 1H), 7.93-7.88 (m, 3H), 7.60-7.58 (m, 1H), 7.27-7.24 (m, 2H), 6.93-6.90 (m, 3H), 6.84-6.82 (m, 1H), 4.45 (d, J=5.5 Hz, 2H), 3.74 (s, 3H), 3.56-3.55 (m, 4H), 3.46 (bs, 2H), 2.42-2.36 (m, 4H); ESI MS m/z 534 [C₂₈H₂₈FN₅O₃S+H]⁺; HPLC (Method A) 93.6% (AUC), t_{R}=12.67 min.

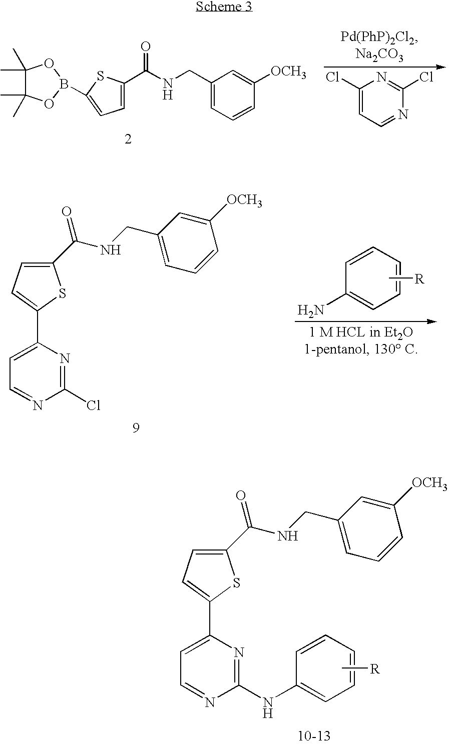

### Synthesis of 9

To a solution of 2 (0.37 g, 0.99 mmol) in DME (4 mL) and ethanol (2 mL) was added 2,4-dichloropyrimidine (0.49 g, 2.9 mmol), Pd(Ph₃P)₂Cl₂ (34 mg, 0.049 mmol) and 2 N Na₂CO₃ (0.75 mL). The resulting mixture was heated at 80° C. for 16 h. The reaction was cooled to rt and partitioned between H₂O (30 mL) and ethyl acetate (30 mL). The layers were separated and the organic layer was dried over Na₂SO₄, filtered and concentrated. The residue was purified by column chromatography (silica gel, 0-50% ethyl acetate/heptane) to obtain 9 (0.21 g, 58%) as a white solid: ESI MS m/z 360 [C₁₇H₁₄ClN₃O₂S+H]⁺.

### Synthesis of Example 10

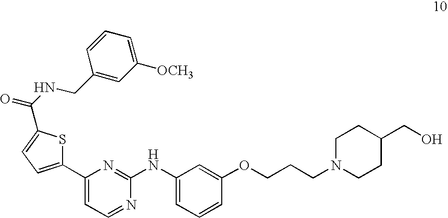

A solution of 1 N HCl in diethyl ether (0.7 mL, 0.67 mmol) was added dropwise to a solution of aniline (160 mg, 0.61 mmol) and 9 (240 mg, 0.67 mmol) in 2-propanol (5 mL). The reaction mixture was heated to reflux and stirred for 18 h. The reaction was cooled to room temperature, concentrated under reduced pressure, and the crude solid was diluted in methylene chloride (6 mL) and trifluoroacetic acid (1 mL). The solution was stirred for 15 min and neutralized carefully with satd. aq. NaHCO₃. The organic layer was separated, dried over Na₂SO₄ and concentrated under reduced pressure. The crude product was purified by chromatography (silica gel, 90:9:1 methylene chloride/methanol/concentrated ammonium hydroxide) to afford 10 (142 mg, 40%) as a yellow solid: mp 80-82° C.; ¹H NMR (500 MHz, DMSO-d₆) δ 9.69 (s, 1H), 9.18-9.16 (m, 1H), 8.55-8.54 (m, 1H), 8.00-7.99 (m, 1H), 7.87-7.86 (m, 1H), 7.63 (s, 1H), 7.39-7.38 (m, 1H), 7.31-7.29 (m, 1H), 7.27-7.24 (m, 1H), 7.17 (t, J=8.1 Hz, 1H), 6.91-6.89 (m, 2H), 6.84-6.82 (m, 1H), 6.54-6.52 (m, 1H), 4.46-4.44 (m, 2H), 4.37-4.35 (m, 1H), 4.05-4.03 (m, 2H), 3.74 (s, 3H), 3.21-3.19 (m, 2H), 2.86-2.84 (m, 2H), 2.42-2.39 (m, 2H), 1.92-1.86 (m, 2H), 1.83-1.79 (m, 2H), 1.59-1.57 (m, 2H), 1.30-1.28 (m, 1H), 1.10-1.05 (m, 2H); ESI MS m/z 588 [C₃₂H₃₇N₅O₄S+H]⁺; HPLC (Method A) 98.0% (AUC), t_{R}=11.75 min.

### Synthesis of Example 11

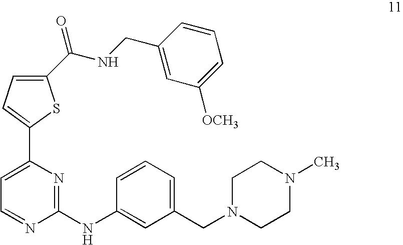

This compound was prepared by the same procedure described for 10. The crude product was purified by chromatography (silica gel, 0-20% methanol/methylene chloride w/1% conc. NH₄OH) to afford 11 (240 mg, 77%) as a yellow solid: ¹H NMR (500 MHz, DMSO-d₆) δ 9.67 (s, 1H), 9.18-9.16 (m, 1H), 8.53 (d, J=5.0 Hz, 1H), 7.99 (d, J=4.0 Hz, 1H), 7.87-7.85 (m, 2H), 7.67-7.66 (m, 1H), 7.35 (d, J=5.0 Hz, 1H), 7.31-7.29 (m, 2H), 6.93-6.83 (m, 4H), 4.45 (d, J=6.0 Hz, 2H), 3.74 (s, 3H), 3.45 (s, 2H), 2.50-2.25 (m, 8H), 2.16 (s, 3H); ESI MS m/z 529 [C₂₉H₃₂N₆O₂S+H]⁺; HPLC (Method A) 98.1% (AUC), t_{R}=10.58 min.

### Synthesis of Example 12

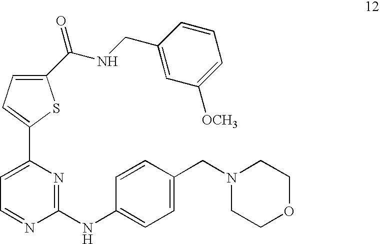

This compound was prepared by the same procedure described for 10. The crude product was purified by chromatography (silica gel, 0-20% methanol/methylene chloride w/1% conc. NH₄OH) to afford 12 (16 mg, 6%) as a yellow solid: ¹H NMR (500 MHz, CDCl₃) δ 8.43 (d, J=5.0 Hz, 1H), 7.65-7.63 (m, 2H), 7.53 (d, J=4.0 Hz, 1H), 7.34-7.25 (m, 3H), 7.19 (s, 1H), 7.04 (d, J=5.0 Hz, 1H), 6.96 (d, J=7.5 Hz, 1H), 6.95 (s, 1H), 6.87-6.85 (m, 1H), 6.33-6.30 (m, 1H), 4.64 (d, J=5.5 Hz, 2H), 3.81 (s, 3H), 3.77-3.60 (m, 4H), 3.58 (s, 2H), 2.52-2.45 (m, 4H); ESI MS m/z 516 [C₂₈H₂₉N₅O₃S+H]⁺.

### Synthesis of Example 13

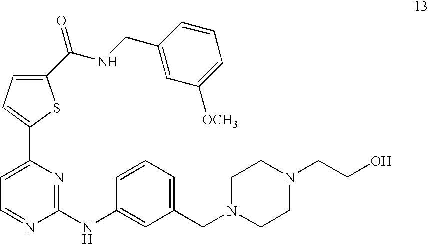

This compound was prepared by the same procedure described for 10. The crude product was purified by chromatography (silica gel, 0-20% methanol/methylene chloride w/1% conc. NH₄OH) to afford 13 (45 mg, 16%) as a yellow solid: ¹H NMR (500 MHz, DMSO-d₆) δ 9.69 (s, 1H), 9.17 (t, J=6.0 Hz, 1H), 8.54 (d, J=5.0 Hz, 1H), 8.00 (d, J=4.0 Hz, 1H), 7.87-7.85 (m, 2H), 7.68-7.67 (m, 1H), 7.36 (d, J=5.0 Hz, 1H), 7.31-7.23 (m, 2H), 6.91-6.82 (m, 4H), 4.45 (d, J=6.0 Hz, 2H), 4.33 (bs, 1H), 3.74 (s, 3H), 3.49-3.44 (m, 4H), 2.52-2.32 (m, 10H); ESI MS m/z 559 [C₃₀H₃₄N₆O₃S+H]⁺; HPLC (Method A) 97.7% (AUC), t_{R}=10.25 min.

### Biological Data

### Aurora A Scintillation Proximity Assay (SPA) Compound Screening Assay AESOP Protocol #AP2106v1:

Full length Aurora A kinase (2-403) was expressed in baculovirus/Sf9 system containing a N-terminal his tag and purified to >70% purity by affinity chromatography.

The assay was run in Nunc (#264724) 384-well white plates containing test compounds which were dissolved and serially diluted in 100% DMSO, 0.1 ul of test compound was added to assay plates prior to addition of the assay components. For use in the enzyme assay 10 ul of a 2× (10 nM) enzyme stock solution was added to 10 ul of a 2× reaction mixture solution containing 50 mM HEPES, pH 7.5, 4 uM ATP, 12 mM MgCl2, 2 uM biotin-Ahx-RARRRLSFFFFAKKK-OH, 10 mM DTT, 0.15 mg/ml BSA, 0.01% Tween-20 and 0.05 uCi gamma-33P-ATP/assay. After the addition of the enzyme and reaction mixture the assay plates were incubated for ˜75 minutes followed by the addition 50 ul of SPA bead solution (containing 0.06 mg of SPA beads, 50 mM EDTA in PBS), the plates were sealed and allowed to settle overnight and read in a Packard Topcount microtiter plate reader.

For dose response curves, data were normalized and expressed as % inhibition using the formula 100*(1−(U−C2)/(C1−C2)) where U is the unknown value, C1 is the average of the high signal (0% inhibition) and C2 is the average of the low signal (100% inhibition) control wells. Curve fitting was performed with the following equation: y=A+((B−A)/(1+(10̂x/10̂C)̂D)), where A is the minimum response, B is the maximum response, C is the log 10×C50, and D is the slope. The results for each compound were recorded as plC50 values (−C in the above equation).

### Aurora B Scintillation Proximity Assay (SPA) Compound Screening Assay: AESOP Protocol #AP2800v1:

Full length Aurora B kinase (2-403) used for the assay was obtained from the University of Dundee Collaboration, clone DU1773. The protein is expressed in baculovirus/Sf9 system containing a N-terminal his tag. 50 nM okadaic acid was included during the last hour of expression prior to purification to increase its catalytic activity. The enzyme was purified to >60% by affinity chromatography and is preactivated in the presence of the protein INCENP, ATP and MgCl2.

The assay was run in Nunc (#264724) 384-well white plates containing test compounds which were dissolved and serially diluted in 100% DMSO, 0.1 ul of test compound was added to assay plates prior to addition of the assay components. For use in the enzyme assay 10 ul of a 2× (10 nM) enzyme stock solution wass added to 10 ul of a 2× reaction mixture solution containing 50 mM HEPES, pH 7.5, 4 uM ATP, 12 mM MgCl2, 6 mM MnCl2, 2 uM biotin-Ahx-RARRRLSFFFFAKKK-OH, 10 mM DTT, 0.15 mg/ml BSA, 0.01% Tween-20 and 0.05 uCi gamma-33P-ATP/assay. After the addition of the enzyme and reaction mixture the assay plates were incubated for ˜120 minutes followed by the addition 50 ul of SPA bead solution (containing 0.06 mg of SPA beads, 50 mM EDTA in PBS), the plates were sealed and allowed to settle overnight and read in a Packard Topcount microtiter plate reader.

For dose response curves, data were normalized and expressed as % inhibition using the formula 100*(1−(U−C2)/(C1−C2)) where U is the unknown value, C1 is the average of the high signal (0% inhibition) and C2 is the average of the low signal (100% inhibition) control wells. Curve fitting was performed with the following equation: y=A+((B−A)/(1+(10̂x/10̂C)̂D)), where A is the minimum response, B is the maximum response, C is the log 10×C50, and D is the slope. The results for each compound were recorded as pIC50 values (−C in the above equation).

All exemplified Examples 1—were run with the recited (or similar) Aurora kinase assays and showed inhibitory activity versus Aurora with a pIC₅₀ of 5.0 or greater.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims.

What is claimed is:

1. A compound of Formula (I): 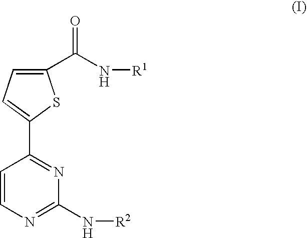
wherein
R¹ represents: 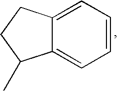
C₁₋₃ alkylene)ₘ⁻—C₄₋₇cycloalkyl, where m is 0 or 1 and the cycloalkyl group is optionally substituted by C₁₋ hydroxyalkyl, a 5 membered heteroaryl group, optionally substituted by one or more C₁₋₃alkyl, —C₁₋₃alkyleneCN,
—C₁₋₃ alkylenepyrdinyl, —C₁₋₃alkyeneindolyl, —(C₁₋₃alkylene)ₙphenyl, where n is 0 or 1, the phenyl group is optionally fused to a 5 or 6 membered heterocyclic group or is substituted by one or more substituents independently selected from —C₁₋₆hydroxyalkyl, —C₁₋₆alkyl, —C₁₋₆ haloalkyl, —C₁₋₆alkoxy, —C₁₋₆haloalkoxy, -halogen, —OH, —COOH, —COOC₁₋₃alkyl, —NHCOC₁₋₃ alkyl, —NHSO₂C₁₋₃alkyl, —CONR^{a}R^{b}, —NR^{c}R^{d}, —SO₂NR^{e}R^{f}, —(CH₂)₄OH, —C₁₋₆alkyleneNR⁶R⁷, wherein the alkylene group is optionally substituted by phenyl;
R^{a}, R^{b}, R^{c}, R^{d}, R^{e} and R^{f} are each independently selected from H or —C₁₋₃alkyl;
R⁶ and R⁷ are independently HC₁₋₃ alkyl or R⁵ and R⁶ together with the nitrogen to which they are joined form a 6 membered heterocyclic ring, optionally containing a further heteroatom selected from O or N and optionally substituted by C₁₋₃alkyl;
R² is 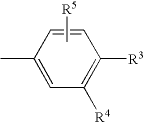
wherein
R³ and R⁴ together form a group selected from: 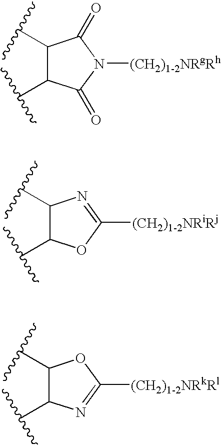
wherein R^{g}R^{h}, Rⁱ, R^{j}, R^{k} and R^{l} are independently H or —C₁₋₃akyl;
or one of R³ and R⁴ is H, CH₃ or halogen and the other is a substituent selected from —OH, -phenyl (substituted by —C₁₋₃alkyleneNR^{m}Rⁿ), halogen or a group R⁸R⁹;
R^{m}Rⁿ are independently H or —C₁₋₃alkyl;
R⁸ is a bond (i.e. is absent), —O—, —CO—, —COO—, —C₁₋₃alkyleneNHCO—, —NHCO—, —SO₂—, —CONHC₁₋₃alkylene, —NHCOC₁₋₃alkylene-, —OC₁₋₃alkylene-, or —C₁₋₃alkylene-;
R⁹ is 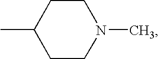
-pyridinyl, —C₁₋₆alkyl, —C₁₋₆haloalkyl or —NR¹⁰R¹¹;
R¹⁰ and R¹¹, are independently H, —C₁₋₃alkyl, —(CH₂)₁₋₃NR^{o}R^{p}, or R¹⁰ and R¹¹, together with the N to which they are joined form a 5 or 6 membered heterocyclic or heteroaryl ring, each of which heterocyclic or heteroaryl ring optionally contains further heteroatoms independently selected from O or N and optionally substituted by C₁₋₃ alkyl, ═O, OH, C₁₋₃ hydroxyalkyl, —SO₂C₁₋₃alkyl;
R^{o}R^{p} are independently H or C₁₋₃alkyl;
R⁵ is H or methyl;
or a salt or solvate thereof.

2. A compound according to claim 1 wherein R¹ is —(CH₂)₀₋₁ cyclohexyl wherein the cyclohexyl is substituted by —CH₂OH), or —(CH₂)₀₋₃ phenyl wherein the phenyl group is optionally mono or disubstituted by substituents independently selected from —C₁₋₃alkoxy, —C₁₋₃ haloalkoxy, —OH, —F, —Cl, —C₁₋₃ hydroxyalkyl, —N(CH₃)₂, —NHCOCH₃, —NHSO₂CH₃, —COOCH₃, —COOH, —CONH₂, —CONH CH₃), —CH(CH₃)phenyl, —C₁₋₆alkyleneN(CH₃)₂, —CH₂indolyl, —(CH₂)₄OH, —CH₂CN, C₀₋₃alkylenepyridyl,

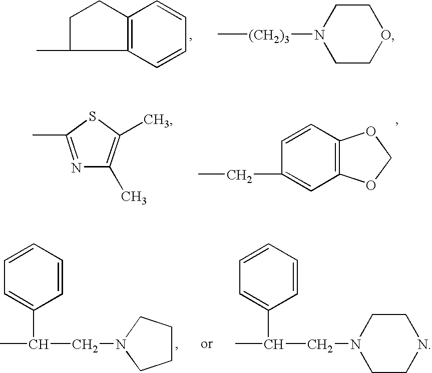

3. A compound according to claim 1, wherein R¹ is —C₁₋₃alkylene phenyl, wherein the phenyl is optionally substituted by one or more substituents independently selected from —C₁₋₃alkoxy, —C₁₋₃haloalkoxy, —OH, —F, —Cl, —C₁₋₃ hydroxyalkyl, —N(CH₃)₂, —NHCOCH₃, —NHSO₂CH₃, —COOCH₃, —COOH, —CONH₂, —CONH CH₃).

4. A compound according to any of claim 1, wherein R¹ —CH₂phenyl, wherein the phenyl is optionally mono substituted by —OMe.

5. A compound according to claim 1, wherein R² is 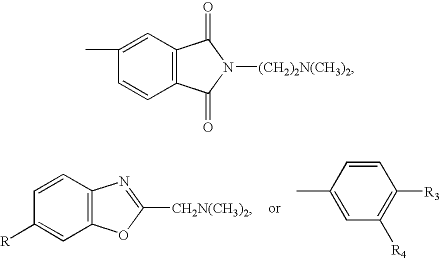
wherein one of R³ and R⁴ is H and the other is selected from —F, —Cl, —OH, —phenylCH₂N(CH₃)₂, —R⁸R⁹, wherein R⁸ and R⁹ are as defined above.

6. A compound according to claim 1, wherein R⁸ is a bond (ie is absent), —O—, NHCO(CH₂)₂, —OCH₃—, —CO—, NHCOCH₂—, CH₂—, OCH₂CH₂—, —CONHCH₂CH₂—CONHCH₂, —CON(CH₃)—, —SO₂—, or —COO—.

7. A compound according to claim 1, wherein R⁸ is —O—, —C₁₋₃ alkylene-, or —OC₁₋₃ alkylene-.

8. A compound according to claim 1, wherein R9 is —CH₃, —N(CH₃)₂, Cl, F, OH,

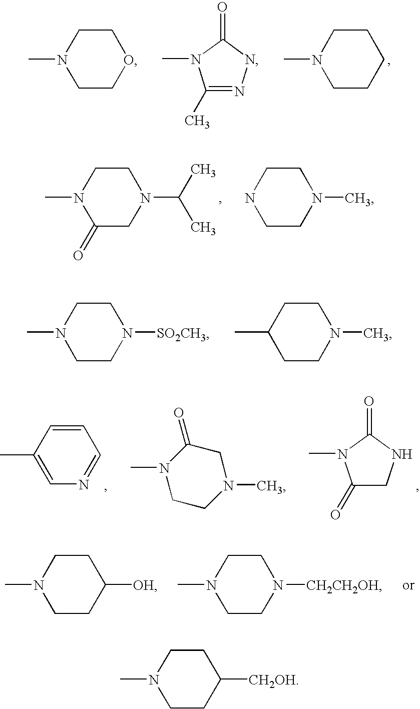

9. A compound according to claim 8, wherein R⁹ is

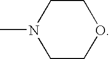

10. A compound according to claim 1, wherein R⁸R⁹ is —OCH₃

11. A compound of Formula 1 or a salt or solvate thereof selected from the group consisting of 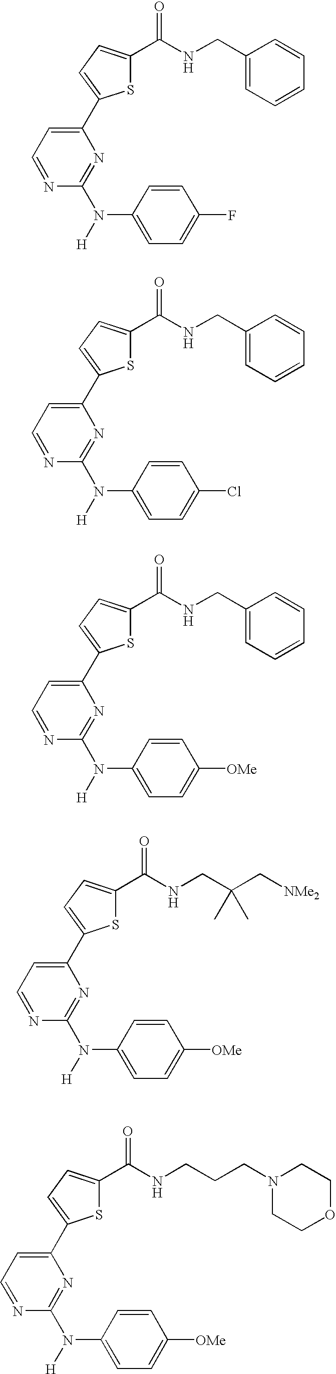 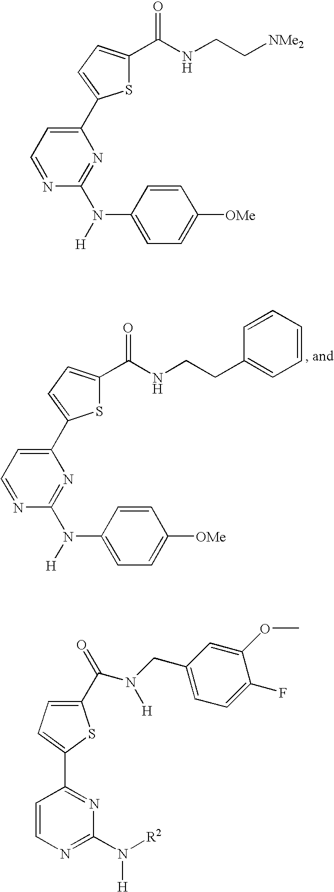
or a compound of Formula 1 or a salt or solvate thereof wherein
R²NH₂ is 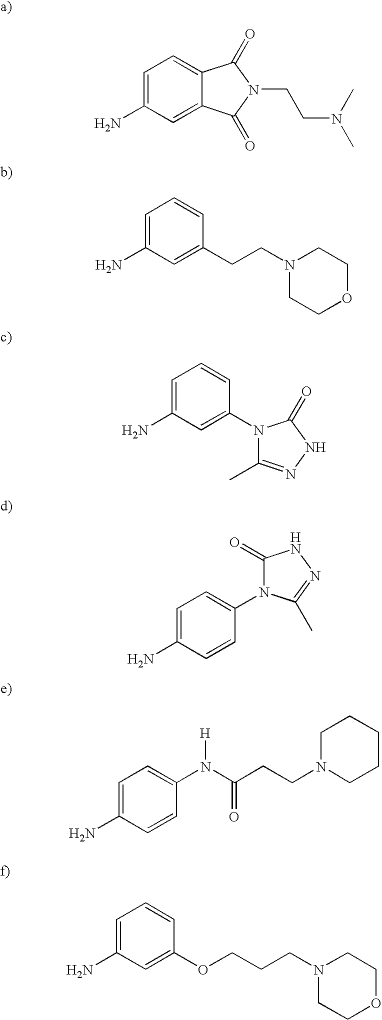 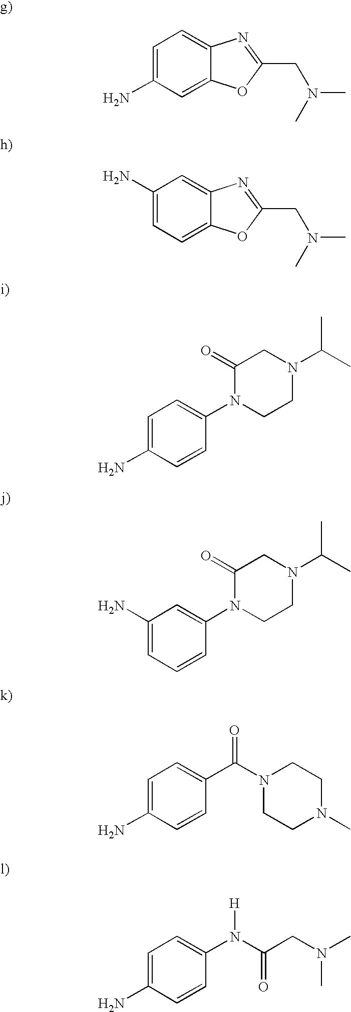 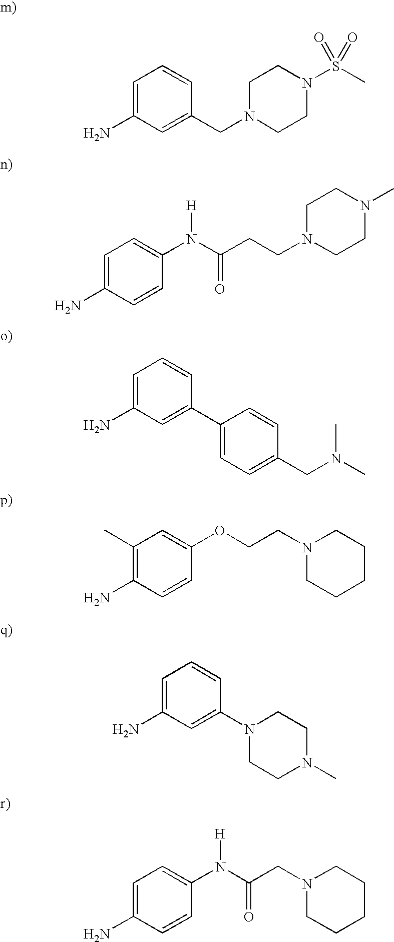 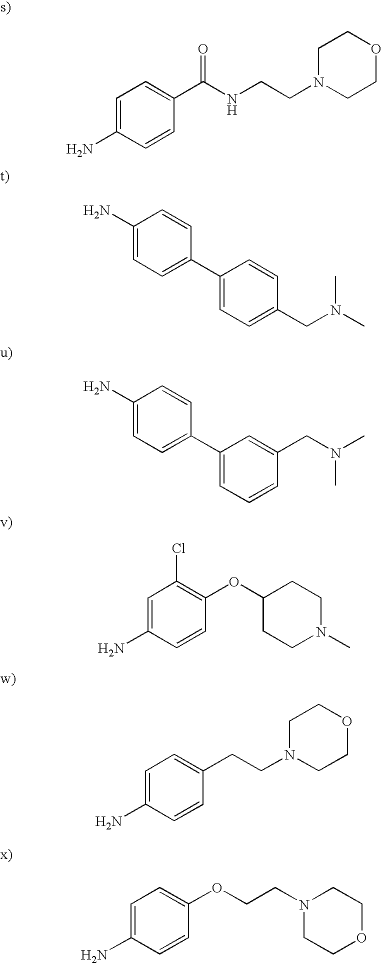 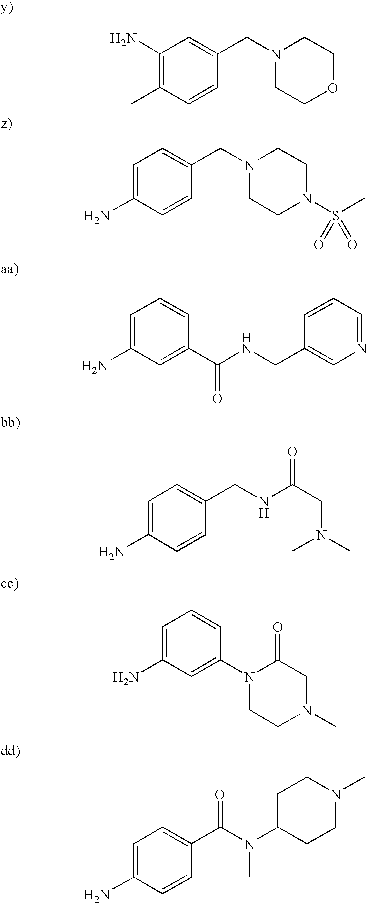 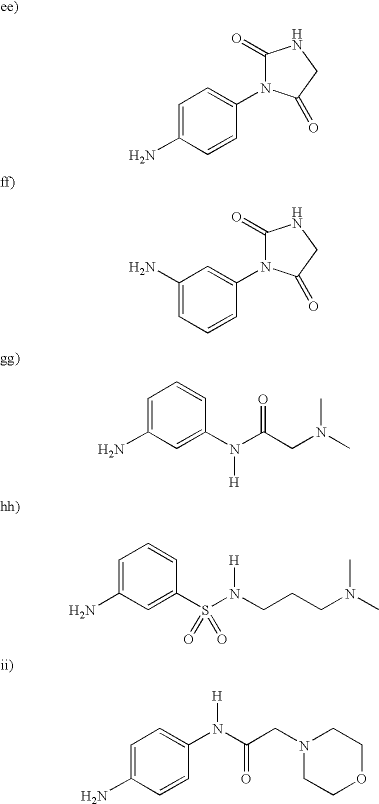
or a compound of Formula 1 or a salt or solvate thereof selected from:

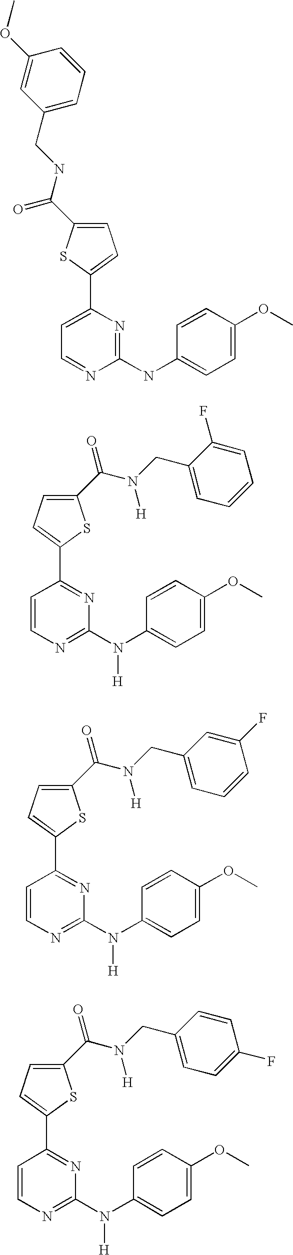

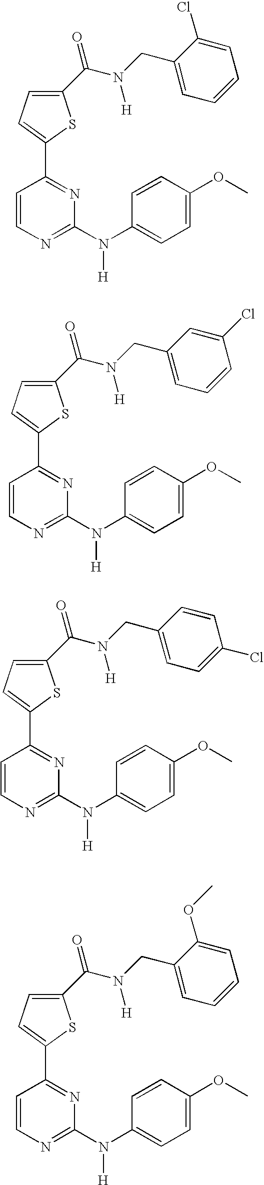

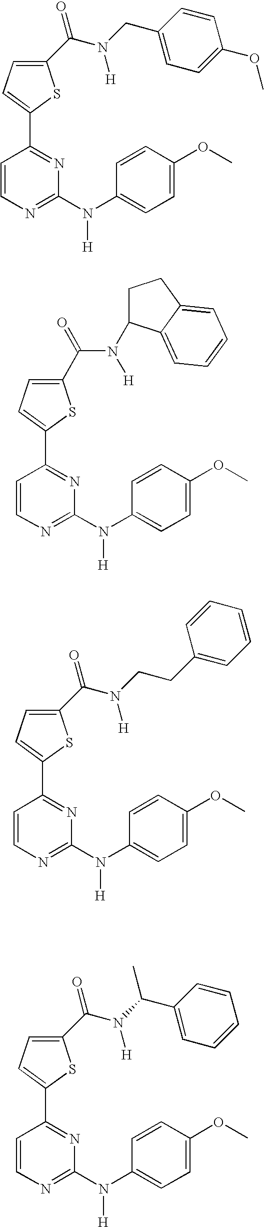

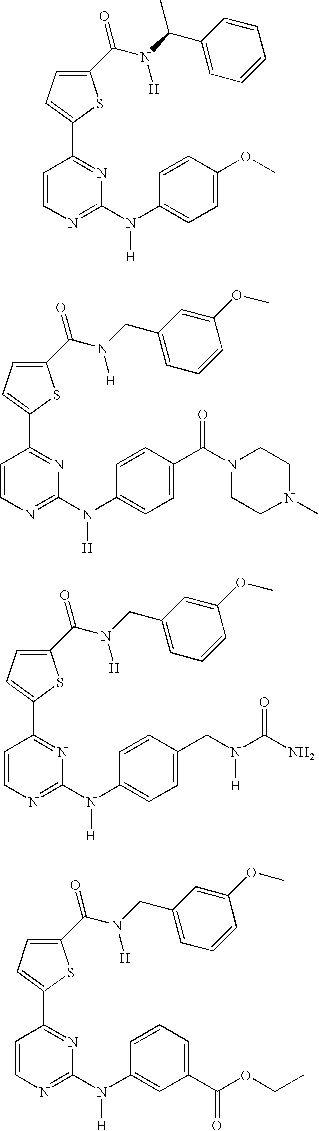

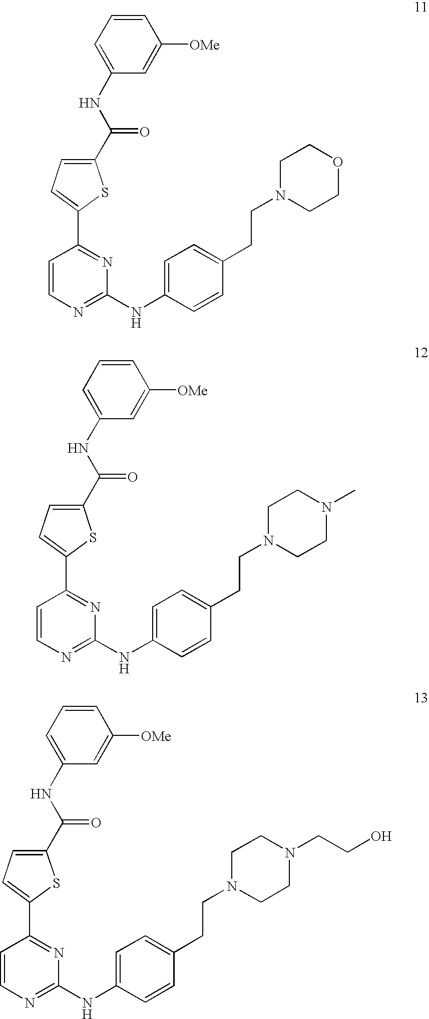

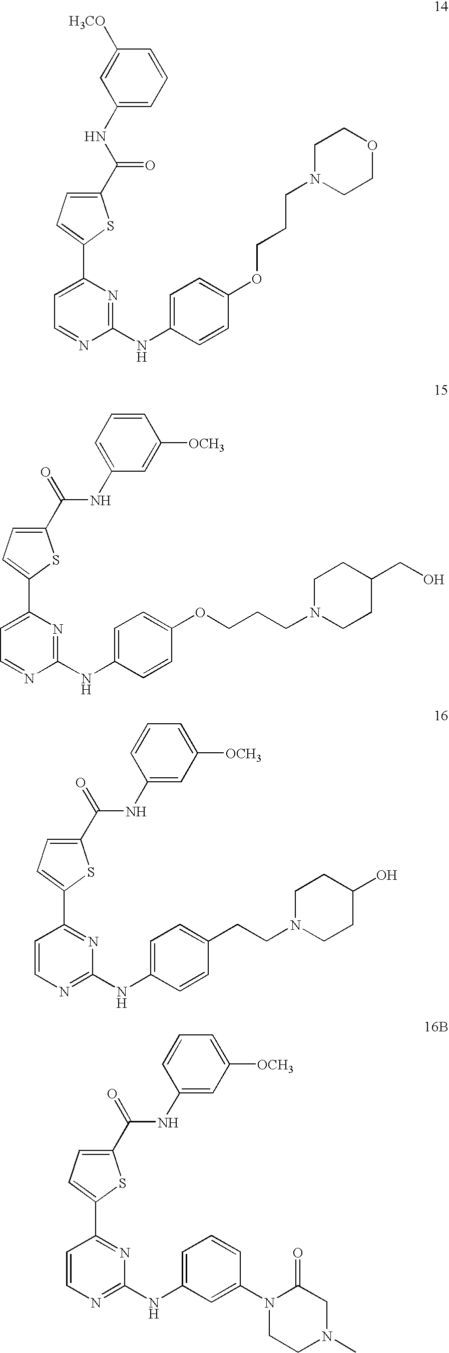

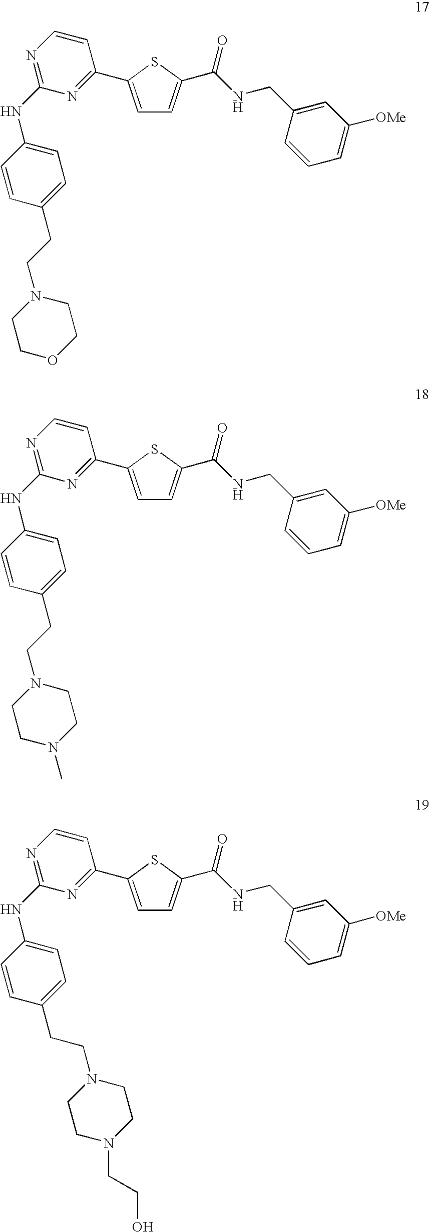

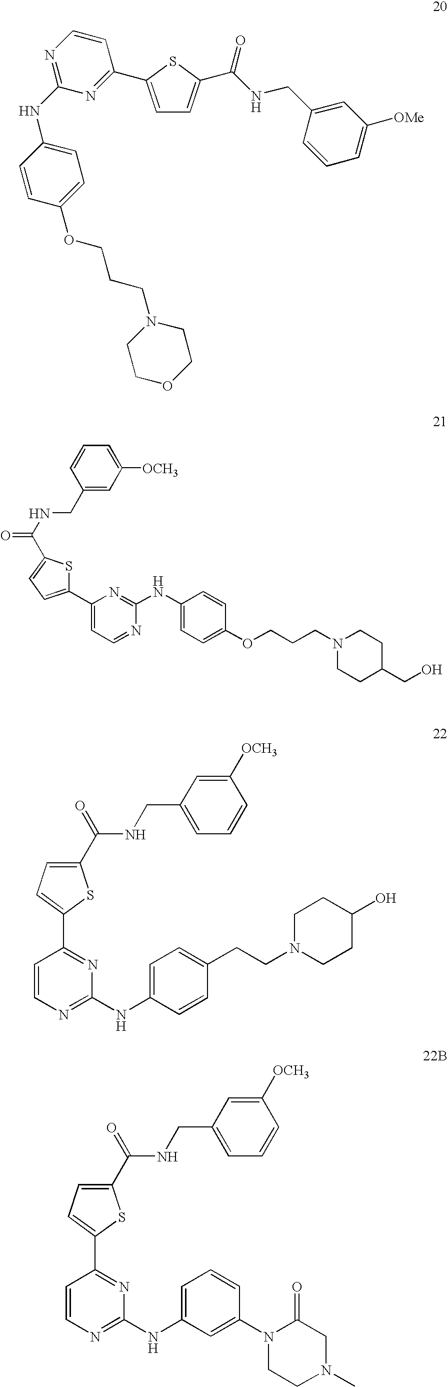

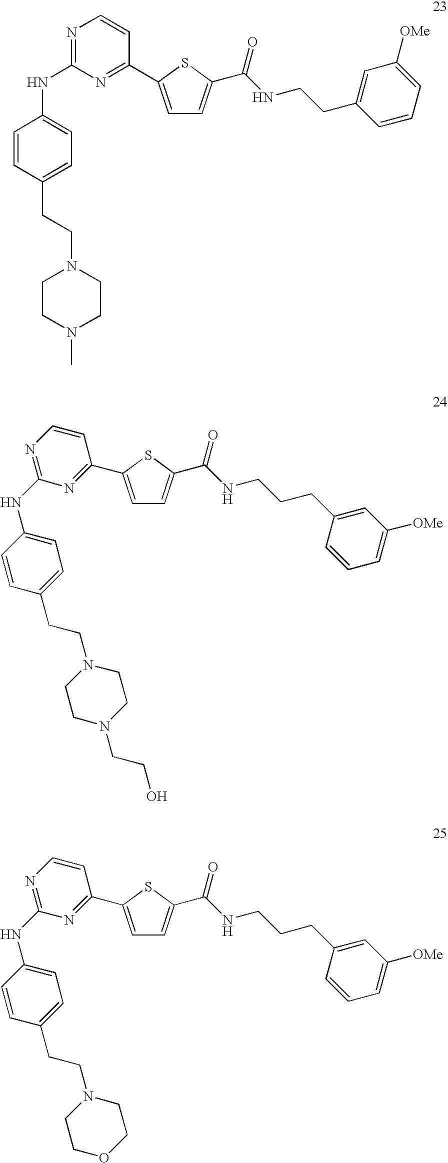

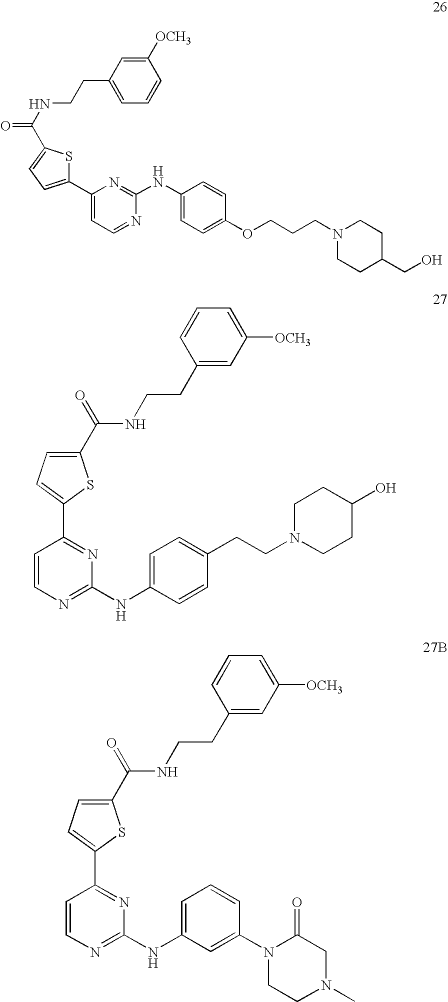

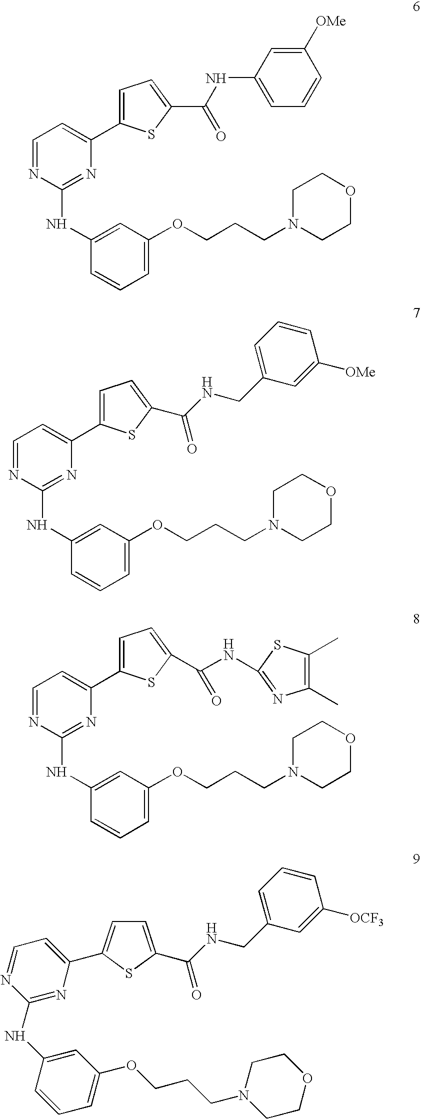

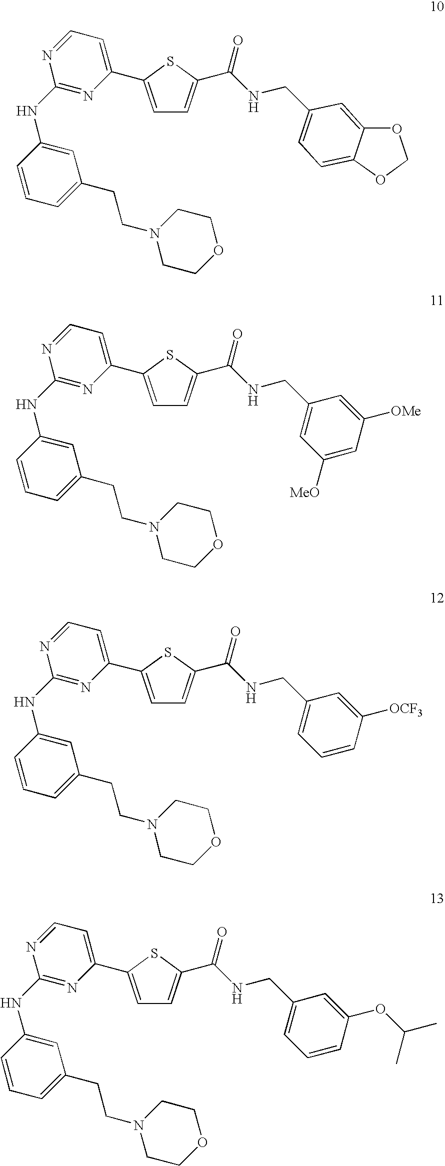

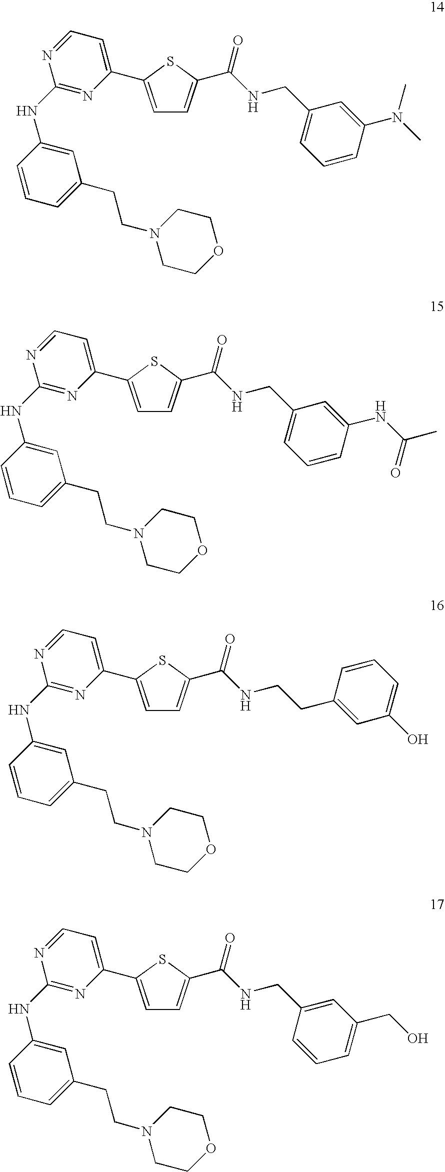

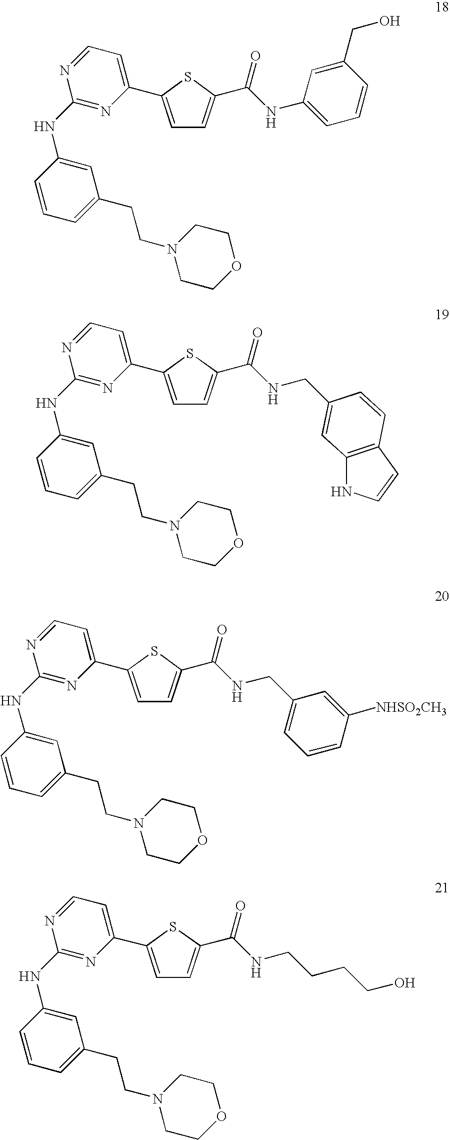

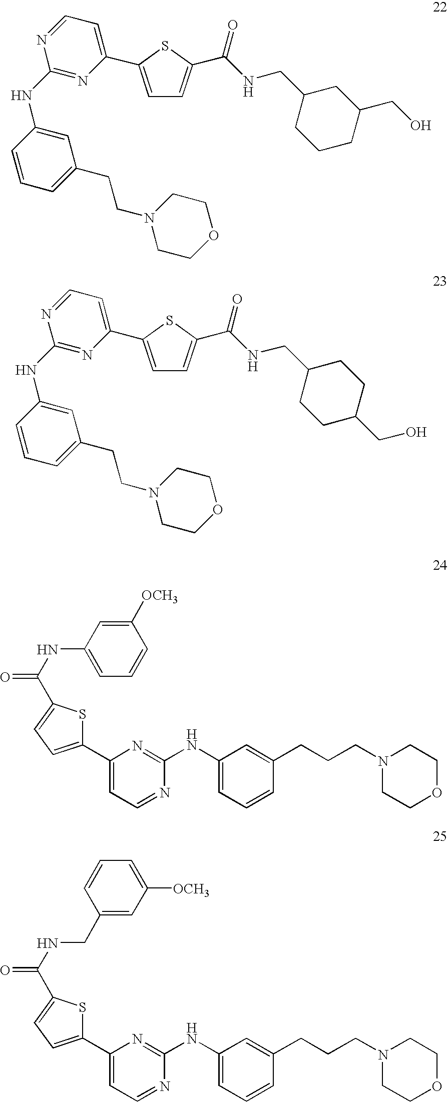

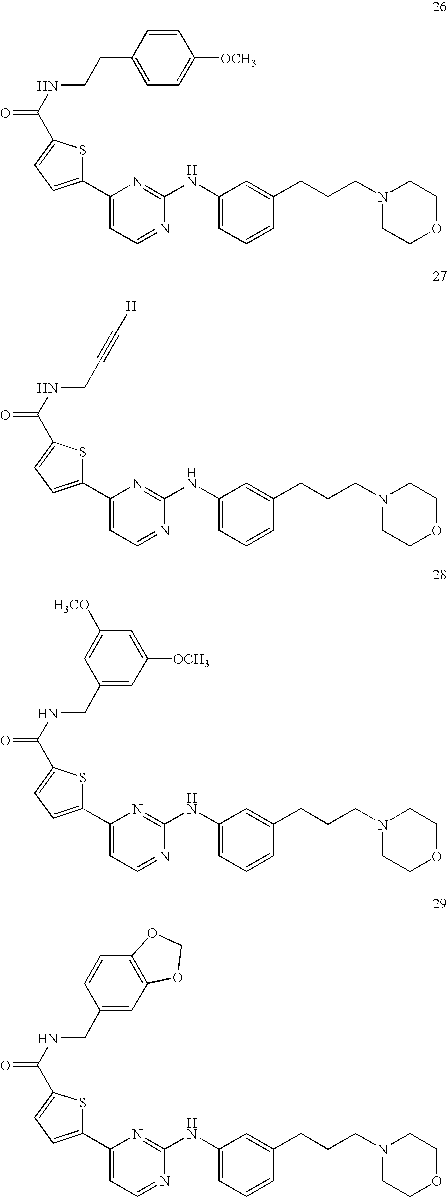

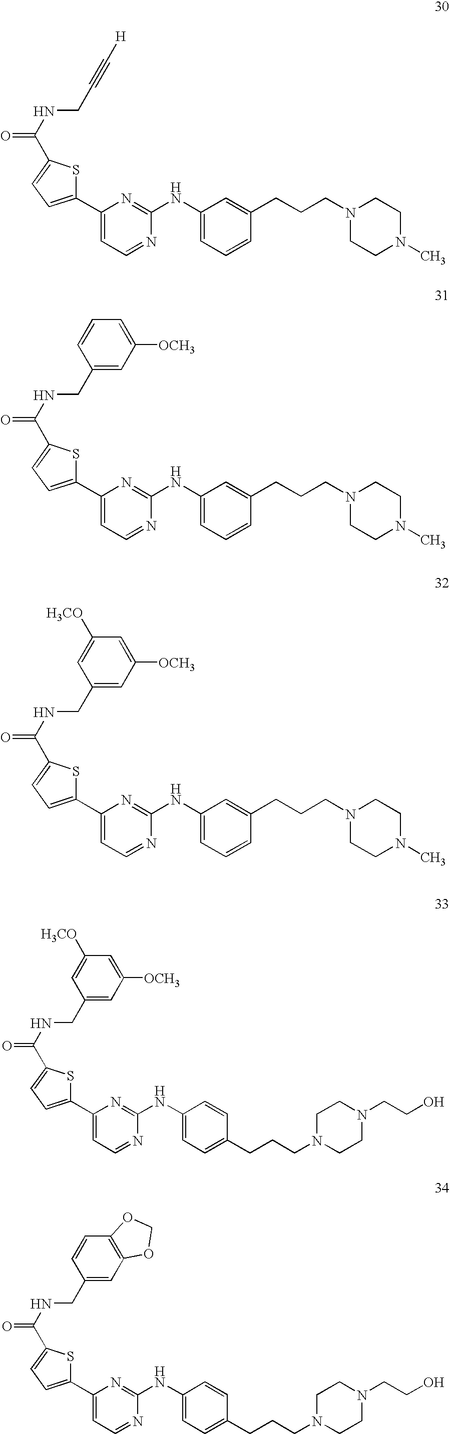

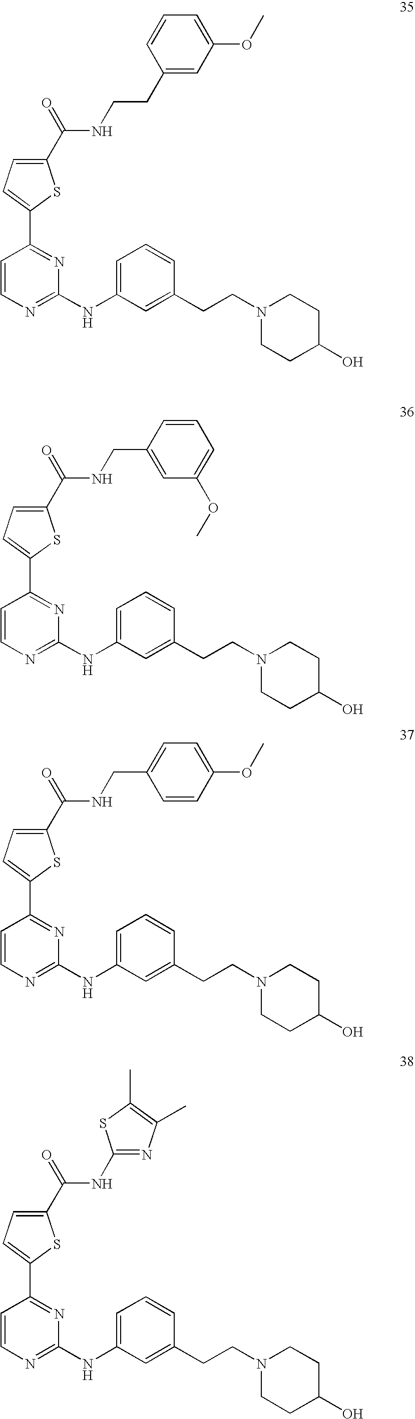

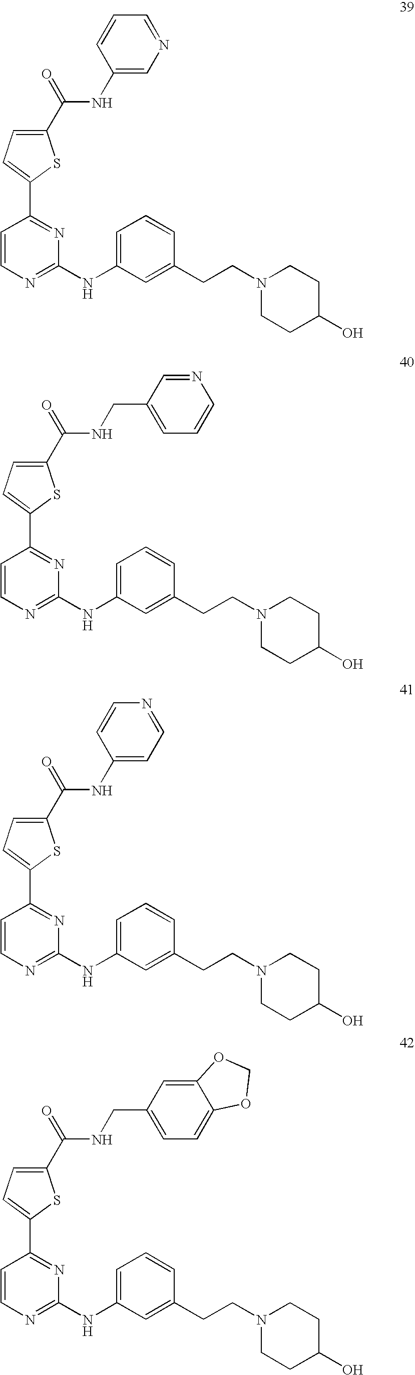

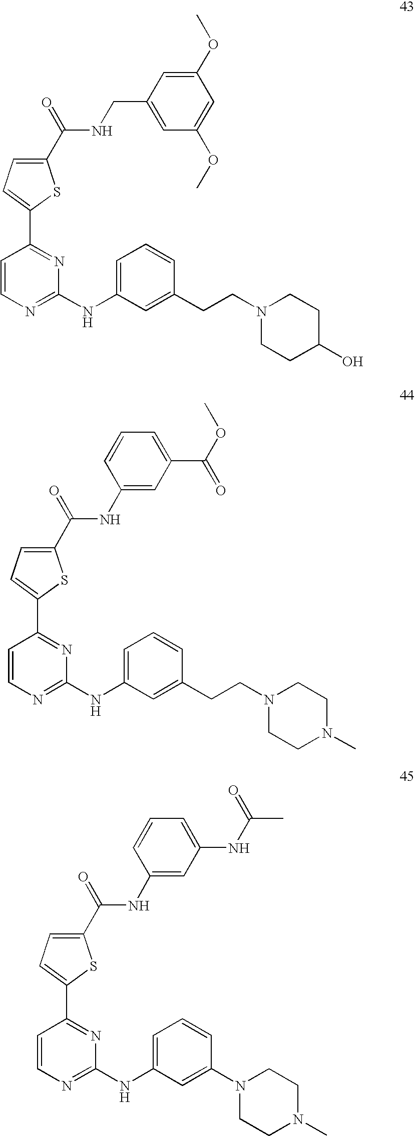

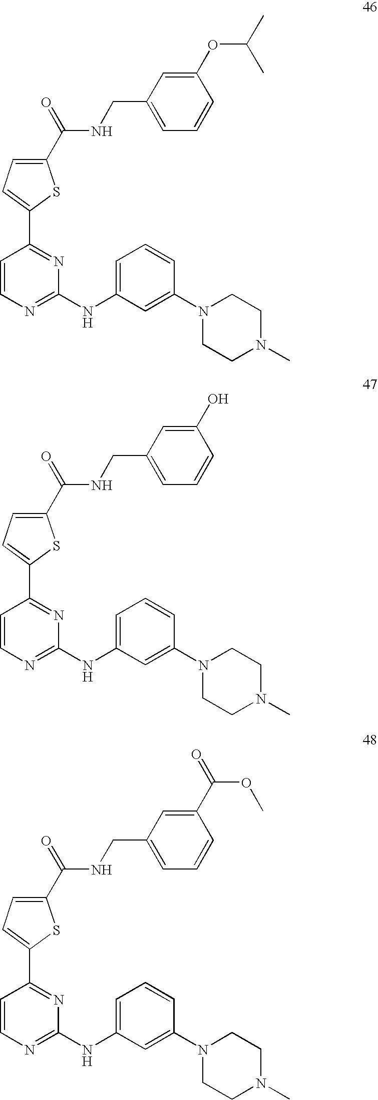

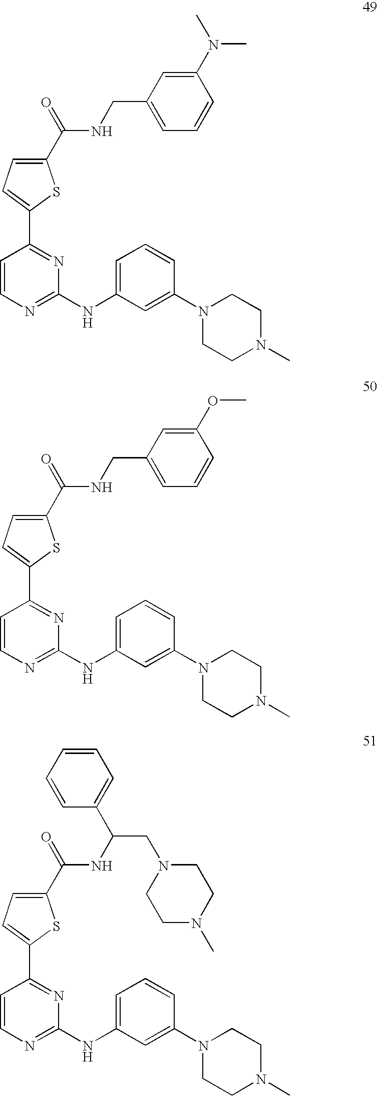

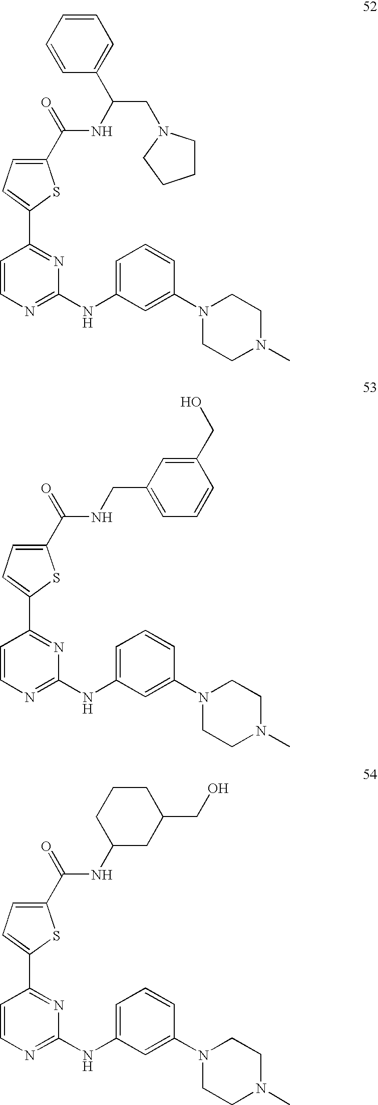

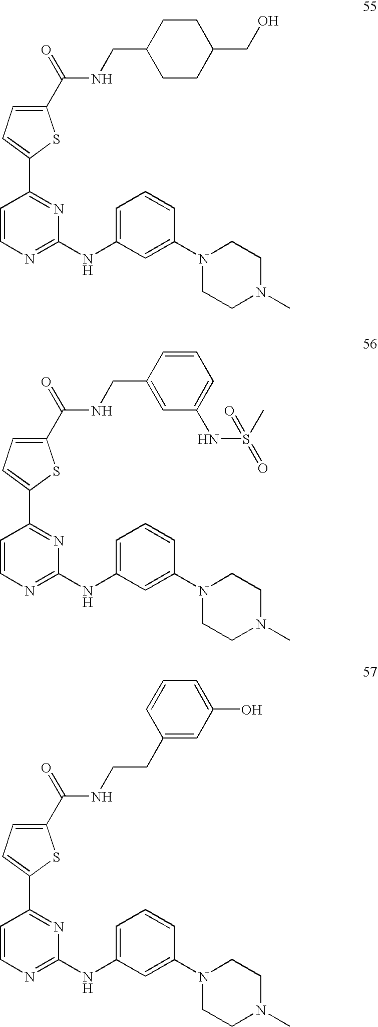

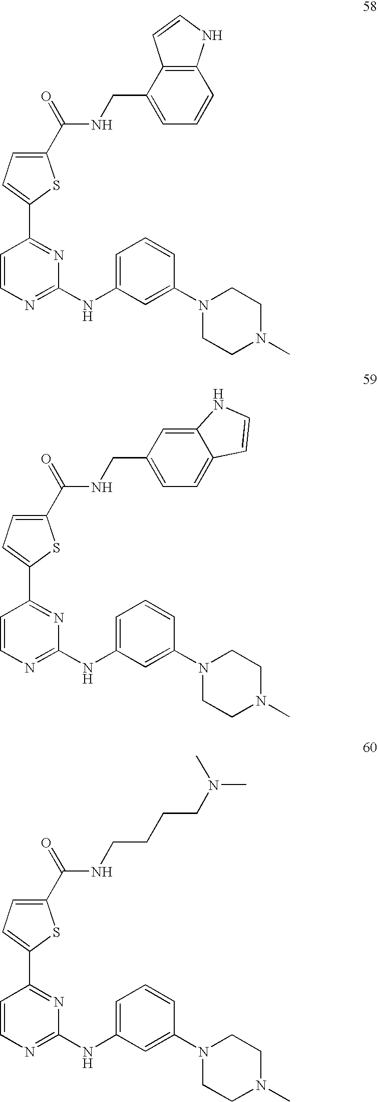

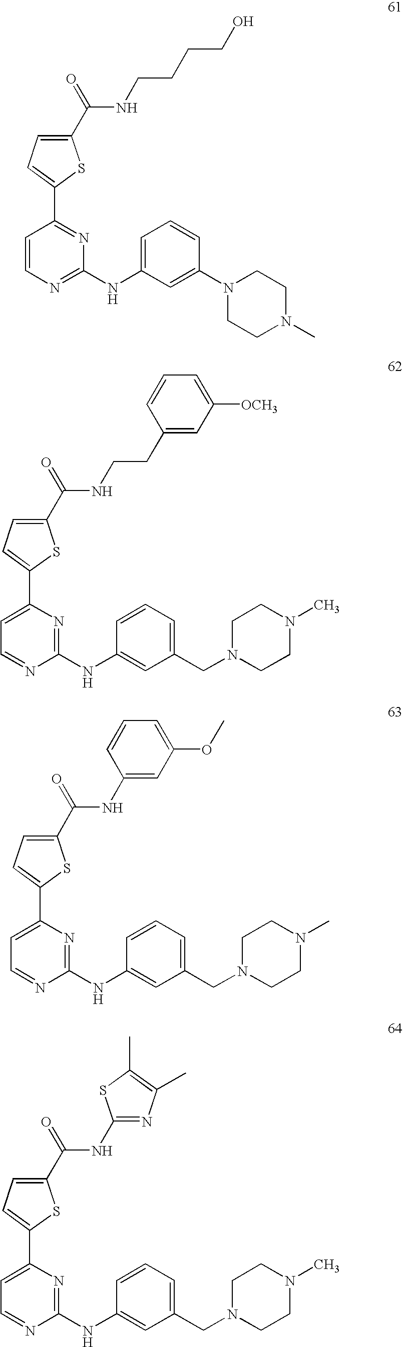

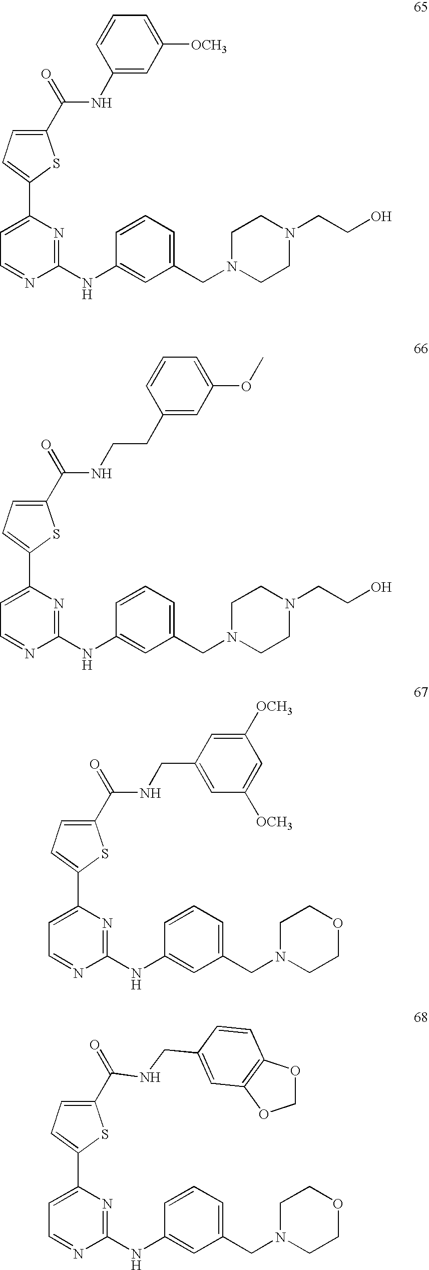

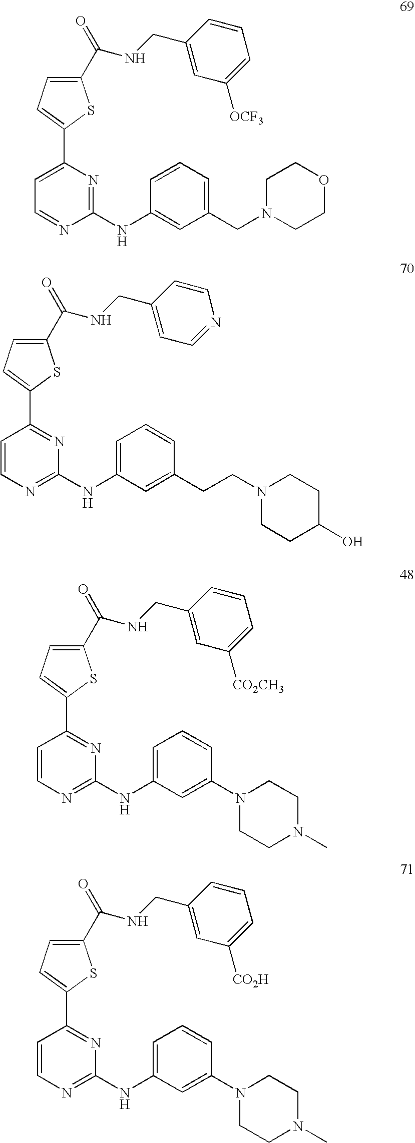

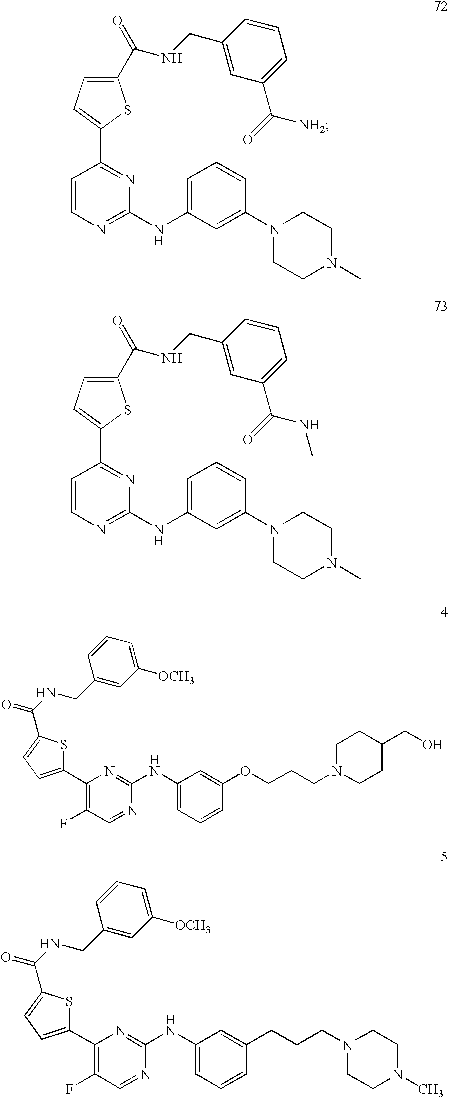

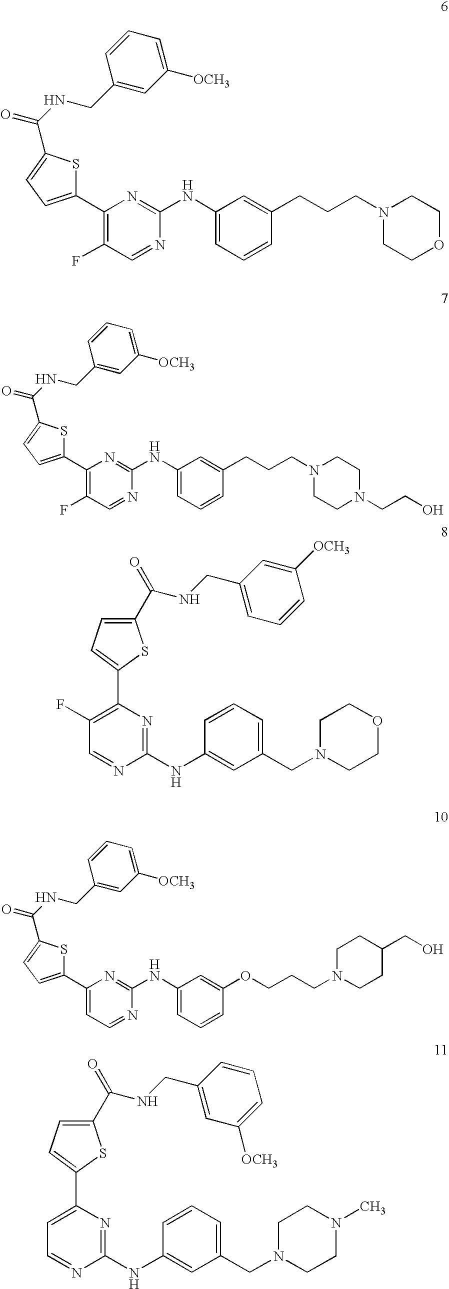

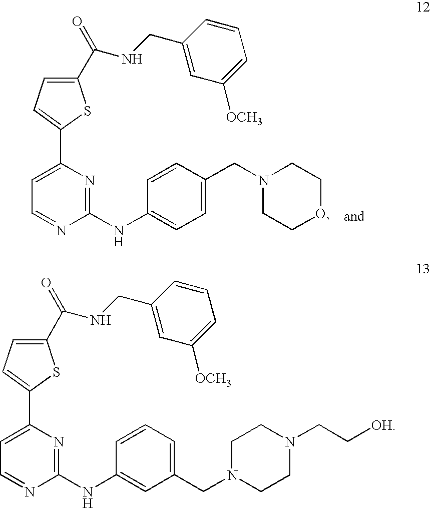

12. A pharmaceutical composition, comprising: a therapeutically effective amount of a compound as claimed in claim 1, and one or more of pharmaceutically acceptable carriers, diluents and excipients.

13-19. (canceled)

20. A pharmaceutical composition, comprising: a therapeutically effective amount of a compound as claimed in claim 11, and one or more of pharmaceutically acceptable carriers, diluents and excipients.

21. A method of treating a disorder in a mammal, said disorder being mediated by inappropriate Aurora kinase activity, comprising administering to said mammal a compound as claimed in claim 1.

22. A method of treating a disorder in a mammal, said disorder being mediated by inappropriate Aurora kinase activity, comprising administering to said mammal a compound as claimed in claim 11.

23. A method of treating cancer in a mammal, comprising administering to said mammal a compound as claimed in claim 1.

24. A method of treating cancer in a mammal, comprising administering to said mammal a compound as claimed in claim 1.
